SEQUENCE LISTING (4 sequences)

SEQ ID NO: 1 - DNA, 19 bp, Artificial Sequence
  source: /note=“Description of Artificial Sequence Synthetic primer”
ctgtgccttgggtggcttt

SEQ ID NO: 2 - DNA, 24 bp, Artificial Sequence
  source: /note=“Description of Artificial Sequence Synthetic primer”
aaggaaagaagtcagaaggcaaaa

SEQ ID NO: 3 - DNA, 25 bp, Artificial Sequence
  source: /note=“Description of Artificial Sequence Synthetic probe”
tcctttataagggtcgatgtccatg

SEQ ID NO: 4 - protein, 36 aa, Artificial Sequence
  source: /note=“Description of Artificial Sequence Synthetic polypeptide”
YTSLIHSLIEESQNQQEKNEQELLELDKWASLWNWF

(19) United States
(12) Patent Application Publication
(10) Pub. No.: US 2022/0033406 A1
(43) Pub. Date: Feb. 3, 2022

(54) CYCLOPENTYL NUCLEOSIDE ANALOGS AS ANTI-VIRALS

(71) Applicant: Janssen BioPharma, Inc., South San Francisco, CA (US)

(72) Inventors: Minghong ZHONG, San Bruno, CA (US); Guangyi WANG, Irvine, CA (US)

(21) Appl. No.: 17/413,327

(22) PCT Filed: Dec. 4, 2019
(86) PCT No.: PCT/IB2019/060431

§ 371 Date: Jun. 11, 2021

Related U.S. Application Data
(60) Provisional application No. 62/778,818, filed on Dec. 12, 2018.

(51) Int. Cl. C07D 487/04; C07D 239/10; C07F 9/6561; C07F 9/6512; A61K 45/06; A61K 31/685; A61K 31/513; A61K 31/519; A61K 31/522; C07D 239/22; A61P 31/20

(52) U.S. Cl. CPC C07D 487/04; C07D 239/10; C07F 9/65616; C07F 9/6512; C07F 9/6561; A61P 31/20; A61K 31/685; A61K 31/513; A61K 31/519; A61K 31/522; C07D 239/22; A61K 45/06

(57) ABSTRACT

Described herein are cyclopentyl nucleoside analogs of Formula (I), pharmaceutical compositions that include one or more cyclopentyl nucleoside analogs and methods of using the same to treat HBV, HDV and/or HIV. (I)

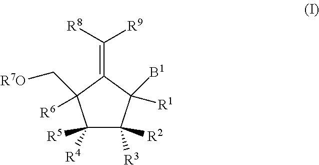

### FIELD

The present application relates to the fields of chemistry, biochemistry and medicine. More particularly, disclosed herein are cyclopentyl nucleoside analogs, pharmaceutical compositions that include one or more cyclopentyl nucleoside analogs and methods of synthesizing the same. Also disclosed herein are methods of treating viral diseases and/or conditions with a cyclopentyl nucleoside analog, alone or in combination therapy with one or more other agents.

### DESCRIPTION

Nucleoside analogs are a class of compounds that have been shown to exert antiviral activity both in vitro and in vivo, and thus, have been the subject of widespread research for the treatment of viral infections. Nucleoside analogs can be converted by host or viral enzymes to their respective active moieties, which, in turn, may inhibit polymerases involved in viral or cell proliferation. The activation occurs by a variety of mechanisms, such as the addition of one or more phosphate groups and, or in combination with, other metabolic processes.

### SUMMARY

Some embodiments described herein relate to a compound of Formula (I), or a pharmaceutically acceptable salt thereof. Other embodiments described herein related to a pharmaceutical composition that includes a compound of Formula (I), or a pharmaceutically acceptable salt thereof.

Some embodiments described herein relate to a method of treating a HBV and/or HDV infection that can include administering to a subject identified as suffering from the HBV and/or HDV infection an effective amount of a compound described herein (for example, a compound of Formula (I), or a pharmaceutically acceptable salt thereof), or a pharmaceutical composition that includes an effective amount of a compound described herein (such as, a compound of Formula (I), or a pharmaceutically acceptable salt thereof). Other embodiments described herein relate to using a compound described herein (for example, a compound of Formula (I), or a pharmaceutically acceptable salt thereof) in the manufacture of a medicament for treating a HBV and/or HDV infection. Still other embodiments described herein relate to the use of a compound described herein (for example, a compound of Formula (I), or a pharmaceutically acceptable salt thereof), or a pharmaceutical composition that includes a compound described herein (such as, a compound of Formula (I), or a pharmaceutically acceptable salt thereof) for treating a HBV and/or HDV infection.

Some embodiments disclosed herein relate to a method of treating a HBV and/or HDV infection that can include contacting a cell infected with the HBV and/or HDV with an effective amount of a compound described herein (for example, a compound of Formula (I), or a pharmaceutically acceptable salt thereof), or a pharmaceutical composition that includes an effective amount of a compound described herein. Other embodiments described herein relate to using a compound described herein (for example, a compound of Formula (I), or a pharmaceutically acceptable salt thereof) in the manufacture of a medicament for treating a HBV and/or HDV infection that can include contacting a cell infected with the HBV and/or HDV with an effective amount of said compound(s) and/or pharmaceutical composition. Still other embodiments described herein relate to the use of a compound described herein (for example, a compound of Formula (I), or a pharmaceutically acceptable salt thereof), or a pharmaceutical composition that includes a compound described herein for treating a HBV and/or HDV infection, wherein the use includes contacting a cell infected with the HBV and/or HDV with an effective amount of said compound(s) and/or pharmaceutical composition.

Some embodiments disclosed herein relate to a method of inhibiting replication of HBV and/or HDV that can include contacting a cell infected with the HBV and/or HDV with an effective amount of a compound described herein (for example, a compound of Formula (I), or a pharmaceutically acceptable salt thereof), or a pharmaceutical composition that includes a compound described herein (for example, a compound of Formula (I), or a pharmaceutically acceptable salt thereof). Other embodiments described herein relate to using a compound described herein (such as, a compound of Formula (I), or a pharmaceutically acceptable salt thereof) in the manufacture of a medicament for inhibiting replication of HBV and/or HDV that can include contacting a cell infected with HBV and/or HDV with an effective amount of said compound(s) and/or pharmaceutical composition. Still other embodiments described herein relate to the use of a compound described herein (for example, a compound of Formula (I), or a pharmaceutically acceptable salt thereof), or a pharmaceutical composition that includes an effective amount of a compound described herein (such as, a compound of Formula (I), or a pharmaceutically acceptable salt thereof), for inhibiting replication of HBV and/or HDV, wherein the use includes contacting a cell infected with the HBV and/or HDV with an effective amount of said compound(s) and/or pharmaceutical composition.

Some embodiments described herein relate to a method of treating a HIV infection that can include administering to a subject identified as suffering from the HIV infection an effective amount of a compound described herein (for example, a compound of Formula (I), or a pharmaceutically acceptable salt thereof), or a pharmaceutical composition that includes an effective amount of a compound described herein (such as, a compound of Formula (I), or a pharmaceutically acceptable salt thereof). Other embodiments described herein relate to using a compound described herein (for example, a compound of Formula (I), or a pharmaceutically acceptable salt thereof), in the manufacture of a medicament for treating a HIV infection. Still other embodiments described herein relate to the use of a compound described herein (such as, a compound of Formula (I), or a pharmaceutically acceptable salt thereof), or a pharmaceutical composition that includes an effective amount of a compound described herein (for example, a compound of Formula (I), or a pharmaceutically acceptable salt thereof) for treating a HIV infection.

Some embodiments disclosed herein relate to a method of treating a HIV infection that can include contacting a cell infected with the HIV with an effective amount of a compound described herein (for example, a compound of Formula (I), or a pharmaceutically acceptable salt thereof), or a pharmaceutical composition that includes an effective amount of a compound described herein. Other embodiments described herein relate to using a compound described herein (for example, a compound of Formula (I), or a pharmaceutically acceptable salt thereof) in the manufacture of a medicament for treating a WV infection that can include contacting a cell infected with the WV with an effective amount of said compound(s) and/or pharmaceutical composition. Still other embodiments described herein relate to the use of a compound described herein (for example, a compound of Formula (I), or a pharmaceutically acceptable salt thereof), or a pharmaceutical composition that includes a compound described herein for treating a HIV infection, wherein the use includes contacting a cell infected with the HIV with an effective amount of said compound(s) and/or pharmaceutical composition.

Some embodiments disclosed herein relate to a method of inhibiting replication of HIV that can include contacting a cell infected with the HIV with an effective amount of a compound described herein (for example, a compound of Formula (I), or a pharmaceutically acceptable salt thereof), or a pharmaceutical composition that includes a compound described herein (for example, a compound of Formula (I), or a pharmaceutically acceptable salt thereof). Other embodiments described herein relate to using a compound described herein (such as, a compound of Formula (I), or a pharmaceutically acceptable salt thereof) in the manufacture of a medicament for inhibiting replication of HIV that can include contacting a cell infected with HIV with an effective amount of said compound(s) and/or pharmaceutical composition. Still other embodiments described herein relate to the use of a compound described herein (for example, a compound of Formula (I), or a pharmaceutically acceptable salt thereof), or a pharmaceutical composition that includes an effective amount of a compound described herein (such as, a compound of Formula (I), or a pharmaceutically acceptable salt thereof), for inhibiting replication of HIV, wherein the use includes contacting a cell infected with the HIV with an effective amount of said compound(s) and/or pharmaceutical composition.

### BRIEF DESCRIPTION OF THE DRAWINGS

FIG. 1 shows example non-nucleoside reverse transcriptase inhibitors (NNRTI's).

FIG. 2 shows example nucleoside reverse transcriptase inhibitor (NRTI's).

FIG. 3A shows example HIV protease inhibitors. FIG. 3B shows additional HIV, HBV and/or HDV protease inhibitors.

FIG. 4A shows HIV fusion/entry inhibitors. FIG. 4B shows HBV and/or HDV fusion/entry inhibitors.

FIG. 5 shows HIV integrase strand transfer inhibitor (INSTI's).

FIG. 6A shows additional HIV antiviral compounds. FIG. 6B shows additional antiviral compounds.

FIG. 7 shows example HIV, HBV and/or HDV viral maturation inhibitors.

FIG. 8 shows example HIV, HBV and/or HDV capsid assembly modulators.

FIG. 9 shows example anti-HBV and/or anti-HDV farnesoid X receptor (FXR) agonists.

FIG. 10 shows example anti-HBV and/or anti-HDV tumor necrosis factor (TNF)/cyclophilin inhibitors.

FIG. 11 shows example anti-HBV and/or anti-HDV toll-like receptor (TLR) agonists.

FIG. 12 shows example HBV and/or HDV polymerase inhibitors.

FIG. 13 shows example HBV and/or HDV vaccines.

### DETAILED DESCRIPTION

The Hepadnavirus family is a family of enveloped viruses utilizing partially double-stranded, partially single-stranded circular DNA genomes. This family includes a group of viruses that cause liver disease in various organisms, and is divided between two genera: the Avihepadnaviruses, affecting birds, and the Orthohepdnaviruses, affecting mammals. Hepatitis B is a causative agent of acute/chronic hepatitis, and has a partially double-stranded 3.2 kb circular DNA from which four proteins are synthesized: the core, polymerase, surface antigen and X-gene product.

During hepatitis infection, HBV virions enter hepatocytes through a receptor-mediated process. Viral replication occurs through a multi-step mechanism. First, the circular, partially double-stranded DNA genome is transcribed by the host cell machinery, and then the full length RNA transcript is packaged into viral procapsids. The transcript is then reverse-transcribed within the capsid by the P protein, utilizing the P protein's intrinsic protein priming activity. The RNA component is then degraded by an intrinsic RNase H activity of the P protein, to yield a full-length minus-strand circular DNA. Finally, a subsequent partial plus-strand DNA is synthesized to yield the final viral genome assembly.

Viral capsids also may release the circular, partially double stranded genome into the nucleus of host cells, where synthesis of the complementary strand to the single stranded region is completed and the remaining viral ends are ligated to form the covalently closed circular DNA (cccDNA), which persists in host cell nuclei and can be passed on to daughter cells during cell division. The presence of the cccDNA gives rise to the risk of viral reemergence throughout the life of the host organism. Additionally, HBV carriers can transmit the disease for many years. Immunosuppressed individuals are especially at risk for the establishment of persistent (chronic) or latent HBV infection.

HDV is a subviral satellite of HBV, and thus, may only propagate in the presence of HBV. See, e.g., Shieh, et al., Nature, 329(6137), pp. 343-346 (1987). Replication of the single-stranded circular RNA HDV genome produces two forms of a RNA-binding protein known as the long and small delta antigens (Ag). After entering a hepatocyte, the virus is uncoated and the nucleocapsid translocated to the nucleus. The virus then uses the host cell's RNA polymerases, which treat the RNA genome as dsDNA due to its tertiary structure. Three forms of RNA are produced during replication: circular genomic RNA, circular complementary antigenomic RNA and a linear polyadenylated antigenomic RNA.

HBV and HDV are primarily transmitted by blood or mucosal contact, including by sexual activity. Infection with HBV and/or HDV leads to a wide spectrum of liver disease ranging from acute (including fulminant hepatic failure) to chronic hepatitis, cirrhosis and hepatocellular carcinoma. Acute HBV and/or HDV infection can be asymptomatic, or present with symptomatic acute effects, including fever, headaches, joint aches, and diarrhea, leading to the more severe symptoms of liver enlargement and/or jaundice associated with conjugated hyperbilirubinemia and cholestasis. Most adults infected with the virus recover, but 5%-10% are unable to clear the virus and become chronically infected. Many chronically infected individuals have persistent mild liver disease (latent HBV and/or HDV), presenting with lymphoid aggregates and bile duct damage, steatosis and/or increased fibrosis that may lead to cirrhosis. Others with chronic HBV and/or HDV infection develop the active disease, which can lead to life-threatening conditions such as cirrhosis and liver cancer. Some subjects with latent HBV and/or HDV may relapse and develop acute hepatitis.

HIV is a lentivirus that belongs to the Retroviridae family. HIV is an enveloped virus with a core consisting of two copies of a positive single-stranded RNA. HIV relies upon reverse transcriptase for reverse transcription of RNA into DNA, which becomes incorporated into host genome as a provirus. HIV uses viral glycoprotein 120 (gp 120) to bind to and infect CD4+T lymphocytes. An increase in viral plasma load corresponds to a decrease in CD4+T lymphocyte counts. Normal CD4+T lymphocyte levels are from about 500 to 1,200 cells/mL. Two types of HIV have been characterized, HIV-1 and HIV-2. HIV-1 is more virulent and more infective, and has a global prevalence, whereas HIV-2 is less virulent and is geographically confined.

### Definitions

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there are a plurality of definitions for a term herein, those in this section prevail unless stated otherwise.

As used herein, anwhat is y “R” group(s) such as, without limitation, R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²² and R²³ represent substituents that can be attached to the indicated atom. An R group may be substituted or unsubstituted. If two “R” groups are described as being “taken together” the R groups and the atoms they are attached to can form a cycloalkyl, cycloalkenyl, aryl, heteroaryl or heterocyclyl. For example, without limitation, if R^{a} and R^{b} of an NR^{a} R^{b} group are indicated to be “taken together,” it means that they are covalently bonded to one another to form a ring:

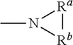

In addition, if two “R” groups are described as being “taken together” with the atom(s) to which they are attached to form a ring as an alternative, the R groups are not limited to the variables or substituents defined previously, when the R group are not taken together.

Whenever a group is described as being “optionally substituted” that group may be unsubstituted or substituted with one or more of the indicated substituents. Likewise, when a group is described as being “unsubstituted or substituted” if substituted, the substituent(s) may be selected from one or more of the indicated substituents. If no substituents are indicated, it is meant that the indicated “optionally substituted” or “substituted” group may be substituted with one or more group(s) individually and independently selected from alkyl, alkenyl, alkynyl, cycloalkyl, cycloalkenyl, aryl, heteroaryl, heterocyclyl, aryl(alkyl), heteroaryl(alkyl), heterocyclyl(alkyl), hydroxy, alkoxy, acyl, cyano, halogen, thiocarbonyl, O-carbamyl, N-carbamyl, O-thiocarbamyl, N-thiocarbamyl, C-amido, N-amido, S-sulfonamido, N-sulfonamido, C-carboxy, O-carboxy, isocyanato, thiocyanato, isothiocyanato, nitro, silyl, sulfenyl, sulfinyl, sulfonyl, haloalkyl, haloalkoxy, trihalomethanesulfonyl, trihalomethanesulfonamido, an amino, a mono-substituted amine group and a di-substituted amine group. The number and type of atoms present in each of the groups of this paragraph are as defined herein, unless stated otherwise.

As used herein, “Ca to C” in which “a” and “b” are integers refer to the number of carbon atoms in an alkyl, alkenyl or alkynyl group, or the number of carbon atoms in the ring of a cycloalkyl, cycloalkenyl, aryl, heteroaryl or heterocyclyl group. That is, the alkyl, alkenyl, alkynyl, ring(s) of the cycloalkyl, ring(s) of the cycloalkenyl, ring(s) of the aryl, ring(s) of the heteroaryl or ring(s) of the heterocyclyl can contain from “a” to “b”, inclusive, carbon atoms. Thus, for example, a “C₁ to C₄ alkyl” group refers to all alkyl groups having from 1 to 4 carbons, that is, CH₃-, CH₃CH₂-, CH₃CH₂CH₂-, (CH₃)₂CH—, CH₃CH₂CH₂CH₂-, CH₃CH₂CH(CH₃)— and (CH₃)₃C—. If no “a” and “b” are designated with regard to an alkyl, alkenyl, alkynyl, cycloalkyl cycloalkenyl, aryl, heteroaryl or heterocyclyl group, the broadest range described in these definitions is to be assumed.

As used herein, “alkyl” refers to a straight or branched hydrocarbon chain that comprises a fully saturated (no double or triple bonds) hydrocarbon group. The alkyl group may have 1 to 20 carbon atoms (whenever it appears herein, a numerical range such as “1 to 20” refers to each integer in the given range; for example, “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 10 carbon atoms. The alkyl group could also be a lower alkyl having 1 to 6 carbon atoms. The alkyl group of the compounds may be designated as “C₁-C₄ alkyl” or similar designations. By way of example only, “C₁-C₄ alkyl” indicates that there are one to four carbon atoms in the alkyl chain, i.e., the alkyl chain is selected from methyl, ethyl, propyl, iso-propyl, n-butyl, iso-butyl, sec-butyl and t-butyl. Typical alkyl groups include, but are in no way limited to, methyl, ethyl, propyl, isopropyl, butyl, isobutyl, tertiary butyl, pentyl and hexyl. The alkyl group may be substituted or unsubstituted.

As used herein, “alkenyl” refers to an alkyl group that contains in the straight or branched hydrocarbon chain one or more double bonds. An alkenyl may include 2 to 20 carbon atoms, 2 to 10 carbon atoms or 2 to 6 carbon atoms. Examples of alkenyl groups include allenyl, vinylmethyl and ethenyl. An alkenyl group may be unsubstituted or substituted.

As used herein, “alkynyl” refers to an alkyl group that contains in the straight or branched hydrocarbon chain one or more triple bonds. An alkynyl may include 2 to 20 carbon atoms, 2 to 10 carbon atoms or 2 to 6 carbon atoms. Examples of alkynyls include ethynyl and propynyl. An alkynyl group may be unsubstituted or substituted.

As used herein, “cycloalkyl” refers to a completely saturated (no double or triple bonds) mono- or multi-cyclic hydrocarbon ring system. When composed of two or more rings, the rings may be joined together in a fused fashion. Cycloalkyl groups can contain 3 to 10 atoms in the ring(s), 3 to 8 atoms in the ring(s) or 3 to 6 atoms in the ring(s). A cycloalkyl group may be unsubstituted or substituted. Typical cycloalkyl groups include, but are in no way limited to, cyclopropyl, cyclobutyl, cyclopentyl, cyclohexyl, cycloheptyl and cyclooctyl.

As used herein, “cycloalkenyl” refers to a mono- or multi-cyclic hydrocarbon ring system that contains one or more double bonds in at least one ring; although, if there is more than one, the double bonds cannot form a fully delocalized pi-electron system throughout all the rings (otherwise the group would be “aryl,” as defined herein). When composed of two or more rings, the rings may be connected together in a fused fashion. Cycloalkenyl groups can contain 3 to 10 atoms in the ring(s) or 3 to 8 atoms in the ring(s). A cycloalkenyl group may be unsubstituted or substituted.

As used herein, “aryl” refers to a carbocyclic (all carbon) monocyclic or multicyclic aromatic ring system (including fused ring systems where two carbocyclic rings share a chemical bond) that has a fully delocalized pi-electron system throughout all the rings. The number of carbon atoms in an aryl group can vary. For example, the aryl group can be a C₆-C₁₄ aryl group, a C₆-C₁₀ aryl group, or a C₆ aryl group. Examples of aryl groups include, but are not limited to, phenyl, naphthyl and azulene. An aryl group may be substituted or unsubstituted.

As used herein, “heteroaryl” refers to a monocyclic, bicyclic and tricyclic aromatic ring system (a ring system with fully delocalized pi-electron system) that contain(s) one or more heteroatoms (for example, 1 to 5 heteroatoms), that is, an element other than carbon, including but not limited to, nitrogen, oxygen and sulfur. The number of atoms in the ring(s) of a heteroaryl group can vary. For example, the heteroaryl group can contain 4 to 14 atoms in the ring(s), 5 to 10 atoms in the ring(s) or 5 to 6 atoms in the ring(s). Furthermore, the term “heteroaryl” includes fused ring systems where two rings, such as at least one aryl ring and at least one heteroaryl ring, or at least two heteroaryl rings, share at least one chemical bond. Examples of heteroaryl rings include, but are not limited to, furan, furazan, thiophene, benzothiophene, phthalazine, pyrrole, oxazole, benzoxazole, 1,2,3-oxadiazole, 1,2,4-oxadiazole, thiazole, 1,2,3-thiadiazole, 1,2,4-thiadiazole, benzothiazole, imidazole, benzimidazole, indole, indazole, pyrazole, benzopyrazole, isoxazole, benzoisoxazole, isothiazole, triazole, benzotriazole, thiadiazole, tetrazole, pyridine, pyridazine, pyrimidine, pyrazine, purine, pteridine, quinoline, isoquinoline, quinazoline, quinoxaline, cinnoline and triazine. A heteroaryl group may be substituted or unsubstituted.

As used herein, “heterocyclyl” or “heteroalicyclyl” refers to three-, four-, five-, six-, seven-, eight-, nine-, ten-, up to 18-membered monocyclic, bicyclic and tricyclic ring system wherein carbon atoms together with from 1 to 5 heteroatoms constitute said ring system. A heterocycle may optionally contain one or more unsaturated bonds situated in such a way, however, that a fully delocalized pi-electron system does not occur throughout all the rings. The heteroatom(s) is an element other than carbon including, but not limited to, oxygen, sulfur and nitrogen. A heterocycle may further contain one or more carbonyl or thiocarbonyl functionalities, so as to make the definition include oxo-systems and thio-systems such as lactams, lactones, cyclic imides, cyclic thioimides and cyclic carbamates. When composed of two or more rings, the rings may be joined together in a fused fashion. Additionally, any nitrogens in a heteroalicyclic may be quaternized. Heterocyclyl or heteroalicyclic groups may be unsubstituted or substituted. Examples of such “heterocyclyl” or “heteroalicyclyl” groups include but are not limited to, 1,3-dioxin, 1,3-dioxane, 1,4-dioxane, 1,2-dioxolane, 1,3-dioxolane, 1,4-dioxolane, 1,3-oxathiane, 1,4-oxathiin, 1,3-oxathiolane, 1,3-dithiole, 1,3-dithiolane, 1,4-oxathiane, tetrahydro-1,4-thiazine, 2H-1,2-oxazine, maleimide, succinimide, barbituric acid, thiobarbituric acid, dioxopiperazine, hydantoin, dihydrouracil, trioxane, hexahydro-1,3,5-triazine, imidazoline, imidazolidine, isoxazoline, isoxazolidine, oxazoline, oxazolidine, oxazolidinone, thiazoline, thiazolidine, morpholine, oxirane, piperidine N-Oxide, piperidine, piperazine, pyrrolidine, pyrrolidone, pyrrolidione, 4-piperidone, pyrazoline, pyrazolidine, 2-oxopyrrolidine, tetrahydropyran, 4H-pyran, tetrahydrothiopyran, thiamorpholine, thiamorpholine sulfoxide, thiamorpholine sulfone and their benzo-fused analogs (e.g., benzimidazolidinone, tetrahydroquinoline and/or 3,4-methylenedioxyphenyl).

As used herein, “aryl(alkyl)” refers to an aryl group connected, as a substituent, via an alkylene group. The alkylene and aryl group of an aryl(alkyl) may be substituted or unsubstituted. Examples include but are not limited to benzyl, 2-phenyl(alkyl), 3-phenyl(alkyl) and naphthyl(alkyl).

As used herein, “heteroaryl(alkyl)” refers to a heteroaryl group connected, as a substituent, via an alkylene group. The alkylene and heteroaryl group of heteroaryl(alkyl) may be substituted or unsubstituted. Examples include but are not limited to 2-thienyl(alkyl), 3-thienyl(alkyl), furyl(alkyl), thienyl(alkyl), pyrrolyl(alkyl), pyridyl(alkyl), isoxazolyl(alkyl), imidazolyl(alkyl) and their benzo-fused analogs.

A “(heterocyclyl)alkyl” refers to a heterocyclic group connected, as a substituent, via an alkylene group. The alkylene and heterocyclyl of a heterocyclyl(alkyl) may be substituted or unsubstituted. Examples include but are not limited tetrahydro-2H-pyran-4-yl(methyl), piperidin-4-yl(ethyl), piperidin-4-yl(propyl), tetrahydro-2H-thiopyran-4-yl(methyl) and 1,3-thiazinan-4-yl(methyl).

“Alkylene groups” are straight-chained —CH₂— tethering groups having between one and ten carbon atoms, one to five carbon atoms or one to three carbon atoms that form bonds to connect molecular fragments via their terminal carbon atoms. Examples include, but are not limited to, methylene (—CH₂-), ethylene (—CH₂CH₂-), propylene (—CH₂CH₂CH₂-), butylene (—CH₂CH₂CH₂CH₂-) and pentylene (—CH₂CH₂CH₂CH₂CH₂-). An alkylene group can be substituted by replacing one or more hydrogen of the alkylene group with a substituent(s) listed under the definition of “optionally substituted.”

As used herein, “alkoxy” refers to the formula —OR wherein R is an alkyl, an alkenyl, an alkynyl, a cycloalkyl, a cycloalkenyl, aryl, heteroaryl, heterocyclyl, aralkyl, heteroaryl(alkyl) or heterocyclyl(alkyl) is defined herein. A non-limiting list of alkoxys are methoxy, ethoxy, n-propoxy, 1-methylethoxy(isopropoxy), n-butoxy, iso-butoxy, sec-butoxy, tert-butoxy, phenoxy and benzoxy. An alkoxy may be substituted or unsubstituted.

As used herein, “acyl” refers to a hydrogen, an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl) connected, as substituents, via a carbonyl group. Examples include formyl, acetyl, propanoyl, benzoyl and acryl. An acyl may be substituted or unsubstituted.

As used herein, “hydroxyalkyl” refers to an alkyl group in which one or more of the hydrogen or deuterium atoms are replaced by a hydroxy group. Exemplary hydroxyalkyl groups include but are not limited to, 2-hydroxyethyl, 3-hydroxypropyl, 2-hydroxypropyl and 2,2-dihydroxyethyl. A hydroxyalkyl may be substituted or unsubstituted.

As used herein, “haloalkyl” refers to an alkyl group in which one or more of the hydrogen atoms are replaced by a halogen (for example, mono-haloalkyl, di-haloalkyl and tri-haloalkyl). Such groups include but are not limited to, chloromethyl, fluoromethyl, difluoromethyl, trifluoromethyl, 1-chloro-2-fluoromethyl and 2-fluoroisobutyl. A haloalkyl may be substituted or unsubstituted.

As used herein, “haloalkoxy” refers to an —O-alkyl group in which one or more of the hydrogen atoms are replaced by a halogen (for example, mono-haloalkoxy, di-haloalkoxy and tri-haloalkoxy). Such groups include but are not limited to, chloromethoxy, fluoromethoxy, difluoromethoxy, trifluoromethoxy, 1-chloro-2-fluoromethoxy and 2-fluoroisobutoxy. A haloalkoxy may be substituted or unsubstituted.

A “sulfenyl” group refers to an “—SR” group in which R can be hydrogen, deuterium, alkyl, alkenyl, alkynyl, cycloalkyl, cycloalkenyl, aryl, heteroaryl, heterocyclyl, aryl(alkyl), heteroaryl(alkyl) or heterocyclyl(alkyl). A sulfenyl may be substituted or unsubstituted.

A “sulfinyl” group refers to an “—S(═O)—R” group in which R can be the same as defined with respect to sulfenyl. A sulfinyl may be substituted or unsubstituted.

A “sulfonyl” group refers to an “SO₂R” group in which R can be the same as defined with respect to sulfenyl. A sulfonyl may be substituted or unsubstituted.

An “O-carboxy” group refers to a “RC(═O)O-” group in which R can be hydrogen, deuterium, alkyl, alkenyl, alkynyl, cycloalkyl, cycloalkenyl, aryl, heteroaryl, heterocyclyl, aryl(alkyl), heteroaryl(alkyl) or heterocyclyl(alkyl), as defined herein. An O-carboxy may be substituted or unsubstituted.

The terms “ester” and “C-carboxy” refer to a “—C(═O)OR” group in which R can be the same as defined with respect to O-carboxy. An ester and C-carboxy may be substituted or unsubstituted.

A “thiocarbonyl” group refers to a “—C(═S)R” group in which R can be the same as defined with respect to O-carboxy. A thiocarbonyl may be substituted or unsubstituted.

A “trihalomethanesulfonyl” group refers to an “X₃CSO₂-” group wherein each X is a halogen.

A “trihalomethanesulfonamido” group refers to an “X₃CS(O)₂N(R_{A})—” group wherein each X is a halogen, and R_{A} is hydrogen, deuterium, alkyl, alkenyl, alkynyl, cycloalkyl, cycloalkenyl, aryl, heteroaryl, heterocyclyl, aryl(alkyl), heteroaryl(alkyl) or heterocyclyl(alkyl).

The term “amino” as used herein refers to a —NH₂ group.

As used herein, the term “hydroxy” refers to a —OH group.

A “cyano” group refers to a “—CN” group.

The term “azido” as used herein refers to a —N₃ group.

An “isocyanato” group refers to a “—NCO” group.

A “thiocyanato” group refers to a “—CNS” group.

An “isothiocyanato” group refers to an “—NCS” group.

A “mercapto” group refers to an “—SH” group.

A “carbonyl” group refers to a C═O group.

An “S-sulfonamido” group refers to a “—SO₂N(R_{A}R_{B})” group in which R_{A} and R_{B} can be independently hydrogen, deuterium, alkyl, alkenyl, alkynyl, cycloalkyl, cycloalkenyl, aryl, heteroaryl, heterocyclyl, aryl(alkyl), heteroaryl(alkyl) or heterocyclyl(alkyl). An S-sulfonamido may be substituted or unsubstituted.

An “N-sulfonamido” group refers to a “RSO₂N(R_{A})—” group in which R and R_{A} can be independently hydrogen, deuterium, alkyl, alkenyl, alkynyl, cycloalkyl, cycloalkenyl, aryl, heteroaryl, heterocyclyl, aryl(alkyl), heteroaryl(alkyl) or heterocyclyl(alkyl). An N-sulfonamido may be substituted or unsubstituted.

An “O-carbamyl” group refers to a “—OC(═O)N(R_{A}R_{B})” group in which R_{A} and R_{B} can be independently hydrogen, deuterium, alkyl, alkenyl, alkynyl, cycloalkyl, cycloalkenyl, aryl, heteroaryl, heterocyclyl, aryl(alkyl), heteroaryl(alkyl) or heterocyclyl(alkyl). An O-carbamyl may be substituted or unsubstituted.

An “N-carbamyl” group refers to an “ROC(═O)N(R_{A})—” group in which R and R_{A} can be independently hydrogen, deuterium, alkyl, alkenyl, alkynyl, cycloalkyl, cycloalkenyl, aryl, heteroaryl, heterocyclyl, aryl(alkyl), heteroaryl(alkyl) or heterocyclyl(alkyl). An N-carbamyl may be substituted or unsubstituted.

An “O-thiocarbamyl” group refers to a “—OC(═S)—N(R_{A}R_{B})” group in which R_{A} and R_{B} can be independently hydrogen, deuterium, alkyl, alkenyl, alkynyl, cycloalkyl, cycloalkenyl, aryl, heteroaryl, heterocyclyl, aryl(alkyl), heteroaryl(alkyl) or heterocyclyl(alkyl). An O-thiocarbamyl may be substituted or unsubstituted.

An “N-thiocarbamyl” group refers to an “ROC(═S)N(R_{A})—” group in which R and R_{A} can be independently hydrogen, deuterium, alkyl, alkenyl, alkynyl, cycloalkyl, cycloalkenyl, aryl, heteroaryl, heterocyclyl, aryl(alkyl), heteroaryl(alkyl) or heterocyclyl(alkyl). An N-thiocarbamyl may be substituted or unsubstituted.

A “C-amido” group refers to a “—C(═O)N(R_{A}R_{B})” group in which R_{A} and R_{B} can be independently hydrogen, deuterium, alkyl, alkenyl, alkynyl, cycloalkyl, cycloalkenyl, aryl, heteroaryl, heterocyclyl, aryl(alkyl), heteroaryl(alkyl) or heterocyclyl(alkyl). A C-amido may be substituted or unsubstituted.

An “N-amido” group refers to a “RC(═O)N(R_{A})—” group in which R and R_{A} can be independently hydrogen, deuterium, alkyl, alkenyl, alkynyl, cycloalkyl, cycloalkenyl, aryl, heteroaryl, heterocyclyl, aryl(alkyl), heteroaryl(alkyl) or heterocyclyl(alkyl). An N-amido may be substituted or unsubstituted.

A “mono-substituted amine” group refers to a “—NHR_{A}” group in which R_{A} can be an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl), as defined herein. The R_{A} may be substituted or unsubstituted. Examples of mono-substituted amine groups include, but are not limited to, —NH(methyl), —NH(ethyl), —NH(isopropyl), —NH(phenyl), —NH(benzyl), and the like.

A “di-substituted amine” group refers to a “—NR_{A}R_{B}” group in which R_{A} and R_{B} can be independently an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl), as defined herein. R_{A} and R_{B} can independently be substituted or unsubstituted. Examples of di-substituted amino groups include, but are not limited to, —N(methyl)₂, —N(phenyl)(methyl), —N(ethyl)(methyl), —N(ethyl)₂, —N(isopropyl)₂ and the like.

The term “halogen atom” or “halogen” as used herein, means any one of the radio-stable atoms of column 7 of the Periodic Table of the Elements, such as, fluorine, chlorine, bromine and iodine.

Where the number of substituents is not specified (for example, haloalkyl), there may be one or more substituents present. For example, “haloalkyl” may include one or more of the same or different halogens. As another example, “C₁-C₃ alkoxyphenyl” may include one or more of the same or different alkoxy groups containing one, two or three atoms.

As used herein, the abbreviations for any chemical compounds are, unless indicated otherwise, in accord with their common usage, recognized abbreviations, or the IUPAC-IUB Commission on Biochemical Nomenclature. See, Biochem. 11:942-944 (1972).

As used herein, the term “N-linked heterocyclic base” refers to an optionally substituted nitrogen-containing heterocyclyl or an optionally substituted nitrogen-containing heteroaryl that can be attached via a ring nitrogen. The N-linked heterocyclic base can be monocyclic or multicyclic (such as, bicyclic). When compared of two or more rings, the rings can be connected in a fused-fashion. In some embodiments, the N-linked heterocyclic base can be an optionally substituted N-linked purine-base or an optionally substituted N-linked pyrimidine-base. The term “purine-base” is used herein in its ordinary sense as understood by those skilled in the art, and includes its tautomers. Similarly, the term “pyrimidine-base” is used herein in its ordinary sense as understood by those skilled in the art, and includes its tautomers. A non-limiting list of optionally substituted purine-bases includes purine, adenine, guanine, hypoxanthine, xanthine, alloxanthine, 7-alkylguanine (e.g. 7-methylguanine), theobromine, caffeine, uric acid and isoguanine. Examples of pyrimidine-bases include, but are not limited to, cytosine, thymine, uracil, 5,6-dihydrouracil and 5-alkylcytosine (e.g., 5-methylcytosine). Other non-limiting examples of heterocyclic bases include diaminopurine, 8-oxo-N⁶-alkyladenine (e.g., 8-oxo-N⁶-methyladenine), 7-deazaxanthine, 7-deazaguanine, 7-deazaadenine, N⁴,N⁴-ethanocytosin, N⁶,N⁶-ethano-2,6-diaminopurine, 5-halouracil (e.g., 5-fluorouracil and 5-bromouracil), pseudoisocytosine, isocytosine, isoguanine, and other heterocyclic bases described in U.S. Pat. Nos. 5,432,272 and 7,125,855, which are incorporated herein by reference for the limited purpose of disclosing additional heterocyclic bases.

As used herein, the term “C-linked heterocyclic base” refers to an optionally substituted nitrogen-containing heterocyclyl or an optionally substituted nitrogen-containing heteroaryl that can be attached via a ring carbon. The C-linked heterocyclic base can be monocyclic or multicyclic (for example, bicyclic). When compared of two or more rings, the rings can be connected in a fused-fashion. In some embodiments, the C-linked heterocyclic base can be an optionally substituted imidazo[2,1-f][1,2,4]triazine base or an optionally substituted pyrazolo[1,5-a][1,3,5]triazine base. In some embodiments, a N-linked heterocyclic base and/or a C-linked heterocyclic base can be include an amino or an enol protecting group(s).

The term “—N-linked α-amino acid” refers to an α-amino acid that is attached to the indicated moiety via a main-chain amino or mono-substituted amine group. The —N-linked α-amino acid can be attached via one of the hydrogens that is part of the main-chain amino or mono-substituted amine group such that the —N-linked α-amino acid is attached via the nitrogen of the main-chain amino or mono-substituted amine group. N-linked α-amino acids can be substituted or unsubstituted.

The term “—N-linked α-amino acid ester derivative” refers to an α-amino acid in which a main-chain carboxylic acid group has been converted to an ester group. In some embodiments, the ester group has a formula selected from alkyl-O—C(═O)—, cycloalkyl-O—C(═O)—, aryl-O—C(═O)— and aryl(alkyl)-O—C(═O)—. A non-limiting list of ester groups include substituted and unsubstituted versions of the following: methyl-O—C(═O)—, ethyl-O—C(═O)—, n-propyl-O—C(═O)—, isopropyl-O—C(═O)—, n-butyl-O—C(═O)—, isobutyl-O—C(═O)—, tert-butyl-O—C(═O)—, neopentyl-O—C(═O)—, cyclopropyl-O—C(═O)—, cyclobutyl-O—C(═O)—, cyclopentyl-O—C(═O)—, cyclohexyl-O—C(═O)—, phenyl-O—C(═O)—, benzyl-O—C(═O)— and naphthyl-O—C(═O)—. N-linked α-amino acid ester derivatives can be substituted or unsubstituted.

The term “—O-linked α-amino acid” refers to an α-amino acid that is attached to the indicated moiety via the hydroxy from its main-chain carboxylic acid group. The —O-linked α-amino acid can be attached via the hydrogen that is part of the hydroxy from its main-chain carboxylic acid group such that the —O-linked α-amino acid is attached via the oxygen or the main-chain carboxylic acid group. O-linked amino α-acids can be substituted or unsubstituted.

As used herein, the term “α-amino acid” refers to any amino acid (both standard and non-standard amino acids). Examples of suitable α-amino acids include, but are not limited to, alanine, asparagine, aspartate, cysteine, glutamate, glutamine, glycine, proline, serine, tyrosine, arginine, histidine, isoleucine, leucine, lysine, methionine, phenylalanine, threonine, tryptophan and valine. Additional examples of suitable α-amino acids include, but are not limited to, ornithine, hypusine, 2-aminoisobutyric acid, dehydroalanine, citrulline and norleucine.

As used herein, the term “phosphate” is used in its ordinary sense as understood by those skilled in the art, and includes its protonated forms (for example,

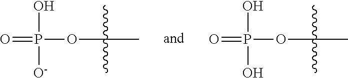 [SMILES: *OP(=O)([O-])O ; *OP(=O)(O)O]

As used herein, the terms “monophosphate,” “diphosphate,” and “triphosphate” are used in their ordinary sense as understood by those skilled in the art, and include protonated forms.

The terms “protecting group” and “protecting groups” as used herein refer to any atom or group of atoms that is added to a molecule in order to prevent existing groups in the molecule from undergoing unwanted chemical reactions. Examples of protecting group moieties are described in T. W. Greene and P. G. M. Wuts, Protective Groups in Organic Synthesis, 3. Ed. John Wiley & Sons, 1999, and in J. F. W. McOmie, Protective Groups in Organic Chemistry Plenum Press, 1973, both of which are hereby incorporated by reference for the limited purpose of disclosing suitable protecting groups. The protecting group moiety may be chosen in such a way, that they are stable to certain reaction conditions and readily removed at a convenient stage using methodology known from the art. A non-limiting list of protecting groups include benzyl; substituted benzyl; alkylcarbonyls and alkoxycarbonyls (e.g., t-butoxycarbonyl (BOC), acetyl, or isobutyryl); arylalkylcarbonyls and arylalkoxycarbonyls (e.g., benzyloxycarbonyl); substituted methyl ether (e.g. methoxymethyl ether); substituted ethyl ether; a substituted benzyl ether; tetrahydropyranyl ether; silyls (e.g., trimethylsilyl, triethylsilyl, triisopropylsilyl, t-butyldimethylsilyl, tri-iso-propylsilyloxymethyl, [2-(trimethylsilyl)ethoxy]methyl or t-butyldiphenylsilyl); esters (e.g. benzoate ester); carbonates (e.g. methoxymethylcarbonate); sulfonates (e.g. tosylate or mesylate); acyclic ketal (e.g. dimethyl acetal); cyclic ketals (e.g., 1,3-dioxane, 1,3-dioxolanes and those described herein); acyclic acetal; cyclic acetal (e.g., those described herein); acyclic hemiacetal; cyclic hemiacetal; cyclic dithioketals (e.g., 1,3-dithiane or 1,3-dithiolane); orthoesters (e.g., those described herein) and triarylmethyl groups (e.g., trityl; monomethoxytrityl (MMTr); 4,4′-dimethoxytrityl (DMTr); 4,4′,4″-trimethoxytrityl (TMTr); and those described herein).

The term “pharmaceutically acceptable salt” refers to a salt of a compound that does not cause significant irritation to an organism to which it is administered and does not abrogate the biological activity and properties of the compound. In some embodiments, the salt is an acid addition salt of the compound. Pharmaceutical salts can be obtained by reacting a compound with inorganic acids such as hydrohalic acid (for example, hydrochloric acid or hydrobromic acid), sulfuric acid, nitric acid and phosphoric acid. Pharmaceutical salts can also be obtained by reacting a compound with an organic acid such as aliphatic or aromatic carboxylic or sulfonic acids, for example formic, acetic, succinic, lactic, malic, tartaric, citric, ascorbic, nicotinic, methanesulfonic, ethanesulfonic, p-toluensulfonic, salicylic or naphthalenesulfonic acid. Pharmaceutical salts can also be obtained by reacting a compound with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, C₁-C₇ alkylamine, cyclohexylamine, triethanolamine, ethylenediamine, and salts with amino acids such as arginine and lysine.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; and use of terms like ‘preferably,’ ‘preferred,’ ‘desired,’ or ‘desirable,’ and words of similar meaning should not be understood as implying that certain features are critical, essential, or even important to the structure or function, but instead as merely intended to highlight alternative or additional features that may or may not be utilized in a particular embodiment. In addition, the term “comprising” is to be interpreted synonymously with the phrases “having at least” or “including at least”. When used in the context of a process, the term “comprising” means that the process includes at least the recited steps, but may include additional steps. When used in the context of a compound, composition or device, the term “comprising” means that the compound, composition or device includes at least the recited features or components, but may also include additional features or components.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

It is understood that, in any compound described herein having one or more chiral centers, if an absolute stereochemistry is not expressly indicated, then each center may independently be of R-configuration or S-configuration or a mixture thereof. Where the compounds described herein have at least one chiral center, they may accordingly exist as enantiomers. Where the compounds possess two or more chiral centers, they may additionally exist as diastereomers. Thus, the compounds provided herein may be enantiomerically pure, enantiomerically enriched, racemic mixture, diastereomerically pure, diastereomerically enriched, or a stereoisomeric mixture. In addition, it is understood that, in any compound described herein having one or more double bond(s) generating geometrical isomers that can be defined as E or Z, each double bond may independently be E or Z a mixture thereof. It is to be understood that all such isomers and mixtures thereof are encompassed, unless stated otherwise.

Likewise, it is understood that, in any compound described, all tautomeric forms are also intended to be included. For example all tautomers of heterocyclic bases known in the art are intended to be included, including tautomers of natural and non-natural purine-bases and pyrimidine-bases.

It is to be understood that where compounds disclosed herein have unfilled valencies, then the valencies are to be filled with hydrogens or isotopes thereof, for example, hydrogen-1 (protium) and hydrogen-2 (deuterium).

It is understood that the compounds described herein can be labeled isotopically. Substitution with isotopes such as deuterium may afford certain therapeutic advantages resulting from greater metabolic stability, such as, for example, increased in vivo half-life or reduced dosage requirements. Each chemical element as represented in a compound structure may include any isotope of said element. For example, at any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise.

It is understood that the compounds, methods and combinations described herein include crystalline forms (also known as polymorphs, which include the different crystal packing arrangements of the same elemental composition of a compound), amorphous phases, solvates and hydrates. In some embodiments, the compounds described herein (including those described in methods and combinations) exist in solvated forms with pharmaceutically acceptable solvents such as water, ethanol, or the like. In other embodiments, the compounds described herein (including those described in methods and combinations) exist in unsolvated form. Solvates contain either stoichiometric or non-stoichiometric amounts of a solvent, and may be formed during the process of crystallization with pharmaceutically acceptable solvents such as water, ethanol, or the like. Hydrates are formed when the solvent is water, or alcoholates are formed when the solvent is alcohol. In general, the solvated forms are considered equivalent to the unsolvated forms for the purposes of the compounds and methods provided herein.

Where a range of values is provided, it is understood that the upper and lower limit, and each intervening value between the upper and lower limit of the range is encompassed within the embodiments.

### Compounds

Some embodiments disclosed herein relate to a compound of Formula (I), or a pharmaceutically acceptable salt thereof:

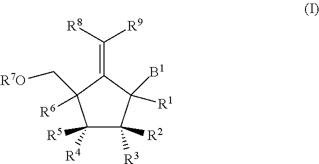

wherein: B¹ can be an optionally substituted C-linked heterocyclic base or an optionally substituted N-linked heterocyclic base; R¹ can be selected from hydrogen, halogen, cyano, an optionally substituted C₁₋₆ alkyl, an unsubstituted C₂₋₆ alkenyl and an unsubstituted C₂₋₆ alkynyl, wherein when the C₁₋₆ alkyl is substituted, the C₁₋₆ alkyl can be substituted with at least one halogen; R² can be hydrogen or fluoro; R³ can be hydrogen or fluoro; R⁴ can be selected from hydrogen, halogen, hydroxy, cyano and an optionally substituted C₁₋₄ alkyl, wherein when the C₁₋₄ alkyl is substituted, the C₁₋₄ alkyl can be substituted with a hydroxy or at least one halogen; R⁵ can be hydrogen or hydroxy; R⁶ can be selected from hydrogen, halogen, cyano, an optionally substituted C₁₋₄ alkyl, an optionally substituted C₂₋₄ alkenyl and an unsubstituted C₂₋₄ alkynyl, wherein when the C₁₋₄ alkyl or the C₂₋₄ alkenyl are substituted, the C₁₋₄ alkyl and C₂₋₄ alkenyl can be independently substituted with at least one halogen; R⁷ can be selected from hydrogen, an optionally substituted acyl, an optionally substituted O-linked α-amino acid,

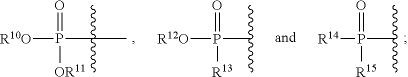

R¹⁰ and R¹¹ can be independently selected from absent, hydrogen,

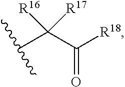

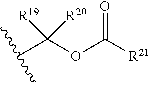

and

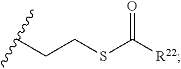

### or R¹⁰ can be

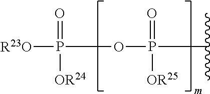

and R¹¹ can be absent or hydrogen; R¹² can be absent, hydrogen, an optionally substituted aryl or an optionally substituted heteroaryl; R¹³ can be an optionally substituted N-linked α-amino acid or an optionally substituted N-linked α-amino acid ester derivative; R¹⁴ and R¹⁵ can be independently an optionally substituted N-linked α-amino acid or an optionally substituted N-linked α-amino acid ester derivative; R¹⁶, R¹⁷, R¹⁹ and R²⁰ can be independently selected from hydrogen, an optionally substituted C₁₋₂₄ alkyl and an optionally substituted aryl; R¹⁸ and R²¹ can be independently selected from hydrogen, an optionally substituted C₁₋₂₄ alkyl, an optionally substituted aryl, an optionally substituted —O—C₁₋₂₄ alkyl and an optionally substituted —O-aryl; R²² can be selected from hydrogen, an optionally substituted C₁₋₂₄ alkyl and an optionally substituted aryl; R²³, R²⁴ and R²⁵ can be independently absent or hydrogen; R⁸ and R⁹ are independently hydrogen or halogen; and m can be 0 or 1.

The orientation of the substituents attached to the cyclopentyl ring can vary. For example, the following Formulae (Ia), (Ib), (Ic) and (Id) are each an example of an embodiment of a compound of Formula (I).

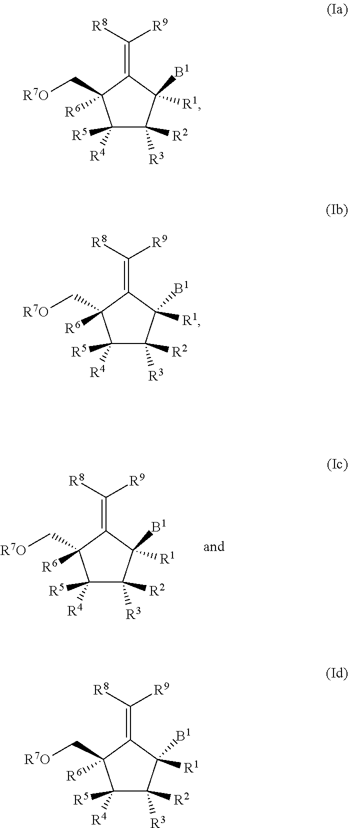

(Id), or a pharmaceutically acceptable salt of any of the foregoing.

A variety of groups can be attached to the cyclopentyl ring. In some embodiments, R⁶ can be halogen. For example, R⁶ can be fluoro. In other embodiments, R⁶ can be cyano. In still other embodiments, R⁶ can be a substituted or unsubstituted, saturated or unsaturated hydrocarbon that includes 1 to 4 carbons. In some embodiments, R⁶ can be an optionally substituted C₁₋₄ alkyl, wherein when the C₁₋₄ alkyl is substituted, the C₁₋₄ alkyl can be substituted with at least one halogen. Examples of suitable C₁₋₄ alkyls include methyl, ethyl, n-propyl, isopropyl, n-butyl, sec-butyl, iso-butyl and tert-butyl. In some embodiments, R⁶ can be an unsubstituted C₁₋₄ alkyl, such as those described herein. In other embodiments, R⁶ can be a substituted C₁₋₄ alkyl, wherein the C₁₋₄ alkyl can be substituted with at least one halogen. For example, R⁶ can be a C₁₋₄ alkyl substituted with 1, 2 or 3 halogens, such as fluoro or chloro. When R⁶ is substituted with one halogen (for example, F or Cl), R⁶ can be a mono-substituted-halogenated C₁₋₄ alkyl. In some embodiments, R⁶ can be a fluoro-substituted C₁₋₄ alkyl. In other embodiments, R⁶ can be a chloro-substituted C₁₋₄ alkyl. A non-limiting list of halogen-substituted C₁₋₄ alkyls include —CH₂F or —CH₂Cl. In some embodiments, the hydrocarbon at R⁶ can include a double and/or a triple bond(s). For example, in some embodiments, R⁶ can be an optionally substituted C₂₋₄ alkenyl, wherein when the C₂₋₄ alkenyl is substituted, the C₂₋₄ alkenyl can be substituted with a halogen. As when a substituted C₁₋₄ alkyl group is present at R⁶, a substituted C₂₋₄ alkenyl can be substituted with 1, 2 or 3 halogens, such as fluoro or chloro. For example, in some embodiments, R⁶ can be a fluoro-substituted C₂₋₄ alkenyl. In other embodiments, R⁶ can be a chloro-substituted C₂₋₄ alkenyl. In some embodiments, R⁶ can be an unsubstituted C₂₋₄ alkenyl. Exemplary C₂₋₄ alkenyls include ethenyl, 1-propenyl, 2-propenyl, 1-butenyl, 2-butenyl and 3-butenyl. In some embodiments, R⁶ can be hydrogen.

The groups attached to the 2′-position of the cyclopentyl ring can vary. In some embodiments, R² can be hydrogen. In other embodiments, R² can be fluoro. In some embodiments, R³ can be hydrogen. In other embodiments, R³ can be fluoro. In some embodiments, R² and R³ can be each hydrogen. In other embodiments, the 2′-position can be disubstituted when R² and R³ are each fluoro. In still other embodiments, R² can be hydrogen; and R³ can be fluoro. In yet still other embodiments, R² can be fluoro; and R³ can be hydrogen.

The groups attached to the 3′-position of the cyclopenyl ring can also vary. In some embodiments, R⁴ can be halogen. The halogen can be F, Cl, Br or I. In some embodiments, R⁴ can be F. In other embodiments, R⁴ can be Cl. In some embodiments, R⁴ can be hydroxy (—OH). In other embodiments, R⁴ can be cyano (—CN). In still other embodiments, R⁴ can be an optionally substituted C₁₋₄ alkyl, wherein when the C₁₋₄ alkyl is substituted, the C₁₋₄ alkyl can be substituted with a hydroxy or at least one halogen. In some embodiments, R⁴ can be an unsubstituted C₁₋₄ alkyl (such as methyl, ethyl, n-propyl, iso-propyl, n-butyl, iso-butyl, sec-butyl and tert-butyl). In other embodiments, R⁴ can be a substituted C₁₋₄ alkyl, wherein the C₁₋₄ alkyl can be substituted with at least one halogen. For example, R⁴ can be a C₁₋₄ alkyl substituted with 1, 2 or 3 halogens, such as fluoro or chloro. When R⁴ is substituted with one halogen (for example, F or Cl), R⁴ can be a mono-substituted-halogenated C₁₋₄ alkyl. In some embodiments, R⁴ can be a fluoro-substituted C₁₋₄ alkyl. In other embodiments, R⁴ can be a chloro-substituted C₁₋₄ alkyl. In various embodiments, the fluoro-substituted C₁₋₄ alkyl can be a mono-substituted, fluoro-substituted C₁₋₄ alkyl, such as CH₂F. In various other embodiments, the chloro-substituted C₁₋₄ alkyl can be a mono-substituted, chloro-substituted C₁₋₄ alkyl, such as CH₂Cl. In some embodiments, R⁴ can be a C₁₋₄ alkyl substituted with one or more hydroxy groups. As an example, R⁴ can be a mono-substituted with hydroxy. In various embodiments, R⁴ can be —CH₂OH. In some embodiments, R⁴ can be a C₁₋₄ alkyl substituted with 1 or 2 hydroxy groups and 1 or 2 halogens (such as F or C₁). In other embodiments, R⁴ can be hydrogen. In various embodiments, including those of this paragraphs, R⁵ can be hydrogen. In other various embodiments, including those of this paragraphs, R⁵ can be hydroxy.

As described herein, R⁸ and R⁹ can be independently hydrogen or halogen. In some embodiments, R⁸ and R⁹ can be each hydrogen such that substituent attached to the cyclopentyl ring is ═CH₂. In other embodiments, R⁸ and R⁹ can be each halogen. When R¹ and R⁹ are each halogen, the halogens can be the same or different. For example, R⁸ and R⁹ can be each fluoro, or one of R⁸ and R⁹ can be fluoro and the other of R⁸ and R⁹ can be chloro. In still other embodiments, one of R⁸ and R⁹ can be hydrogen, and the other of R¹ and R⁹ can be halogen. In various embodiments, when one or both of R⁸ and R⁹ are halogen, the halogen(s) can be fluoro. Examples of substituents attached to the cyclopentyl ring that include a halogen include, but are not limited to, the following: ═CF₂, ═CCl₂,═CFH, ═CClH and ═CClF.

As with other positions on the cyclopentyl ring, the carbon on which B¹ is attached can be further substituted or unsubstituted. In some embodiments, R¹ can be hydrogen. In other embodiments, R¹ can be halogen. Suitable halogens are described herein. For example, R¹ can be fluoro. In still other embodiments, R¹ can be cyano. In yet still other embodiments, R¹ can be an optionally substituted C₁₋₆ alkyl, wherein when the C₁₋₆ alkyl is substituted, the C₁₋₆ alkyl can be substituted with at least one halogen. When R¹ is an unsubstituted C₁₋₆ alkyl, R¹ can be methyl, ethyl, n-propyl, iso-propyl, n-butyl, iso-butyl, sec-butyl, tert-butyl, pentyl (branched or straight-chained) or hexyl (branched or straight-chained) in various embodiments described herein. In various embodiments, when R¹ is substituted, the C₁₋₆ alkyl can be substituted with one or more halogens (such as 1, 2, 3, 4, 5 or 6 halogens). Examples of suitable halogens are described herein. In some embodiments, R¹ can be a mono-halogenated C₁₋₆ alkyl. In other embodiments, R¹ can be a per-halogenated C₁₋₆ alkyl. Exemplary halogenated C₁₋₆ alkyls for R¹ include —CH₂F, —CH₂Cl, —CHF₂, —CHCl₂, —CF₃, —CCl₃, —CH₂CH₂F, CH₂CF₃, —CH₂CHClF, —CHFCH₂F and —CHClCH₂F. In some embodiments, R¹ can be an unsubstituted C₂₋₆ alkenyl. In other embodiments, R¹ can be an unsubstituted C₂₋₆ alkynyl. When R¹ is an unsaturated C₂₋₆ hydrocarbon, in various embodiments, R¹ can be ethenyl, ethynyl or —CH₂—CH═CH₂.

Compounds of Formula (I), or a pharmaceutically acceptable salt thereof, can be referred to as cyclopentyl nucleoside analogs. In some embodiments, R⁷ can be hydrogen. When R⁷ is hydrogen, a compound of Formula (I), or a pharmaceutically acceptable salt thereof, can be a cyclopentyl nucleoside.

In some embodiments, R⁷ can be

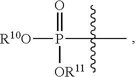

wherein m can be 0 or 1; and R¹⁰, R¹¹, R²³, R²⁴ and R²⁵ can be independently absent or hydrogen. When R⁷ is

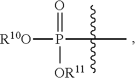

wherein m can be 0 or 1; R⁷ can be

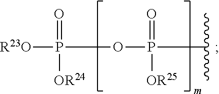

and R¹¹, R²³, R²⁴ and R²⁵ can be independently absent or hydrogen, a compound of Formula (I), or a pharmaceutically acceptable salt thereof, can be a cyclopentyl nucleotide mono-, di- and/or tri-phosphate. Those skilled in the art understand that when R⁷ is

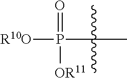

and R¹⁰ and R¹¹ are independently absent or hydrogen, a compound of Formula (I), or a pharmaceutically acceptable salt thereof, can be a mono-phosphate. Those skilled in the art also understand that when R⁷ is

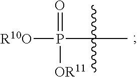

### R¹⁰ is

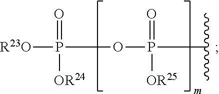

R¹¹, R²³, R²⁴ and R²⁵ can be independently absent or hydrogen; and m is 0 or 1, a compound of Formula (I), or a pharmaceutically acceptable salt thereof, can be a di-phosphate (m is 0) or tri-phosphate (m is 1). When any one of R¹⁰, R¹¹, R²³, R²⁴ and R²⁵ are absent, those skilled in the art understand that the respective oxygen to which R¹⁰, R¹¹, R²³, R²⁴ and R²⁵ are shown attached will have an associated negative charge. For example, when R¹⁰ and R¹¹ are each absent, R⁷ can be

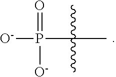 [SMILES: *P(=O)([O-])[O-]]

As further examples, when R⁷ is

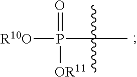

### R¹⁰ is

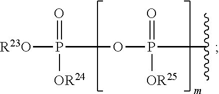

R¹¹, R²³, R²⁴ and R²⁵ are absent; and m is 0 or 1, a compound of Formula (I), or a pharmaceutically acceptable salt thereof, R⁷ can have the following structures:

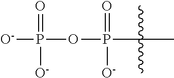 [SMILES: *P(=O)([O-])OP(=O)([O-])[O-]]

(m is 0)

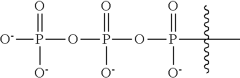 [SMILES: *P(=O)([O-])OP(=O)([O-])OP(=O)([O-])[O-]]

(m is 1).

Compounds of Formula (I), or a pharmaceutically acceptable salt thereof, can include prodrug group(s). The prodrug group(s) can be present at the position equivalent to R⁷. In some embodiments, R⁷ can be an optionally substituted acyl. In some embodiments, the acyl can be unsubstituted. In other embodiments, the acyl can be substituted. An example structure of the optionally substituted acyl can be —C(═O)R²⁶, wherein R²⁶ can be an optionally substituted C₁₋₁₂ alkyl, an optionally substituted monocyclic C₃₋₈ cycloalkyl or an optionally substituted phenyl. In some embodiments, R²⁶ can be an unsubstituted C₁₋₁₂ alkyl. In other embodiments, R²⁶ can be an unsubstituted monocyclic C₃₋₈ cycloalkyl. In still other embodiments, R²⁶ can be an unsubstituted phenyl. In some embodiments, R⁷ can be —C(═O)R²⁶, wherein R²⁶ can be an unsubstituted C₁₋₆ alkyl.

In some embodiments, R⁷ can be an optionally substituted O-linked α-amino acid. Examples of O-linked α-amino acids include alanine, asparagine, aspartate, cysteine, glutamate, glutamine, glycine, proline, serine, tyrosine, arginine, histidine, isoleucine, leucine, lysine, methionine, phenylalanine, threonine, tryptophan and valine. In various embodiments, the O-linked α-amino acid can be unsubstituted. In various other embodiments, the O-linked α-amino acid can be substituted. In some embodiments, R⁷ can be selected from unsubstituted O-linked alanine, unsubstituted O-linked valine, unsubstituted O-linked leucine and unsubstituted O-linked glycine. The α-amino acid can be a natural α-amino acid. Examples of suitable optionally substituted O-linked α-amino acids include the following:

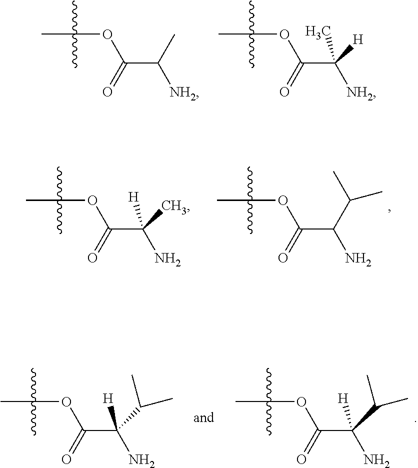

In some embodiments, R⁷ can be

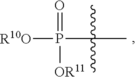

wherein one of R¹⁰ and R¹¹ can be absent, hydrogen or

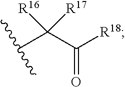

the other of R¹⁰ and R¹¹ can be

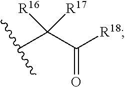

R¹⁶ and R¹⁷ can be independently selected from hydrogen, an optionally substituted C₁₋₂₄ alkyl and an optionally substituted aryl; and R⁸ can be selected from hydrogen, an optionally substituted C₁₋₂₄ alkyl, an optionally substituted aryl, an optionally substituted —O—C₁₋₂₄ alkyl and an optionally substituted —O-aryl. In other embodiments, R⁷ can be

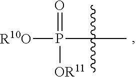

wherein R¹⁰ and R¹¹ can be each

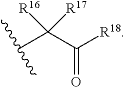

In various embodiments, when one or both of R¹⁰ and R¹¹ are

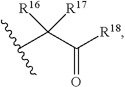

R¹⁶ and R¹⁷ can be each hydrogen; and R¹⁸ can be an unsubstituted C₁₋₂₄ alkyl. In other embodiments, at least one of R¹⁶ and R¹⁷ can be an optionally substituted C₁₋₂₄ alkyl or an optionally substituted aryl. In some embodiments, R⁸ can be an optionally substituted C₁₋₂₄ alkyl. In some embodiments, R¹⁸ can be an unsubstituted C₁₋₄ alkyl. In other embodiments, R¹⁸ can be an optionally substituted aryl. In still other embodiments, R¹⁸ can be an optionally substituted —O—C₁₋₂₄ alkyl, an optionally substituted —O-aryl, an optionally substituted —O-heteroaryl or an optionally substituted —O-monocyclic heterocyclyl. In some embodiments, R¹⁸ can be an unsubstituted —O—C₁₋₄ alkyl.

In some embodiments, R⁷ can be

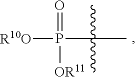

wherein one of R¹⁰ and R¹¹ can be absent, hydrogen or

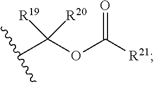

the other of R¹⁰ and R¹¹ can be

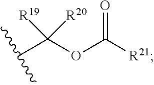

R¹⁹ and R²⁰ can be independently selected from hydrogen, an optionally substituted C₁₋₂₄ alkyl and an optionally substituted aryl; and R²¹ can be selected from hydrogen, an optionally substituted C₁₋₂₄ alkyl, an optionally substituted aryl, an optionally substituted —O—C₁₋₂₄ alkyl and an optionally substituted —O-aryl. In other embodiments, R⁷ can be

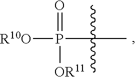

wherein R¹⁰ and R¹¹ can be each

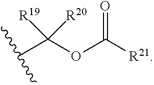

In various embodiments, when one or both of R¹⁰ and R¹¹ are

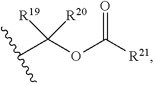

R¹⁹ and R²⁰ can be each hydrogen; and R²¹ can be an unsubstituted C₁₋₂₄ alkyl. In various other embodiments, when one or both of R¹⁰ and R¹¹ are

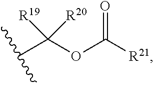

R¹⁹ and R²⁰ can be each hydrogen; and R²¹ can be an unsubstituted —O—C₁₋₂₄ alkyl. In some embodiments, R¹⁹ and R²⁰ can be hydrogen. In other embodiments, at least one of R¹⁹ and R²⁰ can be an optionally substituted C₁₋₂₄ alkyl or an optionally substituted aryl. In some embodiments, R²¹ can be an optionally substituted C₁₋₂₄ alkyl. In some embodiments, R²¹ can be an unsubstituted C₁₋₄ alkyl. In other embodiments, R²¹ can be an optionally substituted aryl. In still other embodiments, R²¹ can be an optionally substituted —O—C₁₋₂₄ alkyl, an optionally substituted —O-aryl, an optionally substituted —O— heteroaryl or an optionally substituted —O-monocyclic heterocyclyl. In some embodiments, R²¹ can be an unsubstituted —O—C₁₋₄ alkyl. In some embodiments, one or both of R¹⁰ and R¹¹ can be a pivaloyloxymethyl (POM) group. In some embodiments, R¹⁰ and R¹¹ can be each a pivaloyloxymethyl (POM) group, and form a bis(pivaloyloxymethyl) (bis(POM)) prodrug. In some embodiments, one or both of R¹⁰ and R¹¹ can be an isopropyloxycarbonyloxymethyl (POC) group. In some embodiments, R¹⁰ and R¹¹ each can be an isopropyloxycarbonyloxymethyl (POC) group, and form a bis(isopropyloxycarbonyloxymethyl) (bis(POC)) prodrug.

In some embodiments, R⁷ can be

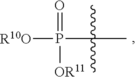

wherein one of R¹⁰ and R¹¹ can be absent, hydrogen or

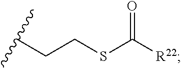

the other of R¹⁰ and R¹¹ can be

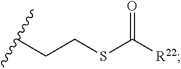

and R²² can be selected from hydrogen, an optionally substituted C₁₋₂₄ alkyl and an optionally substituted aryl. In other embodiments, R⁷ can be

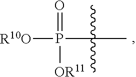

wherein R¹⁰ and R¹¹ can be each

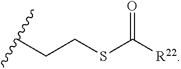

In various embodiments, R²² can be a substituted C₁₋₂₄ alkyl. In various other embodiments, R²² can be an unsubstituted C₁₋₂₄ alkyl. In still various other embodiments, R²² can be an unsubstituted C₁₋₄ alkyl. In some embodiments, R¹⁰ and R¹¹ can be each a S-acylthioethyl (SATE) group and form a SATE ester prodrug. In some embodiments, R¹⁰ and R¹¹ can be each

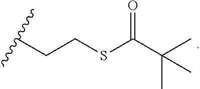 [SMILES: *CCSC(=O)C(C)(C)C]

In some embodiments, R⁷ can be

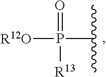

wherein R¹² can be absent, hydrogen, an optionally substituted aryl or an optionally substituted heteroaryl; and R¹³ can be an optionally substituted N-linked α-amino acid or an optionally substituted N-linked α-amino acid ester derivative. In some embodiments, R¹² can be an optionally substituted phenyl. In other embodiments, R¹² can be an optionally substituted naphthyl. In still other embodiments, R¹² can be an unsubstituted phenyl. In yet still other embodiments, R¹² can be an unsubstituted naphthyl. In some embodiments, R¹² can be an optionally substituted heteroaryl, such as an optionally substituted monocyclic heteroaryl.

In some embodiments, R¹³ can be an optionally substituted N-linked α-amino acid. In some embodiments, R¹³ can be an optionally substituted N-linked α-amino acid ester derivative. Various α-amino acids are known to those skilled in the art, and include alanine, asparagine, aspartate, cysteine, glutamate, glutamine, glycine, proline, serine, tyrosine, arginine, histidine, isoleucine, leucine, lysine, methionine, phenylalanine, threonine, tryptophan and valine. The ester derivatives of the N-linked α-amino acid ester derivative can have one of the following structures: C₁₋₆ alkyl-O—C(═O)—, C₃₋₆ cycloalkyl-O—C(═O)—, phenyl-O—C(═O)—, naphthyl-O—C(═O)— and benzyl-O—C(═O)—. In some embodiments, the N-linked α-amino acid ester derivative can be a C₁₋₆ alkyl, C₃₋₆ cycloalkyl, phenyl, naphthyl or benzyl ester of alanine, asparagine, aspartate, cysteine, glutamate, glutamine, glycine, proline, serine, tyrosine, arginine, histidine, isoleucine, leucine, lysine, methionine, phenylalanine, threonine, tryptophan or valine. In some embodiments, R¹³ can be N-linked alanine, N-linked alanine isopropyl ester, N-linked alanine cyclohexyl ester or N-linked alanine neopentyl ester. In some embodiments, R¹² can be an unsubstituted phenyl; and R¹³ can be C₁₋₆ alkyl-O—C(═O)—, C₃₋₆ cycloalkyl-O—C(═O)—, phenyl-O—C(═O)—, naphthyl-O—C(═O)— or benzyl-O—C(═O)— ester of N-linked alanine, N-linked glycine, N-valine, N-linked leucine or N-linked isoleucine. In some embodiments, when R⁷ is

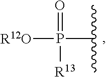

a compound of Formula (I), or a pharmaceutically acceptable salt thereof, can be a phosphoramidate prodrug, such as an aryl phosphoramidate prodrug.

In some embodiments, R⁷ can be

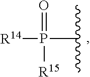

wherein R¹⁴ and R¹⁵ can be independently an optionally substituted N-linked α-amino acid ester derivative. In various embodiments, the α-amino acid portion of the optionally substituted N-linked α-amino acid ester derivative can be selected from alanine, asparagine, aspartate, cysteine, glutamate, glutamine, glycine, proline, serine, tyrosine, arginine, histidine, isoleucine, leucine, lysine, methionine, phenylalanine, threonine, tryptophan and valine. As described herein, the ester portion of the α-amino acid ester derivative can have various structures. In some embodiments, the ester portion of the N-linked α-amino acid ester derivative can have one of the following structures: C₁₋₆ alkyl-O—C(═O)—, C₃₋₆ cycloalkyl-O—C(═O)—, phenyl-O—C(═O)—, naphthyl-O—C(═O)— and benzyl-O—C(═O)—. In some embodiments, R¹⁴ and R¹⁵ can be independently selected from N-linked alanine, N-linked alanine isopropyl ester, N-linked alanine cyclohexyl ester or N-linked alanine neopentyl ester. In some embodiments, R¹⁴ and R¹⁵ can be each independently C₁₋₆ alkyl-O—C(═O)—, C₃₋₆ cycloalkyl-O—C(═O)—, phenyl-O—C(═O)—, naphthyl-O—C(═O)— or benzyl-O—C(═O)— ester of N-linked alanine, N-linked glycine, N-valine, N-linked leucine or N-linked isoleucine. In some embodiments, R¹⁴ and R¹⁵ can be the same. In other embodiments, R¹⁴ and R¹⁵ can be different. In some embodiments, when R⁷ can be

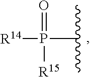

a compound of Formula (I), or a pharmaceutically acceptable salt thereof, can be an optionally substituted phosphonic diamide prodrug.

Examples of suitable N-linked α-amino acid ester derivative groups that can present at R³, R¹⁴ and/or R¹⁵ include the following:

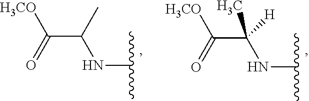

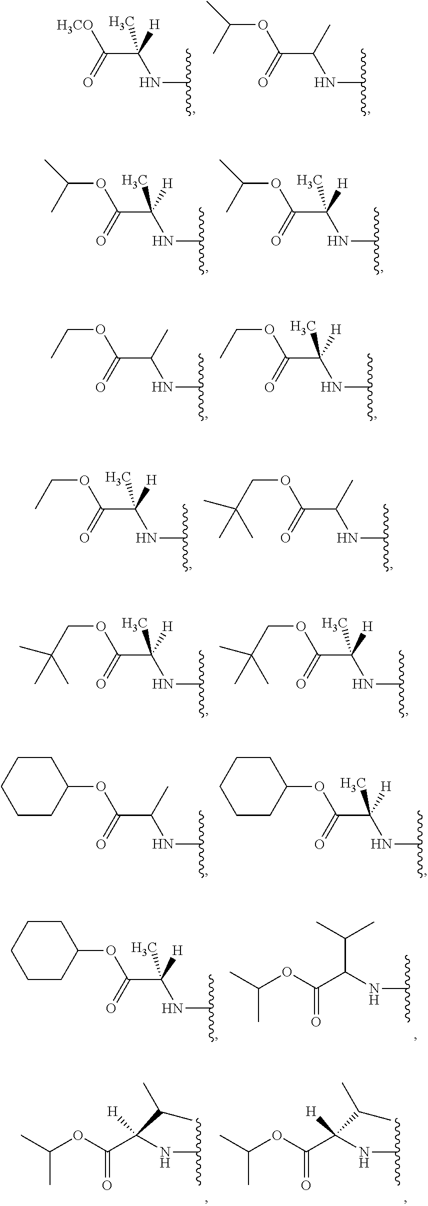

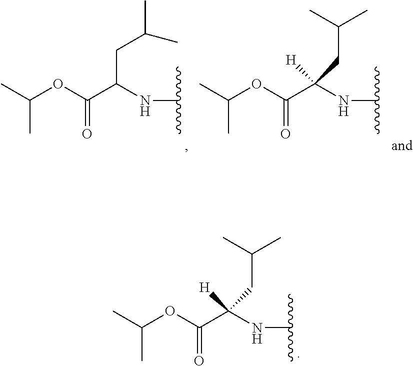

The heterocyclic base, B¹, present on a compound of Formula (I), or a pharmaceutically acceptable salt thereof, can be attached through a nitrogen (an optionally substituted N-linked heterocyclic base) or a carbon (an optionally substituted C-linked heterocyclic base). In some embodiments, B¹ can be an optionally substituted N-linked heterocyclic base. In some embodiments, B¹ can be an optionally substituted C-linked heterocyclic base.

When B¹ is an optionally substituted N-linked heterocyclic base, B¹ can be in various embodiments, an optionally substituted purine. In other various embodiments, B¹ can be an optionally substituted pyrimidine. In some embodiments, B¹ can be a substituted guanine, a substituted adenine, a substituted thymine, a substituted cytosine or a substituted uracil. In other embodiments, B¹ can be an unsubstituted guanine, an unsubstituted adenine, an unsubstituted thymine, an unsubstituted cytosine or an unsubstituted uracil.

In some embodiments, B¹ can be selected from:

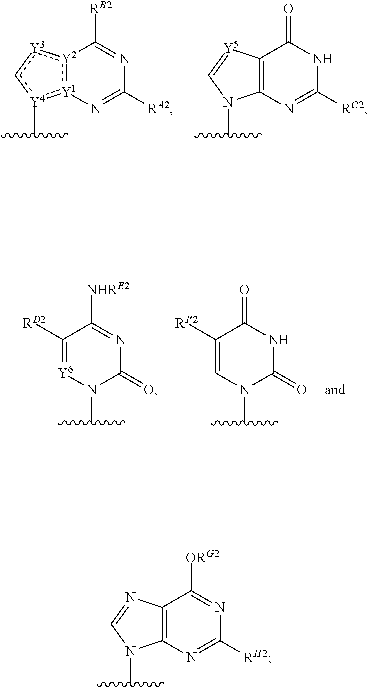

wherein: R^{A2} can be selected from hydrogen, halogen and NHR^{J2}, wherein R^{J2} can be selected from hydrogen, —C(═O)R^{K2} and —C(═O)OR^{L2}; R^{B2} can be halogen or NHR^{W2}, wherein R^{W2} can be selected from hydrogen, an optionally substituted C₁₋₆ alkyl, an optionally substituted C₂₋₆ alkenyl, an optionally substituted C₃₋₈ cycloalkyl, —C(═O)R^{M2} and —C(═O)OR^{N2}; R^{C2} can be hydrogen or NHR^{O2}, wherein R^{O2} can be selected from hydrogen, —C(═O)R^{P2} and —C(═O)OR^{Q2}; R^{D2} can be selected from hydrogen, deuterium, halogen, an optionally substituted C₁₋₆ alkyl, an optionally substituted C₂₋₆ alkenyl and an optionally substituted C₂₋₆ alkynyl; R^{E2} can be selected from hydrogen, hydroxy, an optionally substituted C₁₋₆ alkyl, an optionally substituted C₃₋₈ cycloalkyl, —C(═O)R^{R2} and —C(═O)OR^{S2}; R^{F2} can be selected from hydrogen, halogen, an optionally substituted C₁₋₆ alkyl, an optionally substituted C₂₋₆ alkenyl and an optionally substituted C₂₋₆ alkynyl; Y¹, Y² and Y⁴ can be independently N (nitrogen) or C (carbon), provided that at least one of Y¹, Y² and Y⁴ is N; Y³ can be N (nitrogen) or CR^{I2}, wherein R¹² can be selected from hydrogen, halogen, an unsubstituted C₁₋₆-alkyl, an unsubstituted C₂₋₆-alkenyl and an unsubstituted C₂₋₆-alkynyl; Y⁵ and Y⁶ can be independently N (nitrogen) or CH; each [symbol] can be independently a single or double bond, provided that the single bonds and the double bonds are situated in the ring so that each ring is aromatic; R^{G2} can be an optionally substituted C₁₋₆ alkyl; R^{H2} can be hydrogen or NHR^{T2}, wherein R^{T2} can be independently selected from hydrogen, —C(═O)R^{U2} and —C(═O)OR^{V2}; and R^{K2}, R^{L2}, R^{M2}, R^{N2}, R^{P2}, R^{Q2} R^{R2}, R^{S2}, R^{U2} and R^{V2} can be independently selected from an unsubstituted C₁₋₆ alkyl, an unsubstituted C₂₋₆ alkenyl, an unsubstituted C₂₋₆ alkynyl, an optionally substituted C₃₋₆ cycloalkyl, an optionally substituted C₃₋₆ cycloalkenyl, an optionally substituted C₆₋₁₀ aryl, an optionally substituted heteroaryl, an optionally substituted heterocyclyl, an optionally substituted aryl(C₁₋₆ alkyl), an optionally substituted heteroaryl(C₁₋₆ alkyl) and an optionally substituted heterocyclyl(C₁₋₆ alkyl).

Examples of suitable B¹ groups include the following:

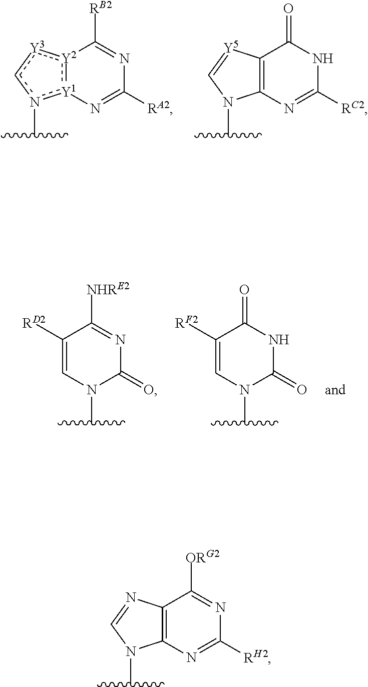

wherein R^{A2}, R^{B2}, R^{C2}, R^{D2}, R^{E2}, R^{F2}, R^{G2}, R^{H2}, Y¹, Y², Y³ and Y⁵ are provided herein. In some embodiments, B¹ can be

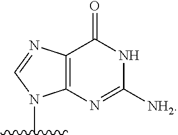 [SMILES: *n1cnc2c(=O)[nH]c(N)nc21]

In other embodiments, B¹ can be

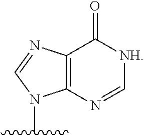 [SMILES: *n1cnc2c(=O)[nH]cnc21]

In still other embodiments, B¹ can be

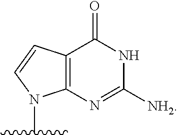 [SMILES: *n1ccc2c(=O)[nH]c(N)nc21]

In yet still other embodiments, B¹ can be

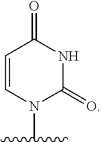 [SMILES: *n1ccc(=O)[nH]c1=O]

In some embodiments, B¹ can be

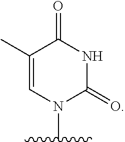 [SMILES: *n1cc(C)c(=O)[nH]c1=O]

In other embodiments, B¹ can be

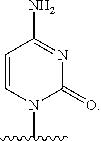 [SMILES: *n1ccc(N)nc1=O]

In still other embodiments, B¹ can be

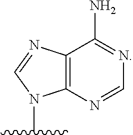 [SMILES: *n1cnc2c(N)ncnc21]

In yet still other embodiments, B¹ can be

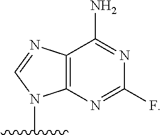 [SMILES: *n1cnc2c(N)nc(F)nc21]

In some embodiments, B¹ can be

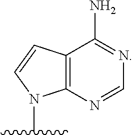 [SMILES: *n1ccc2c(N)ncnc21]

In other embodiments, B¹ can be

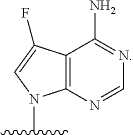 [SMILES: *n1cc(F)c2c(N)ncnc21]

In still other embodiments, B¹ can be

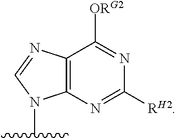

### When B¹ is

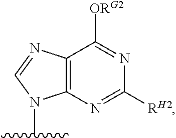

in various embodiments, R^{G2} can be an unsubstituted ethyl and R^{H2} can be NH₂.

When B¹ is an optionally substituted C-linked heterocyclic base, in various embodiments, B¹ can have the structure

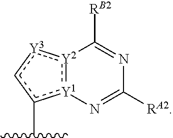

In some embodiments, B¹ can be selected from

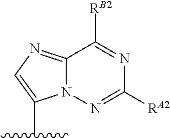

and

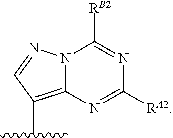

For example, B¹ can be

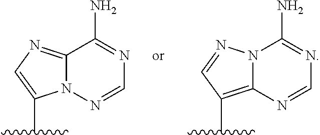 [SMILES: *c1cnc2c(N)ncnn12 ; *c1cnn2c(N)ncnc12]

In some embodiments, a compound of Formula (I), or a pharmaceutically acceptable salt thereof, can have a structure selected from:

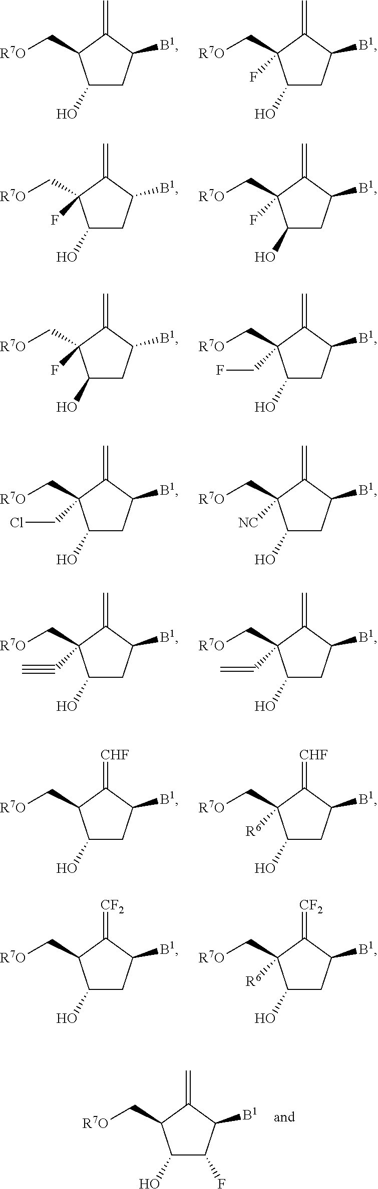

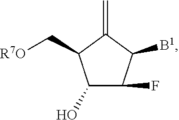

or a pharmaceutically acceptable salt of any of the foregoing. In some embodiments of this paragraph, B¹ can be an optionally substituted N-linked heterocyclic base. In some embodiments of this paragraph, B¹ can be an optionally substituted C-linked heterocyclic base. In some embodiments of this paragraph, B¹ can be an optionally substituted purine base. In other embodiments of this paragraph, B¹ can be an optionally substituted pyrimidine base. In some embodiments of this paragraph, B¹ can be guanine. In other embodiments of this paragraph, B¹ can be thymine. In still other embodiments of this paragraph, B¹ can be cytosine. In yet still other embodiments of this paragraph, B¹ can be uracil. In some embodiments of this paragraph, B¹ can be adenine. In some embodiments of this paragraph, R⁷ can be hydrogen. In other embodiments of this paragraph, R⁷ can be an optionally substituted acyl. In still other embodiments of this paragraph, R⁷ can be mono-, di- or tri-phosphate. In yet other embodiments of this paragraph, R⁷ can be phosphoramidate prodrug, such as an aryl phosphoramidate prodrug. In some embodiments of this paragraph, R⁷ can be an acyloxyalkyl ester phosphate prodrug. In other embodiments of this paragraph, R⁷ can be a S-acylthioethyl (SATE) prodrug. In still other embodiments, R⁷ can be a phosphonic diamide prodrug. In some embodiments of this paragraph, R⁷ can be an optionally substituted O-linked α-amino acid, such as one of those described herein. In some embodiments of this paragraph, R⁶ can be selected from halogen, cyano, an optionally substituted C₁₋₄ alkyl, an optionally substituted C₂₋₄ alkenyl and an unsubstituted C₂₋₄ alkynyl, wherein when the C₁₋₄ alkyl or the C₂₋₄ alkenyl are substituted, the C₁₋₄ alkyl and C₂₋₄ alkenyl are independently substituted with at least one halogen. In some embodiments of this paragraph, R⁶ can be selected from fluoro, cyano, an unsubstituted C₁₋₄ alkyl, —(CH₂)₁₋₄F (such as —CH₂F), —(CH₂)₁₋₄C₁ (such as —CH₂Cl), an unsubstituted C₂₋₄ alkenyl and an unsubstituted C₂₋₄ alkynyl.

Examples of suitable compounds of Formula (I), or a pharmaceutically acceptable salt thereof, include, but are not limited to the following:

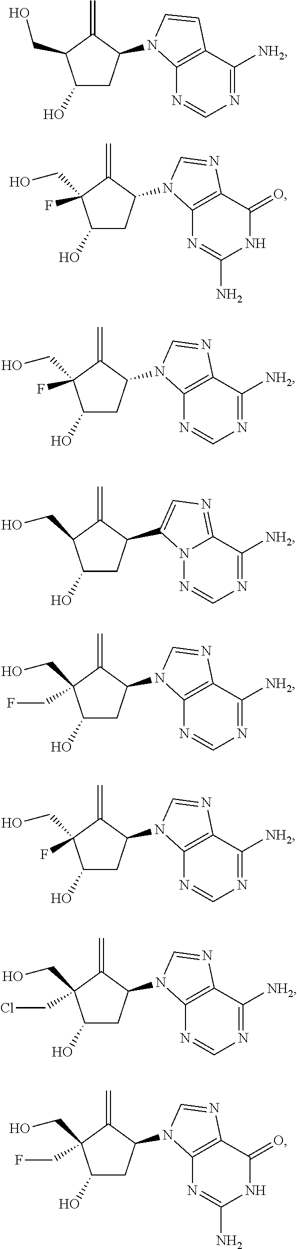

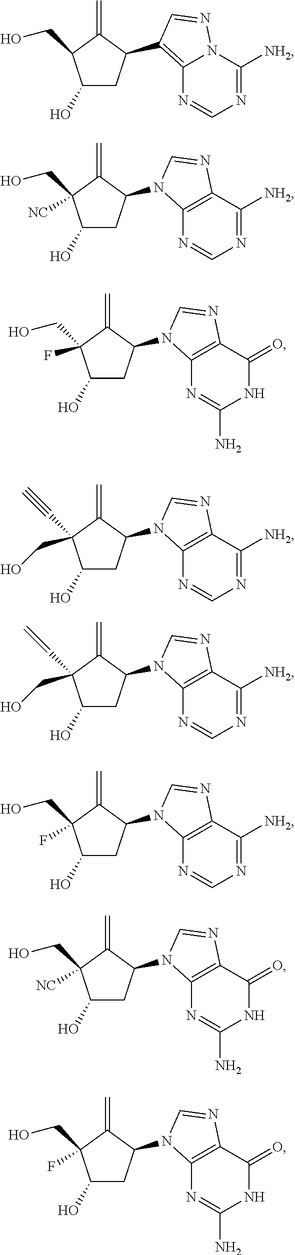

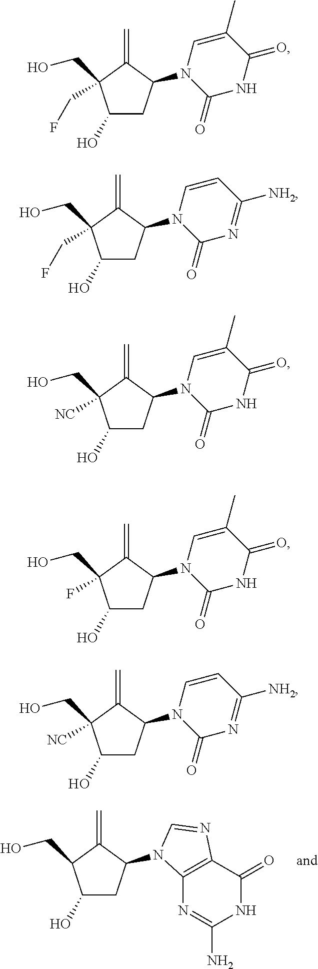

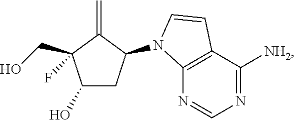 [SMILES: C=C1[C@@H](n2ccc3c(N)ncnc32)C[C@H](O)[C@@]1(F)CO]

or a pharmaceutically acceptable salt of any of the foregoing.

Additional examples of suitable compounds of Formula (I) include, but are not limited to the following:

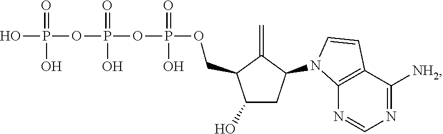 [SMILES: C=C1[C@H](COP(=O)(O)OP(=O)(O)OP(=O)(O)O)[C@@H](O)C[C@@H]1n1ccc2c(N)ncnc21]

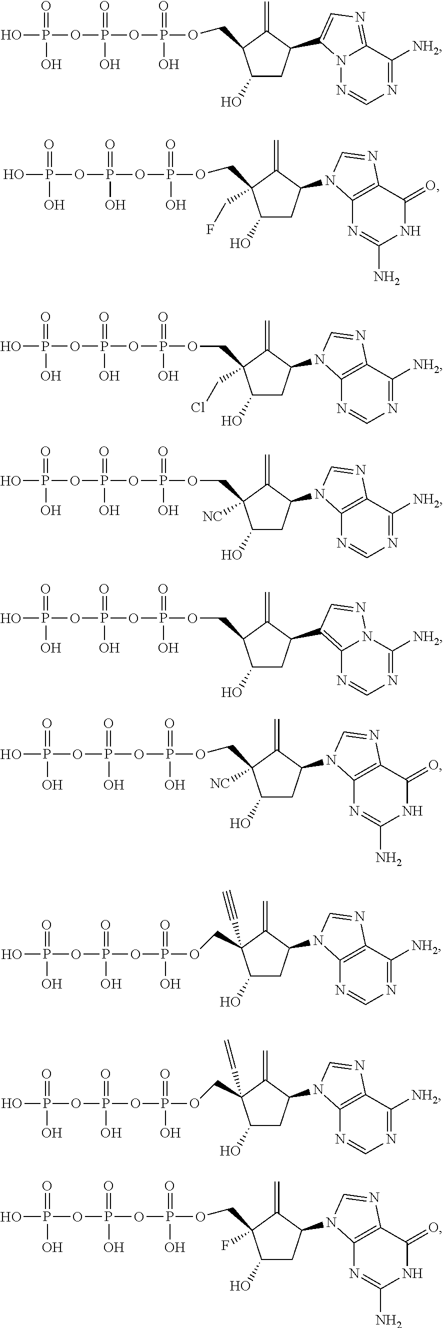

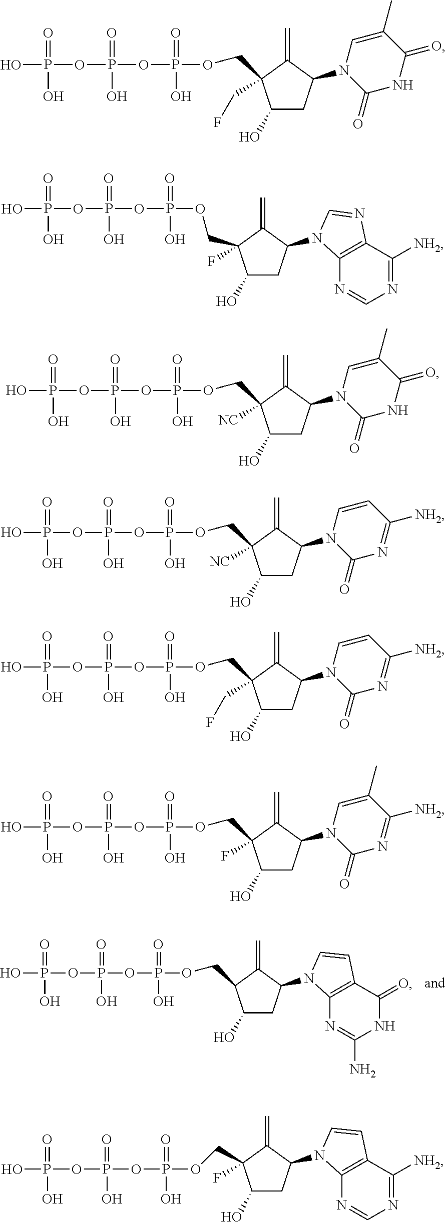

or a pharmaceutically acceptable salt of any of the foregoing.

Even further examples of suitable compounds of Formula (I) include, but are not limited to the following:

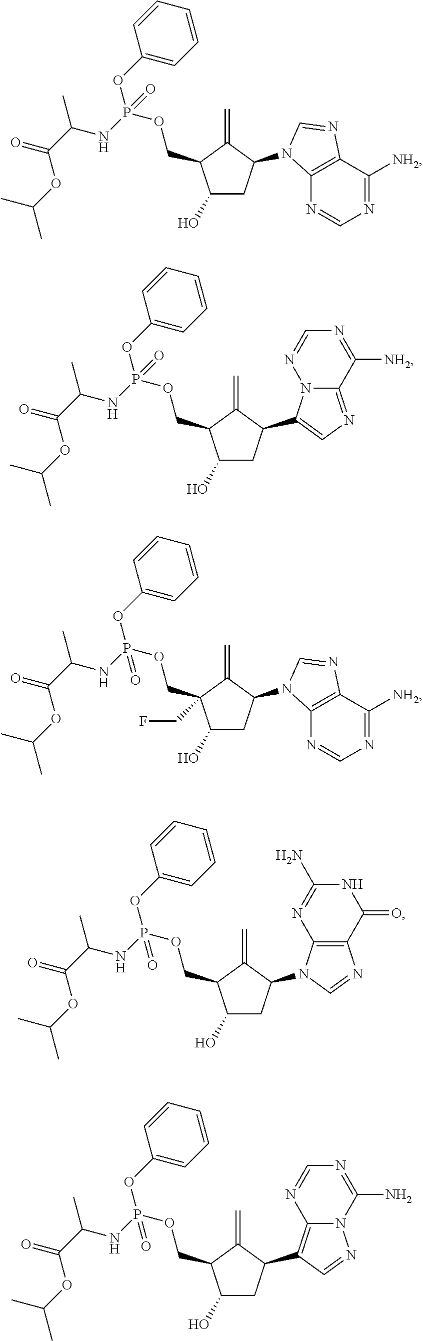 [SMILES: C=C1[C@H](COP(=O)(NC(C)C(=O)OC(C)C)Oc2ccccc2)[C@@H](O)C[C@@H]1n1cnc2c(N)ncnc21 ; C=C1[C@@H](c2cnc3c(N)ncnn23)C[C@H](O)[C@H]1COP(=O)(NC(C)C(=O)OC(C)C)Oc1ccccc1 ; C=C1[C@@H](n2cnc3c(N)ncnc32)C[C@H](O)[C@@]1(CF)COP(=O)(NC(C)C(=O)OC(C)C)Oc1ccccc1 ; C=C1[C@H](COP(=O)(NC(C)C(=O)OC(C)C)Oc2ccccc2)[C@@H](O)C[C@@H]1n1cnc2c(=O)[nH]c(N)nc21 ; C=C1[C@@H](c2cnn3c(N)ncnc23)C[C@H](O)[C@H]1COP(=O)(NC(C)C(=O)OC(C)C)Oc1ccccc1]

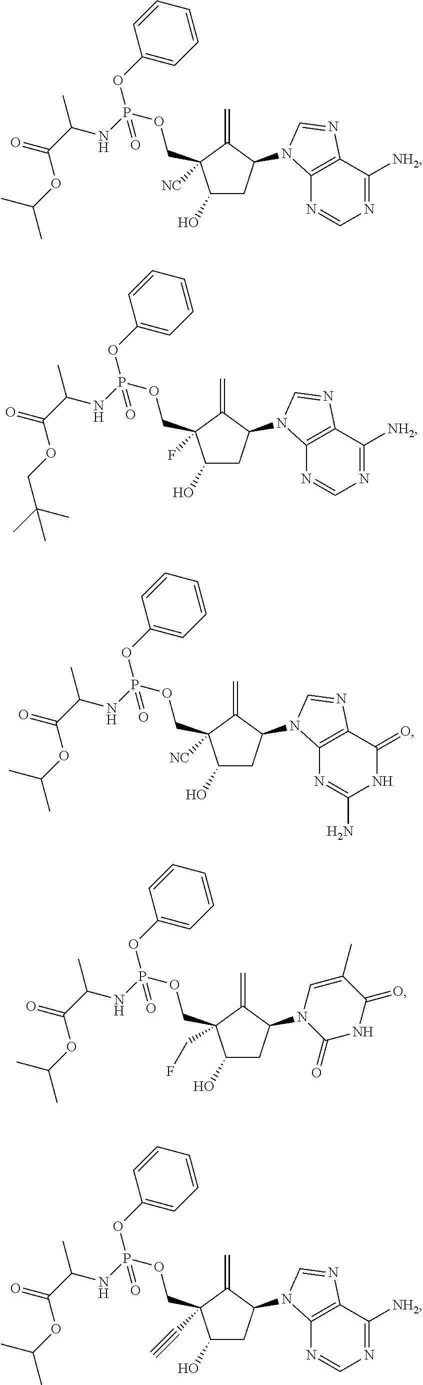 [SMILES: C=C1[C@@H](n2cnc3c(N)ncnc32)C[C@H](O)[C@@]1(C#N)COP(=O)(NC(C)C(=O)OC(C)C)Oc1ccccc1 ; C=C1[C@@H](n2cnc3c(N)ncnc32)C[C@H](O)[C@@]1(F)COP(=O)(NC(C)C(=O)OCC(C)(C)C)Oc1ccccc1 ; C=C1[C@@H](n2cnc3c(=O)[nH]c(N)nc32)C[C@H](O)[C@@]1(C#N)COP(=O)(NC(C)C(=O)OC(C)C)Oc1ccccc1 ; C=C1[C@@H](n2cc(C)c(=O)[nH]c2=O)C[C@H](O)[C@@]1(CF)COP(=O)(NC(C)C(=O)OC(C)C)Oc1ccccc1 ; C#C[C@]1(COP(=O)(NC(C)C(=O)OC(C)C)Oc2ccccc2)C(=C)[C@@H](n2cnc3c(N)ncnc32)C[C@@H]1O]

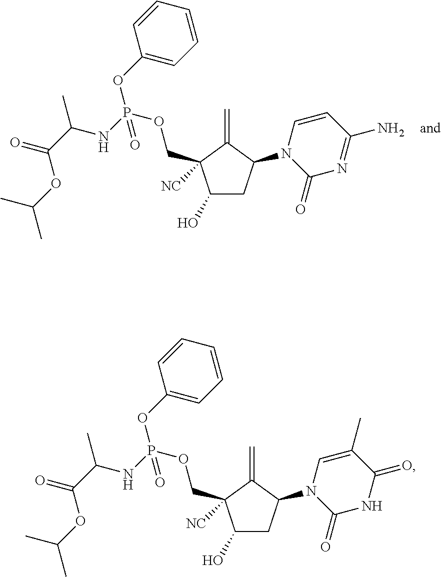 [SMILES: C=C1[C@@H](n2ccc(N)nc2=O)C[C@H](O)[C@@]1(C#N)COP(=O)(NC(C)C(=O)OC(C)C)Oc1ccccc1 ; C=C1[C@@H](n2cc(C)c(=O)[nH]c2=O)C[C@H](O)[C@@]1(C#N)COP(=O)(NC(C)C(=O)OC(C)C)Oc1ccccc1]

or a pharmaceutically acceptable salt of any of the foregoing.

In some embodiments, a compound of Formula (I), or a pharmaceutically acceptable salt thereof, is not a compound of (i), or a pharmaceutically acceptable salt thereof, wherein when R¹ is hydrogen; R² is hydrogen or fluoro; R³ is hydrogen or fluoro; R⁴ is hydroxy; R⁵ is hydrogen; R⁶ is hydrogen or fluoro; R⁸ and R⁹ are each hydro en and B¹ is selected from the group consisting of

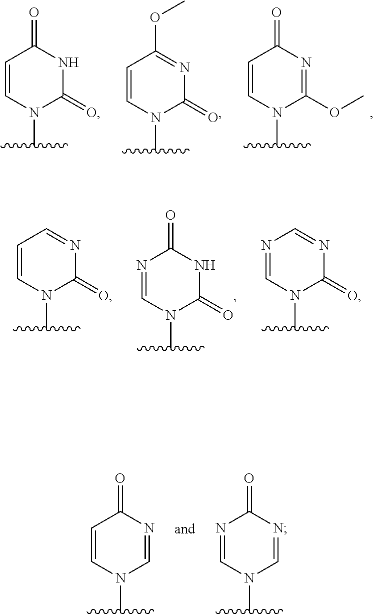

then R⁷ is not selected from the group consisting of: (a) hydrogen; (b)

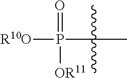

wherein R¹⁰ and R¹¹ are each hydrogen or each absent; (c)

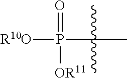

wherein R¹⁰ is

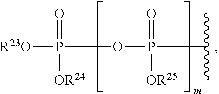

R¹¹, R²³, R²⁴ or R²⁵ are independently absent or hydrogen, and m is 0 or 1; and (d)

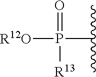

wherein R¹² is an unsubstituted phenyl or an unsubstituted naphthyl, and R¹³ is alanine isopropyl ester, alanine isobutyl ester or alanine neopentyl ester. In some embodiments, a compound of Formula (I), or a pharmaceutically acceptable salt thereof, is not a compound of (ii), or a pharmaceutically acceptable salt thereof, when R¹ is hydrogen; R⁴ is hydroxy; R⁵ is hydrogen; R⁶ is hydrogen; R⁸ and R⁹ are each hydrogen; B¹ is selected from the group consisting of

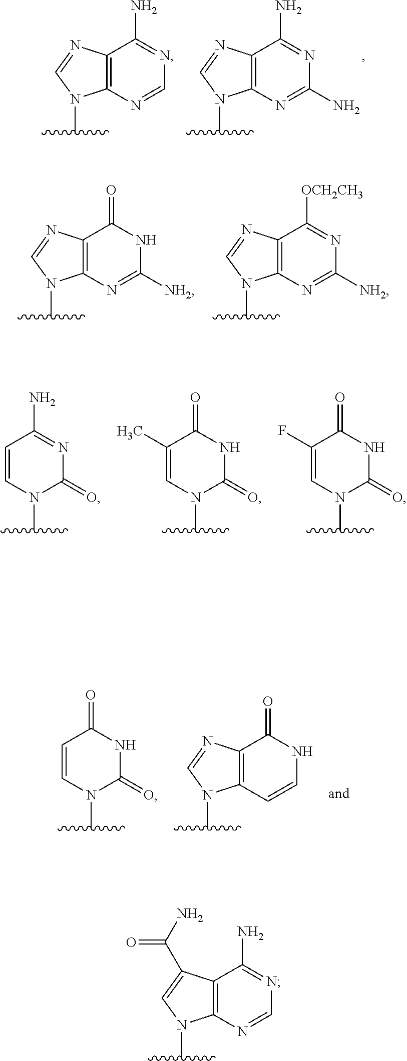

R⁷ is selected from the group consisting of: (a)

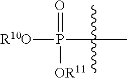

wherein R¹⁰ is

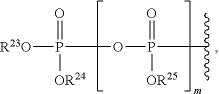

R¹¹, R²³, R²⁴ or R²⁵ are independently absent or hydrogen, and m is 1; and (b)

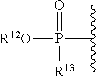

wherein R¹² is an unsubstituted phenyl, and R¹³ is alanine isopropyl ester, then (a) R² is not hydrogen when R³ are fluoro; and (b) R² is not fluoro when R³ are hydrogen. In some embodiments, a compound of Formula (I), or a pharmaceutically acceptable salt thereof, is not a compound of (iii), or a pharmaceutically acceptable salt thereof, when R¹ is hydrogen; R⁴ is hydroxy; R⁵ is hydrogen; R⁶ is hydrogen; R⁷ is hydrogen and R⁸ and R⁹ are each hydrogen; then B¹ is not selected from the group consisting of

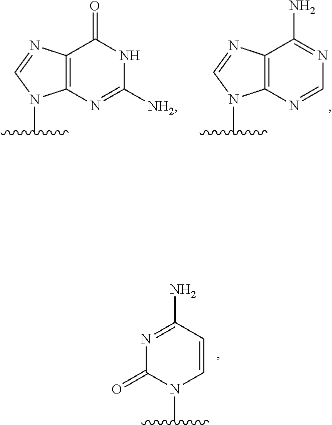 [SMILES: *n1cnc2c(=O)[nH]c(N)nc21 ; *n1cnc2c(N)ncnc21 ; *n1ccc(N)nc1=O]

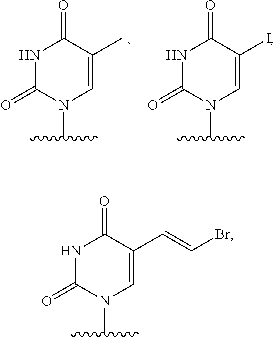

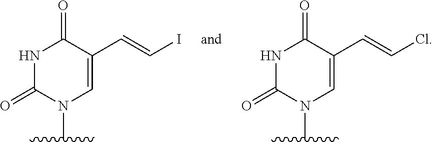 [SMILES: *n1cc(/C=C/I)c(=O)[nH]c1=O ; *n1cc(/C=C/Cl)c(=O)[nH]c1=O]

In some embodiments, B¹ is not uridine. In some embodiments, B¹ is not cytosine. In some embodiments, B¹ is not thymidine. In some embodiments, B¹ is not guanine. In some embodiments, B¹ is not adenine. In some embodiments, B¹ is not a substituted uridine. In some embodiments, R⁸ and R⁹ are not each hydrogen. In some embodiments, R⁷ is not

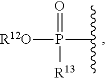

wherein R¹² is a substituted or unsubstituted phenyl or a substituted or unsubstituted naphthyl. In some embodiments, R⁷ is not

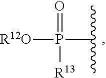

wherein R¹² is an unsubstituted phenyl or an unsubstituted naphthyl; and R¹³ is N-linked alanine or an ester derivative of N-linked alanine (such as N-linked alanine isopropyl ester, N-linked alanine isobutyl ester and N-linked alanine neopentyl ester). In some embodiments, R⁷ is hydrogen. In some embodiments, R² is not hydrogen. In some embodiments, R² is not fluoro. In some embodiments, R³ is not hydrogen. In some embodiments, R³ is not fluoro. In some embodiments, R² is not hydrogen when R³ is fluoro. In some embodiments, R² is not fluoro when R³ is hydrogen. In some embodiments, R² and R³ are not each fluoro. In some embodiments, R⁶ is not hydrogen. In some embodiments, R⁶ is not halogen (for example, fluoro). In some embodiments, R⁵ is not hydroxy. In some embodiments, a compound of Formula (I), or a pharmaceutically acceptable salt thereof, is not a compound, or a pharmaceutically acceptable salt thereof, provide in WO 2017/040895 (published Mar. 9, 2017), WO 2017040892 (published Mar. 9, 2017), WO 2016/182936 (published Nov. 17, 2016), WO 2016/182935 (published Nov. 17, 2016), WO 2016/134056 (published Aug. 25, 2016), WO 2016/134054 (published Aug. 25, 2016), WO 2017/165489 (published Sep. 28, 2017) WO 2015/077360 (published May 28, 2015), U.S. Publication No. 2016/0280729 (published Sep. 29, 2016), WO 2012/094248 (published Jul. 12, 2012), U.S. Pat. No. 9,156,874 (issued Aug. 15, 2013), WO 2012/094248 (published Jul. 12, 2012), U.S. Pat. No. 9,095,599 (issued Aug. 4, 2015), U.S. Publication No. US 2014/0038916 (published Feb. 6, 2014), WO 2010/091386 (published Aug. 12, 2010), U.S. Pat. No. 8,609,627 (issued Dec. 17, 2013), U.S. Pat. No. 9,173,893 (issued Nov. 3, 2015), WO 2010/036407 (published Apr. 1, 2010), WO 2010/030858 (published Mar. 18, 2010), U.S. Pat. No. 8,163,707 (issued Apr. 24, 2012), WO 2008/089105 (published Jul. 24, 2008), U.S. Pat. No. 8,440,813 (issued May 14, 2013), WO 2003/072757 (published Sep. 4, 2003), U.S. Pat. No. 7,285,658 (issued Oct. 23, 2007), U.S. Pat. No. 7,598,230 (Issued Oct. 6, 2009) and/or U.S. Pat. No. 7,807,653 (issued Oct. 5, 2010).

Exemplary compounds useful in methods provided herein are described by reference to the illustrative synthetic schemes for their general preparation below and the specific examples that follow. One skilled in the art will recognize that, to obtain the various compounds herein, starting materials may be suitably selected so that the ultimately desired substituents will be carried through the reaction scheme with or without protection as appropriate to yield the desired product. Alternatively, it may be necessary or desirable to employ, in the place of the ultimately desired substituent, a suitable group that may be carried through the reaction scheme and replaced as appropriate with the desired substituent. Unless otherwise specified, the variables are as defined above in reference to Formula (I). Reactions may be performed between the melting point and the reflux temperature of the solvent, and preferably between 0° C. and the reflux temperature of the solvent. Reactions may be heated employing conventional heating or microwave heating. Reactions may also be conducted in sealed pressure vessels above the normal reflux temperature of the solvent.

Exemplary compounds useful in methods provided herein are described by reference to the illustrative synthetic schemes for their general preparation below and the specific examples to follow.

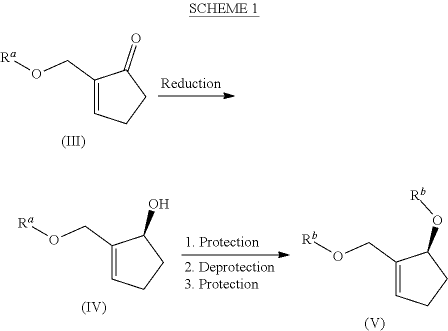

According to SCHEME 1, a chiral cyclopentanol compound of formula (IV) is prepared by a symmetric reduction of a commercially available or synthetically accessible cyclopentenone compound of formula (III), where R^{a} is trityl (triphenylmethyl). For example, a catalytic chiral catalyst such as methyl-CBS catalyst and borohydride reducing agent such as borane dimethylsulfide (BMS: BH₃.Me₂S) gives rise to (S)-2-((trityloxy)methyl)cyclopent-2-en-1-ol. Benzyl protection of a compound of formula (IV), employing benzyl bromide or benzyl chloride, and a suitable alkali metal hydride base such as NaH, KH, or LiH, preferably NaH; in a suitable solvent such as DMF and the like; tetrabutylammonium iodide (TBAI); at temperatures ranging from 0° C. to room temperature; provides the benzyl protected alcohol. Deprotection of the trityl protecting group employing trifluoroacetic acid and triethylsilane, followed by subsequent Bn protection affords the bis-benzyl protected compound of formula (V), where R^{b} is Bn.

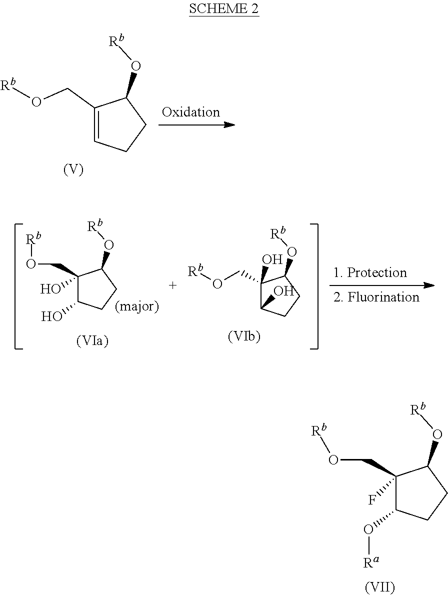

According to SCHEME 2, the oxidation of carbon-carbon double bond of a compound of formula (V), where R^{b} is Bn, is carried out with an oxidizing agent which provides selective hydroxylation of the carbon-carbon double bond to provide the 1,2-diol compounds of formula (VIa) and (VIb). Preferably, dihydroxylation is achieved using osmium tetroxide as a catalyst and an oxidant such as NMO (N-methyl morpholine-N-Oxide) in a solvent mixture such as THF/water, at ambient temperature. Trityl protection of the alcohol mixture of compounds of formulas (VIa and VIb) is achieved according to procedures known to one skilled in the art, for example, using [chloro(diphenyl)methyl]benzene (triphenylmethyl chloride, trityl chloride or (TrCl)); a catalyst such as AgNO₃; a tertiary organic base such as pyridine, collidine, and the like; in a suitable solvent such as THF, and the like. Subsequent fluorination by treatment with a fluorinating agent such as DAST, in a suitable solvent such as DCM, and the like, provides a compound of formula (VII), where R^{b} is Bn, and R^{a} is Trt.

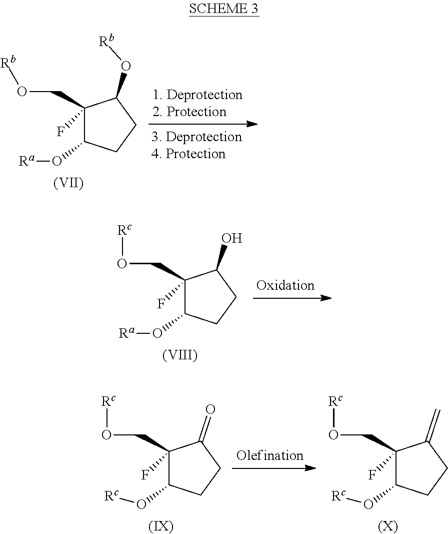

According to SCHEME 3, employing methods known to one skilled in the art, a series of deprotection and re-protection steps of a compound of formula (VII), where R^{b} is Bn, and R^{a} is Trt, affords a compound of formula (VIII), where R^{c} is TBS. For example, deprotection of the Trt is achieved employing trifluoroacetic acid/triethyl silane; TBS protection of the corresponding alcohol is achieved employing TBS-C₁/imidazole; Bn deprotection is achieved under hydrogenolytic conditions; and TBS protection to finish. Oxidation of an alcohol compound of formula (VIII), where R^{c} is TBS, is achieved with a suitable oxidizing agent, such as Dess-Martin periodinane. In a preferred embodiment, a compound of formula (VIII) is treated with Dess-Martin periodinane; in a suitable solvent such as dichloromethane, and the like; at temperatures ranging from about 0° C. to about 25° C.; for a period of approximately 0.5 to 4 hours; to produce a compound of formula (IX). An olefin compound of formula (X) is prepared from a compound of formula (IX) using an olefinating agent such as Tebbe's reagent or a Wittig type reagent such as methyltriphenylphosphonium bromide; with a base such as potassium t-butoxide, potassium tert-pentoxide, and the like; in an organic solvent such as THF, toluene, and the like. In a preferred method, the solvent is toluene and the base is potassium tert-pentoxide.

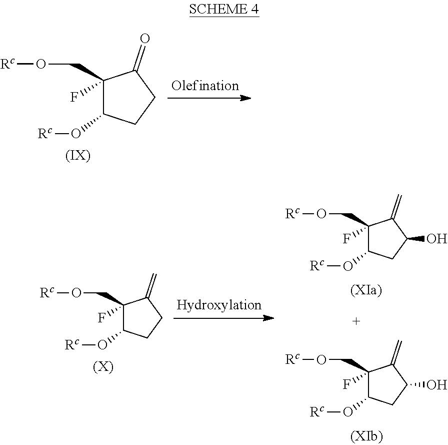

According to SCHEME 4, a compound of formula (IX), where R^{C} is TBS, is reacted in a Wittig type olefination reaction as previously described to provide an olefin compound of formula (X). Hydroxylation of a compound of formula (X), is preferably carried out in a suitable solvent with selenium dioxide as oxidizing agent. The reaction is conducted with or without the presence of a hydroperoxide, e.g. hydrogen peroxide or an alkyl hydroperoxide, e.g. t-butylhydroperoxide (TBHP); in a suitable solvent such as dichloromethane, chloroform, or pyridine, or mixtures thereof; at a temperature ranging from about 0° to 25° C.; for a period of about 2 to 24 h; to provide compounds of formula (XIa) and (XIb). A compound of formula (XIa) is converted to a compound of (XIb) by first protection of the hydroxyl moiety with a para-nitrobenzoic acid (PNBA) protecting group, then deprotection with ammonia/MeOH.

In a similar fashion (2R,3S)-3-(benzyloxy)-2-((benzyloxy)methyl)-2-fluorocyclopentan-1-one is reacted in a Wittig type olefination reaction as previously described to provides ((((1R,2S)-2-(benzyloxy)-1-fluoro-5-methylenecyclopentyl)methoxy)methyl)benzene. Subsequent hydroxylation employing conditions previously described provides (3R,4S)-4-(benzyloxy)-3-((benzyloxy)methyl)-3-fluoro-2-methylenecyclopentan-1-ol.

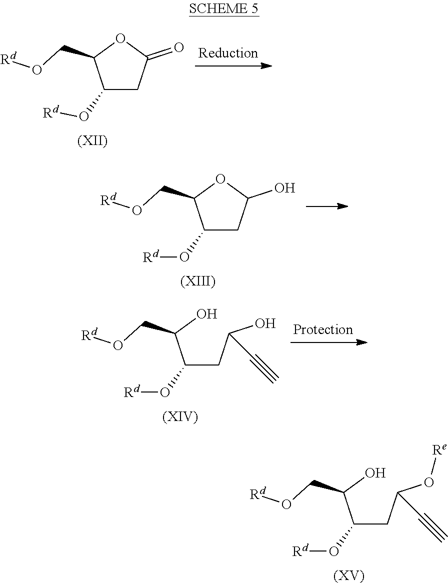

According to SCHEME 5, a commercially available or synthetically accessible compound of formula (XII), where R^{d} is a protecting group such as pivaloyl (Piv), TIPS (triisopropylsilyl), and the like, is reduced employing conditions known to one skilled the art. For example, a compound of formula (XII) is reduced employing a reducing agent such as diisobutylaluminium hydride (DIBAL-H); in a suitable solvent such as THF and the like; at a temperature of about −70° C.; to provide a lactol compound of formula (XIII), where R^{d} is Piv or TIPS (triisopropylsilyl). A diol compound of formula (XIV) is prepared by reaction of a lactol compound of formula (XIII) with ethynyl magnesium bromide; in a suitable solvent such as THF; at temperatures ranging from −70° C. to 30° C. The propargyl alcohol compound of formula (XIV) is protected by reaction with ethyl chloroformate; a base such as pyridine, and the like; in a suitable solvent such as DCM; at a temperature of about 0° C., to provide a compound of formula (XV), where R^{e} is CO₂Et.

The propargyl alcohol compound of formula (XIV) is protected by variety of suitable reagents, including tert-butyl-chloro-dimethyl-silane in the presence of a base such as imidazole; in a suitable organic solvent such as DMF, DCM, and the like; to provide a compound of formula (XV), where R^{e} is TBS.

In a similar fashion, 2,3-O-isopropylidene-beta-D-ribofuranose is reacted with ethynyl magnesium bromide as previously described to provide 1-((4S,5R)-5-((R)-1-hydroxy-2-((4-methoxyphenyl)diphenylmethoxy)ethyl)-2,2-dimethyl-1,3-dioxolan-4-yl)prop-2-yn-1-ol. TBS protection, as previously described provides (1R)-1-((4R,5R)-5-(1-((tert-butyldimethylsilyl)oxy)prop-2-yn-1-yl)-2,2-dimethyl-1,3-dioxolan-4-yl)-2-((4-methoxyphenyl)diphenylmethoxy)ethan-1-ol.

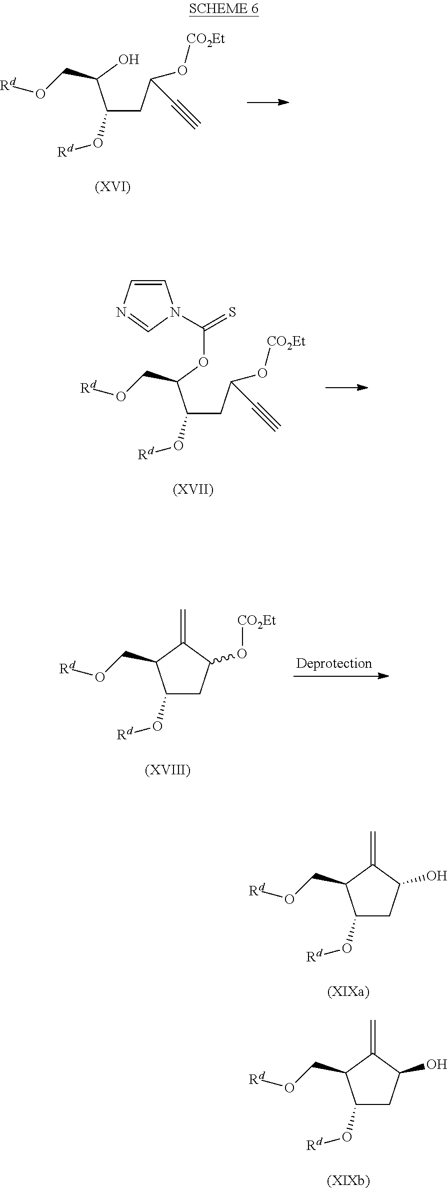

According to SCHEME 6, conversion of a compound of formula (XVI), where R^{d} is TIPS, to the thiocarbonyl imidazolide derivative compound of formula (XVII) is achieved by reaction of a compound of formula (XVI) with 1,1′-thiocarbonylimidazole (TCDI); in a suitable solvent such as DCM, and the like; to provide a thiocarbonyl imidazolide compound of formula (XVII). The subsequent Barton free radical deoxygenation affords a compound of formula (XVIII). For example, a compound of formula (XVII) is reacted with a radical initiator such as azobisisobutyronitrile (AIBN), and the like; tri-n-butyltin hydride; in a suitable solvent such as toluene and the like; at temperatures ranging from 30° C. to 110° C.; to afford the desired compound of formula (XVIII). Deprotection of a compound of formula (XVIII), employing conditions known to one skilled in the art provides compounds of formula (XIXa) and (XIXb).

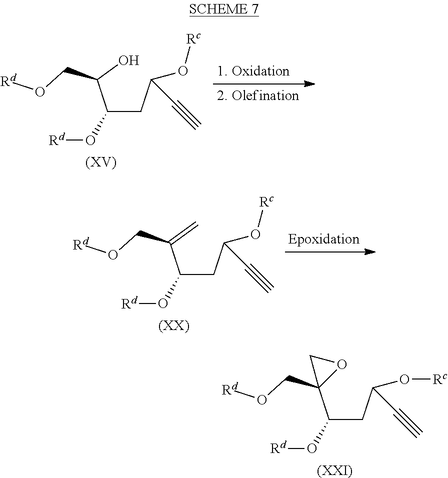

According to SCHEME 7, a compound of formula (XV), where R^{d} is pivaloyl (Piv), and R^{c} is TBS, is oxidized employing Dess-Martin periodinane conditions as previously described. Subsequent olefination of an α-hydroxy ketone compound employing conditions previously described provides a compound of formula (XX). For example, reaction with bromo methyl triphenylphosphorane; a base such as n-BuLi; in a suitable solvent such as THF; provides a compound of formula (XX). Epoxidation of the terminal olefin of a compound of formula (XX) is achieved with a reagent such as m-CPBA, and the like; in a suitable solvent such as DCM; at temperatures ranging from 0° C. to 45° C.; to provide a compound of formula (XXI).

In a similar fashion, (1R)-1-((4R,5R)-5-(1-((tert-butyldimethylsilyl)oxy)prop-2-yn-1-yl)-2,2-dimethyl-1,3-dioxolan-4-yl)-2-((4-methoxyphenyl)diphenylmethoxy)ethan-1-ol is oxidized employing Dess-Martin periodinane conditions previously described to provide 1-((4S,5R)-5-((S)-1-((tert-butyldimethylsilyl)oxy)prop-2-yn-1-yl)-2,2-dimethyl-1,3-dioxolan-4-yl)-2-((4-methoxyphenyl)diphenylmethoxy)ethan-1-one. Olefination of 1-((4S,5R)-5-((S)-1-((tert-butyldimethylsilyl)oxy)prop-2-yn-1-yl)-2,2-dimethyl-1,3-dioxolan-4-yl)-2-((4-methoxyphenyl)diphenylmethoxy)ethan-1-one under conditions previously described provides tert-butyl(((S)-1-((4R,5R)-5-(3-((4-methoxyphenyl)diphenylmethoxy)prop-1-en-2-yl)-2,2-dimethyl-1,3-dioxolan-4-yl)prop-2-yn-1-yl)oxy)dimethylsilane. Epoxidation of tert-Butyl(((S)-1-((4R,5R)-5-(3-((4-methoxyphenyl)diphenylmethoxy)prop-1-en-2-yl)-2,2-dimethyl-1,3-dioxolan-4-yl)prop-2-yn-1-yl)oxy)dimethylsilane under conditions previously described provides tert-Butyl(((1S)-1-((4R,5S)-5-(2-(((4-methoxyphenyl)diphenylmethoxy)methyl)oxiran-2-yl)-2,2-dimethyl-1,3-dioxolan-4-yl)prop-2-yn-1-yl)oxy)dimethylsilane.

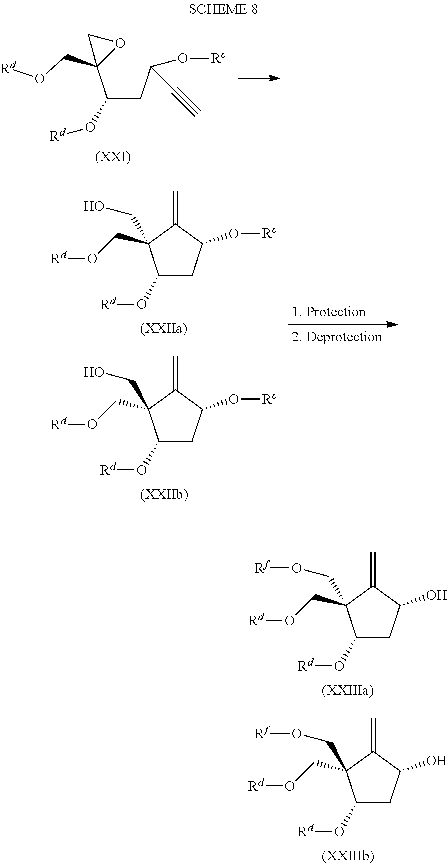

According to SCHEME 8, a compound of formula (XXI), where R^{d} is Piv and R^{c} is TBS, undergoes catalytic radicalary cyclization using titanocene dichloride (Cp₂TiCl₂) as catalyst; in the presence of Mn/2,4,6-collidine HCl or Zn/2,4,6-collidine/trimethylsilyl chloride; provides compounds of formula (XXIIa) and (XXIIb). Protection with DMTr is achieved by reaction with 4,4′-dimethoxytrityl chloride (DMTrCl); in the presence of pyridine, dimethylpyridine, or 2,4,6-trimethylpyridine (collidine); AgNO₃; in a suitable solvent such as DCM, and the like. Deprotection of TBS protecting group employing conditions known to one skilled in the art, provides a compound of formula (XIIIa) and (XIIIb), where R^{d} is Piv and R^{f} is DMTr.

In a similar fashion, tert-Butyl(((1S)-1-((4R,5S)-5-(2-(((4-methoxyphenyl)diphenylmethoxy)methyl)oxiran-2-yl)-2,2-dimethyl-1,3-dioxolan-4-yl)prop-2-yn-1-yl)oxy)dimethylsilane undergoes catalytic radicalary cyclization as previously described to provide ((3aR,4S,6S,6aR)-6-((tert-Butyldimethylsilyl)oxy)-4-(((4-methoxyphenyl)diphenylmethoxy)methyl)-2,2-dimethyl-5-methylenetetrahydro-4H-cyclopenta[d][1,3]dioxol-4-yl)methanol.

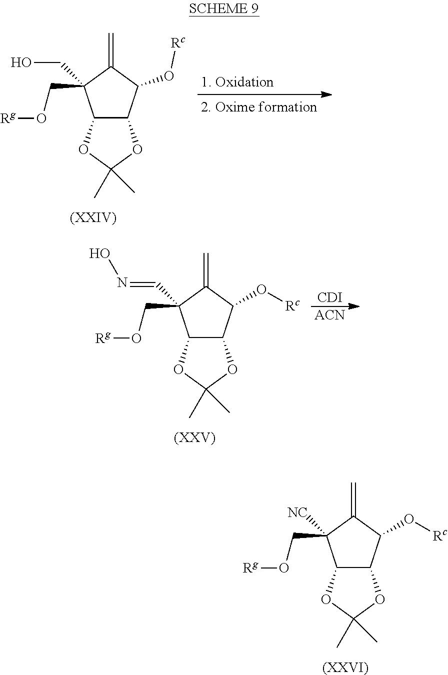

According to SCHEME 9, an alcohol compound of formula (XXIV) where R^{g} is MMTr (monomethoxytrityl) and R^{c} is TBS; is oxidized under conditions previously described to provide the aldehyde intermediate. Subsequent oxime formation employing conditions known to one skilled in the art, provides a compound of formula (XXV). For example, the aldehyde intermediate is reacted with hydroxylamine hydrochloride; in a solvent such as pyridine; to provide an aldoxime compound of formula (XXV). An oxime compound of formula (XXV) is dehydrated employing conventional oxime dehydration methodology. For example, reaction of an oxime compound of formula (XXV) with 1,1′-carbonyldiimidazole (CDI); in a suitable solvent such as ACN, and the like; at temperatures ranging from 0° C. to 30° C.; to provide a nitrile compound of formula (XXVI), where R⁹ is MMTr (monomethoxytrityl) and R^{c} is TBS.

In a similar fashion 1-((1S,3S,4S)-4-(bis(4-methoxyphenyl)(phenyl)methoxy)-3-((bis(4-methoxyphenyl)(phenyl)methoxy)methyl)-3-(hydroxymethyl)-2-methylenecyclopentyl)-5-methylpyrimidine-2,4(1H,3H)-dione is oxidized employing conditions previously described to provide (1S,3S,5S)-5-(bis(4-methoxyphenyl)(phenyl)methoxy)-1-((bis(4-methoxyphenyl)(phenyl)methoxy)methyl)-3-(5-methyl-2,4-dioxo-3,4-dihydropyrimidin-1(2H)-yl)-2-methylenecyclopentane-1-carbaldehyde. Oxime formation employing conditions previously described provides (E)-5-(bis(4-methoxyphenyl)(phenyl)methoxy)-1-((bis(4-methoxyphenyl)(phenyl)methoxy)methyl)-3-(5-methyl-2,4-dioxo-3,4-dihydropyrimidin-1(2H)-yl)-2-methylenecyclopentane-1-carbaldehyde oxime. Dehydration of the oxime affords (1S,3S,5S)-5-(bis(4-methoxyphenyl)(phenyl)methoxy)-1-((bis(4-methoxyphenyl)(phenyl)methoxy)methyl)-3-(5-methyl-2,4-dioxo-3,4-dihydropyrimidin-1(2H)-yl)-2-methylenecyclopentane-1-carbonitrile.

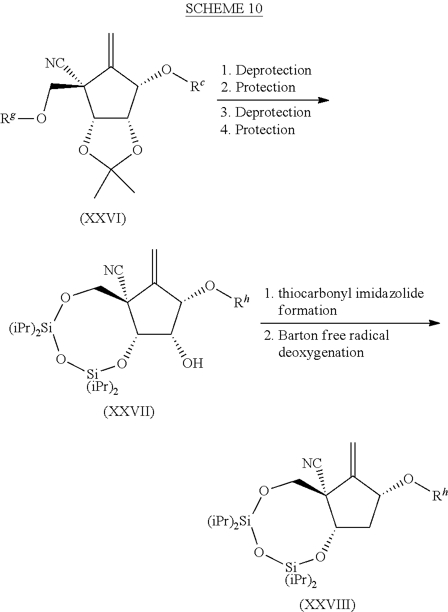

According to SCHEME 10, a cyclic acetal compound of formula (XXVI), where R^{g} is MMTr (monomethoxytrityl) and R^{c} is TBS, undergoes deprotection and re-protection employing conditions known to one skilled in the art or as previously described, to form a cyclic silyl ether compound of formula (XXVII), where R^{h} is Bz (benzoyl). A compound of formula (XXVIII) is formed in two steps from a compound of formula (XXVII). In a first step, formation of the thiocarbonyl imidaxolide employing conditions previously described, followed by a second step free radical deoxygenation employing conditions previously described provides a compound of formula (XXVIII), where R^{h} is Bz (benzoyl). Deprotection of the benzoyl protecting group employing conditions known to one skilled in the art provides (6aS,8R,9aS)-8-hydroxy-2,2,4,4-tetraisopropyl-7-methylenetetrahydrocyclopenta[f][1,3,5,2,4]trioxadisilocine-6a(6H)-carbonitrile.

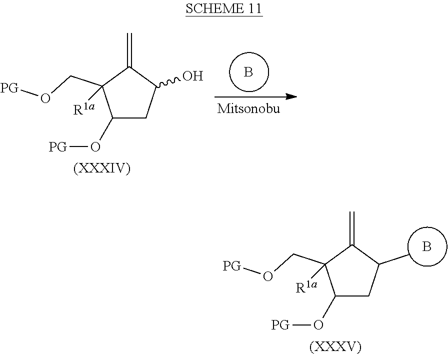

Commercially available or synthetically accessible nucleobases, modified nucleobases, or nucleobase analogs of formula ring B, are protected (and deprotected) employing established methodologies, such as those described in T. W. Greene and P. G. M. Wuts, “Protective Groups in Organic Synthesis,” 3 ed., John Wiley & Sons, 1999.

For example, thymine is benzoyl protected employing benzoyl chloride (BzCl); a base such as pyridine; in a solvent such as THF and the like; to afford the bis Bz protected 1,3-dibenzoyl-5-methylpyrimidine-2,4(1H,3H)-dione. Selective deprotection of one of the Bz protecting groups is achieved using K₂CO₃; in a solvent such as dioxane; to afford 3-benzoyl-5-methylpyrimidine-2,4(1H,3H)-dione.

Selective carbamate protection of the exocyclic amino group of 7H-pyrrolo[2,3-d]pyrimidin-4-amine is achieved under conditions known to one skilled in the art. For example, reaction of 7H-pyrrolo[2,3-d]pyrimidin-4-amine with di-tert-butyl dicarbonate (Boc₂O); DMAP; in a suitable solvent such as THF, and the like; provides the tri-BOC protected compound N,N,N-Tri-BOC-7H-pyrrolo[2,3-d]pyrimidin-4-amine. Selective mild deprotection of one of the BOC protecting groups is achieved using aq NaHCO₃, in a suitable solvent such as MeOH, to afford N,N-Di-BOC-7H-pyrrolo[2,3-d]pyrimidin-4-amine.

The exocyclic amine of guanine is selectively protected by, for example, treatment with isobutyric anhydride; at temperatures ranging from 30° C. to 155° C.; in a suitable solvent such as DMF; for a period of about for 4 hours; to afford N-(6-oxo-6,9-dihydro-1H-purin-2-yl)isobutyramide. Acylation of N-(6-oxo-6,9-dihydro-1H-purin-2-yl)isobutyramide, employing an acylating reagent selected from an acyl derivative, an acyl halide such as acetyl chloride and the like, and an acid anhydride such as acetic anhydride, propionic anhydride, and the like; in a suitable solvent such as DMF; affords N-(9-acetyl-6-oxo-6,9-dihydro-1H-purin-2-yl)isobutyramide. The 4-oxo moiety of N-(9-acetyl-6-oxo-6,9-dihydro-1H-purin-2-yl)isobutyramide is protected as the diphenylcarbamate species by using, for example, diphenylcarbamoyl chloride to provide 9-acetyl-2-isobutyramido-9H-purin-6-yl diphenylcarbamate. Deprotection of the acetyl protecting group is achieved in EtOH/water at a temperature of about 100° C., for a period of about 2 h, to provide 2-Isobutyramido-9H-purin-6-yl diphenylcarbamate.

According to SCHEME 11, a compound of formula (XXXIV) (as well as formulas (XIa), (XIb), (XIXa), (XIXb), (XXIIIa), (XXIIIb), and (6aS,8R,9aS)-8-hydroxy-2,2,4,4-tetraisopropyl-7-methylenetetrahydrocyclopenta[f][1,3,5,2,4]trioxadisilocine-6a(6H)-carbonitrile), where PG is a suitable protecting group, and R^{1a} is H, F, CH₂O-DMTr, CN, is reacted with ring B, where ring B is a nitrogen linked nucleobase, modified nucleobase, or nucleobase analog; under Mitsonobu conditions. For example, using triphenylphosphine; a base such as di-tert-butyl azodicarboxylate (DIAD), diethyl azodicarboxylate (DEAD) and the like; in a solvent such as THF, ACN, dioxane, or a mixture thereof; at a temperature ranging from 25 to 110° C.; to provide a compound of formula (XXXV).

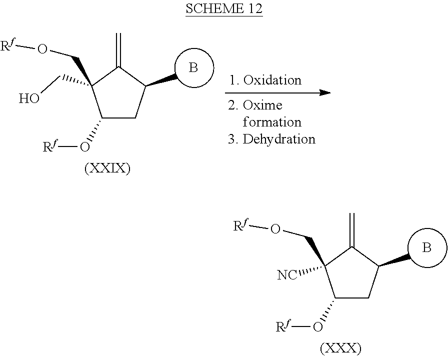

Employing methods described in SCHEME 9, compounds of formula (XXIX), where R^{f} is DMTr, and ring B is a nitrogen linked nucleobase, modified nucleobase, or nucleobase analog; is oxidized employing conditions previously described. Oxime formation and dehydration methods previously described provides a cyano compound of formula (XXX).

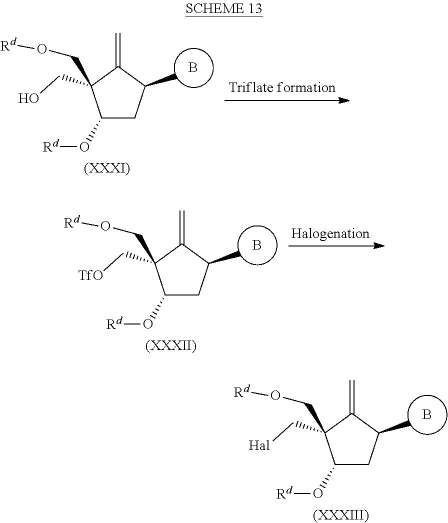

According to SCHEME 13, activation of the alcohol compound of formula (XXXI), where R^{d} is Piv, and ring B is a nitrogen linked nucleobase, modified nucleobase, or nucleobase analog such as 6-chloro-9l²-purine, adenine, thymine, uracil, and the like, (each optionally protected with a suitable protecting group such as Bz, BOC, and the like); with triflic anhydride (TfO₂); pyridine, in a suitable solvent such as DCM, and the like; provides a compound of formula (XXXII). Subsequent nucleophilic substitution reaction of a sulfonate compound of formula (XXXII), employing methods known to one skilled in the art provides a compound of formula (XXXIIII). For example, reaction with tetrabutylammonium fluoride (TBAF), in a suitable solvent such as THF, and the like, provides a compound of formula (XXXIII), where Hal is F. A compound of formula (XXXII) is reacted with LiCl, in a solvent such as DMF, at a temperature of about 40° C. to provide a compound of formula (XXXIII), where Hal is Cl.

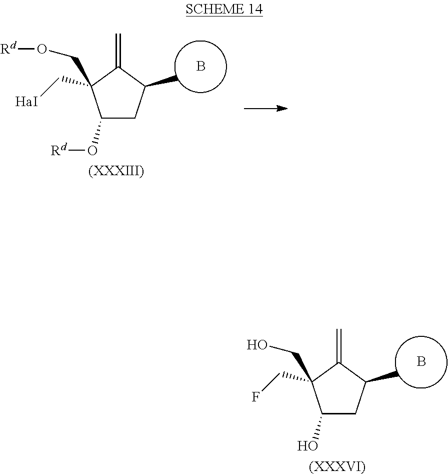

According to SCHEME 14, 4-amino-1-((1S,3S,4S)-3-(fluoromethyl)-4-hydroxy-3-(hydroxymethyl)-2-methylenecyclopentyl)pyrimidin-2(1H)-one is prepared from a compound of formula (XXXIII), where R^{d} is Piv, Hal is F, and ring B is the nucleobase uracil, by first reaction with 2,4,6-triisopropylbenzenesulfonyl chloride (TPSCl) in the presence of dimethylaminopyridine (DMAP); triethylamine; in a suitable solvent such as acetonitrile and the like; subsequent ammonolysis with ammonium hydroxide, NH₃.H₂O, or a strong amine; in the presence of a suitable inert organic solvent; at temperatures in the range of about from 10° C. to 50° C.; for about from 1 to 12 h. Deprotection of the Piv protecting group employing conditions known to one skilled in the art, and as described, provides 4-amino-1-((1S,3S,4S)-3-(fluoromethyl)-4-hydroxy-3-(hydroxymethyl)-2-methylenecyclopentyl)pyrimidin-2(1H)-one.

A compound of formula (XXXIII), where R^{d} is Piv, Hal is F, and ring B is the nucleobase analog 6-chloro-9λ²-purine, is reacted under ammonolysis conditions, for example, reaction with ammonia; in a suitable solvent such as THF; at temperatures in the range of about from 10° C. to 50° C.; for about from 1 to 12 h. Subsequent deprotection of the Piv protecting group provides (1S,2S,4S)-4-(6-amino-9H-purin-9-yl)-2-(fluoromethyl)-2-(hydroxymethyl)-3-methylenecyclopentan-1-ol.

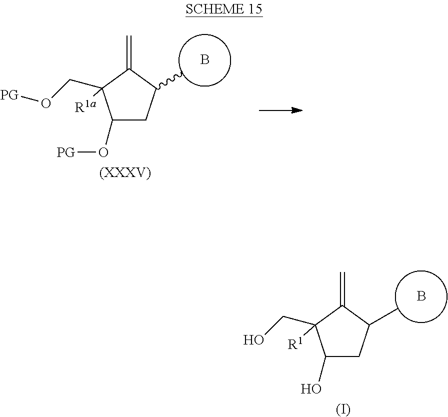

Compounds of formula (XXXV) that have protecting groups are deprotected following established methodologies, such as those described in T. W. Greene and P. G. M. Wuts, “Protective Groups in Organic Synthesis,” 3 ed., John Wiley & Sons, 1999, to provide compounds of Formula (I).

For example, compounds with BOC protecting groups are cleaved under standard acidic conditions, such as TFA, HCl, and the like. Deprotection of TBS is achieved employing tetrabutylammonium fluoride (TBAF). Deprotection of Bz is achieved with ammonia or alkali metal alkoxide in alcohol, preferably ammonia in methanol or sodium alkoxide in methanol, at a temperature of from −2° C. to 100° C., preferably from 25° C. to 80° C., for a period of from 5 minutes to 3 days, preferably from 30 minutes to 4 hours. Deprotection of benzyl group is achieved under hydrogenolytic conditions. Ester (Piv. CO₂Et, and the like) cleavage is achieved under basic conditions, such as exposure of the ester to a methanolic solution of sodium methoxide, NaOH/MeOH, and the like. Mild detritylation of a monomethoxytrityl (MMtr) and 4,4-dimethoxytrityl (DMTr) is achieved under conditions known to one skilled in the art. For example, employing an acid such as trichloroacetic acid (TCA), trifluoroacetic acid (TFA), and the like, in a suitable solvent such as DCM, and the like, at room temperature, for a period of 1-3 h. Deprotection of the isobutyl carbamate is achieved under neutral or mildly basic conditions such as NaOH/MeOH.

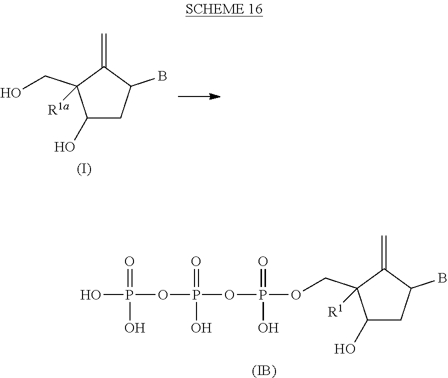

According to SCHEME 16, a nucleoside triphosphate compound of Formula (IB), is prepared from a nucleoside compound of Formula (I), employing conditions known to one skilled in the art. For example, reaction of the nucleoside of Formula (I), with trimethyl phosphate, triethyl phosphate, and the like; phosphoryl chloride; and N-methylimidazole to provide the corresponding nucleoside monophosphate intermediate. Subsequent reaction of the nucleoside monophosphate with the tetrabutylammonium salt of pyrophosphate, in a suitable solvent such as DMF, and the like, provides the triphosphate of Formula (IB).

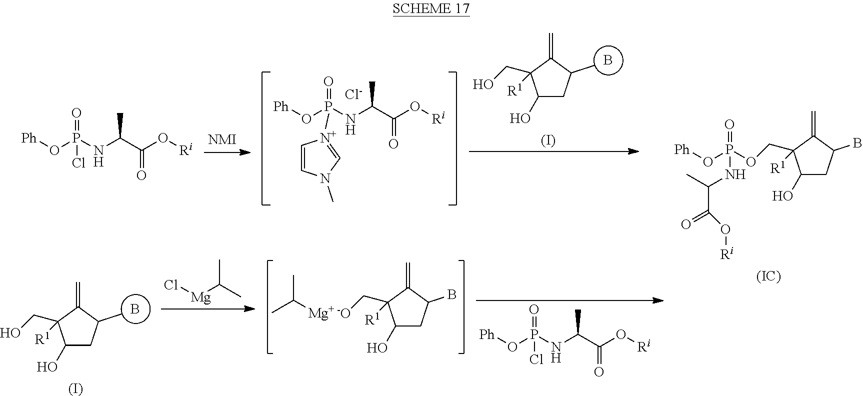

According to SCHEME 17, aryloxyphosphoramidate nucleoside prodrug compounds of Formula (IC) are prepared by coupling of nucleosides compounds of Formula (I) with phosphorochloridate by either activation of the imidazolium intermediate with NMI (N-methylimidazole) or by 5′-deprotonation of the nucleoside with isoPrMgCl, t-BuMgCl, and the like, and subsequent substitution with the chlorophosphoramidate. It is noteworthy that these different synthetic approaches generally lead to approximate 1:1 mixtures of compounds of Formula (IC) as diastereoisomers at the phosphorus center (Sₚ and Rₚ isomers); where Rⁱ is C₁₋₈alkyl.

Compounds of Formula (I) may be converted to their corresponding salts using methods known to one of ordinary skill in the art. For example, an amine of Formula (I) is treated with trifluoroacetic acid, HCl, or citric acid in a solvent such as Et₂O, CH₂Cl₂, THF, MeOH, chloroform, or isopropanol to provide the corresponding salt form. Alternately, trifluoroacetic acid or formic acid salts are obtained as a result of reverse phase HPLC purification conditions. Crystalline forms of pharmaceutically acceptable salts of compounds of Formula (I) may be obtained in crystalline form by recrystallization from polar solvents (including mixtures of polar solvents and aqueous mixtures of polar solvents) or from non-polar solvents (including mixtures of non-polar solvents).

Where the compounds according to this invention have at least one chiral center, they may accordingly exist as enantiomers. Where the compounds possess two or more chiral centers, they may additionally exist as diastereomers. It is to be understood that all such isomers and mixtures thereof are encompassed within the scope of the present invention.

Compounds prepared according to the schemes described above may be obtained as single forms, such as single enantiomers, by form-specific synthesis, or by resolution. Compounds prepared according to the schemes above may alternately be obtained as mixtures of various forms, such as racemic (1:1) or non-racemic (not 1:1) mixtures. Where racemic and non-racemic mixtures of enantiomers are obtained, single enantiomers may be isolated using conventional separation methods known to one of ordinary skill in the art, such as chiral chromatography, recrystallization, diastereomeric salt formation, derivatization into diastereomeric adducts, biotransformation, or enzymatic transformation. Where regioisomeric or diastereomeric mixtures are obtained, as applicable, single isomers may be separated using conventional methods such as chromatography or crystallization.

In some embodiments, varying the substituents on a compound described herein, such as a compound of Formula (I), or a pharmaceutically acceptable salt thereof, can result in the phosphorous being a chiral center. In some embodiments, the phosphorous can be in the (R)-configuration. In some embodiments, the phosphorous can be in the (S)-configuration. Examples of the two configurations are:

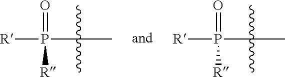

In some embodiments, a compound of Formula (I), or a pharmaceutically acceptable salt thereof, can be enriched in (R) or (S) configuration with respect to the phosphorous. For example, one of the (R) and (S) configuration with respect to the phosphorous atom can be present in an amount >50%, ≥75%, ≥90%, ≥95% or ≥99% compared to the amount of the other of the (R) or (S) configuration with respect to the phosphorous atom.

By neutralizing the charge on the phosphonate moiety of Formula (I), or a pharmaceutically acceptable salt thereof, penetration of the cell membrane may be facilitated as a result of the increased lipophilicity of the compound. Once absorbed and taken inside the cell, the groups attached to the phosphorus can be easily removed by esterases, proteases and/or other enzymes. In some embodiments, the groups attached to the phosphorus can be removed by simple hydrolysis. Inside the cell, the phosphonate thus released may then be metabolized by cellular enzymes to the monophosphonate or the active diphosphonate (for example, a phosphono diphosphate). Furthermore, in some embodiments, varying the substituents on a compound described herein, such as a compound of Formula (I), or a pharmaceutically acceptable salt thereof, can help maintain the efficacy of the compound by reducing undesirable effects.

In some embodiments, a compound of Formula (I), or a pharmaceutically acceptable salt thereof, can act as a chain terminator of a virus and inhibit the virus' replication, wherein the virus can be HBV, HDV and/or HIV. For example, compounds of Formula (I), or a pharmaceutically acceptable salt thereof, can be incorporated into a DNA chain of the virus (such as HBV, HDV and/or HIV) and then no further elongation is observed to occur.

In some embodiments, a compound of Formula (I), or a pharmaceutically acceptable salt thereof, can have increased metabolic and/or plasma stability. In some embodiments, a compound of Formula (I), or a pharmaceutically acceptable salt thereof, can be more resistant to hydrolysis and/or more resistant to enzymatic transformations. For example, a compound of Formula (I), or a pharmaceutically acceptable salt thereof, can have increased metabolic stability, increased plasma stability, and/or can be more resistant to hydrolysis. In some embodiments, a compound of Formula (I), or a pharmaceutically acceptable salt thereof, can have improved properties. A non-limiting list of example properties include, but are not limited to, increased biological half-life, increased bioavailability, increase potency, a sustained in vivo response, increased dosing intervals, decreased dosing amounts, decreased cytotoxicity, reduction in required amounts for treating disease conditions, reduction in viral load, reduction in plasma viral load, increase CD4+T lymphocyte counts, reduction in time to seroconversion (i.e., the virus becomes undetectable in patient serum), increased sustained viral response, a reduction of morbidity or mortality in clinical outcomes, decrease in or prevention of opportunistic infections, increased subject compliance, and compatibility with other medications. In some embodiments, a compound of Formula (I), or a pharmaceutically acceptable salt thereof, can have a biological half-life of greater than 24 hours. In some embodiments, a compound of Formula (I), or a pharmaceutically acceptable salt thereof, can have more potent antiviral activity (for example, a lower EC₅₀ in an HIV, HBV and/or HDV replicon assay) as compared to the current standard of care.

### Pharmaceutical Compositions

Some embodiments described herein relate to a pharmaceutical composition, that can include an effective amount of one or more compounds described herein (e.g., a compound of Formula (I), or a pharmaceutically acceptable salt thereof) and a pharmaceutically acceptable carrier, diluent, excipient or combination thereof.

In some embodiments, the pharmaceutical composition can include a single diastereomer of a compound of Formula (I), or a pharmaceutically acceptable salt thereof, (for example, a single diastereomer is present in the pharmaceutical composition at a concentration of greater than 99% compared to the total concentration of the other diastereomers). In other embodiments, the pharmaceutical composition can include a mixture of diastereomers of a compound of Formula (I), or a pharmaceutically acceptable salt thereof. For example, the pharmaceutical composition can include a concentration of one diastereomer of >50%, ≥60%, ≥70%, ≥80%, ≥90%, ≥95%, or ≥98%, as compared to the total concentration of the other diastereomers. In some embodiments, the pharmaceutical composition includes a 1:1 mixture of two diastereomers of a compound of Formula (I), or a pharmaceutically acceptable salt thereof.

The term “pharmaceutical composition” refers to a mixture of one or more compounds disclosed herein with other chemical components, such as diluents or carriers. The pharmaceutical composition facilitates administration of the compound to an organism. Pharmaceutical compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid and salicylic acid. Pharmaceutical compositions will generally be tailored to the specific intended route of administration. A pharmaceutical composition is suitable for human and/or veterinary applications.

The term “physiologically acceptable” defines a carrier, diluent or excipient that does not abrogate the biological activity and properties of the compound.

As used herein, a “carrier” refers to a compound that facilitates the incorporation of a compound into cells or tissues. For example, without limitation, dimethyl sulfoxide (DMSO) is a commonly utilized carrier that facilitates the uptake of many organic compounds into cells or tissues of a subject.

As used herein, a “diluent” refers to an ingredient in a pharmaceutical composition that lacks pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the composition of human blood.

As used herein, an “excipient” refers to an inert substance that is added to a pharmaceutical composition to provide, without limitation, bulk, consistency, stability, binding ability, lubrication, disintegrating ability etc., to the composition. A “diluent” is a type of excipient.

The pharmaceutical compositions described herein can be administered to a human patient per se, or in pharmaceutical compositions where they are mixed with other active ingredients, as in combination therapy, or carriers, diluents, excipients or combinations thereof. Proper formulation is dependent upon the route of administration chosen. Techniques for formulation and administration of the compounds described herein are known to those skilled in the art.

The pharmaceutical compositions disclosed herein may be manufactured in a manner that is itself known, e.g., by means of conventional mixing, dissolving, granulating, dragee-making, levigating, emulsifying, encapsulating, entrapping or tableting processes. Additionally, the active ingredients are contained in an amount effective to achieve its intended purpose. Many of the compounds used in the pharmaceutical combinations disclosed herein may be provided as salts with pharmaceutically compatible counterions.

Multiple techniques of administering a compound exist in the art including, but not limited to, oral, rectal, topical, aerosol, injection and parenteral delivery, including intramuscular, subcutaneous, intravenous, intramedullary injections, intrathecal, direct intraventricular, intraperitoneal, intranasal and intraocular injections.

One may also administer the compound in a local rather than systemic manner, for example, via injection of the compound directly into the infected area, often in a depot or sustained release formulation. Furthermore, one may administer the compound in a targeted drug delivery system, for example, in a liposome coated with a tissue-specific antibody. The liposomes may be targeted to and taken up selectively by the organ.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions that can include a compound described herein formulated in a compatible pharmaceutical carrier may also be prepared, placed in an appropriate container, and labeled for treatment of an indicated condition.

### Methods of Use

Some embodiments disclosed herein relate to a method of treating and/or ameliorating a disease or condition that can include administering to a subject an effective amount of one or more compounds described herein, such as a compound of Formula (I), or a pharmaceutically acceptable salt thereof, or a pharmaceutical composition that includes a compound described herein, or a pharmaceutically acceptable salt thereof. Other embodiments disclosed herein relate to a method of treating and/or ameliorating a disease or condition that can include administering to a subject identified as suffering from the disease or condition an effective amount of one or more compounds described herein, such as a compound of Formula (I), or a pharmaceutically acceptable salt thereof, or a pharmaceutical composition that includes a compound described herein, or a pharmaceutically acceptable salt thereof.

Some embodiments described herein relate to a method of treating a HBV and/or HDV infection that can include administering to a subject identified as suffering from the HBV and/or HDV infection an effective amount of a compound described herein (for example, a compound of Formula (I), or a pharmaceutically acceptable salt thereof), or a pharmaceutical composition that includes an effective amount of a compound described herein (such as, a compound of Formula (I), or a pharmaceutically acceptable salt thereof). Other embodiments described herein relate to using a compound described herein (for example, a compound of Formula (I), or a pharmaceutically acceptable salt thereof) in the manufacture of a medicament for treating a HBV and/or HDV infection. Still other embodiments described herein relate to the use of a compound described herein (for example, a compound of Formula (I), or a pharmaceutically acceptable salt thereof), or a pharmaceutical composition that includes a compound described herein (such as, a compound of Formula (I), or a pharmaceutically acceptable salt thereof) for treating a HBV and/or HDV infection.

Some embodiments disclosed herein relate to a method of treating a HBV and/or HDV infection that can include contacting a cell infected with the HBV and/or HDV with an effective amount of a compound described herein (for example, a compound of Formula (I), or a pharmaceutically acceptable salt thereof), or a pharmaceutical composition that includes an effective amount of a compound described herein. Other embodiments described herein relate to using a compound described herein (for example, a compound of Formula (I), or a pharmaceutically acceptable salt thereof) in the manufacture of a medicament for treating a HBV and/or HDV infection that can include contacting a cell infected with the HBV and/or HDV with an effective amount of said compound(s) and/or pharmaceutical composition described herein. Still other embodiments described herein relate to the use of a compound described herein (for example, a compound of Formula (I), or a pharmaceutically acceptable salt thereof), or a pharmaceutical composition that includes a compound described herein for treating a HBV and/or HDV infection, wherein the use includes contacting a cell infected with the HBV and/or HDV with an effective amount of said compound(s) and/or pharmaceutical composition described herein.

Some embodiments disclosed herein relate to a method of inhibiting replication of HBV and/or HDV that can include contacting a cell infected with the HBV and/or HDV with an effective amount of a compound described herein (for example, a compound of Formula (I), or a pharmaceutically acceptable salt thereof), or a pharmaceutical composition that includes a compound described herein (for example, a compound of Formula (I), or a pharmaceutically acceptable salt thereof). Other embodiments described herein relate to using a compound described herein (such as, a compound of Formula (I), or a pharmaceutically acceptable salt thereof) in the manufacture of a medicament for inhibiting replication of HBV and/or HDV that can include contacting a cell infected with HBV and/or HDV with an effective amount of said compound(s) and/or pharmaceutical composition described herein. Still other embodiments described herein relate to the use of a compound described herein (for example, a compound of Formula (I), or a pharmaceutically acceptable salt thereof), or a pharmaceutical composition that includes an effective amount of a compound described herein (such as, a compound of Formula (I), or a pharmaceutically acceptable salt thereof), for inhibiting replication of HBV and/or HDV, wherein the use includes contacting a cell infected with the HBV and/or HDV with an effective amount of said compound(s) and/or pharmaceutical composition described herein.

In some embodiments, the HBV infection can be an acute HBV infection. In some embodiments, the HBV infection can be a chronic HBV infection.

Some embodiments disclosed herein relate to a method of treating liver cirrhosis that is developed because of a HBV and/or HDV infection that can include administering to a subject suffering from liver cirrhosis and/or contacting a cell infected with the HBV and/or HDV in a subject suffering from liver cirrhosis with an effective amount of a compound described herein (for example, a compound of Formula (I), or a pharmaceutically acceptable salt thereof), or a pharmaceutical composition that includes an effective amount of a compound described herein. Other embodiments described herein relate to using a compound described herein (for example, a compound of Formula (I), or a pharmaceutically acceptable salt thereof) in the manufacture of a medicament for treating liver cirrhosis with an effective amount of said compound(s) and/or pharmaceutical composition described herein. Still other embodiments described herein relate to the use of a compound described herein (for example, a compound of Formula (I), or a pharmaceutically acceptable salt thereof), or a pharmaceutical composition that includes a compound described herein for treating liver cirrhosis.

Some embodiments disclosed herein relate to a method of treating liver cancer (such as hepatocellular carcinoma) that is developed because of a HBV and/or HDV infection that can include administering to a subject suffering from liver cancer and/or contacting a cell infected with the HBV and/or HDV in a subject suffering from liver cancer with an effective amount of a compound described herein (for example, a compound of Formula (I), or a pharmaceutically acceptable salt thereof), or a pharmaceutical composition that includes an effective amount of a compound described herein. Other embodiments described herein relate to using a compound described herein (for example, a compound of Formula (I), or a pharmaceutically acceptable salt thereof) in the manufacture of a medicament for treating liver cancer (such as hepatocellular carcinoma) with an effective amount of said compound(s) and/or pharmaceutical composition described herein. Still other embodiments described herein relate to the use of a compound described herein (for example, a compound of Formula (I), or a pharmaceutically acceptable salt thereof), or a pharmaceutical composition that includes a compound described herein for treating liver cancer (such as hepatocellular carcinoma).

Some embodiments disclosed herein relate to a method of treating liver failure that is developed because of a HBV and/or HDV infection that can include administering to a subject suffering from liver failure and/or contacting a cell infected with the HBV and/or HDV in a subject suffering from liver failure with an effective amount of a compound described herein (for example, a compound of Formula (I), or a pharmaceutically acceptable salt thereof), or a pharmaceutical composition that includes an effective amount of a compound described herein. Other embodiments described herein relate to using a compound described herein (for example, a compound of Formula (I), or a pharmaceutically acceptable salt thereof) in the manufacture of a medicament for treating liver failure with an effective amount of said compound(s) and/or pharmaceutical composition described herein. Still other embodiments described herein relate to the use of a compound described herein (for example, a compound of Formula (I), or a pharmaceutically acceptable salt thereof), or a pharmaceutical composition that includes a compound described herein for treating liver failure.

Various indicators for determining the effectiveness of a method for treating an HBV and/or HDV infection are also known to those skilled in the art. Examples of suitable indicators include, but are not limited to, a reduction in viral load indicated by reduction in HBV DNA (or load), HBV surface antigen (HBsAg) and HBV e-antigen (HBeAg), a reduction in plasma viral load, a reduction in viral replication, a reduction in time to seroconversion (virus undetectable in patient serum), an increase in the rate of sustained viral response to therapy, an improvement in hepatic function, and/or a reduction of morbidity or mortality in clinical outcomes.

In some embodiments, an effective amount of a compound of Formula (I), or a pharmaceutically acceptable salt thereof, is an amount that is effective to reduce HBV and/or HDV viral load to undetectable levels, for example, to about 10 to about 50, or to about 15 to about 25 international units/mL serum, or to less than about 20 international units/mL serum. In some embodiments, an effective amount of a compound of Formula (I), or a pharmaceutically acceptable salt thereof, is an amount that is effective to reduce HBV and/or HDV viral load compared to the HBV and/or HDV viral load before being provided the compound of Formula (I), or a pharmaceutically acceptable salt thereof. In some embodiments, an effective amount of a compound of Formula (I), or a pharmaceutically acceptable salt thereof, can be an amount that is effective to reduce HBV and/or HDV viral load to lower than about 20 international units/mL serum. In some embodiments, an effective amount of a compound of Formula (I), or a pharmaceutically acceptable salt thereof, is an amount that is effective to achieve a reduction in HBV and/or HDV viral load in the serum of the subject to an undetectable level and/or in the range of about 1.5-log to about a 2.5-log reduction, about a 3-log to about a 4-log reduction, or a greater than about 5-log reduction compared to the viral load before being provided the compound of Formula (I), or a pharmaceutically acceptable salt thereof. For example, the HBV and/or HDV viral load can be measured before being provided the compound of Formula (I), or a pharmaceutically acceptable salt thereof, and again after completion of at least a portion of the treatment regime with the compound of Formula (I), or a pharmaceutically acceptable salt thereof (for example, 1 month after initiation or completion).

In some embodiments, a compound of Formula (I), or a pharmaceutically acceptable salt thereof, can result in at least a 1, 2, 3, 4, 5, 10, 15, 20, 25, 50, 75, 100-fold or more reduction in the replication of HBV and/or HDV relative to pre-treatment levels in a subject, as determined after completion of, or completion of at least a portion of, the treatment regime (for example, 1 month after initiation or completion). In some embodiments, a compound of Formula (I), or a pharmaceutically acceptable salt thereof, can result in a reduction of the replication of HBV and/or HDV relative to pre-treatment levels in the range of more than 1 fold, about 2 to about 5 fold, about 10 to about 20 fold, about 15 to about 40 fold, or about 50 to about 100 fold. In some embodiments, a compound of Formula (I), or a pharmaceutically acceptable salt thereof, can result in a reduction of HBV and/or HDV replication in the range of more than 0.5 log, 1 to 1.5 log, 1.5 log to 2 log, 2 log to 2.5 log, 2.5 to 3 log, 3 log to 3.5 log or 3.5 to 4 log more reduction of HBV and/or HDV replication compared to the reduction of HBV and/or HDV replication achieved by the standard of care of HBV and/or HDV, administered according to the standard of care, or may achieve the same reduction as that standard of care therapy in a shorter period of time, for example, in one month, two months, or three months, as compared to the reduction achieved after six months of standard of care therapy.

In some embodiments, an effective amount of a compound of Formula (I), or a pharmaceutically acceptable salt thereof, is an amount that is effective to achieve a sustained virologic response, for example, non-detectable or substantially non-detectable HBV and/or HDV DNA load (e.g., less than about 25, or less than about 15 international units per milliliter serum) is found in the subject's serum for a period of at least about one month, at least about two months, at least about three months, at least about four months, at least about five months, or at least about six months following cessation of therapy.

In some embodiments, an effective amount of a compound of Formula (I), or a pharmaceutically acceptable salt thereof, can reduce the HBV and/or HDV viral load by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, or more, compared to the viral load a subject treated with standard of care, in an untreated subject or a placebo-treated subject. Methods of detecting HBV and/or HDV viral load are known to those skilled in the art and include immunological-based methods, e.g., enzyme-linked immunosorbent assays (ELISA), radioimmunoassays, and the like, that detect HBV and/or HDV antibodies and other markers indicative of HBV and/or HDV viral load, and combinations thereof.

Some embodiments described herein relate to a method of inhibiting HIV activity that can include contacting a cell infected with HIV with an effective amount of a compound of Formula (I), or a pharmaceutically acceptable salt thereof. Some embodiments described herein relate to a method of inhibiting HIV activity that can include administering to a subject infected with HIV an effective amount of a compound of Formula (I), or a pharmaceutically acceptable salt thereof. In some embodiments, a compound of Formula (I), or a pharmaceutically acceptable salt thereof, can inhibit a viral reverse transcriptase, and thus, inhibit the transcription of HIV RNA to DNA. In some embodiments, a compound of Formula (I), or a pharmaceutically acceptable salt thereof, can inhibit an HIV integrase. In some embodiments, a compound of Formula (I), or a pharmaceutically acceptable salt thereof, can inhibit viral envelop glycoprotein 120 (gp 120).

Some embodiments described herein relate to a method of treating a HIV infection that can include administering to a subject identified as suffering from the HIV infection an effective amount of a compound described herein (for example, a compound of Formula (I), or a pharmaceutically acceptable salt thereof), or a pharmaceutical composition that includes an effective amount of a compound described herein (such as, a compound of Formula (I), or a pharmaceutically acceptable salt thereof). Other embodiments described herein relate to using a compound described herein (for example, a compound of Formula (I), or a pharmaceutically acceptable salt thereof), in the manufacture of a medicament for treating a HIV infection. Still other embodiments described herein relate to the use of a compound described herein (such as, a compound of Formula (I), or a pharmaceutically acceptable salt thereof), or a pharmaceutical composition that includes an effective amount of a compound described herein (for example, a compound of Formula (I), or a pharmaceutically acceptable salt thereof) for treating a HIV infection.

Some embodiments disclosed herein relate to a method of treating a HIV infection that can include contacting a cell infected with the HIV with an effective amount of a compound described herein (for example, a compound of Formula (I), or a pharmaceutically acceptable salt thereof), or a pharmaceutical composition that includes an effective amount of a compound described herein. Other embodiments described herein relate to using a compound described herein (for example, a compound of Formula (I), or a pharmaceutically acceptable salt thereof) in the manufacture of a medicament for treating a HIV infection that can include contacting a cell infected with the HIV with an effective amount of said compound(s) and/or pharmaceutical composition. Still other embodiments described herein relate to the use of a compound described herein (for example, a compound of Formula (I), or a pharmaceutically acceptable salt thereof), or a pharmaceutical composition that includes a compound described herein for treating a HIV infection, wherein the use includes contacting a cell infected with the HIV with an effective amount of said compound(s) and/or pharmaceutical composition.

Some embodiments disclosed herein relate to a method of inhibiting replication of HIV that can include contacting a cell infected with the HIV with an effective amount of a compound described herein (for example, a compound of Formula (I), or a pharmaceutically acceptable salt thereof), or a pharmaceutical composition that includes a compound described herein (for example, a compound of Formula (I), or a pharmaceutically acceptable salt thereof). Other embodiments described herein relate to using a compound described herein (such as, a compound of Formula (I), or a pharmaceutically acceptable salt thereof) in the manufacture of a medicament for inhibiting replication of HIV that can include contacting a cell infected with HIV with an effective amount of said compound(s) and/or pharmaceutical composition. Still other embodiments described herein relate to the use of a compound described herein (for example, a compound of Formula (I), or a pharmaceutically acceptable salt thereof), or a pharmaceutical composition that includes an effective amount of a compound described herein (such as, a compound of Formula (I), or a pharmaceutically acceptable salt thereof), for inhibiting replication of HIV, wherein the use includes contacting a cell infected with the HIV with an effective amount of said compound(s) and/or pharmaceutical composition.

In some embodiments described herein, when the infection is caused by HIV, and/or the virus is HIV, the subject suffers from an opportunistic infection (OI). OIs take advantage of the subjects weakened immune system. In some embodiments described herein, a subject having a CD4+T lymphocyte count of less than about 200 cells/mL is an at increased risk of developing an OI. In some embodiments, OIs occur when the CD4+T lymphocyte count is less than about 500 cells/mL. In some embodiments, an OI occurs when an HIV viral load is greater than about 100,000 copies/mL. In some embodiments, HIV viral loads and/or CD4+T lymphocyte counts can be determined by conventional standard of care methodologies, for example, through HIV immunoassay detection assays for the detection of HIV antibodies and/or HIV p24 antigen.

Some embodiments described herein relate to a method of treating an OI related to a HIV infection selected from candidiasis, bronchitis, pneumonitis, esophagitis, invasive cervical cancer, coccidioidomycosis, cryptococcosis, chronic intestinal cryptosporidiosis, cytomegalovirus disease, encephalopathy, herpes simplex, histoplasmosis, chronic intestinal isosporiasis, Kaposi's sarcoma, lymphoma, Mycobacterium avium complex, tuberculosis, Pneumocystis carinii pneumonia, progressive multifocal leukoencephalopathy, salmonella septicemia, toxoplasmosis of brain, and wasting syndrome in a subject suffering from one or more of the aforementioned conditions that can include providing to the subject an effective amount of a compound or a pharmaceutical composition described herein (for example, a compound of Formula (I), or a pharmaceutically acceptable salt thereof). Some embodiments described herein relate to a method of preventing and/or treating one or more OI in a subject having a HIV infection that can include providing to the subject an effective amount of a compound or a pharmaceutical composition described herein (for example, a compound of Formula (I), or a pharmaceutically acceptable salt thereof). Also contemplated is a method for reducing or eliminating one or more OI in a subject having an HIV infection by providing to the subject an effective amount of a compound or a pharmaceutical composition described herein (for example, a compound of Formula (I), or a pharmaceutically acceptable salt thereof). In some embodiments, this method can include slowing or halting the progression of an OI. In other embodiments, the course of the OI can be reversed, and stasis or improvement in the infection is contemplated. In some embodiments, one or more of candidiasis, bronchitis, pneumonitis, esophagitis, invasive cervical cancer, coccidioidomycosis, cryptococcosis, chronic intestinal cryptosporidiosis, cytomegalovirus disease, encephalopathy, herpes simplex, histoplasmosis, chronic intestinal isosporiasis, Kaposi's sarcoma, lymphoma, Mycobacterium avium complex, tuberculosis, Pneumocystis carinii pneumonia, progressive multifocal leukoencephalopathy, salmonella septicemia, toxoplasmosis of brain, and wasting syndrome can be treated by contacting a cell infected with HIV with an effective amount of a compound described herein (for example, a compound of Formula (I), or a pharmaceutically acceptable salt thereof.)

Two types of HIV have been characterized, HIV-1 and HIV-2. HIV-1 is more virulent and more infective, and has a global prevalence, whereas HIV-2 is less virulent and is geographically confined. In some embodiments, an effective amount of a compound of Formula (I), or a pharmaceutically acceptable salt thereof, or a pharmaceutical composition that includes an effective amount of a compound of Formula (I), or a pharmaceutically acceptable salt thereof, can be effective to treat HIV-1. In some embodiments, an effective amount of a compound of Formula (I), or a pharmaceutically acceptable salt thereof, or a pharmaceutical composition that includes an effective amount of a compound of Formula (I), or a pharmaceutically acceptable salt thereof, can be effective to treat HIV-2. In some embodiments, a compound described herein (for example, a compound of Formula (I), or a pharmaceutically acceptable salt thereof) can be effective to treat both genotypes of HIV (HIV-1 and HIV-2).

Various indicators for determining the effectiveness of a method for treating an HIV infection are known to those skilled in the art. Examples of suitable indicators include, but are not limited to, a reduction in viral load, a reduction in plasma viral load, an increase CD4+T lymphocyte counts, a reduction in viral replication, a reduction in time to seroconversion (virus undetectable in patient serum), an increase in the rate of sustained viral response to therapy, a reduction of morbidity or mortality in clinical outcomes and/or a reduction in the rate of opportunistic infections. Similarly, successful therapy with an effective amount of a compound or a pharmaceutical composition described herein (for example, a compound of Formula (I), or a pharmaceutically acceptable salt thereof) can reduce the incidence of opportunistic infections in HIV infected subjects.

In some embodiments, an effective amount of a compound of Formula (I), or a pharmaceutically acceptable salt thereof, is an amount that is effective to reduce HIV viral titers to undetectable levels, for example, to about 10 to about 50, or to about 15 to about 25 international units/mL serum, or to less than about 20 international units/mL serum. In some embodiments, an effective amount of a compound of Formula (I), or a pharmaceutically acceptable salt thereof, is an amount that is effective to reduce HIV viral load compared to the HIV viral load before being provided the compound of Formula (I), or a pharmaceutically acceptable salt thereof. In some embodiments, an effective amount of a compound of Formula (I), or a pharmaceutically acceptable salt thereof, can be an amount that is effective to reduce HIV viral load to lower than about 20 international units/mL serum. In some embodiments, an effective amount of a compound of Formula (I), or a pharmaceutically acceptable salt thereof, is an amount that is effective to achieve a reduction in HIV viral titer in the serum of the subject in the range of about 1.5-log to about a 2.5-log reduction, about a 3-log to about a 4-log reduction, or a greater than about 5-log reduction compared to the viral load before being provided the compound of Formula (I), or a pharmaceutically acceptable salt thereof. For example, the HIV viral load can be measured before being provided the compound of Formula (I), or a pharmaceutically acceptable salt thereof, and again after completion of the treatment regime with the compound of Formula (I), or a pharmaceutically acceptable salt thereof (for example, 1 month after completion).

In some embodiments, an effective amount of a compound of Formula (I), or a pharmaceutically acceptable salt thereof, is an amount that is effective to increase CD4+T lymphocyte counts from less than about 200 cells/mL to greater than about 1,200 cells/mL. In some embodiments, an effective amount of a compound of Formula (I), or a pharmaceutically acceptable salt thereof, is an amount that is effective to increase CD4+T lymphocyte counts from less than about 200 cells/mL to greater than about 500 cells/mL.

In some embodiments, a compound of Formula (I), or a pharmaceutically acceptable salt thereof, can result in at least a 1, 2, 3, 4, 5, 10, 15, 20, 25, 50, 75, 100-fold or more reduction in the replication of the human immunodeficiency virus relative to pre-treatment levels in a subject, as determined after completion of the treatment regime (for example, 1 month after completion). In some embodiments, a compound of Formula (I), or a pharmaceutically acceptable salt thereof, can result in a reduction of the replication of the human immunodeficiency virus relative to pre-treatment levels in the range of about 2 to about 5 fold, about 10 to about 20 fold, about 15 to about 40 fold, or about 50 to about 100 fold. In some embodiments, a compound of Formula (I), or a pharmaceutically acceptable salt thereof, can result in a reduction of the human immunodeficiency virus replication in the range of 1 to 1.5 log, 1.5 log to 2 log, 2 log to 2.5 log, 2.5 to 3 log, 3 log to 3.5 log or 3.5 to 4 log more reduction of the human immunodeficiency virus replication compared to the reduction of the human immunodeficiency virus reduction achieved by standard of care therapy, such as therapy including ritonavir in combination with etravirine, or may achieve the same reduction as that standard of care therapy in a shorter period of time, for example, in one month, two months, or three months, as compared to the reduction achieved after six months of standard of care therapy.

In some embodiments, an effective amount of a compound of Formula (I), or a pharmaceutically acceptable salt thereof, is an amount that is effective to achieve a sustained viral response, for example, non-detectable or substantially non-detectable HIV RNA (e.g., less than about 25, or less than about 15 international units per milliliter serum) is found in the subject's serum for a period of at least about one month, at least about two months, at least about three months, at least about four months, at least about five months, or at least about six months following cessation of therapy.

In some embodiments, an effective amount of a compound of Formula (I), or a pharmaceutically acceptable salt thereof, can reduce the HIV viral load by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, or more, compared to the viral load in an untreated subject, or to a placebo-treated subject. Methods of detecting HIV viral load are known to those skilled in the art and include immunological-based methods, e.g., enzyme-linked immunosorbent assays (ELISA), radioimmunoassays, and the like, that detect HIV-1 and/or HIV-2 antibodies, HIV-1 p24 antigen, and other markers indicative of HIV viral load, and combinations thereof.

Subjects who are clinically diagnosed with HBV, HDV and/or HIV infection include “naïve” subjects (e.g., subjects not previously treated for HBV, HDV and/or HIV, particularly those who have not previously received ART for HIV, including ritonavir-based therapy) and individuals who have failed prior treatment for HBV, HDV and/or HIV (“treatment failure” subjects). Treatment failure subjects include “non-responders” (for HIV, these are subjects in whom the HIV titer was not significantly or sufficiently reduced by a previous treatment for HIV (≤0.5 log IU/mL)), for example, a previous ART, including ritonavir or other therapy; and “relapsers” (for HIV, subjects who were previously treated for HIV, for example, who received a previous ART whose HIV titer decreased, and subsequently increased). Further examples of subjects include subjects with an acute HBV and/or HDV infection, subjects with a chronic HBV and/or HDV, and subjects who are asymptomatic.

In some embodiments, a compound of Formula (I), or a pharmaceutically acceptable salt thereof, can be provided to a treatment failure subject suffering from HBV, HDV and/or HIV. In some embodiments, a compound of Formula (I), or a pharmaceutically acceptable salt thereof, can be provided to a non-responder subject suffering from HBV, HDV and/or HIV. In some embodiments, a compound of Formula (I), or a pharmaceutically acceptable salt thereof, can be provided to a relapsed subject suffering from HBV, HDV and/or HIV. In some embodiments, the subject can be asymptomatic, for example, the subject can be infected with HBV and/or HDV but does not exhibit any symptoms of the viral infection. In some embodiments, the subject can be immunocompromised. In some embodiments, the subject is suffering from at least one of HIV, HBV and/or HDV.

In some embodiments, a compound of Formula (I), or a pharmaceutically acceptable salt thereof, can be provided to a subject suffering from chronic HBV and/or HDV. In some embodiments, a compound of Formula (I), or a pharmaceutically acceptable salt thereof, can be provided to a subject suffering from acute HBV and/or HDV.

After a period of time, infectious agents can develop resistance to one or more therapeutic agents. The term “resistance” as used herein refers to a viral strain displaying a delayed, lessened and/or null response to a therapeutic agent(s). In some instances, the virus sometimes mutates or produces variations that are resistant or partially resistant to certain drugs. For example, after treatment with an antiviral agent, the viral load of a subject infected with a resistant virus may be reduced to a lesser degree compared to the amount in viral load reduction exhibited by a subject infected with a non-resistant strain. In some embodiments, a compound of Formula (I), or a pharmaceutically acceptable salt thereof, can be provided to a subject infected with an HBV and/or HDV strain that is resistant to one or more different anti-HBV and/or anti-HDV agents (for example, an agent used in a conventional standard of care). In some embodiments, development of resistant HBV and/or HDV strains is delayed when a subject is treated with a compound of Formula (I), or a pharmaceutically acceptable salt thereof, compared to the development of HBV and/or HDV strains resistant to other HBV and/or HDV drugs (such as an agent used in a conventional standard of care). In some embodiments, a compound of Formula (I), or a pharmaceutically acceptable salt thereof, can be provided to a subject infected with an HIV strain that is resistant to one or more different anti-HIV agents (for example, an agent used in a conventional standard of care). In some embodiments, development of resistant HIV strains is delayed when a subject is treated with a compound of Formula (I), or a pharmaceutically acceptable salt thereof, compared to the development of HIV strains resistant to other HIV drugs (such as an agent used in a conventional standard of care).

In some embodiments, an effective amount of a compound of Formula (I), or a pharmaceutically acceptable salt thereof, can be provided to a subject for whom other anti-HBV, anti-HDV and/or anti-HIV medications are contraindicated. In some embodiments, a compound of Formula (I), or a pharmaceutically acceptable salt thereof, can be provided to a subject that is hypersensitive to an antiviral agent.

Some subjects being treated for HBV, HDV and/or HIV experience a viral load rebound. The term “viral load rebound” as used herein refers to a sustained increase of viral load (such as ≥0.5 log IU/Ml for HIV) above nadir before the end of treatment. For HIV, nadir is a ≥0.5 log IU/mL decrease from baseline. In some embodiments, a compound of Formula (I), or a pharmaceutically acceptable salt thereof, can be provided to a subject experiencing viral load rebound, or can prevent such viral load rebound when used to treat the subject.

The standard of care for treating HBV, HDV and HIV has been associated with several side effects (also referred to as adverse effects). In some embodiments, a compound of Formula (I), or a pharmaceutically acceptable salt thereof, can decrease the number and/or severity of side effects observed in subjects being treated with the standard of care for a specific virus, such as HBV, HDV and HIV. Examples of side effects for a subject being treated for HBV and/or HDV include, but are not limited to dyspepsia, neuropathy, cough, loss of appetite, lactic acidosis, lipodystrophy, diarrhea, fatigue, insomnia, rash, fever, malaise, tachycardia, chills, headache, arthralgias, myalgias, apathy, nausea, vomiting, cognitive changes, asthenia, and drowsiness. In some embodiments, a compound of Formula (I), or a pharmaceutically acceptable salt thereof, can decrease the number and/or severity of side effects. For example, the number and/or severity of side effects observed in HIV subjects being treated with an ART according to the standard of care. Examples of side effects for a subject being treated for HIV include, but are not limited to loss of appetite, lipodystrophy, diarrhea, fatigue, elevated cholesterol and triglycerides, rash, insomnia, fever, malaise, tachycardia, chills, headache, arthralgias, myalgias, apathy, nausea, vomiting, cognitive changes, asthenia, drowsiness, lack of initiative, irritability, confusion, depression, severe depression, suicidal ideation, anemia, low white blood cell counts, and thinning of hair. In some embodiments, a compound of Formula (I), or a pharmaceutically acceptable salt thereof, can be provided to a subject that discontinued a HBV, HDV and/or HIV therapy because of one or more adverse effects or side effects associated with one or more other anti-HBV, HDV and/or HIV agents (for example, an agent used in a conventional standard of care).

Table 1 provides some embodiments of the percentage improvement obtained using a compound of Formula (I), or a pharmaceutically acceptable salt thereof, as compared to the standard of care for HBV, HDV and/or HIV. Examples include the following: in some embodiments, a compound of Formula (I), or a pharmaceutically acceptable salt thereof, results in a percentage of non-responders that is 10% less than the percentage of non-responders receiving the standard of care. In some embodiments, a compound of Formula (I), or a pharmaceutically acceptable salt thereof, results in a number of side effects that is in the range of about 10% to about 30% less than compared to the number of side effects experienced by a subject receiving the standard of care; and in some embodiments, a compound of Formula (I), or a pharmaceutically acceptable salt thereof, results in a severity of a side effect (such as one of those described herein) that is 25% less than compared to the severity of the same side effect experienced by a subject receiving the standard of care. Methods of quantifying the severity of a side effect are known to those skilled in the art.

TABLE 1

| Percentage | | Percentage | Percentage | | |
|---|---|---|---|---|---|
| of non- | Percentage | of | of viral load | Number of | Severity of |
| responders | of relapsers | resistance | rebound | side effects | side effects |
| 10% less | 10% less | 10% less | 10% less | 10% less | 10% less |
| 25% less | 25% less | 25% less | 25% less | 25% less | 25% less |
| 40% less | 40% less | 40% less | 40% less | 40% less | 40% less |
| 50% less | 50% less | 50% less | 50% less | 50% less | 50% less |
| 60% less | 60% less | 60% less | 60% less | 60% less | 60% less |
| 70% less | 70% less | 70% less | 70% less | 70% less | 70% less |
| 80% less | 80% less | 80% less | 80% less | 80% less | 80% less |
| 90% less | 90% less | 90% less | 90% less | 90% less | 90% less |
| about 10% | about 10% | about 10% | about 10% to | about 10% to | about 10% to |
| to about | to about 30% less | to about 30% less | about 30% less | about 30% less | about 30% less |
| 30% less | | | | | |
| about 20% | about 20% | about 20% | about 20% to | about 20% to | about 20% to |
| to about | to about | to about | about 50% | about 50% | about 50% |
| 50% less | 50% less | 50% less | less | less | less |
| about 30% | about 30% | about 30% | about 30% to | about 30% to | about 30% to |
| to about | to about | to about | about 70% | about 70% | about 70% |
| 70% less | 70% less | 70% less | less | less | less |
| about 20% | about 20% | about 20% | about 20% to | about 20% to | about 20% to |
| to about | to about | to about | about 80% | about 80% | about 80% |
| 80% less | 80% less | 80% less | less | less | less |

As used herein, a “subject” refers to an animal that is the object of treatment, observation or experiment. “Animal” includes cold- and warm-blooded vertebrates and invertebrates such as fish, shellfish, reptiles and, in particular, mammals. “Mammal” includes, without limitation, mice, rats, rabbits, guinea pigs, dogs, cats, sheep, goats, cows, horses, primates, such as monkeys, chimpanzees, and apes, and, in particular, humans. In some embodiments, the subject is human.

As used herein, the terms “treating,” “treatment,” “therapeutic,” or “therapy” do not necessarily mean total cure or abolition of the disease or condition. Any alleviation of any undesired signs or symptoms of a disease or condition, to any extent can be considered treatment and/or therapy. Furthermore, treatment may include acts that may worsen the patient's overall feeling of well-being or appearance.

The terms “therapeutically effective amount” and “effective amount” are used to indicate an amount of an active compound, or pharmaceutical agent, that elicits the biological or medicinal response indicated. For example, an effective amount of compound can be the amount needed to prevent, alleviate or ameliorate symptoms of disease or prolong the survival of the subject being treated. This response may occur in a tissue, system, animal or human and includes alleviation of the signs or symptoms of the disease being treated. Determination of an effective amount is well within the capability of those skilled in the art, in view of the disclosure provided herein. The effective amount of the compounds disclosed herein required as a dose will depend on the route of administration, the type of animal, including human, being treated, and the physical characteristics of the specific animal under consideration. The dose can be tailored to achieve a desired effect, but will depend on such factors as weight, diet, concurrent medication and other factors which those skilled in the medical arts will recognize.

As will be readily apparent to one skilled in the art, the useful in vivo dosage to be administered and the particular mode of administration will vary depending upon the age, weight, the severity of the affliction, and mammalian species treated, the particular compounds employed, and the specific use for which these compounds are employed. The determination of effective dosage levels, that is the dosage levels necessary to achieve the desired result, can be accomplished by one skilled in the art using routine methods, for example, human clinical trials and in vitro studies.

The dosage may range broadly, depending upon the desired effects and the therapeutic indication. Alternatively, dosages may be based and calculated upon the surface area of the patient, as understood by those of skill in the art. Although the exact dosage will be determined on a drug-by-drug basis, in most cases, some generalizations regarding the dosage can be made. The daily dosage regimen for an adult human patient may be, for example, an oral dose of between 0.01 mg and 3000 mg of each active ingredient, preferably between 1 mg and 700 mg, e.g. 5 to 200 mg. The dosage may be a single one or a series of two or more given in the course of one or more days, as is needed by the subject. In some embodiments, the compounds will be administered for a period of continuous therapy, for example for a week or more, or for months or years. In some embodiments, a compound of Formula (I), or a pharmaceutically acceptable salt thereof, can be administered less frequently compared to the frequency of administration of an agent within the standard of care. In some embodiments, a compound of Formula (I), or a pharmaceutically acceptable salt thereof, can be administered one time per day. For example, a compound of Formula (I), or a pharmaceutically acceptable salt thereof, can be administered one time per day to a subject suffering from a HIV infection. In some embodiments, the total time of the treatment regime with a compound of Formula (I), or a pharmaceutically acceptable salt thereof, can be less compared to the total time of the treatment regime with the standard of care.

In instances where human dosages for compounds have been established for at least some condition, those same dosages may be used, or dosages that are between about 0.1% and 500%, more preferably between about 25% and 250% of the established human dosage. Where no human dosage is established, as will be the case for newly-discovered pharmaceutical compositions, a suitable human dosage can be inferred from ED₅₀ or ID₅₀ values, or other appropriate values derived from in vitro or in vivo studies, as qualified by toxicity studies and efficacy studies in animals.

In cases of administration of a pharmaceutically acceptable salt, dosages may be calculated as the free base. As will be understood by those of skill in the art, in certain situations it may be necessary to administer the compounds disclosed herein in amounts that exceed, or even far exceed, the above-stated, preferred dosage range in order to effectively and aggressively treat particularly aggressive diseases or infections.

Dosage amount and interval may be adjusted individually to provide plasma levels of the active moiety which are sufficient to maintain the modulating effects, or minimal effective concentration (MEC). The MEC will vary for each compound but can be estimated from in vitro data. Dosages necessary to achieve the MEC will depend on individual characteristics and route of administration. However, HPLC assays or bioassays can be used to determine plasma concentrations. Dosage intervals can also be determined using MEC value. Compositions should be administered using a regimen which maintains plasma levels above the MEC for 10-90% of the time, preferably between 30-90% and most preferably between 50-90%. In cases of local administration or selective uptake, the effective local concentration of the drug may not be related to plasma concentration.

It should be noted that the attending physician would know how to and when to terminate, interrupt, or adjust administration due to toxicity or organ dysfunctions. Conversely, the attending physician would also know to adjust treatment to higher levels if the clinical response were not adequate (precluding toxicity). The magnitude of an administrated dose in the management of the disorder of interest will vary with the severity of the condition to be treated and to the route of administration. The severity of the condition may, for example, be evaluated, in part, by standard prognostic evaluation methods. Further, the dose and perhaps dose frequency, will also vary according to the age, body weight, and response of the individual patient. A program comparable to that discussed above may be used in veterinary medicine.

Compounds disclosed herein can be evaluated for efficacy and toxicity using known methods. For example, the toxicology of a particular compound, or of a subset of the compounds, sharing certain chemical moieties, may be established by determining in vitro toxicity towards a cell line, such as a mammalian, and preferably human, cell line. The results of such studies are often predictive of toxicity in animals, such as mammals, or more specifically, humans. Alternatively, the toxicity of particular compounds in an animal model, such as mice, rats, rabbits, or monkeys, may be determined using known methods. The efficacy of a particular compound may be established using several recognized methods, such as in vitro methods, animal models, or human clinical trials. When selecting a model to determine efficacy, the skilled artisan can be guided by the state of the art to choose an appropriate model, dose, route of administration and/or regime.

### Combination Therapies

In some embodiments, the compounds disclosed herein, such as a compound of Formula (I), or a pharmaceutically acceptable salt thereof, or a pharmaceutical composition that includes a compound described herein, or a pharmaceutically acceptable salt thereof, can be used in combination with one or more additional agent(s). Examples of additional agents that can be used in combination with a compound of Formula (I), or a pharmaceutically acceptable salt thereof, or a pharmaceutical composition that includes a compound of Formula (I), or a pharmaceutically acceptable salt thereof, can be agents currently used in a conventional standard of care for treating HIV, HBV, and/or HDV. In some embodiments, a compound of Formula (I), or a pharmaceutically acceptable salt thereof, or a pharmaceutical composition that includes a compound of Formula (I), or a pharmaceutically acceptable salt thereof, can be used with one, two, three or more additional agents described herein.

In some embodiments, when the infection is caused by HBV, HBV and/or HIV, the additional therapeutic agent can be an antiretroviral therapy (ART) agent such as a non-nucleoside reverse transcriptase inhibitor (NNRTI), a nucleoside reverse transcriptase inhibitor (NRTI), a polymerase inhibitor, a protease inhibitor (PI), a fusion/entry inhibitor, an interferon, a viral maturation inhibitor, a capsid assembly modulator, a FXR agonist, a TNF/cyclophilin inhibitor, a TLR agonist, a vaccine, an siRNA or ASO covalently closed circular DNA (cccDNA) inhibitor, a gene silencing agent, an HBx inhibitor, a surface antigen (sAg) secretion inhibitor (for example, HBsAg), other HBV antiviral compound, other HDV antiviral compound and/or other HIV antiviral compound, or a pharmaceutically acceptable salt of any of the foregoing.

In some embodiments, a compound of Formula (I), or a pharmaceutically acceptable salt thereof, or a pharmaceutical composition that includes a compound of Formula (I), or a pharmaceutically acceptable salt thereof, can be used in combination with an agent(s) currently used in a conventional standard of care therapy. For example, for the treatment of HBV and/or HDV, a compound disclosed herein can be used in combination with an interferon therapy.

In some embodiments, a compound of Formula (I), or a pharmaceutically acceptable salt thereof, or a pharmaceutical composition that includes a compound of Formula (I), or a pharmaceutically acceptable salt thereof, can be substituted for an agent currently used in a conventional standard of care therapy. For example, for the treatment of HIV, a compound of Formula (I), or a pharmaceutically acceptable salt thereof, or a pharmaceutical composition that includes a compound of Formula (I), or a pharmaceutically acceptable salt thereof, can be used in place of a conventional ART inhibitor.

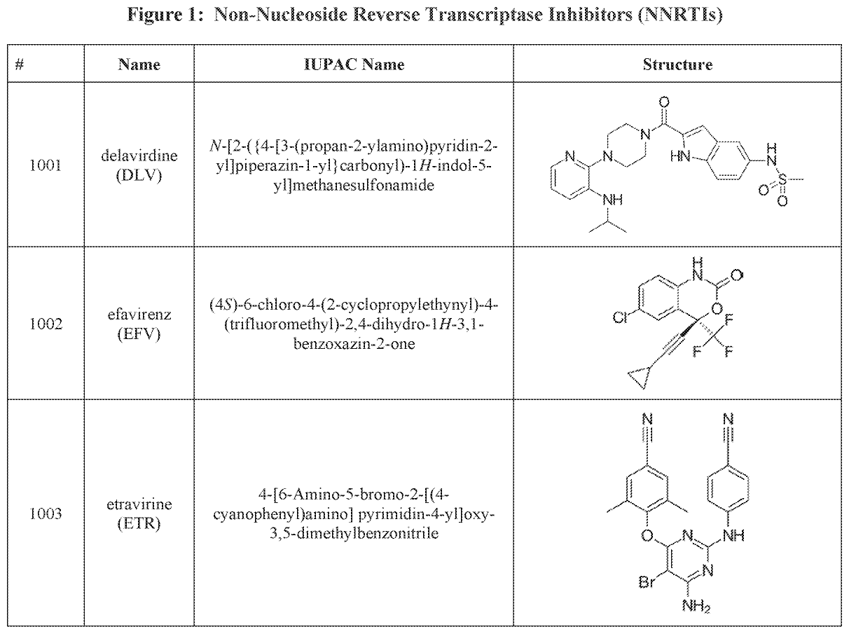

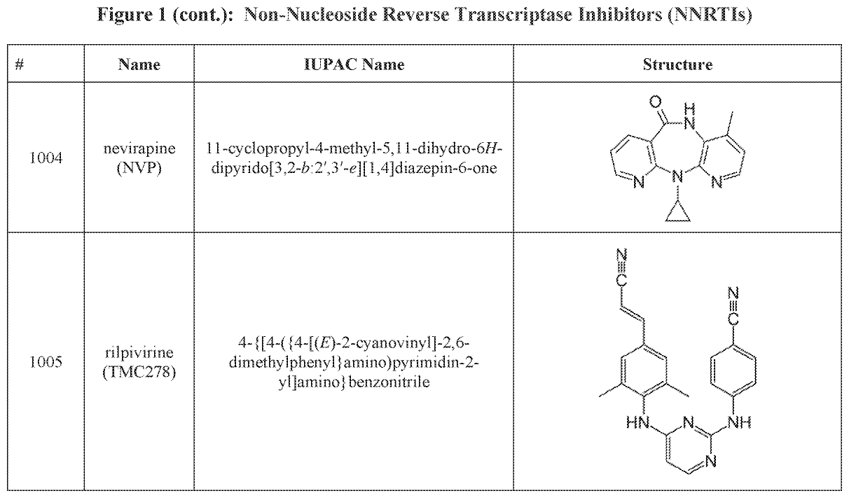

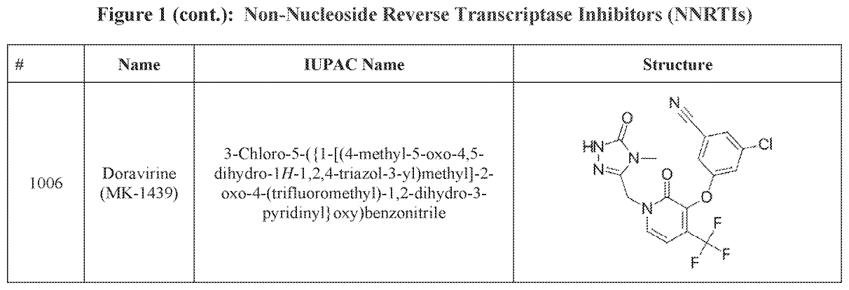

In some embodiments, a compound of Formula (I), or a pharmaceutically acceptable salt thereof, or a pharmaceutical composition that includes a compound of Formula (I), or a pharmaceutically acceptable salt thereof, can be used in combination with a non-nucleoside reverse transcriptase inhibitor (NNRTI). In some embodiments, the NNRTI can inhibit a HBV and/or HDV reverse transcriptase. Examples of suitable NNRTIs include, but are not limited to, delavirdine (Rescriptor®), efavirenz (Sustiva®), etravirine (Intelence®), nevirapine (Viramune®), rilpivirine (Edurant®), doravirine, and pharmaceutically acceptable salts of any of the foregoing, and/or a combination thereof. A non-limiting list of example NNRTIs includes compounds numbered 1001-1006 in FIG. 1.

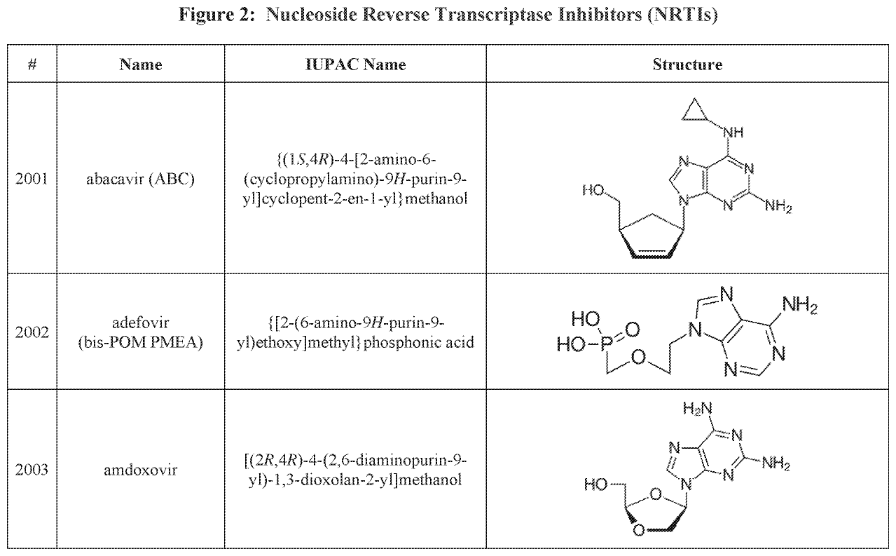

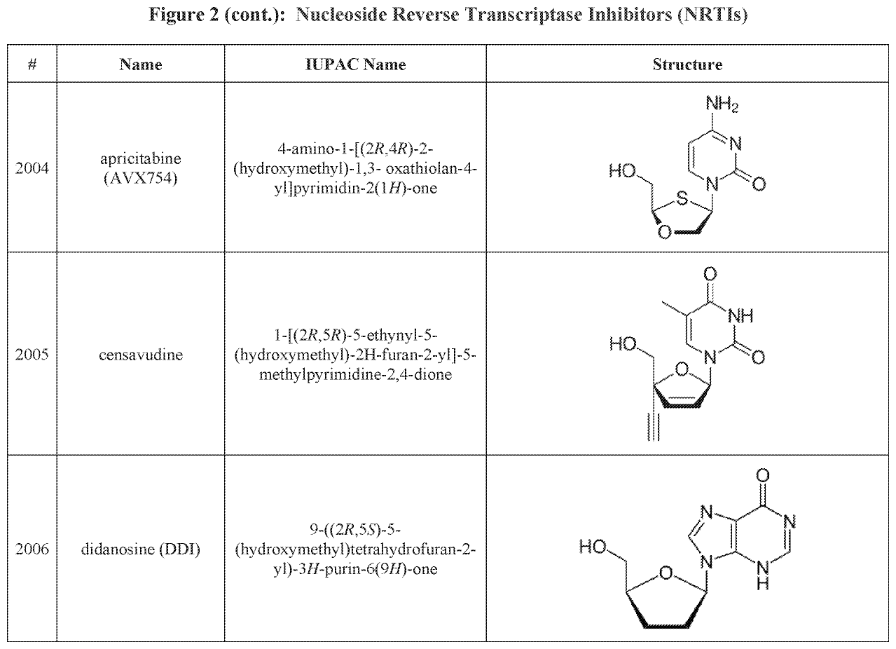

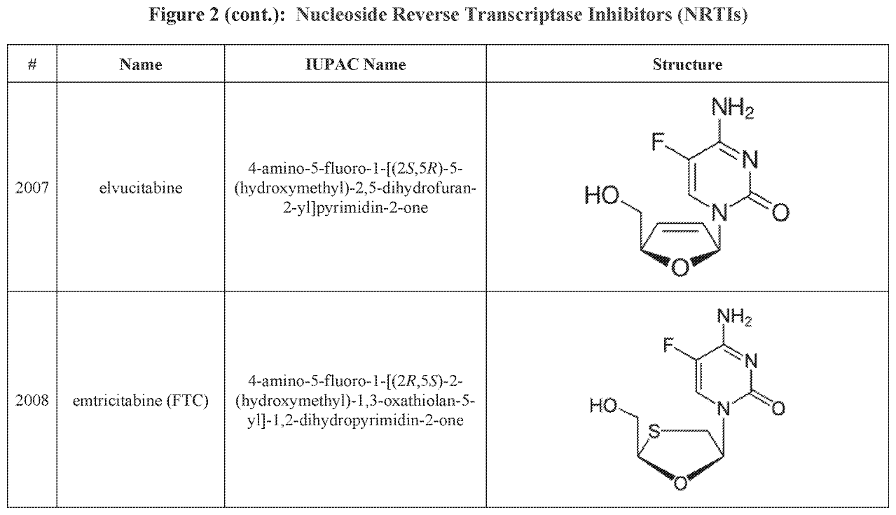

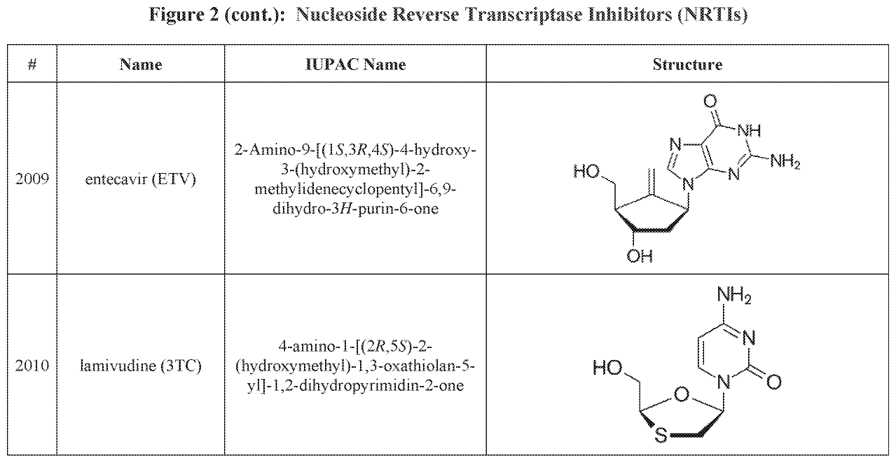

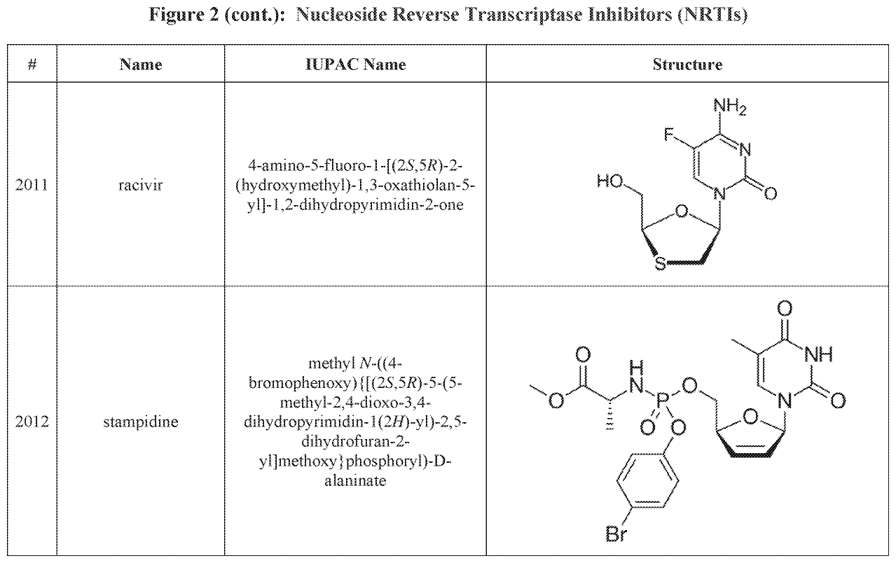

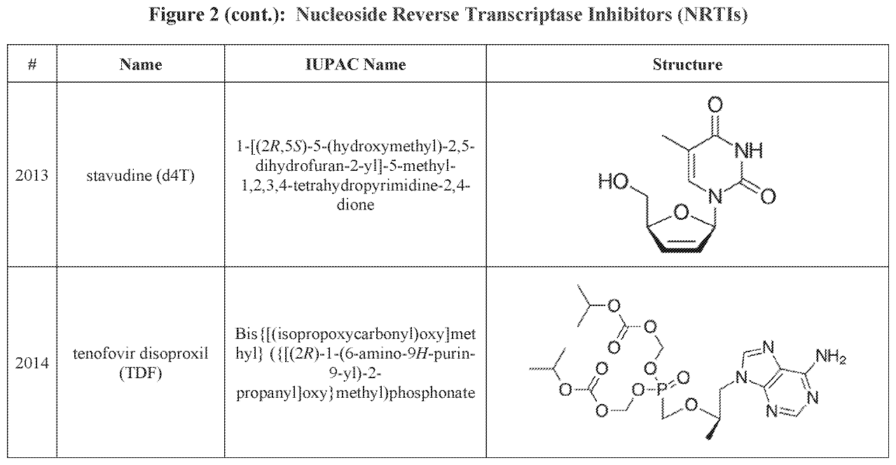

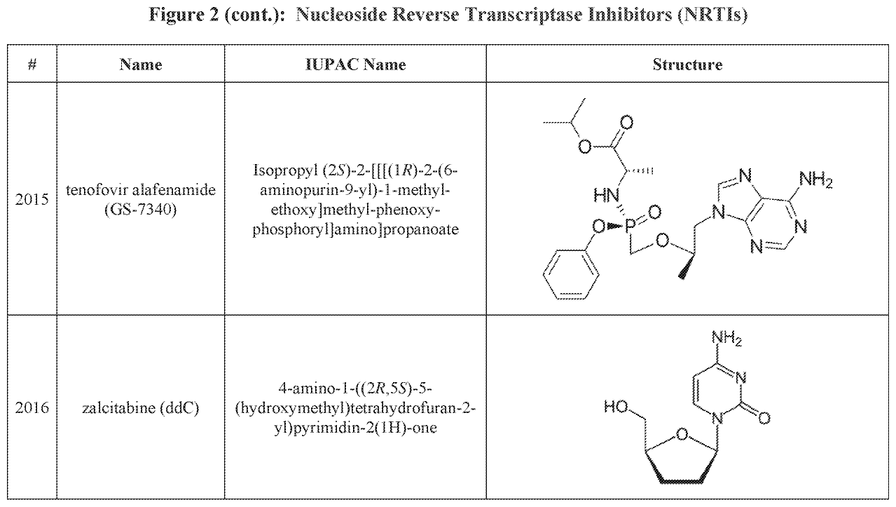

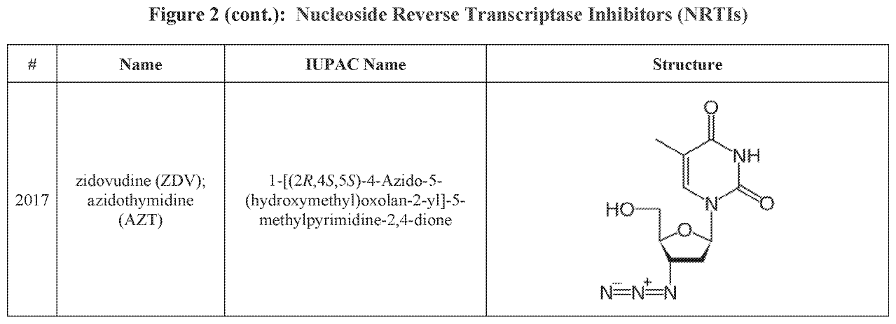

In some embodiments, a compound of Formula (I), or a pharmaceutically acceptable salt thereof, or a pharmaceutical composition that includes a compound of Formula (I), or a pharmaceutically acceptable salt thereof, can be used in combination with a nucleoside reverse transcriptase inhibitor (NRTI). In some embodiments, the NRTI can inhibit a HBV and/or HDV reverse transcriptase. Examples of suitable NRTIs include, but are not limited to, abacavir (Ziagen®), adefovir (Hepsera®), amdoxovir, apricitabine, censavudine, didanosine (Videx®), elvucitabine, emtricitabine (Emtriva®), entecavir (Baraclude®), lamivudine (Epivir®), racivir, stampidine, stavudine (Zerit®), tenofovir disoproxil (including Viread®), tenofovir alafenamide, zalcitabine (Hivid®), zidovudine (Retrovir®), and pharmaceutically acceptable salts of any of the foregoing, and/or a combination thereof. A non-limiting list of example NRTIs includes compounds numbered 2001-2017 in FIG. 2.

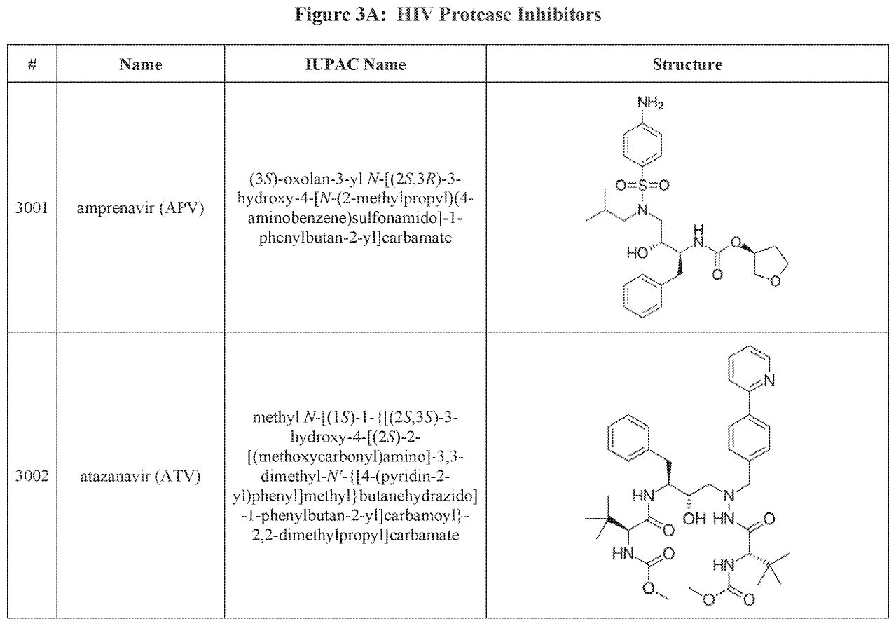

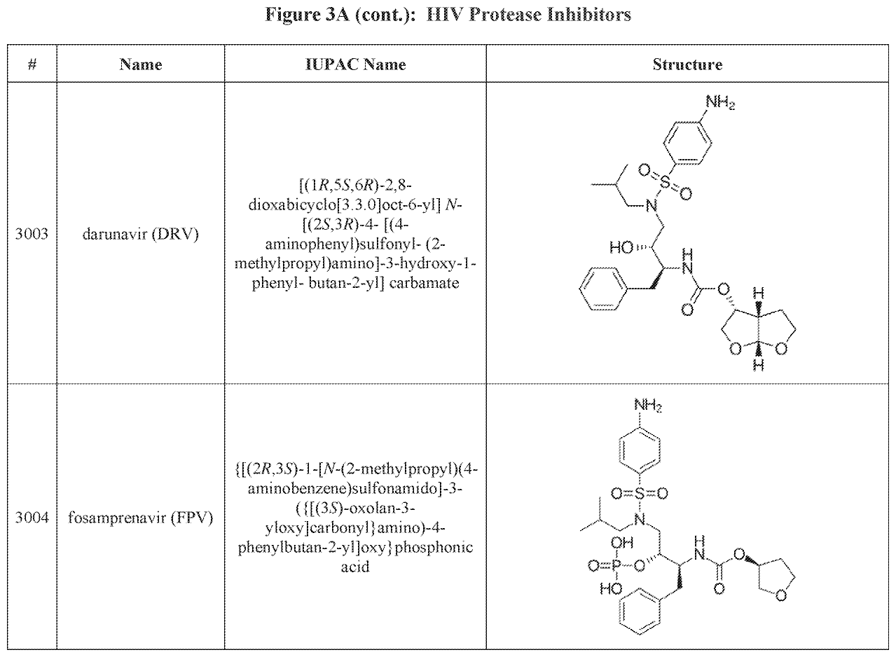

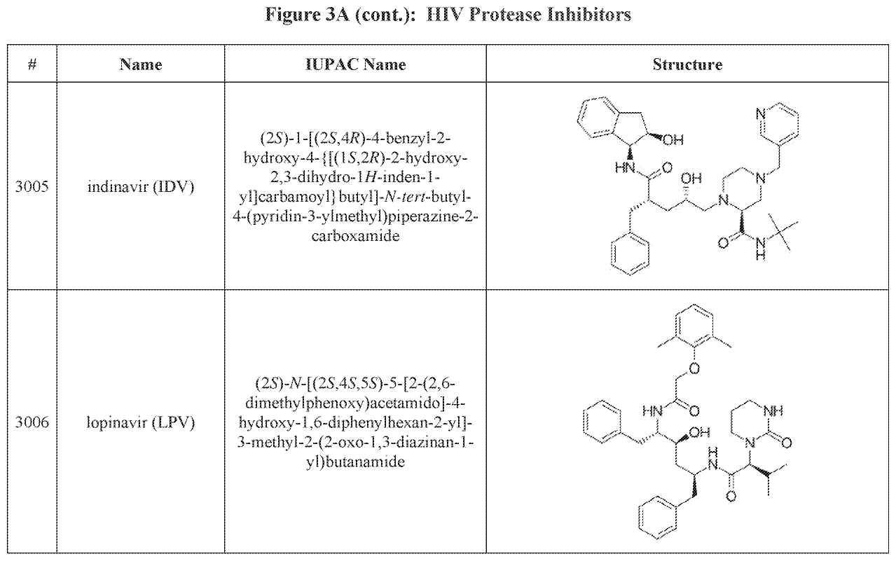

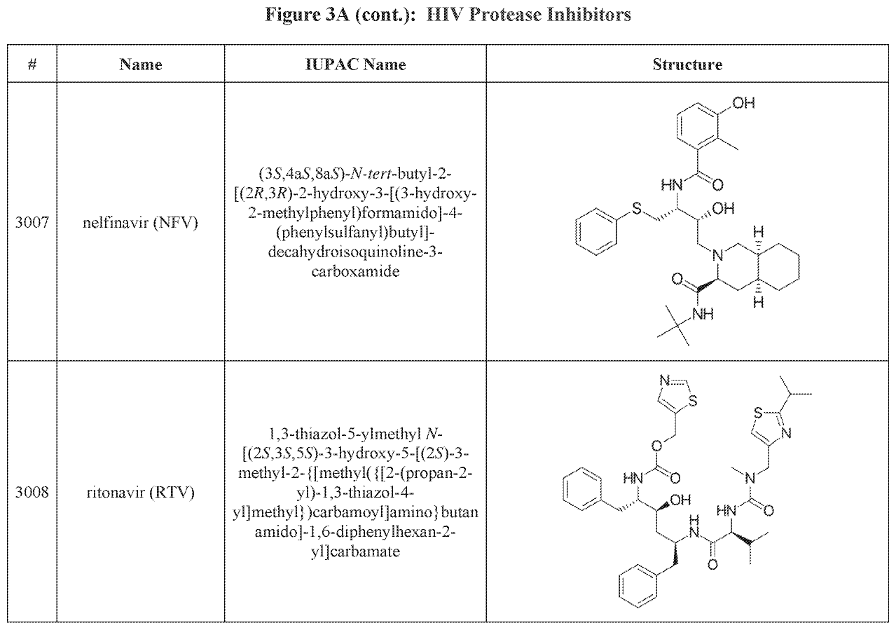

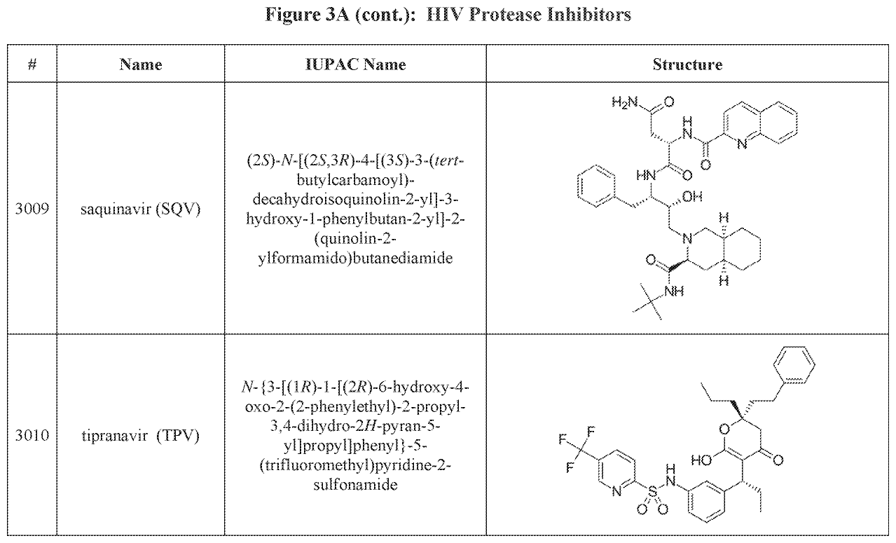

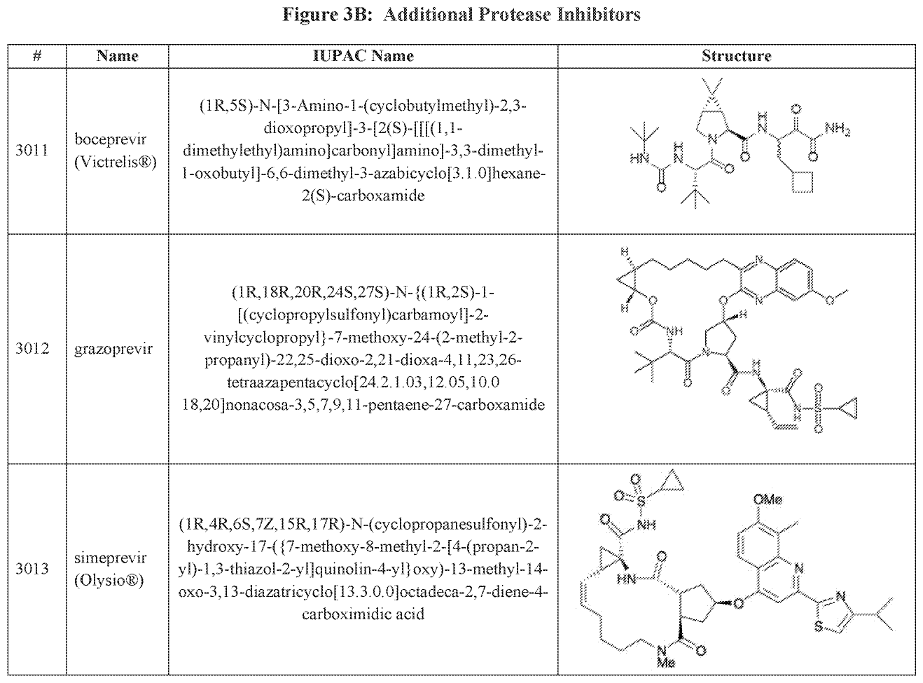

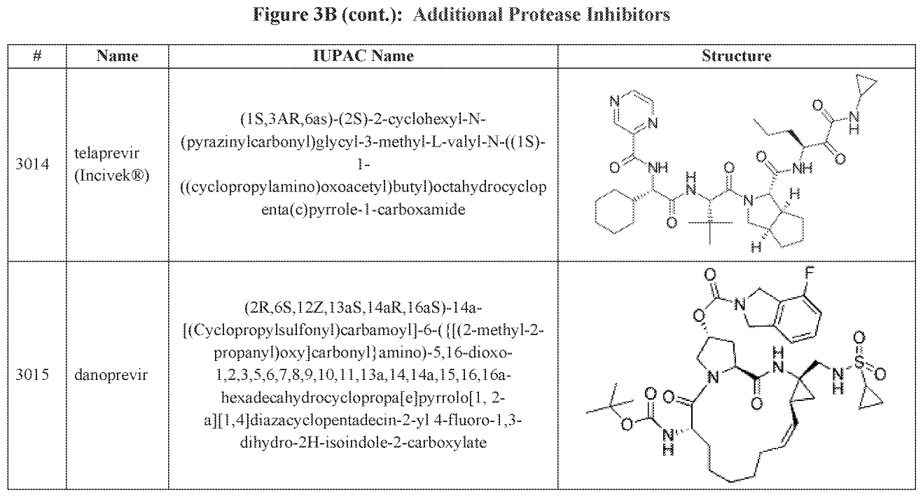

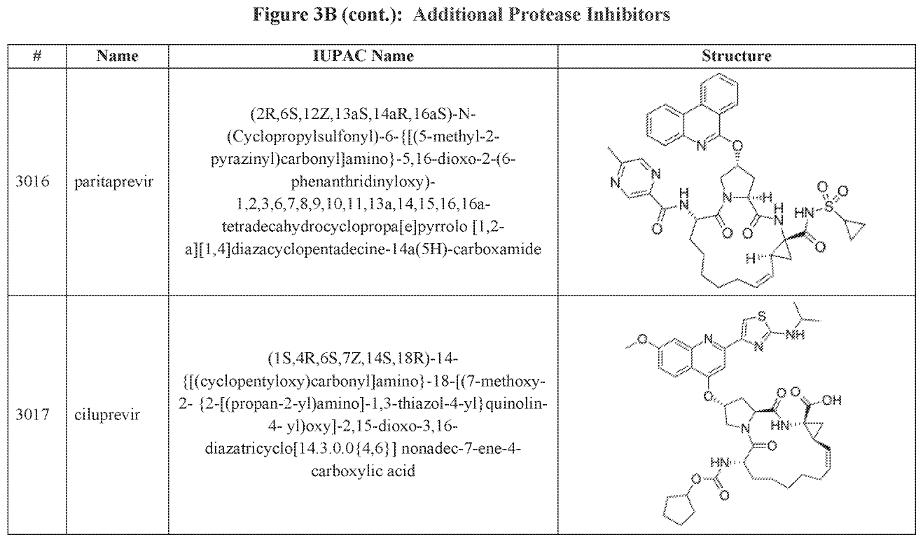

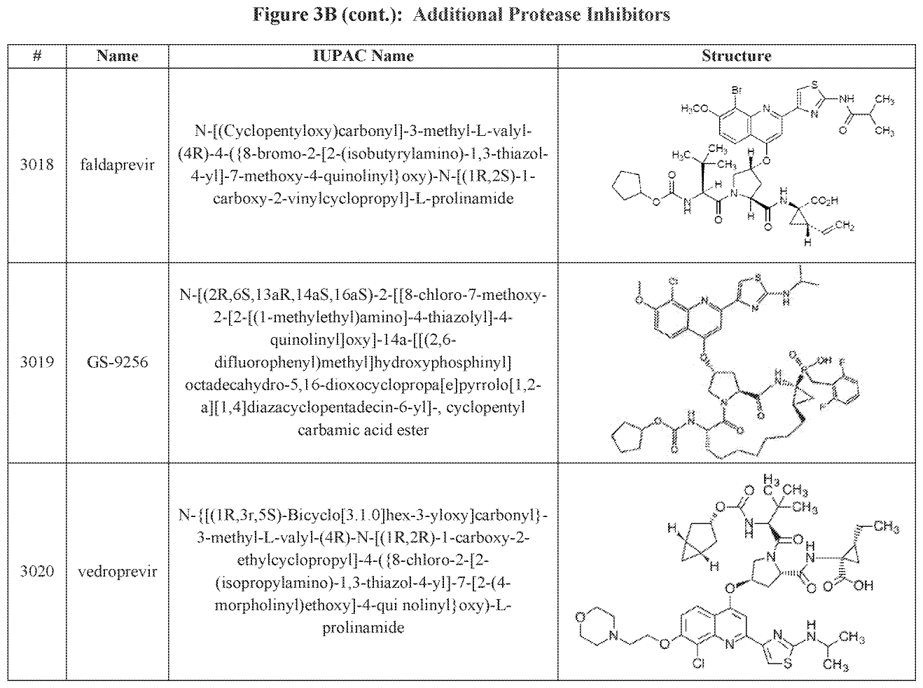

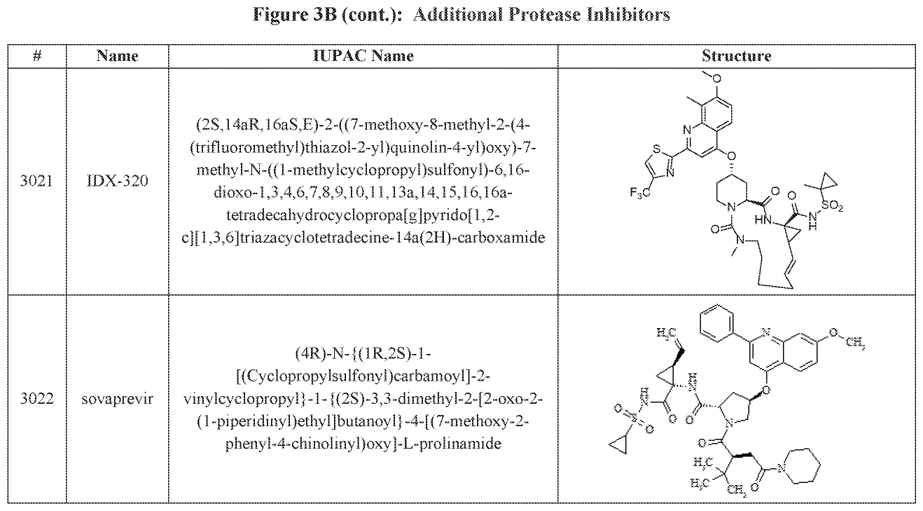

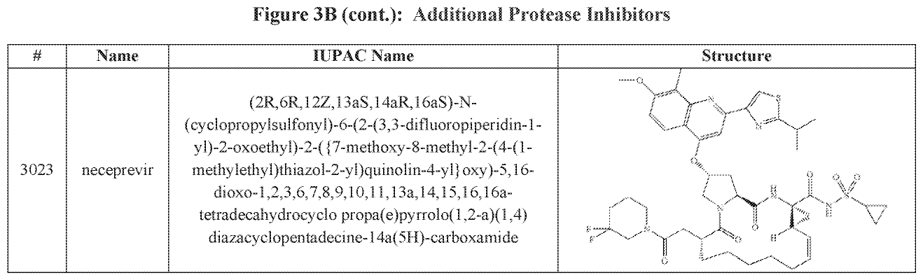

In some embodiments, a compound of Formula (I), or a pharmaceutically acceptable salt thereof, or a pharmaceutical composition that includes a compound of Formula (I), or a pharmaceutically acceptable salt thereof, can be used in combination with a protease inhibitor. In some embodiments, the protease inhibitor can inhibit a HBV and/or HDV protease, for example NS3/4A. A non-limiting list of example protease inhibitors include the following: amprenavir (Agenerase®), asunaprevir (Sunvepra®), atazanavir (Reyataz®), boceprevir (Victrelis®), darunavir (Prezista®), fosamprenavir (Lexiva®; Telzir®), grazoprevir, indinavir (Crixivan®), lopinavir (Kaletra®), nelfinavir (Viracept®), ritonavir (Norvir®), saquinavir (Fortovase®; Invirase®), simeprevir (Olysio®), telaprevir (Incivek®), danoprevir, tipranavir (Aptivus®), ABT-450 (paritaprevir), BILN-2061 (ciluprevir), BI-201335 (faldaprevir), GS-9256, vedroprevir (GS-9451), IDX-320, ACH-1625 (sovaprevir), ACH-2684, and pharmaceutically acceptable salts of any of the foregoing, and/or combinations thereof. A non-limiting list of example protease inhibitors includes compounds numbered 3001-3010 in FIG. 3A and 3011-3023 in FIG. 3B.

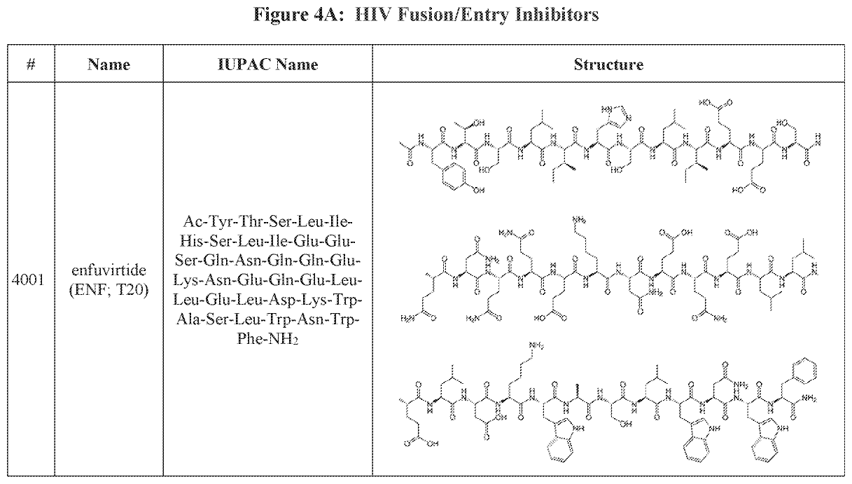

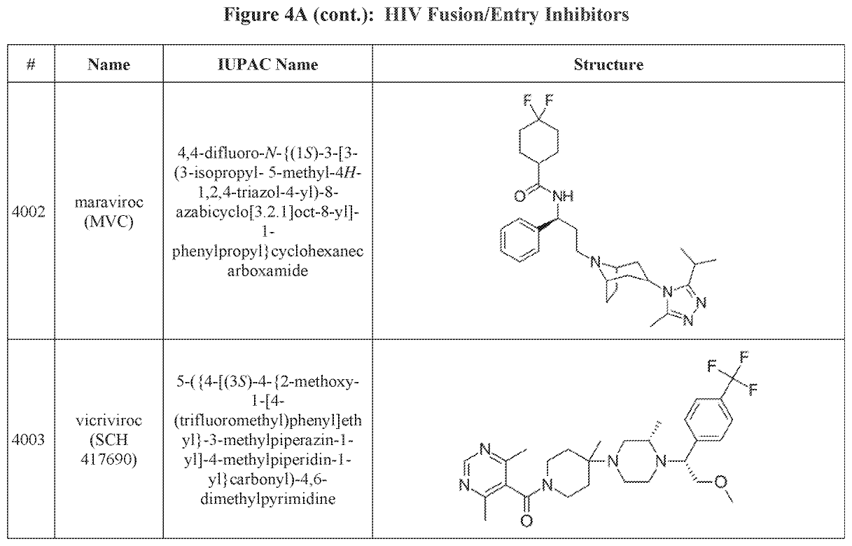

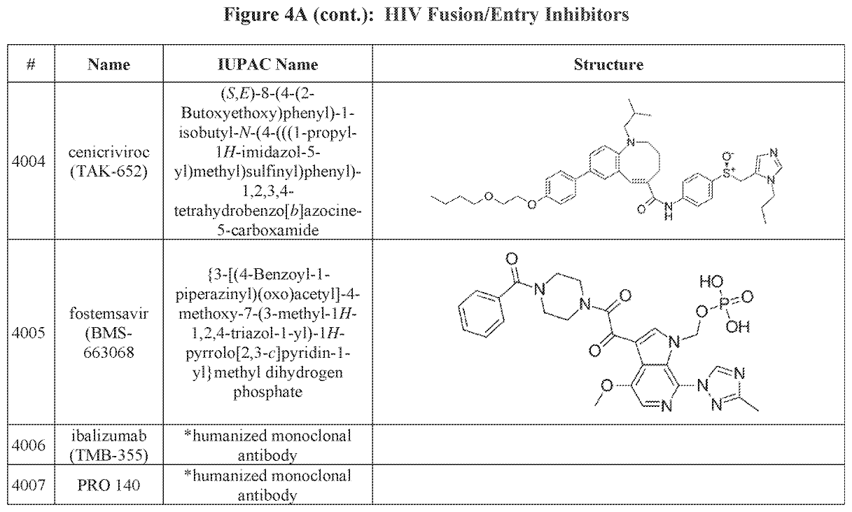

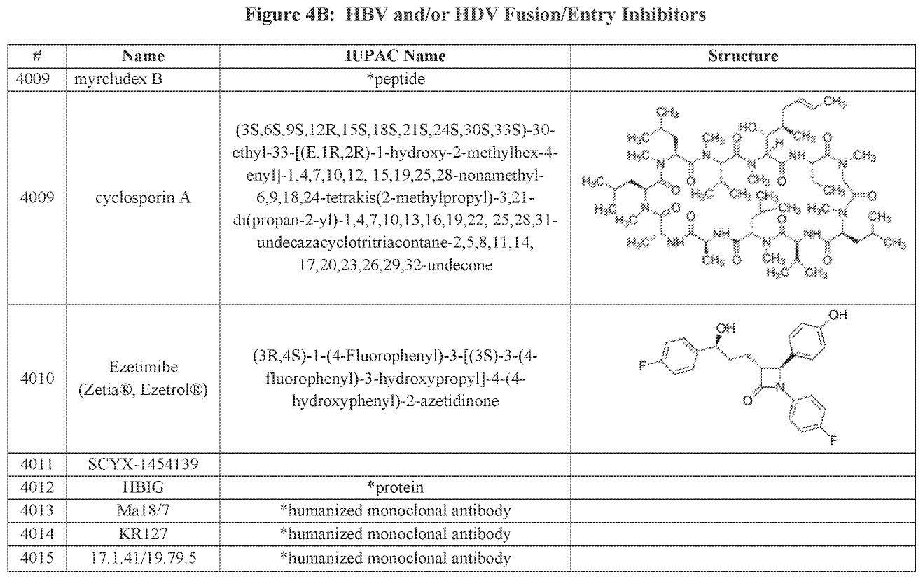

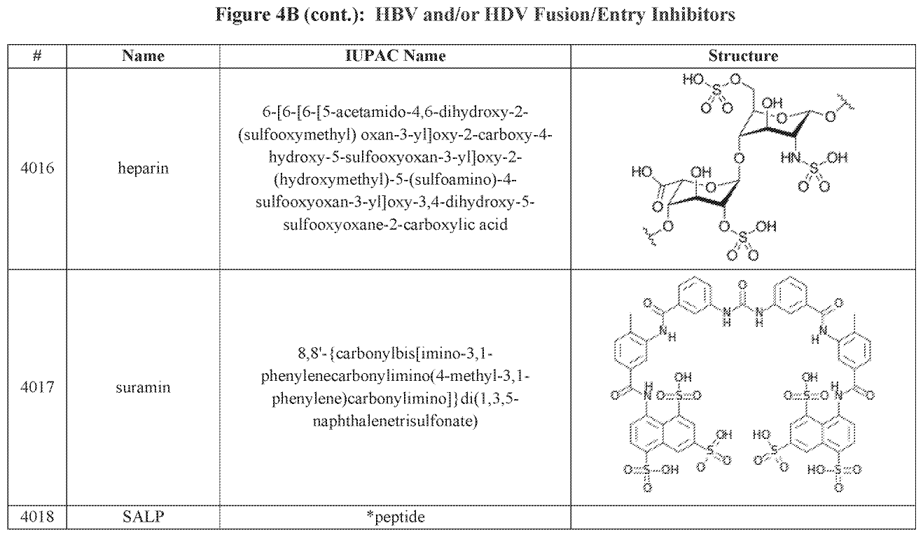

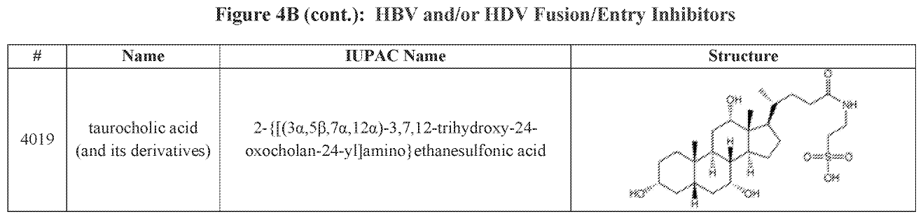

In some embodiments, a compound of Formula (I), or a pharmaceutically acceptable salt thereof, or a pharmaceutical composition that includes a compound of Formula (I), or a pharmaceutically acceptable salt thereof, can be used in combination with an HIV fusion/entry inhibitor. In some embodiments, the HIV fusion/entry inhibitors can block HIV from entering the CD4+T lymphocytes. In some embodiments, the fusion/entry inhibitors, which are also known as CCR5 antagonists, can block proteins on the CD4+T lymphocyte cells that are required for HIV cellular entry. Examples of suitable fusion/entry inhibitors include, but are not limited to, enfuvirtide (Fuzeon®), maraviroc (Selzentry®), vicriviroc, cenicriviroc, fostemsavir, ibalizumab, PRO 140, and pharmaceutically acceptable salts of any of the foregoing, and/or combinations thereof. A non-limiting list of example HIV fusion/entry inhibitors includes compounds numbered 4001-4007 in FIG. 4A.

In some embodiments, a compound of Formula (I), or a pharmaceutically acceptable salt thereof, or a pharmaceutical composition that includes a compound of Formula (I), or a pharmaceutically acceptable salt thereof, can be used in combination with a HBV and/or HDV fusion/entry inhibitor. In some embodiments, the fusion/entry inhibitors can block HBV and/or HDV from entering hepatocytes. In some embodiments, the HBV and/or HDV fusion/entry inhibitors can block proteins on the hepatocytes that are required for HBV and/or HDV cellular entry. In some embodiments, the HBV and/or HDV fusion/entry inhibitors can bind to sodium-taurocholate cotransporting polypeptides. Examples of suitable HBV and/or HDV fusion/entry inhibitors include, but are not limited to, myrcludex B, cyclosporin A, ezetimibe, and SCYX1454139, HBIG, Mal8/7, KR127, 17.1.41/19.79.5, heparin, suramin, SALP, taurocholic acid derivatives, and pharmaceutically acceptable salts of any of the foregoing, and/or combinations thereof. A non-limiting list of example HBV and/or HDV fusion/entry inhibitors includes compounds numbered 4008-4019 in FIG. 4B.

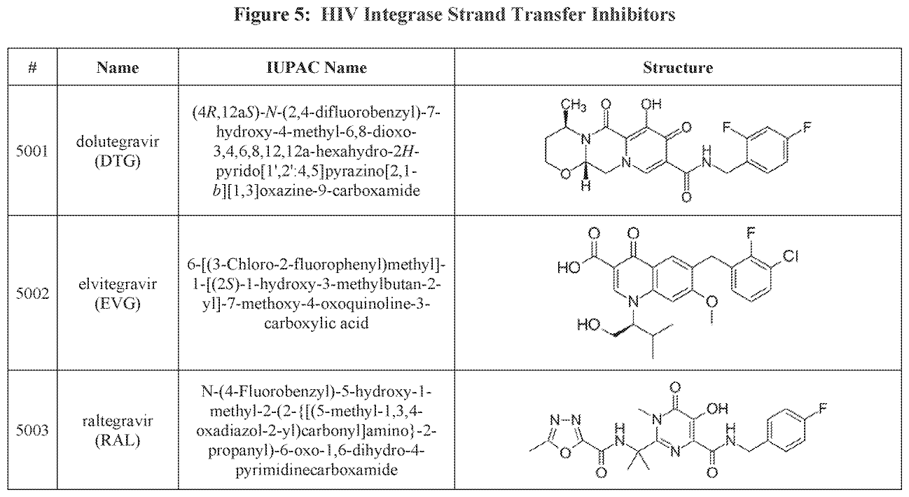

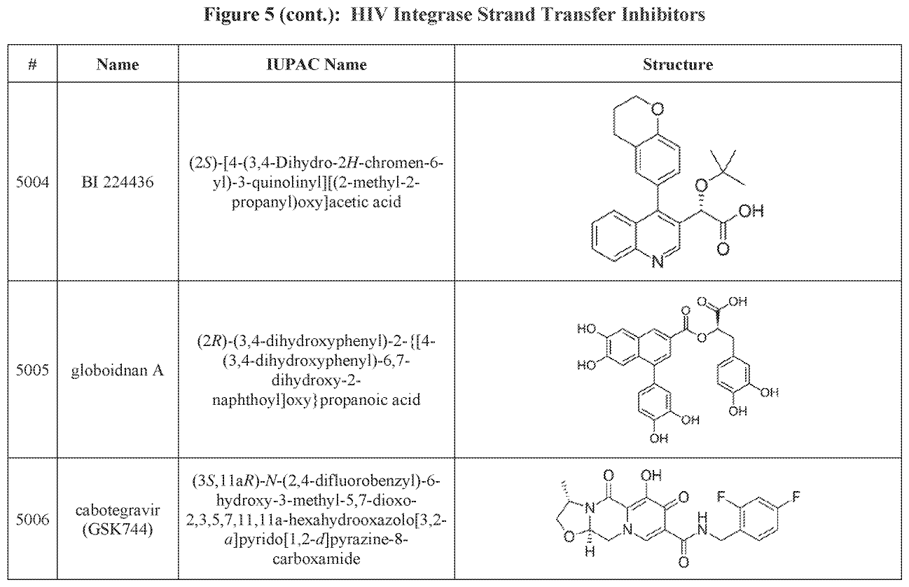

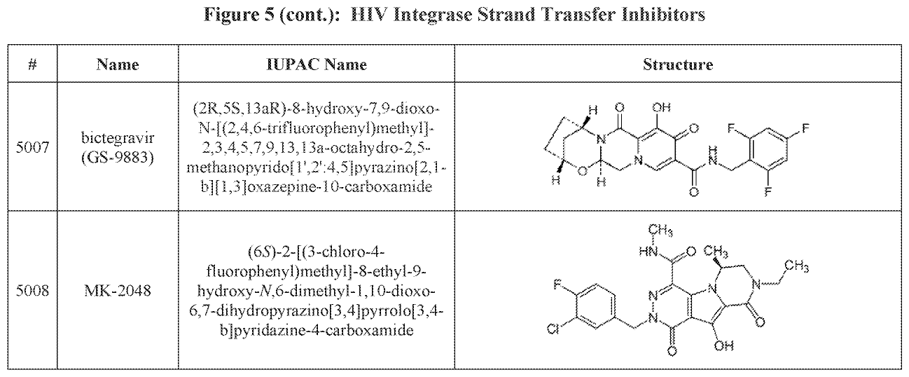

In some embodiments, a compound of Formula (I), or a pharmaceutically acceptable salt thereof, or a pharmaceutical composition that includes a compound of Formula (I), or a pharmaceutically acceptable salt thereof, can be used in combination with an HIV integrase strand transfer inhibitor (INSTI). In some embodiments, the INSTI can block HIV integrase. Examples of INSTIs include, but are not limited to, dolutegravir (Tivicay®), elvitegravir (Strivild®; Vitekta®), raltegravir (Isentress®), BI 224436, globoidnan A, cabotegravir, bictegravir, MK-2048, and pharmaceutically acceptable salts of any of the foregoing, and/or a combination thereof. A non-limiting list of example HIV INSTIs includes compounds numbered 5001-5008 in FIG. 5.

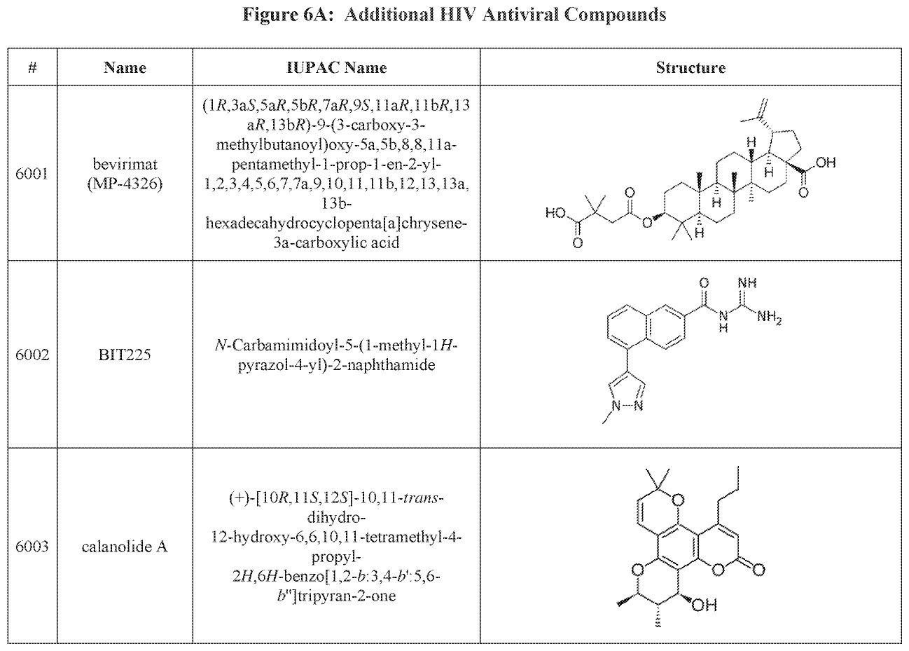

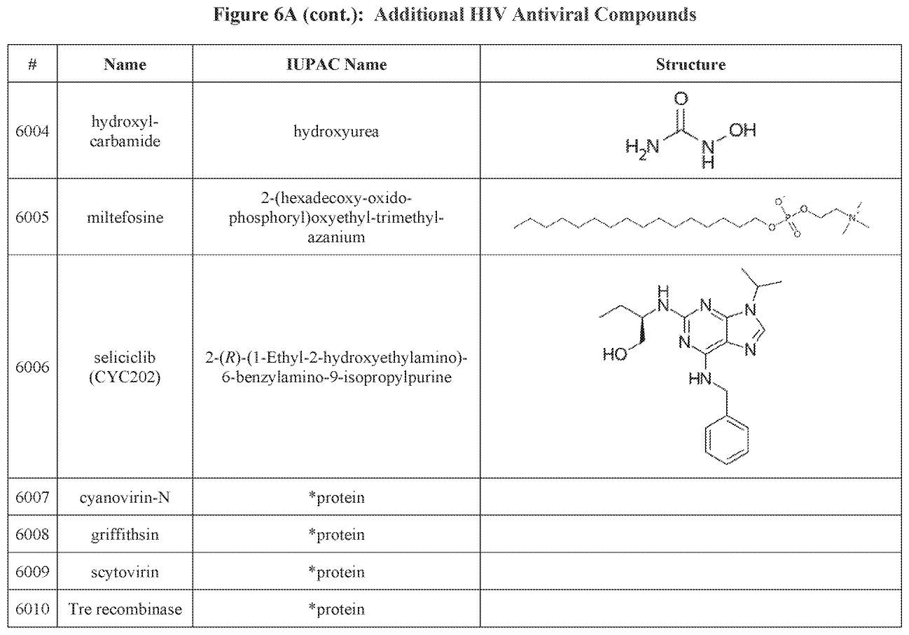

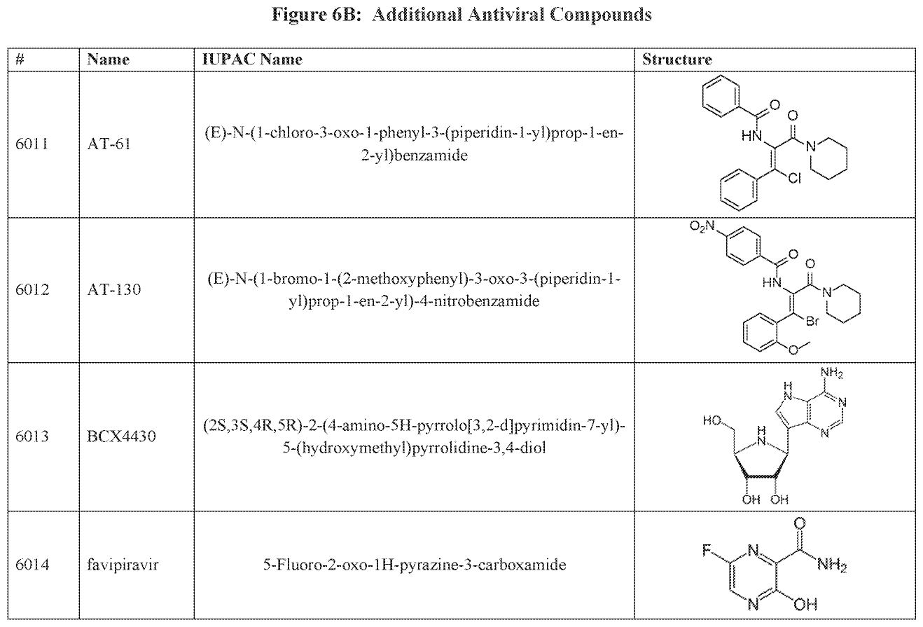

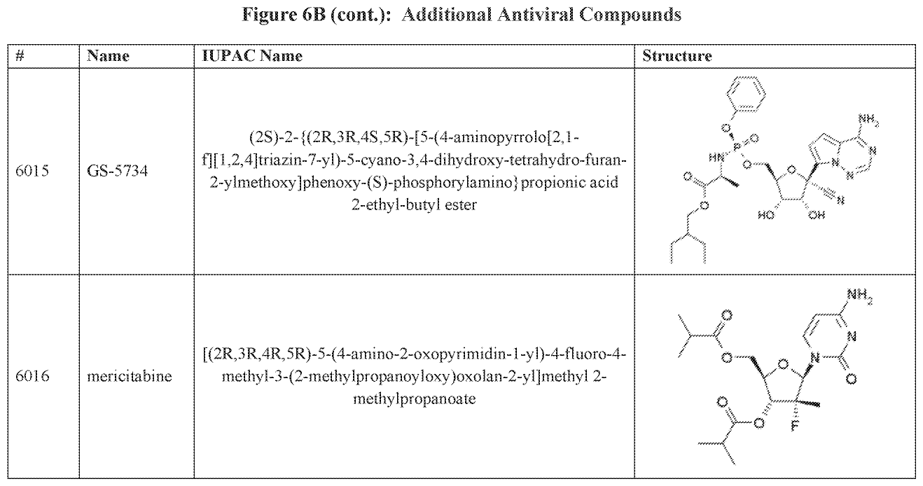

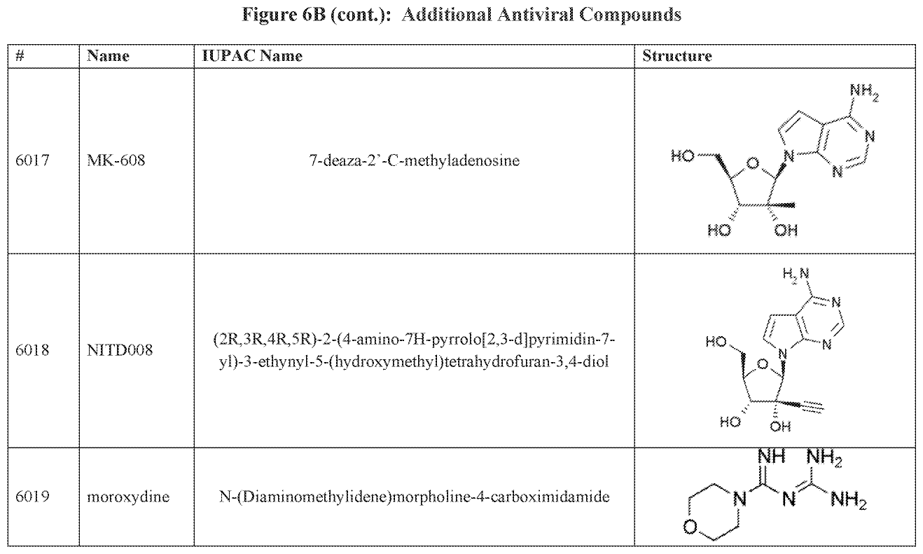

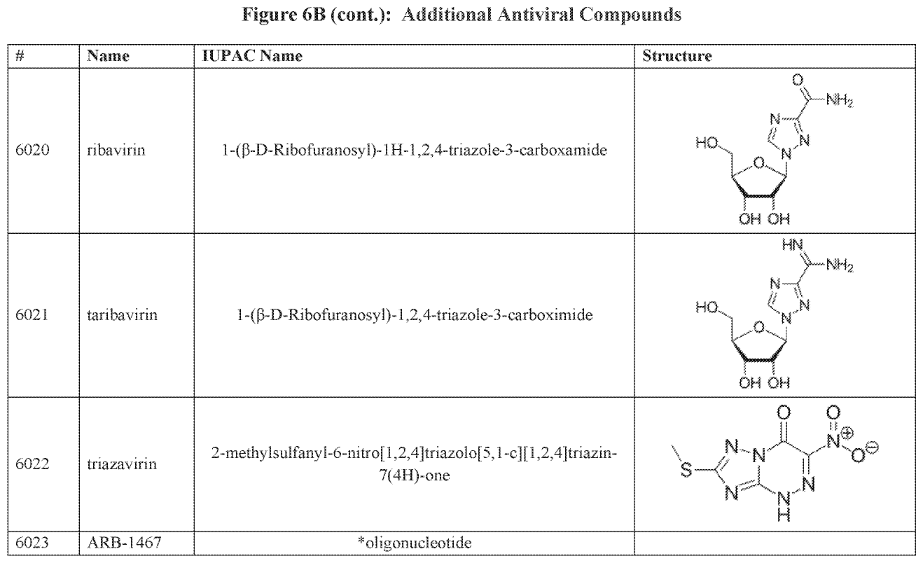

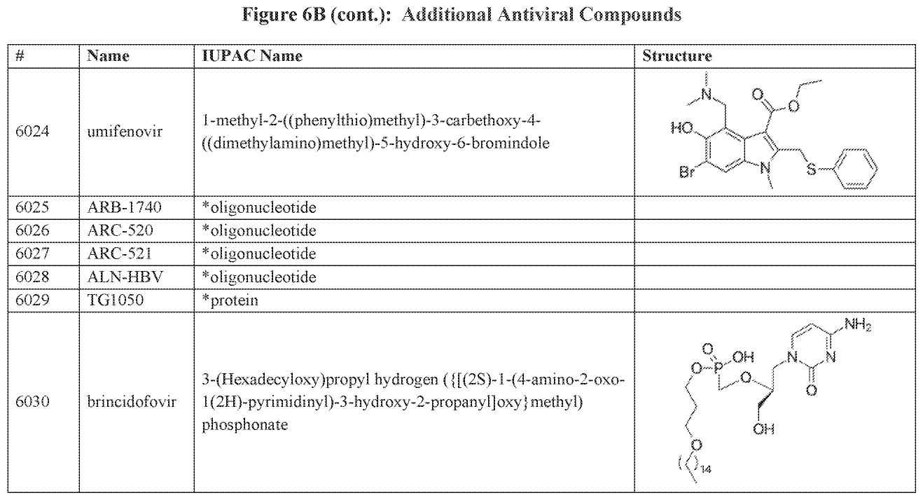

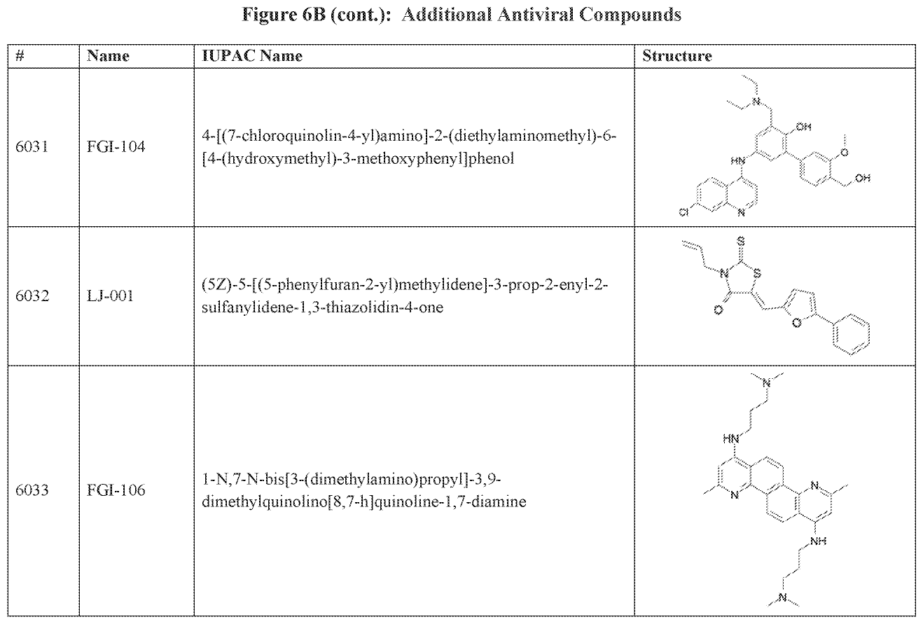

In some embodiments, a compound of Formula (I), or a pharmaceutically acceptable salt thereof, or a pharmaceutical composition that includes a compound of Formula (I), or a pharmaceutically acceptable salt thereof, can be used in combination with other antiviral compounds. Examples of other antiviral compounds include, but are not limited to, bevirimat, BIT225, calanolide A, hydroxycarbamide, miltefosine, seliciclib, cyanovirin-N, griffithsin, scytovirin, BCX4430, favipiravir, GS-5734, mericitabine, MK-608 (7-deaza-2′-C-methyladenosine), NITD008, moroxydine, ribavirin, taribavirin, triazavirin, ARB-1467, ARB-1740, ARC-520, ARC-521, ALN-HBV, TG1050, Tre recombinase, AT-61, AT-130, BCX4430, favipiravir, umifenovir, brincidofovir, FGI-104, LJ-001, FGI-106, and pharmaceutically acceptable salts of any of the foregoing, and/or combinations thereof. A non-limiting list of example other antiviral compounds includes the compounds numbered 6001-6010 in FIG. 6A and 6011-6033 in FIG. 6B. Additional examples of other antiviral compounds include, but are not limited to, an abzyme, an enzyme, a protein, or an antibody. Additional examples of other antiviral compounds include, but are not limited to, ceragenins, including CSA-54, diarylpyrimidines, synergistic enhancers, and zinc finger protein transcription factors, and pharmaceutically acceptable salts of any of the foregoing, and/or combinations thereof.

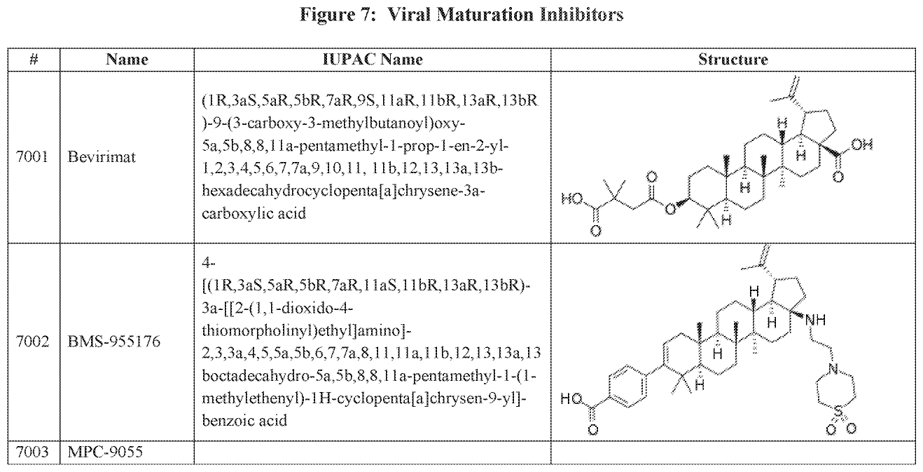

In some embodiments, a compound of Formula (I), or a pharmaceutically acceptable salt thereof, or a pharmaceutical composition that includes a compound of Formula (I), or a pharmaceutically acceptable salt thereof, can be used in combination with a viral maturation inhibitor. In some embodiments, the viral maturation inhibitor can inhibit maturation of HBV and/or HDV. Examples of viral maturation inhibitors include, but are not limited to bevimirat, BMS-955176, MPC-9055, and pharmaceutically acceptable salts of any of the foregoing, and/or combinations thereof. A non-limiting list of example viral maturation inhibitors includes the compounds numbered 7001-7003 in FIG. 7.

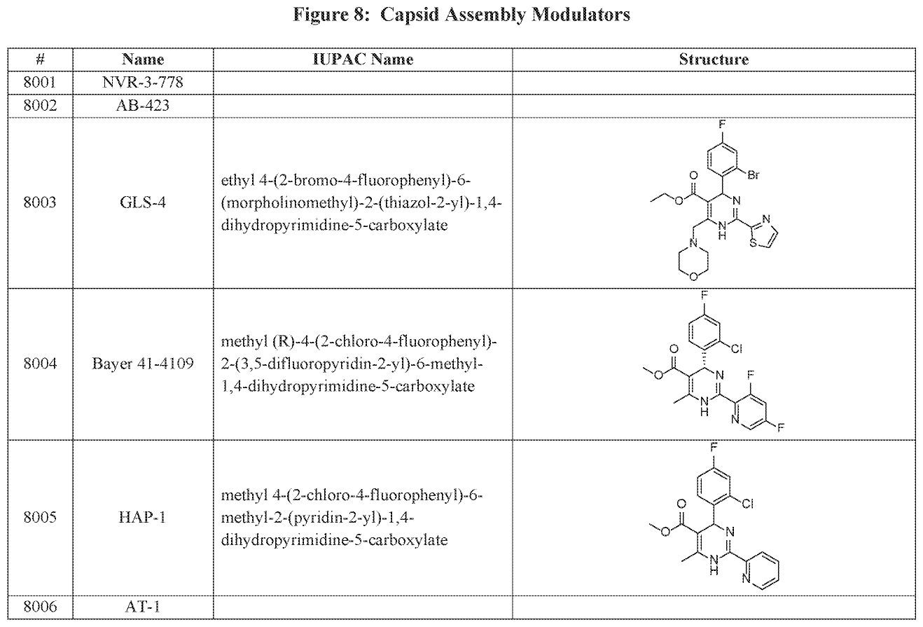

In some embodiments, a compound of Formula (I), or a pharmaceutically acceptable salt thereof, or a pharmaceutical composition that includes a compound of Formula (I), or a pharmaceutically acceptable salt thereof, can be used in combination with a capsid assembly modulator. In some embodiments, the capsid assembly modulator can stabilize the capsid. In some embodiments, the capsid assembly modulator can promote excess capsid assembly. In some embodiments, the capsid assembly modulator can induce formation of non-capsid polymers of capsid peptides. In some embodiments, the capsid assembly modulator can misdirect capsid assembly (e.g., decreasing capsid stability). In some embodiments, the capsid assembly modulator can bind to the HBV and/or HDV core protein. Examples of capsid assembly modulators include, but are not limited to NVR-3-778, AB-423, GLS-4, Bayer 41-4109, HAP-1, AT-1, and pharmaceutically acceptable salts of any of the foregoing, and/or combinations thereof. A non-limiting list of example capsid assembly modulators includes the compounds numbered 8001-8006 in FIG. 8.

In some embodiments, a compound of Formula (I), or a pharmaceutically acceptable salt thereof, or a pharmaceutical composition that includes a compound of Formula (I), or a pharmaceutically acceptable salt thereof, can be used in combination with a FXR agonist. Examples of FXR agonists include, but are not limited to cafestol; chenodeoxychoic acid; cholic acid; obeticholic acid; ursodeoxycholic acid; fexaramine;

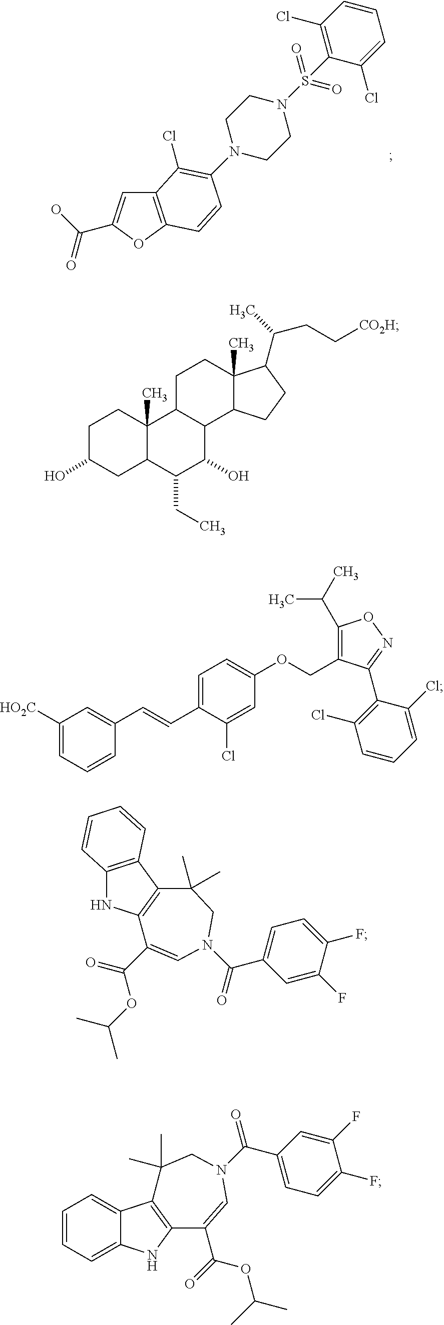

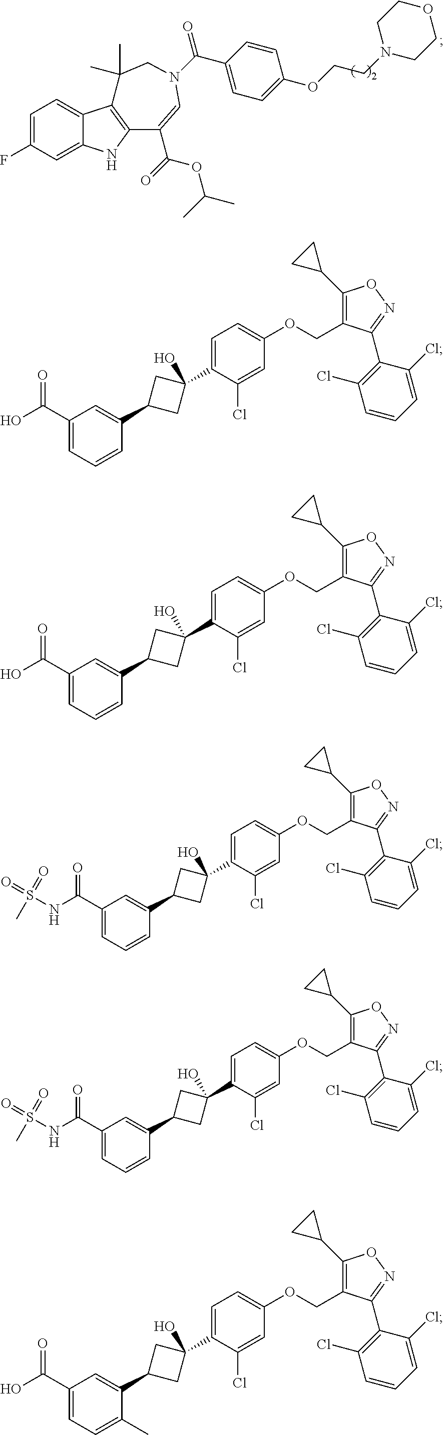

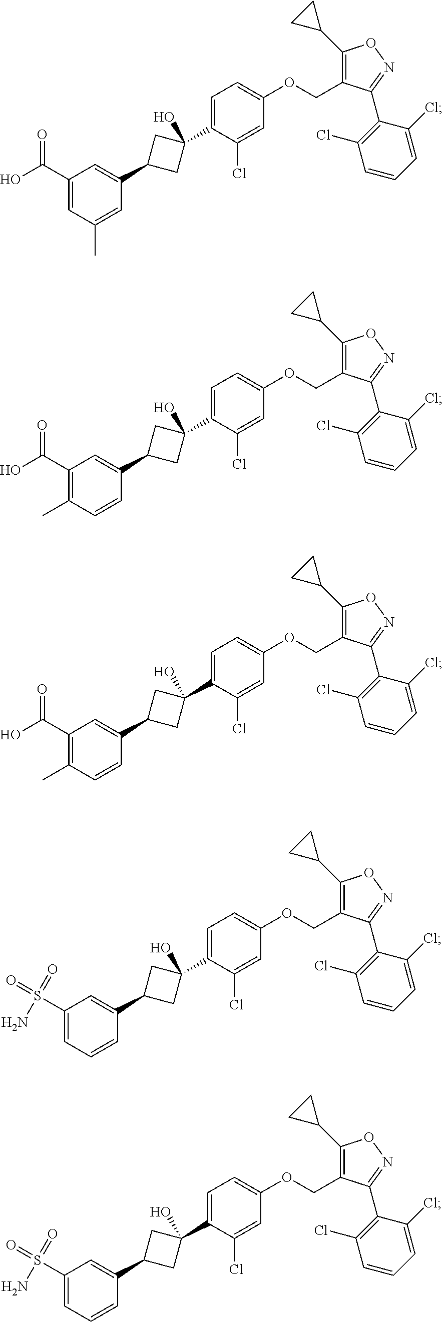 [SMILES: Cc1cc(C(=O)O)cc([C@H]2C[C@](O)(c3ccc(OCc4c(-c5c(Cl)cccc5Cl)noc4C4CC4)cc3Cl)C2)c1 ; Cc1ccc([C@H]2C[C@](O)(c3ccc(OCc4c(-c5c(Cl)cccc5Cl)noc4C4CC4)cc3Cl)C2)cc1C(=O)O ; Cc1ccc([C@H]2C[C@@](O)(c3ccc(OCc4c(-c5c(Cl)cccc5Cl)noc4C4CC4)cc3Cl)C2)cc1C(=O)O ; NS(=O)(=O)c1cccc([C@H]2C[C@](O)(c3ccc(OCc4c(-c5c(Cl)cccc5Cl)noc4C4CC4)cc3Cl)C2)c1 ; NS(=O)(=O)c1cccc([C@H]2C[C@@](O)(c3ccc(OCc4c(-c5c(Cl)cccc5Cl)noc4C4CC4)cc3Cl)C2)c1]

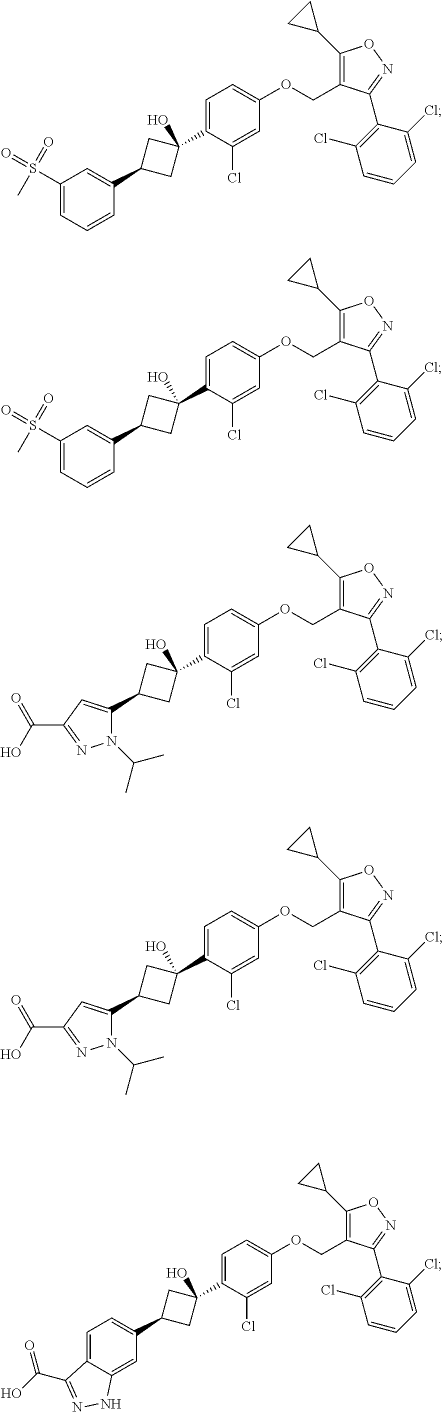 [SMILES: CS(=O)(=O)c1cccc([C@H]2C[C@](O)(c3ccc(OCc4c(-c5c(Cl)cccc5Cl)noc4C4CC4)cc3Cl)C2)c1 ; CS(=O)(=O)c1cccc([C@H]2C[C@@](O)(c3ccc(OCc4c(-c5c(Cl)cccc5Cl)noc4C4CC4)cc3Cl)C2)c1 ; CC(C)n1nc(C(=O)O)cc1[C@H]1C[C@](O)(c2ccc(OCc3c(-c4c(Cl)cccc4Cl)noc3C3CC3)cc2Cl)C1 ; CC(C)n1nc(C(=O)O)cc1[C@H]1C[C@@](O)(c2ccc(OCc3c(-c4c(Cl)cccc4Cl)noc3C3CC3)cc2Cl)C1 ; O=C(O)c1n[nH]c2cc([C@H]3C[C@](O)(c4ccc(OCc5c(-c6c(Cl)cccc6Cl)noc5C5CC5)cc4Cl)C3)ccc12]

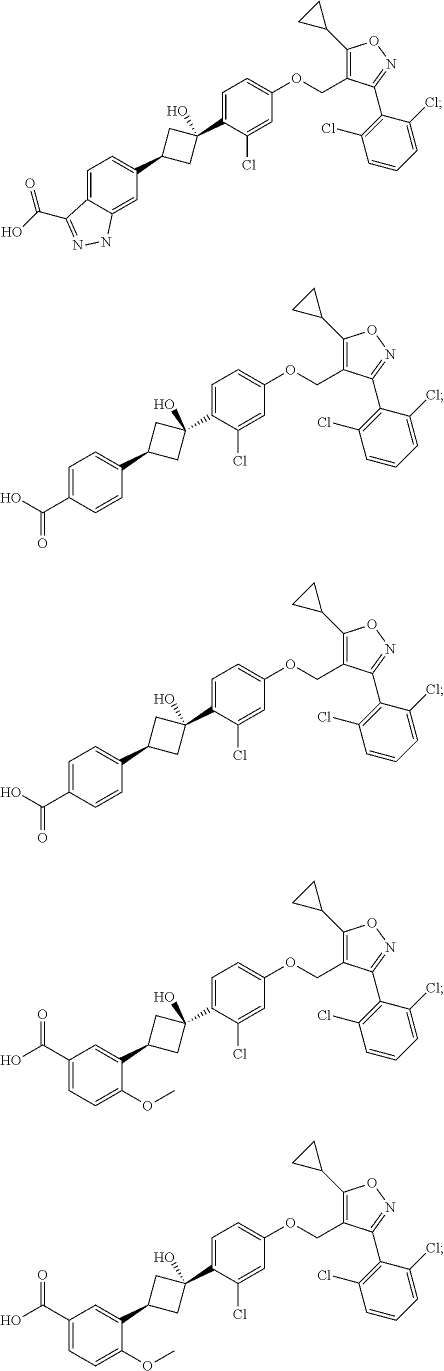 [SMILES: O=C(O)c1n[nH]c2cc([C@H]3C[C@@](O)(c4ccc(OCc5c(-c6c(Cl)cccc6Cl)noc5C5CC5)cc4Cl)C3)ccc12 ; O=C(O)c1ccc([C@H]2C[C@](O)(c3ccc(OCc4c(-c5c(Cl)cccc5Cl)noc4C4CC4)cc3Cl)C2)cc1 ; O=C(O)c1ccc([C@H]2C[C@@](O)(c3ccc(OCc4c(-c5c(Cl)cccc5Cl)noc4C4CC4)cc3Cl)C2)cc1 ; COc1ccc(C(=O)O)cc1[C@H]1C[C@](O)(c2ccc(OCc3c(-c4c(Cl)cccc4Cl)noc3C3CC3)cc2Cl)C1 ; COc1ccc(C(=O)O)cc1[C@H]1C[C@@](O)(c2ccc(OCc3c(-c4c(Cl)cccc4Cl)noc3C3CC3)cc2Cl)C1]

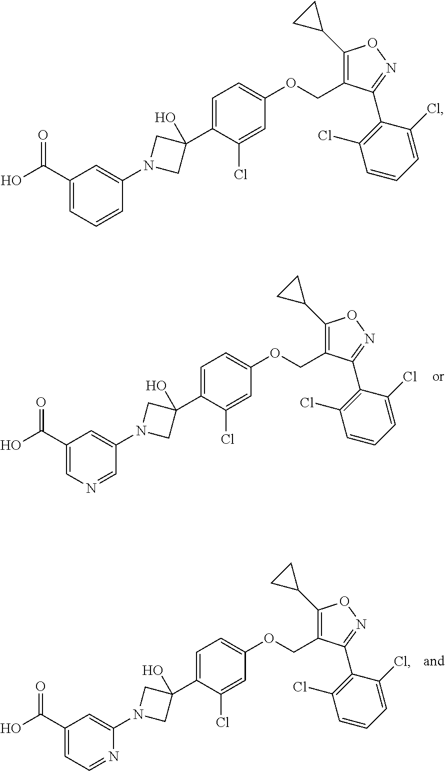 [SMILES: O=C(O)c1cccc(N2CC(O)(c3ccc(OCc4c(-c5c(Cl)cccc5Cl)noc4C4CC4)cc3Cl)C2)c1 ; O=C(O)c1cncc(N2CC(O)(c3ccc(OCc4c(-c5c(Cl)cccc5Cl)noc4C4CC4)cc3Cl)C2)c1 ; O=C(O)c1ccnc(N2CC(O)(c3ccc(OCc4c(-c5c(Cl)cccc5Cl)noc4C4CC4)cc3Cl)C2)c1]

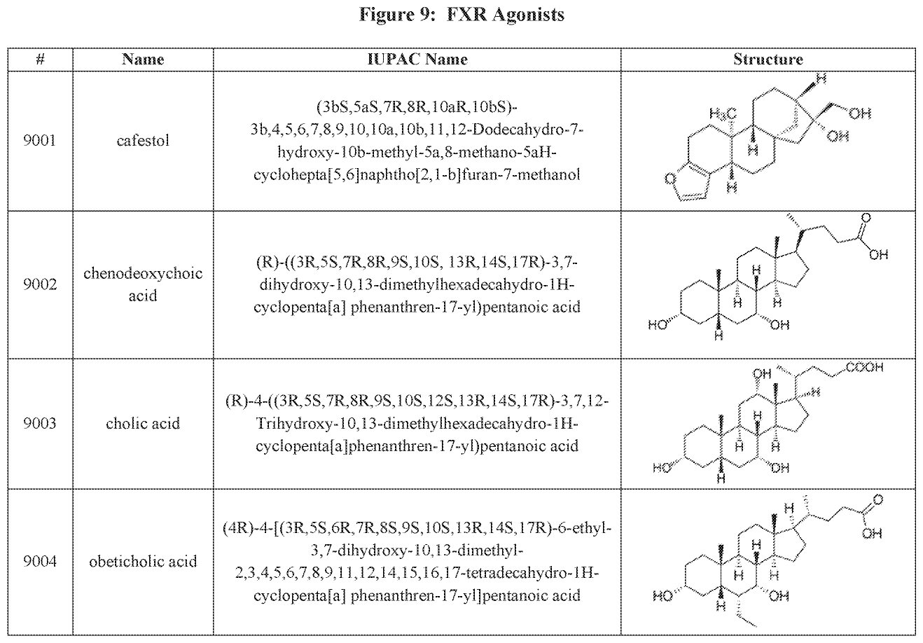

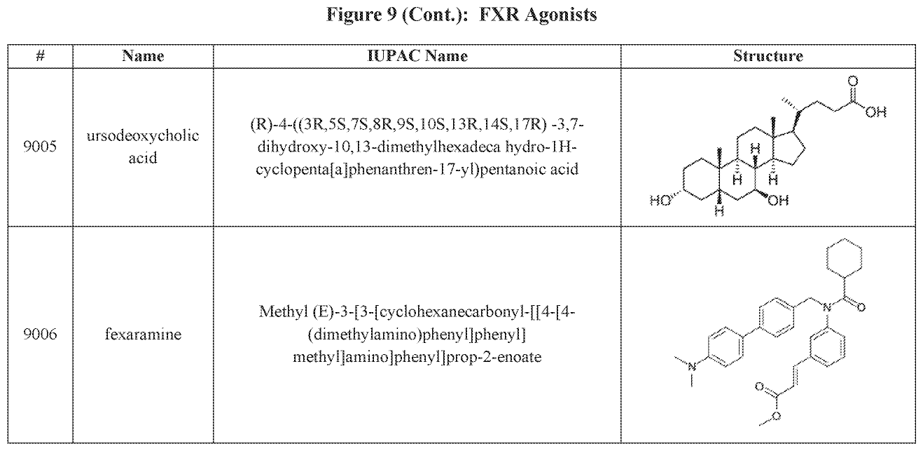

pharmaceutically acceptable salts of any of the foregoing, and/or combinations thereof. A non-limiting list of additional example FXR agonists includes the compounds numbered 9001-9006 in FIG. 9.

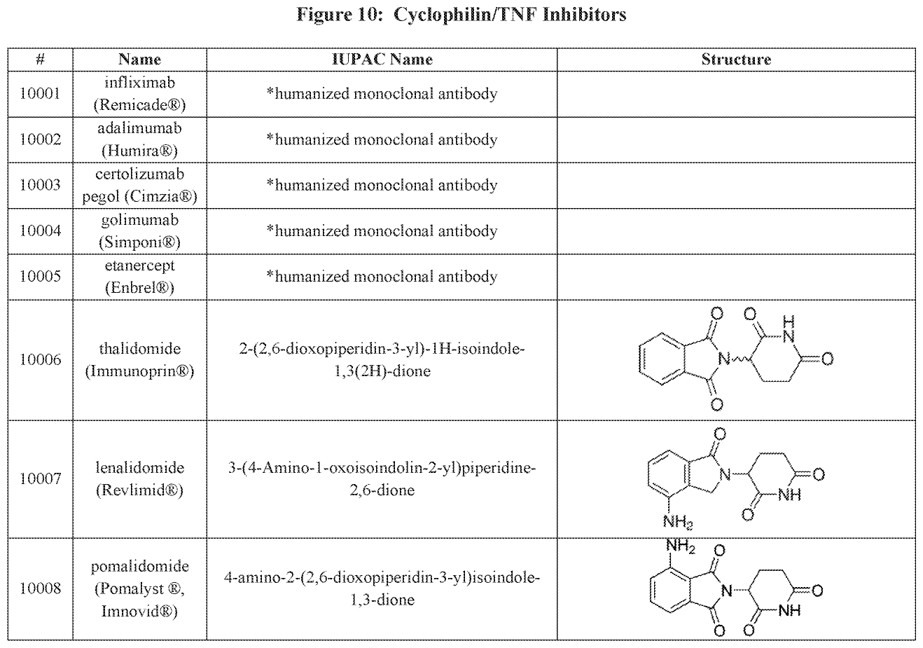

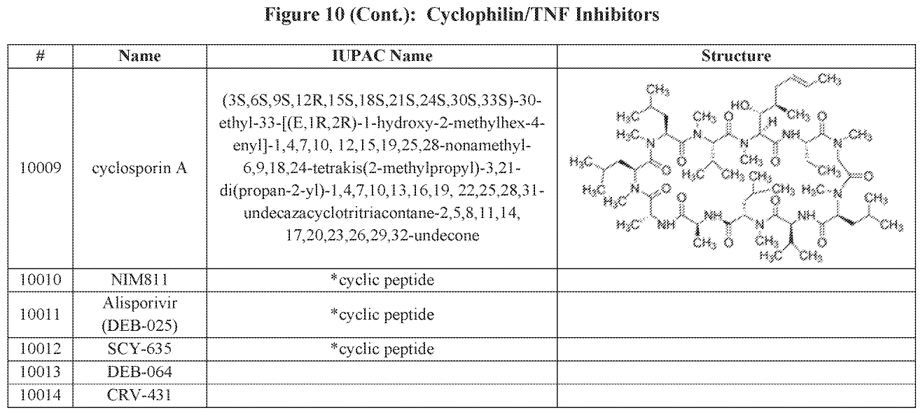

In some embodiments, a compound of Formula (I), or a pharmaceutically acceptable salt thereof, or a pharmaceutical composition that includes a compound of Formula (I), or a pharmaceutically acceptable salt thereof, can be used in combination with a cyclophilin/TNF inhibitor. Examples of cyclophilin/TNF inhibitors include, but are not limited to infliximab (Remicade®), adalimumab (Humira®), certolizumab pegol (Cimzia®), golimumab (Simponi®), etanercept (Enbrel®), thalidomide (Immunoprin®), lenalidomide (Revlimid®), pomalidomide (Pomalyst®, Imnovid®), cyclosporin A, NIM811, Alisporivir (DEB-025), SCY-635, DEB-064, CRV-431, and pharmaceutically acceptable salts of any of the foregoing, and/or combinations thereof. A non-limiting list of example TNF/cyclophilin inhibitors includes the compounds numbered 10001-10014 in FIG. 10.

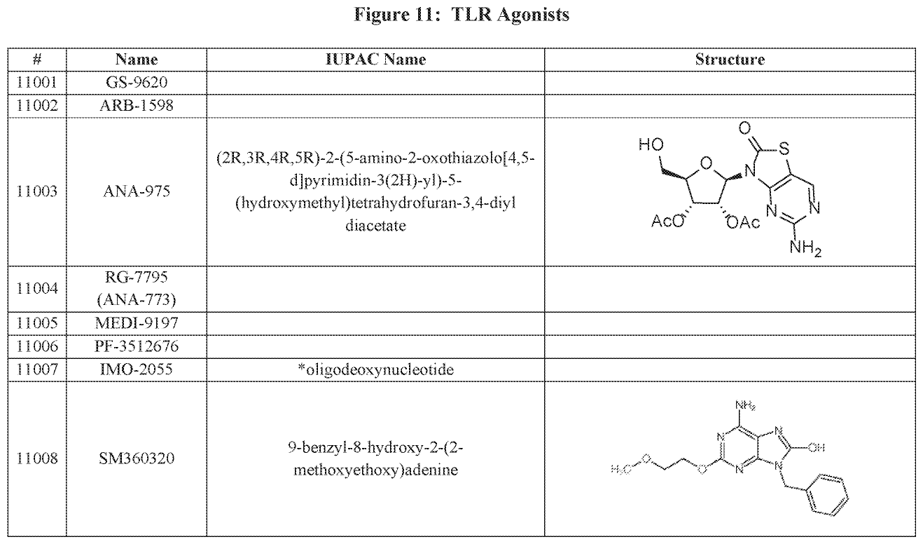

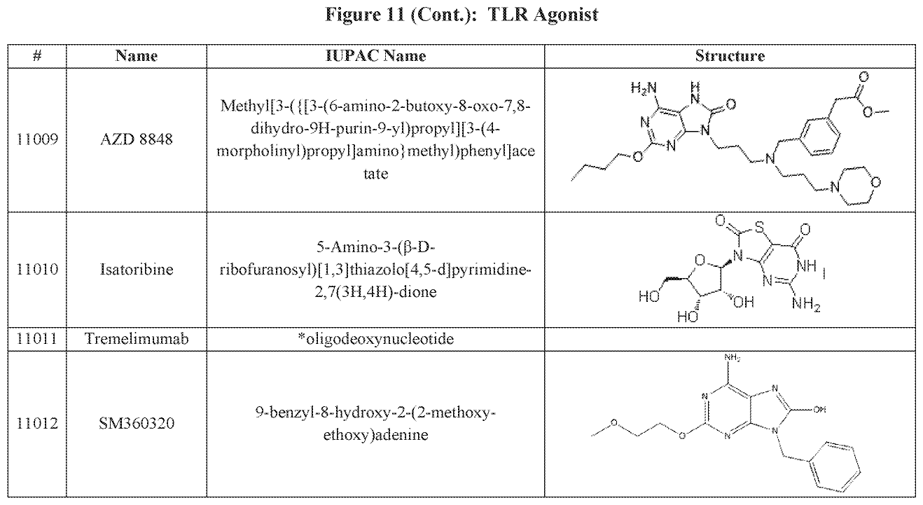

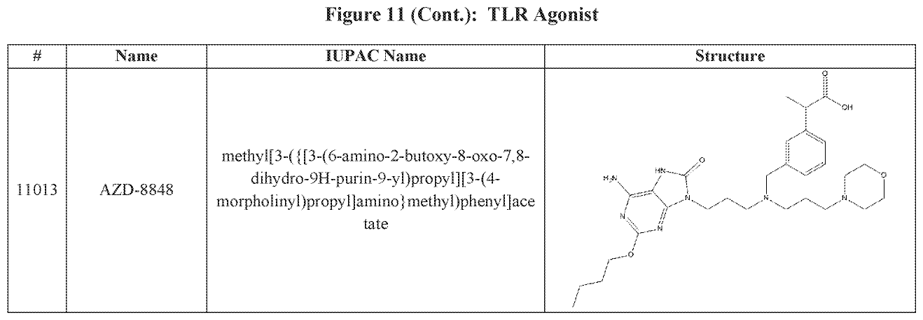

In some embodiments, a compound of Formula (I), or a pharmaceutically acceptable salt thereof, or a pharmaceutical composition that includes a compound of Formula (I), or a pharmaceutically acceptable salt thereof, can be used in combination with a TLR agonist. Examples of TLR agonists include, but are not limited to GS-9620, ARB-1598, ANA-975, RG-7795 (ANA-773), MEDI-9197, PF-3512676, IMO-2055, isatoribine, tremelimumab, SM360320, AZD-8848, and pharmaceutically acceptable salts of any of the foregoing, and/or combinations thereof. A non-limiting list of example TLR agonists includes the compounds numbered 11001-11013 in FIG. 11.

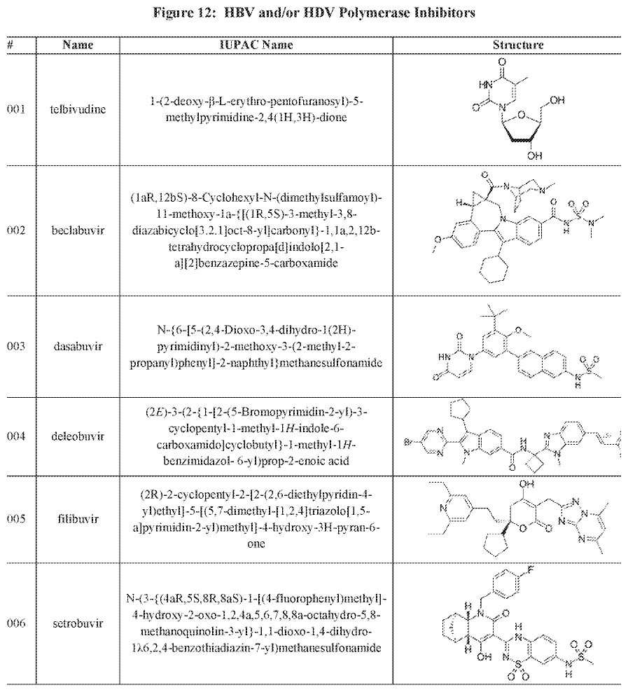

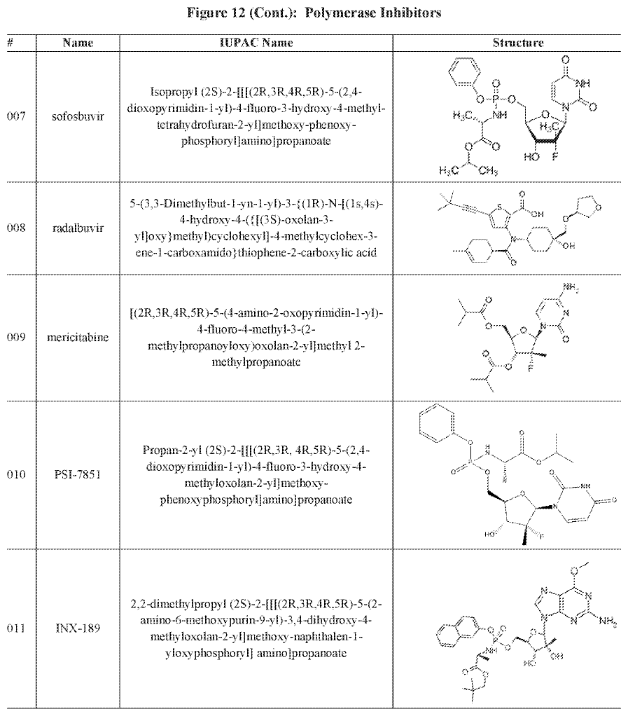

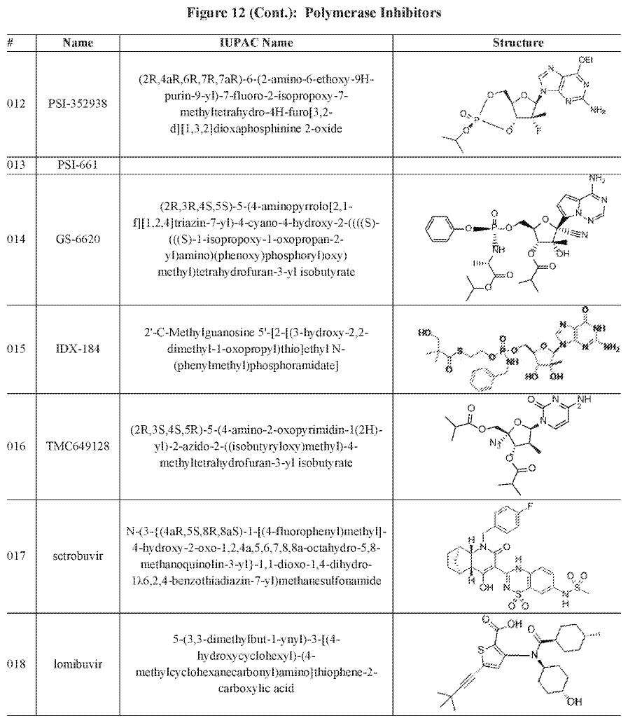

In some embodiments, a compound of Formula (I), or a pharmaceutically acceptable salt thereof, or a pharmaceutical composition that includes a compound of Formula (I), or a pharmaceutically acceptable salt thereof, can be used in combination with a polymerase inhibitor. Examples of polymerase inhibitors include, but are not limited to telbivudine, beclabuvir, dasabuvir, deleobuvir, filibuvir, setrobuvir, sofosbuvir, radalbuvir, RG7128 (mericitabine), PSI-7851, INX-189, PSI-352938, PSI-661, GS-6620, IDX-184, TMC649128, setrobuvir, lomibuvir, nesbuvir, GS-9190 (tegobuvir), VX-497 (merimepodib), ribavirin, acyclovir, atevirapine, famciclovir, valacyclovir, ganciclovir, valganciclovir, cidofovir, JK-05, and pharmaceutically acceptable salts of any of the foregoing, and/or combinations thereof. A non-limiting list of example polymerase inhibitors includes the compounds numbered 12001-12030 in FIG. 12.

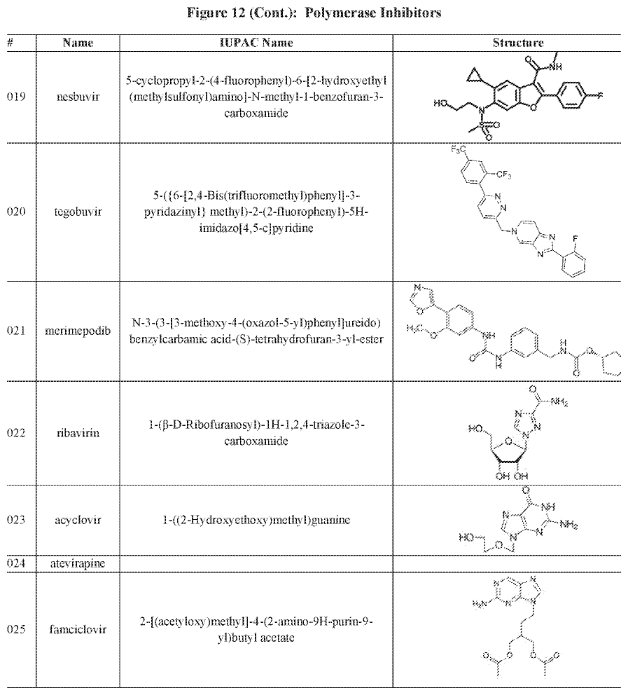

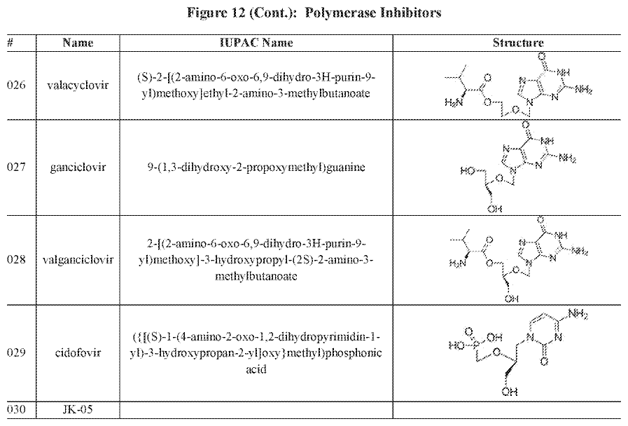

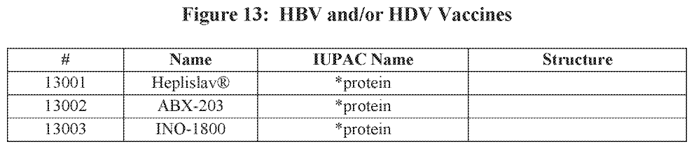

In some embodiments, a compound of Formula (I), or a pharmaceutically acceptable salt thereof, or a pharmaceutical composition that includes a compound of Formula (I), or a pharmaceutically acceptable salt thereof, can be used in combination with a vaccine. Examples of vaccines include, but are not limited to Heplislav®, ABX-203, INO-1800, and pharmaceutically acceptable salts of any of the foregoing, and/or combinations thereof. A non-limiting list of example vaccines includes those numbered 13001-13003 in FIG. 13.

In some embodiments, a compound of Formula (I), or a pharmaceutically acceptable salt thereof, or a pharmaceutical composition that includes a compound of Formula (I), or a pharmaceutically acceptable salt thereof, can be used in combination with an interferon. Examples of interferons include, but are not limited to alpha-interferons, beta-interferons, delta-interferons, omega-interferons, tau-interferons, x-interferons, consensus interferons, and asialo-interferons. Specific non-limiting examples include: interferon alpha 1A, interferon alpha 1B, interferon alpha 2A, interferon alpha 2B, pegylated-interferon alpha 2a (PEGASYS®, Roche), recombinant interferon alpha 2a (ROFERON®, Roche), inhaled interferon alpha 2b (AERX®, Aradigm), pegylated-interferon alpha 2b (ALBUFERON®, Human Genome Sciences/Novartis, PEGINTRON®, Schering), recombinant interferon alpha 2b (INTRON A®, Schering), pegylated interferon alpha 2b (PEG-INTRON®, Schering, VIRAFERONPEG@, Schering), interferon beta-1a (REBIF®, Serono, Inc. and Pfizer), consensus interferon alpha (INFERGEN®, Valeant Pharmaceutical).

In some embodiments, a compound of Formula (I), or a pharmaceutically acceptable salt thereof, or a pharmaceutical composition that includes a compound of Formula (I), or a pharmaceutically acceptable salt thereof, can be used in combination with an siRNA or ASO cccDNA inhibitor. In some embodiments, the an siRNA or ASO cccDNA inhibitor can prevent cccDNA formation, eliminate existing cccDNA, destabilizing existing cccDNA, and/or silence cccDNA transcription.

In some embodiments, a compound of Formula (I), or a pharmaceutically acceptable salt thereof, or a pharmaceutical composition that includes a compound of Formula (I), or a pharmaceutically acceptable salt thereof, can be used in combination with a gene silencing agent. In some embodiments, the gene silencing agent decreases transcription of a target gene or genes. In some embodiments, the gene silencing agent decreases translation of a target gene or genes. In some embodiments, the gene silencing agent can be an oligodeoxynucleotide, a ribozyme, siRNA, a morpholino, or a combination of any of the foregoing.

In some embodiments, a compound of Formula (I), or a pharmaceutically acceptable salt thereof, or a pharmaceutical composition that includes a compound of Formula (I), or a pharmaceutically acceptable salt thereof, can be used in combination with an HBx inhibitor. HBx is a polypeptide encoded by hepadnaviruses that contributes to viral infectivity. In some embodiments, the HBx inhibitor decreases HBx transactivation activity. In some embodiments, the HBx inhibitor blocks or decreases HBx binding to mammalian cellular proteins. In some embodiments, the HBx inhibitor decreases HBx blocks or decreases recruitment of kinases.

In some embodiments, a compound of Formula (I), or a pharmaceutically acceptable salt thereof, or a pharmaceutical composition that includes a compound of Formula (I), or a pharmaceutically acceptable salt thereof, can be used in combination with an HBsAg secretion inhibitor. HBV and HDV surface antigens are proteins found on both new HBV particle and subviral particles. The subviral particles are non-infectious and are secreted in significant excess to infectious virus, potentially exhausting a subject's immune system. In some embodiments, the HBsAg secretion inhibitor can reduce a subject's immune exhaustion due to the surface antigen. In some embodiments, the HBsAg secretion inhibitor can promote a subject's immune response to HBV and/or HDV.

In some embodiments, a compound of Formula (I), or a pharmaceutically acceptable salt thereof, or a pharmaceutical composition that includes a compound of Formula (I), or a pharmaceutically acceptable salt thereof, can be used in combination with a covalently closed circular DNA (cccDNA) inhibitor. In some embodiments, the cccDNA inhibitor can directly bind cccDNA, can inhibit conversion of relaxed circular DNA (rcDNA) to cccDNA, can reduce or silence transcription of cccDNA, and/or can promote elimination of existing cccDNA.

In some embodiments, a compound of Formula (I), or a pharmaceutically acceptable salt thereof, or a pharmaceutical composition that includes a compound of Formula (I), or a pharmaceutically acceptable salt thereof, can be used in combination with a compound of Formula (I), or a pharmaceutically acceptable salt thereof, described in PCT Publication No. WO 2017/156262, filed Mar. 9, 2017.

Some embodiments described herein relate to a method of treating a HBV and/or HDV infection that can include contacting a cell infected with the HBV and/or HDV infection with an effective amount of a compound of Formula (I), or a pharmaceutically acceptable salt thereof, in combination with one or more additional agents, such as those described herein. Other embodiments described herein relate to a method of treating a HBV and/or HDV infection that can include administering to a subject suffering from the HBV and/or HDV infection an effective amount of a compound of Formula (I), or a pharmaceutically acceptable salt thereof, in combination with one or more additional agents, such as those described herein. Still other embodiments described herein relate to a method of inhibiting the replication of a HBV and/or HDV that can include contacting a cell infected with the HBV and/or HDV with an effective amount of a compound of Formula (I), or a pharmaceutically acceptable salt thereof, in combination with one or more additional agents, such as those described herein. Yet still other embodiments described herein relate to a method of inhibiting the replication of a HBV and/or HDV that can include administering to a subject infected with the HBV and/or HDV an effective amount of a compound of Formula (I), or a pharmaceutically acceptable salt thereof, in combination with one or more additional agents, such as those described herein. Examples of additional agents include those described herein, such as, a polymerase inhibitor, a protease inhibitor (PI), a fusion/entry inhibitor, an interferon, a FXR agonist, a TLR agonist, a viral maturation inhibitor, a capsid assembly modulator, a cyclophilin/TNF inhibitor, a vaccine, an siRNA or ASO cccDNA inhibitor, a gene silencing agent, an HBx inhibitor, an HBsAg secretion inhibitor, and another antiviral compound, or a pharmaceutically acceptable salt of any of the foregoing.

Some embodiments described herein relate to a method of treating a HIV infection that can include contacting a cell infected with the HIV infection with an effective amount of a compound of Formula (I), or a pharmaceutically acceptable salt thereof, in combination with one or more additional agents, such as those described herein. Other embodiments described herein relate to a method of treating a HIV infection that can include administering to a subject suffering from the HIV infection an effective amount of a compound of Formula (I), or a pharmaceutically acceptable salt thereof, in combination with one or more additional agents, such as those described herein. Still other embodiments described herein relate to a method of inhibiting the replication of a HIV that can include contacting a cell infected with the HIV with an effective amount of a compound of Formula (I), or a pharmaceutically acceptable salt thereof, in combination with one or more additional agents, such as those described herein. Yet still other embodiments described herein relate to a method of inhibiting the replication of a HIV that can include administering to a subject infected with the HIV an effective amount of a compound of Formula (I), or a pharmaceutically acceptable salt thereof, in combination with one or more additional agents, such as those described herein. Examples of additional agents include those described herein, such as, antiretroviral therapy (ART) agents, such as a non-nucleoside reverse transcriptase inhibitor (NNRTI), a nucleoside reverse transcriptase inhibitor (NRTI), a protease inhibitor (PI), a fusion/entry inhibitor (also called a CCR5 antagonist), an integrase strand transfer inhibitor (INSTI), and an HIV other antiretroviral therapy compound, or a pharmaceutically acceptable salt of any of the foregoing.

In some embodiments, a compound of Formula (I), or a pharmaceutically acceptable salt thereof, can be administered with one or more additional agent(s) together in a single pharmaceutical composition. In some embodiments, a compound of Formula (I), or a pharmaceutically acceptable salt the thereof, can be administered with one or more additional agent(s) as two or more separate pharmaceutical compositions. For example, a compound of Formula (I), or a pharmaceutically acceptable salt thereof, can be administered in one pharmaceutical composition, and at least one of the additional agents can be administered in a second pharmaceutical composition. If there are at least two additional agents, one or more of the additional agents can be in a first pharmaceutical composition that includes a compound of Formula (I), or a pharmaceutically acceptable salt thereof, and at least one of the other additional agent(s) can be in a second pharmaceutical composition.

The dosing amount(s) and dosing schedule(s) when using a compound of Formula (I), or a pharmaceutically acceptable salt thereof, or a pharmaceutical composition that includes a compound of Formula (I), or a pharmaceutically acceptable salt thereof, and one or more additional agents are within the knowledge of those skilled in the art. For example, when performing a conventional standard of care therapy using art-recognized dosing amounts and dosing schedules, a compound of Formula (I), or a pharmaceutically acceptable salt thereof, or a pharmaceutical composition that includes a compound of Formula (I), or a pharmaceutically acceptable salt thereof, can be administered in addition to that therapy, or in place of one of the agents of a combination therapy, using effective amounts and dosing protocols as described herein.

The order of administration of a compound of Formula (I), or a pharmaceutically acceptable salt thereof, with one or more additional agent(s) can vary. In some embodiments, a compound of Formula (I), or a pharmaceutically acceptable salt thereof, can be administered prior to all additional agents. In other embodiments, a compound of Formula (I), or a pharmaceutically acceptable salt thereof, can be administered prior to at least one additional agent. In still other embodiments, a compound of Formula (I), or a pharmaceutically acceptable salt thereof, can be administered concomitantly with one or more additional agent(s). In yet still other embodiments, a compound of Formula (I), or a pharmaceutically acceptable salt thereof, can be administered subsequent to the administration of at least one additional agent. In some embodiments, a compound of Formula (I), or a pharmaceutically acceptable salt thereof, can be administered subsequent to the administration of all additional agents.

In some embodiments, the combination of a compound of Formula (I), or a pharmaceutically acceptable salt thereof, in combination with one or more additional agent(s) in FIGS. 1-13 (including pharmaceutically acceptable salts and prodrugs of any of the foregoing) can result in an additive effect. In some embodiments, the combination of a compound of Formula (I), or a pharmaceutically acceptable salt thereof, in combination with one or more additional agent(s) in FIGS. 1-13 (including pharmaceutically acceptable salts and prodrugs of any of the foregoing) can result in a synergistic effect. In some embodiments, the combination of a compound of Formula (I), or a pharmaceutically acceptable salt thereof, in combination with one or more additional agent(s) in FIGS. 1-13 (including pharmaceutically acceptable salts and prodrugs of any of the foregoing) can result in a strongly synergistic effect. In some embodiments, the combination of a compound of Formula (I), or a pharmaceutically acceptable salt thereof, in combination with one or more additional agent(s) in FIGS. 1-13 (including pharmaceutically acceptable salts and prodrugs of any of the foregoing) is not antagonistic.

As used herein, the term “antagonistic” means that the activity of the combination of compounds is less compared to the sum of the activities of the compounds in combination when the activity of each compound is determined individually (i.e. as a single compound). As used herein, the term “synergistic effect” means that the activity of the combination of compounds is greater than the sum of the individual activities of the compounds in the combination when the activity of each compound is determined individually. As used herein, the term “additive effect” means that the activity of the combination of compounds is about equal to the sum of the individual activities of the compound in the combination when the activity of each compound is determined individually.

A potential advantage of utilizing a compound of Formula (I), or a pharmaceutically acceptable salt thereof, in combination with one or more additional agent(s) in FIGS. 1-13 (including pharmaceutically acceptable salts of any of the foregoing) may be a reduction in the required amount(s) of one or more compounds of FIGS. 1-13 (including pharmaceutically acceptable salts of any of the foregoing) that is effective in treating a disease condition disclosed herein (for example, HBV, HDV and/or HIV), as compared to the amount required to achieve same therapeutic result when one or more compounds of FIGS. 1-13 (including pharmaceutically acceptable salts of any of the foregoing) are administered without a compound of Formula (I), or a pharmaceutically acceptable salt thereof. For example, the amount of a compound in FIGS. 1-13 (including a pharmaceutically acceptable salt of any of the foregoing), can be less compared to the amount of the compound in FIGS. 1-13 (including a pharmaceutically acceptable salt of any of the foregoing), needed to achieve the same viral load reduction when administered as a monotherapy. Another potential advantage of utilizing a compound of Formula (I), or a pharmaceutically acceptable salt thereof, in combination with one or more additional agent(s) in FIGS. 1-13 (including pharmaceutically acceptable salts of any of the foregoing) is that the use of two or more compounds having different mechanism of actions can create a higher barrier to the development of resistant viral strains compared to the barrier when a compound is administered as monotherapy.

Additional advantages of utilizing a compound of Formula (I), or a pharmaceutically acceptable salt thereof, in combination with one or more additional agent(s) in FIGS. 1-13 (including pharmaceutically acceptable salts of any of the foregoing) may include little to no cross resistance between a compound of Formula (I), or a pharmaceutically acceptable salt thereof, and one or more additional agent(s) in FIGS. 1-13 (including pharmaceutically acceptable salts of any of the foregoing) thereof; different routes for elimination of a compound of Formula (I), or a pharmaceutically acceptable salt thereof, and one or more additional agent(s) in FIGS. 1-13 (including pharmaceutically acceptable salts of any of the foregoing); little to no overlapping toxicities between a compound of Formula (I), or a pharmaceutically acceptable salt thereof, and one or more additional agent(s) in FIGS. 1-13 (including pharmaceutically acceptable salts of any of the foregoing); little to no significant effects on cytochrome P450; little to no pharmacokinetic interactions between a compound of Formula (I), or a pharmaceutically acceptable salt thereof, and one or more additional agent(s) in FIGS. 1-13 (including pharmaceutically acceptable salts of any of the foregoing); greater percentage of subjects achieving a sustained viral response compared to when a compound is administered as monotherapy and/or a decrease in treatment time to achieve a sustained viral response compared to when a compound is administered as monotherapy.

### EXAMPLES

The following specific examples are provided to further illustrate various embodiments described herein.

In obtaining the compounds described in the examples below and the corresponding analytical data, the following experimental and analytical protocols were followed unless otherwise indicated.

Unless otherwise stated, reaction mixtures were magnetically stirred at room temperature (r.t.) under a nitrogen atmosphere. Where solutions were “dried,” they were generally dried over a drying agent such as Na₂SO₄ or MgSO₄. Where mixtures, solutions, and extracts were “concentrated”, they were typically concentrated on a rotary evaporator under reduced pressure.

Normal-phase silica gel chromatography (FCC) was performed on silica gel (SiO₂) using prepacked cartridges.

Preparative reverse-phase high performance liquid chromatography (RP HPLC) was performed on: a Gilson 281/215 HPLC with an Xtimate Prep RP18 column (5 μM, 25×150 mm) or an YMC-Actus Triart C18 column (5 μM, 30×100 mm), and a mobile phase of 1% ACN in 0.225% FA was held for 1 min, then a gradient of 1-23% ACN over 9 min, then held at 95% ACN for 2 min, with a flow rate of 25 mL/min.

Mass spectra (MS) were obtained on an Agilent G1969A LCMS-TOF. The mobile phase: 0.1% FA (formic acid) in water (solvent A) and 0.1% FA in ACN (solvent B); Elution Gradient: 0%-30% (solvent B) over 3 minutes and holding at 30% for 1 minute at a flow rate of 1 mL/minute; Column: Xbridge Shield RP 18 5 um, 2.1*50 mm Ion Source: ESI source; Ion Mode: Positive;

Nebulization Gas: Nitrogen; Drying Gas (N2) Flow: 5 l/min; Nebulizer Pressure: 30 psig; Gas Temperature: 325° C. Capillary Voltage: 3.5 KV; Fragmentor Voltage: 50 v.

Nuclear magnetic resonance (NMR) spectra were obtained on Bruker 400 MHz or Varian 400 MHz spectrometers. Definitions for multiplicity are as follows: s=singlet, d=doublet, t=triplet, q=quartet, m=multiplet, br=broad. It will be understood that for compounds comprising an exchangeable proton, said proton may or may not be visible on an NMR spectrum depending on the choice of solvent used for running the NMR spectrum and the concentration of the compound in the solution.

Chemical names were generated using ChemDraw Ultra 12.0, ChemDraw Ultra 14.0 (CambridgeSoft Corp., Cambridge, Mass.) or ACD/Name Version 10.01 (Advanced Chemistry).

Compounds designated as R* or S* are enantiopure compounds where the absolute configuration was not determined.

### Intermediate 1: (1R,3R,4S)-2-Methylene-4-((triisopropylsilyl)oxy)-3-(((triisopropylsilyl)oxy)methyl)cyclopentan-1-ol and (1S,3R,4S)-2-Methylene-4-((triisopropylsilyl)oxy)-3-(((triisopropylsilyl)oxy)methyl)cyclopentan-1-ol

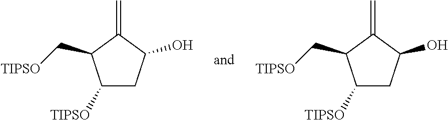

Step A: (4S,5R)-4-((Triisopropylsilyl)oxy)-5-(((triisopropylsilyl)oxy)methyl)dihydrofuran-2(3H)-one. The title compound was prepared according to procedures described in PCT Publication No. WO 2015/056213 (published Apr. 23, 2015).

Step B: (4S,5R)-4-((triisopropylsilyl)oxy)-5-(((triisopropylsilyl)oxy)methyl)tetrahydrofuran-2-ol. To a solution of (4S,5R)-4-((triisopropylsilyl)oxy)-5-(((triisopropylsilyl)oxy)methyl)dihydrofuran-2(3H)-one (10 g, 22.48 mmol) in THF (tetrahydrofuran) (100 mL) was added a solution of DIBAL-H (diisobutylaluminium hydride) (1 M, 56.21 mL) in dropwise at −70° C. over a period of 1 h. under N₂. The temperature was maintained below −55° C. The reaction mixture was stirred at −70° C. for another 2 h. The reaction mixture was quenched with MeOH (methanol) (50 mL) slowly, and diluted with EA (ethyl acetate) (100 mL). The inorganic material was filtered and the filtered cake was washed with EA (100 mL*3). The filtrate was concentrated in vacuum to give a colorless oil. Purification FCC, SiO₂, PE/EA=100/1 to 20/1) afforded the title compound (8.2 g, 81.63% yield) as a colorless oil. ¹H NMR (400 MHz, CDCl₃) δ=5.61-5.30 (m, 1H), 4.77-4.50 (m, 1H), 4.33 (m, 1H), 4.20-4.02 (m, 1H), 3.87-3.59 (m, 2H), 3.44 (m, 1H), 2.24-2.01 (m, 1H), 1.16-1.03 (m, 42H).

Step C: (2R,3S)-1,3-Bis((triisopropylsilyl)oxy)hept-6-yne-2,5-diol. To a solution of (4S,5R)-4-((triisopropylsilyl)oxy)-5-(((triisopropylsilyl)oxy)methyl)tetrahydrofuran-2-ol (8.04 g, 17.99 mmol) in THF (80 mL) was added a solution of bromo (ethynyl) magnesium (0.5 M, 107.94 mL) in dropwise at −70° C. over a period of 0.5 h under N₂. The temperature was maintained below −55° C. The reaction mixture was warmed to 30° C. naturally and stirred at 30° C. for another 2 h. The reaction was quenched by saturated NH₄Cl solution (50 mL) slowly, and then diluted with EA (100 mL). The organic layer was washed with brine (50 mL). The organic layer was dried over anhydrous Na₂SO₄, filtered and concentrated under reduced pressure. Purification (FCC, SiO₂, PE/EA=100/1 to 20/1) afforded the title compound (3.1 g, 36.44% yield) as a colorless oil. ¹H NMR (400 MHz, CDCl₃) δ=4.69-4.70 (m, 1H), 4.13-4.14 (m, 1H), 3.83-3.85 (m, 2H), 3.27-3.29 (m, 1H), 2.73-2.74 (m, 1H), 2.44-2.45 (m, 1H), 2.09 (s, 1H), 2.04-2.09 (m, 2H), 1.05-1.09 (m, 42H).

Step D: Ethyl ((5S,6R)-6-hydroxy-5,7-bis((triisopropylsilyl)oxy)hept-1-yn-3-yl) carbonate. A mixture of (2R,3S)-1,3-bis((triisopropylsilyl)oxy)hept-6-yne-2,5-diol (1 g, 2.11 mmol) and pyridine (501.85 mg, 6.34 mmol, 512.09 μL) in CH₂Cl₂ (10 mL) was added ethyl chloroformate (900 mg, 8.29 mmol, 789.47 μL) and stirred at 0° C. for 2 h. The reaction was quenched by saturated NaHCO₃ solution (20 mL) slowly, and then extracted with DCM (dichloromethane) (30 mL). The organic layer was washed with brine (50 mL), dried over anhydrous Na₂SO₄, filtered and concentrated under reduced pressure. Purification (FCC, SiO₂, PE/EA=100/1 to 20/1) afforded the title compound (800 mg, 69.42% yield) as a colorless oil. ¹H NMR (400 MHz, CDCl₃) δ=5.48-5.50 (m, 1H), 4.20-4.25 (m, 3H), 3.74-3.77 (m, 3H), 2.45-2.52 (s, 2H), 1.30-1.31 (m, 2H), 1.01-1.10 (m, 3H), 1.05-1.07 (m, 42H). LCMS; ESI-MS: m/z 567.20 [M+Na]⁺.

Step E: 0-((6R,7S)-9-Ethynyl-3,3-diisopropyl-2-methyl-11-oxo-7-((triisopropylsilyl)oxy)-4,10,12-trioxa-3-silatetradecan-6-yl) 1H-imidazole-1-carbothioate. To a solution of ethyl ((5S,6R)-6-hydroxy-5,7-bis((triisopropylsilyl)oxy)hept-1-yn-3-yl) carbonate (28.00 g, 51.38 mmol) in DCM (250 mL) was added TCDI (1,1′-thiocarbonyldiimidazole) (91.57 g, 513.85 mmol.). The mixture was stirred at 25° C. for 12 h. The reaction was quenched with water (200 mL), and washed with brine (50 mL), dried over anhydrous Na₂SO₄, filtered and concentrated under reduced pressure. Purification (FCC, SiO₂, PE/EA=20/1 to 5/1) afforded the title compound (10.4 g, 30.90% yield) as a colorless oil. ¹H NMR (400 MHz, CDCl₃) δ=8.33 (d, J=5.60 Hz, 1H), 7.61 (d, J=8.40 Hz, 1H), 7.10-6.94 (m, 1H), 5.75-5.64 (m, 1H), 5.47-5.35 (dd, J=8.40 Hz, J=5.20 Hz, 1H), 4.73-4.51 (m, 1H), 4.31-4.18 (m, 2H), 4.18-4.09 (m, 1H), 3.94 (dd, J=7.00, 10.60 Hz, 1H), 2.56-2.57 (m, 1H), 2.49-2.07 (m, 2H), 1.39-1.31 (m, 3H), 1.14-0.98 (m, 42H). LCMS: ESI-MS: m/z 655.40 [M+1]⁺.

Step F: Ethyl ((3R,4S)-2-methylene-4-((triisopropylsilyl)oxy)-3-(((triisopropylsilyl)oxy)methyl)cyclopentyl) carbonate. To a solution of O-((6R,7S)-9-ethynyl-3,3-diisopropyl-2-methyl-11-oxo-7-((triisopropylsilyl)oxy)-4,10,12-trioxa-3-silatetradecan-6-yl) 1H-imidazole-1-carbothioate (6.00 g, 9.16 mmol) in toluene (100 mL) was added AIBN (azobisisobutyronitrile) (752.04 mg, 4.58 mmol) and tri-n-butyltin hydride (10.66 g, 36.64 mmol, 9.69 mL) at 25° C. The mixture was stirred at 105° C. for 3 h under N₂. The reaction was quenched with KF (1.0 M, 100 mL) solution slowly, and then extracted with EA (100 mL). The organic layer was washed with brine (100 mL), dried over anhydrous Na₂SO₄, filtered, concentrated under reduced pressure. Purification (FCC, SiO₂, PE/EA=300/1 to 100/1) afforded the title compound (3.7 g, 76.37% yield) as a colorless oil. ¹H NMR (400 MHz, CDCl₃) δ=5.27 (br, d, J=3.4 Hz, 3H), 4.49-4.35 (m, 1H), 4.15-4.09 (m, 1H), 3.84-3.72 (m, 2H), 3.71-3.52 (m, 2H), 2.53-2.39 (m, 1H), 2.22 (br, d, J=6.8 Hz, 1H), 1.35-1.31 (m, 3H), 1.06-0.94 (m, 42H).

Step G: (1R,3R,4S)-2-Methylene-4-((triisopropylsilyl)oxy)-3-(((triisopropylsilyl)oxy)methyl)cyclopentan-1-ol and (1S,3R,4S)-2-methylene-4-((triisopropylsilyl)oxy)-3-(((triisopropylsilyl)oxy)methyl)cyclopentan-1-ol. To a solution of ethyl ((3R,4S)-2-methylene-4-((triisopropylsilyl)oxy)-3-(((triisopropylsilyl)oxy)methyl)cyclopentyl) carbonate (900 mg, 1.70 mmol) in EtOH (ethanol) (3 mL) was added NaOEt (289.49 mg, 4.25 mmol). The mixture was stirred at 25° C. for 1 hr. The reaction mixture was concentrated under reduced pressure. The residue was purified by column chromatography (SiO₂, Petroleum ether/Ethyl acetate=100/1 to 5/1) to give (1R,3R,4S)-2-methylene-4-((triisopropylsilyl)oxy)-3-(((triisopropylsilyl)oxy)methyl)cyclopentan-1-ol (230 mg, 29.59% yield) as a colorless oil: ¹H NMR (400 MHz, CDCl₃) δ=5.43 (s, 1H), 5.18 (s, 1H), 4.56 (s, 1H), 4.37 (dd, J=5.40, 10.00 Hz, 1H), 3.65 (dd, J=5.40, 10.40 Hz, 1H), 3.48-3.33 (m, 1H), 3.09 (d, J=11.00 Hz, 1H), 2.88 (s, 1H), 2.08-1.94 (m, 2H), 1.17-1.05 (m, 42H); and (1S,3R,4S)-2-methylene-4-((triisopropylsilyl)oxy)-3-(((triisopropylsilyl)oxy)methyl)cyclopentan-1-ol (95 mg, 12.22% yield) as colorless oil: ¹H NMR (400 MHz, CDCl₃) δ=5.28 (s, 1H), 5.11 (s, 1H), 4.58 (d, J=7.60 Hz, 1H), 4.54-4.48 (m, 1H), 3.87-3.81 (m, 1H), 3.84 (dd, J=4.40, 9.80 Hz, 1H), 3.76-3.69 (m, 1H), 2.62 (s, 1H), 2.15 (d, J=7.80 Hz, 1H), 1.99-1.94 (m, 1H), 1.10-1.01 (m, 42H); and 80 mg of mixture of the title compounds.

### Intermediate 2: (1R,3S,4S)-4-((tert-Butyldimethylsilyl)oxy)-3-(((tert-butyldimethyl)oxy)methyl)-3-fluoro-2-methylenecyclopentan-1-ol

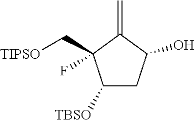

### Method A:

Step A. 2-(Hydroxymethyl)cyclopent-2-en-1-one. To a solution of cyclopent-2-en-1-one (10 g, 121.80 mmol, 10.20 mL) in a mixture of CHCl₃ (150 mL) and MeOH (100 mL) was added HCHO (13.05 g, 160.78 mmol, 11.97 mL). Then Me₂PPh (phenyldimethylphosphine) (841.33 mg, 6.09 mmol) in CHCl₃ (100 mL) was added to the above mixture. The mixture was stirred at 25° C. for 1 h. The resulting mixture was concentrated in vacuum. Purification (FCC, ISCO®; 40 g SepaFlash® Silica Flash Column, Eluent of 10˜70% Ethylacetate/Petroleum ethergradient @ 40 mL/min) afforded the title compound (24 g, 87.87% yield) as a white solid. This reaction was set up in two batches. ¹H-NMR (400 MHz, CDCl₃), S=7.51 (dd, J=1.2, 2.5 Hz, 1H), 4.32-4.19 (m, 2H), 3.09 (br s, 1H), 2.58 (td, J=2.0, 4.4 Hz, 2H), 2.42-2.30 (m, 2H).

Step B: 2-((Trityloxy)methyl)cyclopent-2-en-1-one. 2-(Hydroxymethyl)cyclopent-2-en-1-one (2 g, 17.84 mmol) in DCM (20 mL) was added DMAP (4-dimethylaminopyridine) (392.24 mg, 3.21 mmol), TrtCl (triphenylmethyl chloride) (5.22 g, 18.73 mmol) and Et₃N (2.71 g, 26.76 mmol, 3.72 mL). The mixture was stirred at 25° C. for 16 h. The reaction mixture was quenched by addition of H₂O (50 mL) and then extracted with DCM (50 mL). The combined organic layers were washed with brine (100 mL), dried over anhydrous MgSO₄, and filtered. The filtrate was concentrated under reduced pressure. Purification (FCC, ISCO®; 12 g SepaFlash® Silica Flash Column, Eluent of 5˜30% Ethyl acetate/Petroleum ether gradient @ 40 mL/min) afforded the title compound (4.8 g, 75.92% yield) as a white solid. ¹H-NMR (400 MHz, CDCl₃), δ 7.79 (t, J=1.8 Hz, 1H), 7.51-7.37 (m, 6H), 7.32-7.16 (m, 9H), 3.87 (q, J=2.6 Hz, 2H), 2.72-2.55 (m, 2H), 2.47-2.31 (m, 2H) ESI-MS: m/z 377.0 [M+Na]⁺.

Step C. (S)-2-((Trityloxy)methyl)cyclopent-2-en-1-ol. BH₃-Me₂S (borane dimethylsulfide) (10 M, 5.64 mL, 2 eq.) was dissolved in DCM (35 mL) at 0° C. (3aR)-1-Methyl-3,3-diphenyl-3a,4,5,6-tetrahydropyrrolo[1,2-c][1,3,2]oxazaborole (1 M, 5.64 mL) was added to the above solution and stirred for 1 h. After that, the solution of 2-((trityloxy)methyl)cyclopent-2-en-1-one (10 g, 28.21 mmol) in DCM (75 mL) was added drop-wise at 2 h. at 0° C. The reaction mixture was quenched with H₂O (100 mL) and then extracted with DCM (2×100 mL). The combined organic layers were washed with H₂O (200 mL), dried over MgSO₄, and filtered. The resulting solution was concentrated under reduced pressure. Purification (FCC, ISCO®; 40 g SepaFlash® Silica Flash Column, Eluent of 5˜15% Ethyl acetate/Petroleum\ether gradient@30 mL/min) afforded the title compound (48 g, 79.55%) as a colorless oil. This reaction was set up in 6 batches. ¹H-NMR (400 MHz, CDCl₃), δ 7.47-7.46 (m, 2H), 7.44 (d, J=0.7 Hz, 3H), 7.33-7.20 (m, 10H), 5.94-5.76 (m, 1H), 4.78 (br d, J=6.6 Hz, 1H), 3.88-3.76 (m, 2H), 2.60-2.43 (m, 1H), 2.35-2.19 (m, 2H), 2.14 (br s, 1H), 1.85-1.73 (m, 1H). ESI-MS: m/z 379.1 [M+Na]⁺.

Step D. (S)-(((5-(Benzyloxy)cyclopent-1-en-1-yl)methoxy)methanetriyl)tribenzene. (S)-2-((trityloxy)methyl)cyclopent-2-en-1-ol (46 g, 129.05 mmol) in DMF (dimethylformamide) (460 mL) was treated with NaH (8.26 g, 206.48 mmol, 60% purity) and TBAI (tetrabutylammonium Iodide) (23.83 g, 64.52 mmol) at 0° C. and stirred at 0° C. for 1 h. Benzylbromide (BnBr) (26.49 g, 154.86 mmol, 18.39 mL) was added at 0° C. The reaction mixture was stirred at 25° C. for 12 h. The reaction mixture was quenched by water (300 mL), and extracted with EA (3×300 mL). The organic layer was washed with brine/water (V/V=250 mL/250 mL). After that, the organic layer was dried over anhydrous Na₂SO₄, filtered and concentrated under reduced pressure. Purification (FCC, ISCO®; 40 g SepaFlash® Silica Flash Column, Eluent of 2˜10% Ethyl acetate/Petroleum ether gradient @ 35 mL/min) afforded (S)-(((5-(benzyloxy)cyclopent-1-en-1-yl)methoxy)methanetriyl)tribenzene (110 g, 95.44% yield) as a colorless oil. This reaction was set up in 2 batches. ¹H-NMR (400 MHz, CDCl₃), δ=7.50-7.48 (m, 2H), 7.48-7.46 (m, 3H), 7.39-7.16 (m, 15H), 6.02 (d, J=0.9 Hz, 1H), 4.72-4.64 (m, 1H), 4.57 (s, 1H), 4.53-4.47 (m, 1H), 3.85-3.77 (m, 1H), 3.73-3.65 (m, 1H), 2.57-2.45 (m, 1H), 2.38-2.26 (m, 1H), 2.25-2.15 (m, 1H), 1.93 (tdd, J=4.0, 9.0, 13.2 Hz, 1H). LCMS: ESI-MS: m/z 469.1 [M+Na]⁺.

Step E. (S)-(5-(Benzyloxy)cyclopent-1-en-1-yl)methanol. (S)-(((5-(benzyloxy)cyclopent-1-en-1-yl)methoxy)methanetriyl)tribenzene (15 g, 33.59 mmol) in DCM (47 mL) was treated with Triethylsilane (Et₃SiH) (6.86 g, 59.03 mmol, 9.43 mL) and trifluoroacetic acid (TFA) (3.83 g, 33.59 mmol, 2.49 mL) and the mixture was stirred at 0° C. for 0.5 h. Then additional TFA (1.91 g, 16.79 mmol, 1.24 mL) was added and stirred at 0° C. for 0.5 h. The reaction mixture was washed with saturated solution of NaHCO₃(300 mL), and then extracted with DCM (200 mL). The combined organic layers were washed with brine (300 mL), dried over anhydrous MgSO₄, filtered and concentrated under reduced pressure. Purification (FCC, ISCO®; 12 g SepaFlash® Silica Flash Column, Eluent of 0˜30% Ethyl acetate/Petroleum ether gradient @ 30 mL/min) afforded (S)-(5-(benzyloxy)cyclopent-1-en-1-yl)methanol (15 g, 57.21% yield) as a colorless oil. This reaction was set up in 4 batches. ¹H NMR (400 MHz, CDCl₃) δ 7.38-7.18 (m, 5H), 5.94-5.73 (m, 1H), 4.71-4.66 (m, 1H), 4.62 (d, J=11.7 Hz, 1H), 4.47 (d, J=11.7 Hz, 1H), 4.32-4.20 (m, 2H), 2.55-2.42 (m, 1H), 2.34-2.17 (m, 2H), 2.14 (br, s, 1H), 1.96-1.86 (m, 1H). ESI-MS: m/z 226.8 [M+Na]⁺.

Step F. (S′)-(((5-(Benzyloxy)cyclopent-1-en-1-yl)methoxy)methyl)benzene. (S)-(5-(benzyloxy)cyclopent-1-en-1-yl)methanol (30 g, 146.87 mmol) in dimethylformamide (DMF) (300 mL) was added NaH (8.81 g, 220.30 mmol, 60% purity) and TBAI (27.12 g, 73.43 mmol) at 0° C. The mixture was stirred at 0° C. for 1 h. Then BnBr (37.68 g, 220.30 mmol, 26.17 mL) was added at 0° C. The mixture was stirred at 25° C. for 12 h. The reaction mixture was quenched by water (50 mL), and extracted with EA (100 mL). The organic layer was washed with brine/water (V/V=200 mL/200 mL) and dried over anhydrous Na₂SO₄, filtered and concentrated under reduced pressure. The residue was purified by flash silica gel chromatography (ISCO®; 80 g SepaFlash® Silica Flash Column, Eluent of 0˜30% Ethyl acetate/Petroleum ether gradient @ 40 mL/min) to give (S)-(((5-(benzyloxy)cyclopent-1-en-1-yl)methoxy)methyl)benzene (50 g, crude) as a colorless oil. ¹H NMR (400 MHz, CDCl₃) δ=7.37-7.23 (m, 10H), 5.93 (d, J=1.1 Hz, 1H), 4.70-4.64 (m, 1H), 4.61-4.53 (m, 2H), 4.52-4.46 (m, 2H), 4.25-4.10 (m, 2H), 2.55-2.44 (m, 1H), 2.34-2.25 (m, 1H), 2.25-2.14 (m, 1H), 1.98-1.87 (m, 1H). ESI-MS: m/z 317.0 [M+Na]⁺.

Step G. (1S,2S,5S)-5-(Benzyloxy)-1-((benzyloxy)methyl)cyclopentane-1,2-diol and (1R,2R,5S)-5-(Benzyloxy)-1-((benzyloxy)methyl)cyclopentane-1,2-diol. A sample of OsO₄ (0.1 M, 254.77 mL) and N-methyl-morpholine-N-oxide (NMO) (12.93 g, 110.40 mmol, 11.65 mL) were sequentially added to a solution of (S)-(((5-(benzyloxy)cyclopent-1-en-1-yl)methoxy)methyl)benzene (25 g, 84.92 mmol) in THF (400 mL) and H₂O (62 mL) at 25° C. After stirring at 25° C. for 16 h. The reaction was quenched by addition of Na₂SO₃ (200 g), H₂O (500 mL) and EtOAc (1000 mL). The organic phase was separated and the aqueous phase was extracted with EtOAc (500 mL). The combined organic extracts were dried over anhydrous Na₂SO₄, filtered, and evaporated under reduced pressure. The residue was purified by flash silica gel chromatography (ISCO®; 40 g SepaFlash® Silica Flash Column, Eluent of 0˜30% Ethyl acetate/Petroleum ether gradient @ 30 mL/min) to give (1S,2S,5S)-5-(benzyloxy)-1-((benzyloxy)methyl)cyclopentane-1,2-diol and (1R,2R,5S)-5-(benzyloxy)-1-((benzyloxy)methyl)cyclopentane-1,2-diol. (50 g, 89.64% yield) as a yellow oil. This reaction was set up in 2 batches. ¹H NMR (400 MHz, CDCl₃) δ 7.39-7.32 (m, 4H), 7.31-7.23 (m, 6H), 4.59-4.51 (m, 3H), 4.61 (s, 1H), 4.47-4.39 (m, 1H), 4.14 (br d, J=2.6 Hz, 1H), 3.88-3.85 (m, 1H), 3.84 (d, J=9.5 Hz, 1H), 3.71 (d, J=9.7 Hz, 1H), 3.10 (s, 1H), 1.66-1.55 (m, 3H), 1.70-1.52 (m, 1H). ESI-MS: m/z 351.0 [M+Na]⁺.

Step H. (1R,2S,5S)-2-(Benzyloxy)-1-((benzyloxy)methyl)-5-(trityloxy)cyclopentan-1-ol. (1S,2S,5S)-5-(benzyloxy)-1-((benzyloxy)methyl)cyclopentane-1,2-diol and (1R,2R,5S)-5-(benzyloxy)-1-((benzyloxy)methyl)cyclopentane-1,2-diol (25 g, 76.13 mmol) in DCM (250 mL) was added AgNO₃ (25.86 g, 152.25 mmol) and 2,4,6-trimethylpyridine (27.67 g, 228.38 mmol, 30.18 mL) and [chloro(diphenyl)methyl]benzene (25.47 g, 91.35 mmol). The mixture was stirred at 25° C. for 1.5 h. The reaction mixture was quenched by water (500 mL), and extracted with DCM (500 mL), and the organic layer was washed with 10% acetic acid (AcOH) (500 mL) and sat. NaHCO₃ solution (500 mL). After that, the organic layer was dried over anhydrous Na₂SO₄, filtered and concentrated under reduced pressure. The residue was purified by flash silica gel chromatography (ISCO®; 40 g SepaFlash® Silica Flash Column, Eluent of 0˜15% Ethyl acetate/Petroleum ether gradient @mL/min) to give (1R,2S,5S)-2-(benzyloxy)-1-((benzyloxy)methyl)-5-(trityloxy)cyclopentan-1-ol (60 g, 55.93% yield, 81% purity) as a yellow oil. This reaction was set up for 2 batches. ¹H NMR (400 MHz, CDCl₃) δ=7.46-7.23 (m, 25H), 4.52 (s, 2H), 4.43 (s, 2H), 4.06 (t, J=6.9 Hz, 1H), 3.90-3.81 (m, 1H), 3.22 (s, 2H), 1.99-1.87 (m, 1H), 1.44-1.33 (m, 3H). LCMS:MS: m/z 593.2 [M+Na]⁺.

Step I. ((((1S,2R,3S)-3-(Benzyloxy)-2-((benzyloxy)methyl)-2-fluorocyclopentyl)oxy)methanetriyl)tribenzene. (1R,2S,5S)-2-(benzyloxy)-1-((benzyloxy)methyl)-5-(trityloxy)cyclopentan-1-ol (10 g, 17.52 mmol) in DCM (73 mL) was added diethylaminosulfur trifluoride (DAST) (7.06 g, 43.80 mmol, 5.79 mL). The mixture was stirred at −15° C. for 1.5 h. The reaction mixture was washed with sat. NaHCO₃ solution (600 mL) and extracted with DCM (200 mL). The resulting solution was washed with brine (200 mL), dried over Na₂SO₄, filtered and concentrated under reduced pressure. The residue was purified by flash silica gel chromatography (ISCO®; 120 g SepaFlash® Silica Flash Column, Eluent of 0˜5% Ethyl acetate/Petroleum ether gradient @ 25 mL/min) to give ((((1S,2R,3S)-3-(benzyloxy)-2-((benzyloxy)methyl)-2-fluorocyclopentyl)oxy)methanetriyl)tribenzene (3.6 g, 11.24% yield, 94% purity) as a yellow oil. This reaction was set up in 3 batches. ¹H NMR (400 MHz, CDCl₃) δ 7.54-7.19 (m, 25H), 4.50-4.45 (m, 4H), 4.16-4.12 (m, 2H), 3.54-3.46 (m, 1H), 3.32-3.22 (m, 1H), 2.03-1.95 (m, 1H), 1.38-1.31 (m, 3H). ¹⁹F NMR (376 MHz, CDCl₃) δ=−177.68 (s, 1F); LCMS: ESI-MS, m/z 595.1 [M+Na]⁺.

Step J. (1S,2S,3S)-3-(Benzyloxy)-2-((benzyloxy)methyl)-2-fluorocyclopentan-1-ol. ((((1S,2R,3S)-3-(benzyloxy)-2-((benzyloxy)methyl)-2-fluorocyclopentyl)oxy)methanetriyl)tribenzene (3.5 g, 6.11 mmol) in DCM (10 mL) was treated with Et₃SiH (1.25 g, 10.76 mmol, 1.72 mL) and TFA (696.81 mg, 6.11 mmol, 452.48 L). The mixture was stirred at 0° C. for 0.5 h. Then additional TFA (348.41 mg, 3.06 mmol, 226.24 μL) was added and stirred at 0° C. for 0.5 h. The reaction mixture was washed with saturated solution of NaHCO₃ solution (100 mL) and then extracted with DCM (2×100 mL). The combined organic layers were washed with brine (100 mL), dried over anhydrous MgSO₄, filtered and concentrated under reduced pressure. The residue was purified by flash silica gel chromatography (ISCO®; 12 g SepaFlash® Silica Flash Column, Eluent of 0˜30% Ethyl acetate/Petroleum ether gradient @ 30 mL/min) to give (1S,2S,3S)-3-(benzyloxy)-2-((benzyloxy)methyl)-2-fluorocyclopentan-1-ol (3 g, 74.29% yield, 100% purity) as a colorless oil. This reaction was set up in two batches. ¹H NMR (400 MHz, CDCl₃) δ=7.40-7.18 (m, 10H), 4.96 (d, J=6.3 Hz, 1H), 4.57-4.50 (m, 3H), 4.49-4.43 (m, 1H), 4.04-3.85 (m, 2H), 3.83-3.62 (m, 2H), 2.08-1.87 (m, 2H), 1.61-1.44 (m, 2H). LCMS:ESI-MS: m/z 352.9 [M+Na]⁺.

Step K: (((1S,2R,3S)-3-(Benzyloxy)-2-((benzyloxy)methyl)-2-fluorocyclopentyl)oxy)(tert-butyl)dimethylsilane. To a solution of (1S,2S,3S)-3-(benzyloxy)-2-((benzyloxy)methyl)-2-fluorocyclopentan-1-ol (3.0 g, 9.08 mmol) in DMF (10 mL) was added imidazole (3.71 g, 54.48 mmol) and tert-butyldimethylsilyl chloride (TBSCl) (3.42 g, 22.70 mmol). The mixture was stirred at 25° C. for 12 h. The reaction was quenched with H₂O (50 mL), and extracted with EA (50 mL*2). The organic layer was dried over anhydrous Na₂SO₄, and concentrated at low pressure. The residue was purified by column chromatography (SiO₂, Petroleum ether/Ethyl acetate=100/1 to 10/1) to give (((1S,2R,3S)-3-(benzyloxy)-2-((benzyloxy)methyl)-2-fluorocyclopentyl)oxy)(tert-butyl)dimethylsilane (3.96 g, 98.08% yield) as a colorless oil. LCMS: ESI-MS: m/z 445.2, [M+H]⁺, 467.1 [M+Na]⁺.

Step L: (1S,2R,3S)-3-((tert-Butyldimethylsilyl)oxy)-2-fluoro-2-(hydroxymethyl)cyclopentan-1-ol. To a solution of (((1S,2R,3S)-3-(benzyloxy)-2-((benzyloxy)methyl)-2-fluorocyclopentyl)oxy)(tert-butyl)dimethylsilane (2.0 g, 4.50 mmol) in MeOH (100 mL) was added Pd/C (1.5 g, 10% purity) and acetic acid (HOAc) (675.27 mg, 11.24 mmol, 643.11 μL). The suspension was degassed under vacuum and purged with H₂ several times. The mixture was stirred under H₂ balloon (15 psi) at 25° C. for 12 h. The reaction mixture was filtered off, and the filtrate was concentrated at low pressure. The residue was purified by column chromatography (SiO₂, Petroleum ether (PE)/Ethyl acetate=100/1 to 5/1) to give (1S,2R,3S)-3-((tert-butyldimethylsilyl)oxy)-2-fluoro-2-(hydroxymethyl)cyclopentan-1-ol (1.1 g, 92.49% yield) as yellow oil. ¹H NMR (400 MHz, CD₃OD) δ=4.29-4.10 (m, 2H), 3.91-3.67 (m, 2H), 2.28-2.13 (m, 1H), 2.11-1.97 (m, 1H), 1.65 (dddd, J=1.8, 7.4, 12.6, 18.1 Hz, 1H), 1.53-1.38 (m, 1H), 1.00-0.84 (m, 9H), 0.07-−0.07 (m, 6H). ¹⁹F NMR (376 MHz, CD₃OD) δ=−185.71 (br, dd, J=15.8, 28.2 Hz, 1F).

Step M: (1S,2R,3S)-3-((tert-Butyldimethylsilyl)oxy)-2-(((tert-butyldimethylsilyl)oxy)methyl)-2-fluorocyclopentan-1-ol. To a solution of (1S,2R,3S)-3-((tert-butyldimethylsilyl)oxy)-2-fluoro-2-(hydroxymethyl)cyclopentan-1-ol (1.1 g, 4.16 mmol) in DMF (5 mL) was added imidazole (1.13 g, 16.64 mmol) and TBSCl (940.55 mg, 6.24 mmol). The mixture was stirred at 25° C. for 12 h. The reaction was quenched with H₂O (5 mL). The resulting solution was extracted with ethyl acetate (EA) (2×50 mL). The organic layer was dried over anhydrous Na₂SO₄, and concentrated at low pressure. The residue was purified by column chromatography (SiO₂, Petroleum ether/Ethyl acetate=100/1 to 10/1) to give (1S,2R,3S)-3-((tert-butyldimethylsilyl)oxy)-2-(((tert-butyldimethylsilyl)oxy)methyl)-2-fluorocyclopentan-1-ol (0.912 g, 57.89% yield) as a colorless oil. ¹H NMR (400 MHz, CDCl₃) δ=4.43-4.20 (m, 1H), 4.20-3.96 (m, 2H), 3.05 (br, s, 1H), 2.37-2.17 (m, 1H), 2.04-1.93 (m, 1H), 1.80-1.66 (m, 1H), 1.60-1.40 (m, 1H), 1.37-1.22 (m, 1H), 1.02-0.83 (m, 18H), 0.25-0.01 (m, 12H). ¹⁹F NMR (376 MHz, CDCl₃) δ=−179.49 (s, 1F).

Step N: (2S,3S)-3-((tert-Butyldimethylsilyl)oxy)-2-(((tert-butyldimethylsilyl)oxy)methyl)-2-fluorocyclopentan-1-one. To a solution of (1S,2R,3S)-3-((tert-butyldimethylsilyl)oxy)-2-(((tert-butyldimethylsilyl)oxy)methyl)-2-fluorocyclopentan-1-ol (760 mg, 2.01 mmol) in DCM (15 mL) was added 1,1,1-triacetoxy-1,1-dihydro-1,2-benziodoxol-3(1H)-one (Dess-Martin Periodinane/or DMP) (1.70 g, 4.01 mmol). The mixture was stirred at 25° C. for 2 h. The reaction mixture was concentrated at low pressure. The residue was stirred in EA/PE (10 mL, 10/1) and filtered off. The filtrate was concentrated at low pressure. The residue was purified by column chromatography (SiO₂, Petroleum ether/Ethyl acetate=100/0 to 30/1) to give (2S,3S)-3-((tert-butyldimethylsilyl)oxy)-2-(((tert-butyldimethylsilyl)oxy)methyl)-2-fluorocyclopentan-1-one (0.750 g, 99.21% yield) as a colorless oil. ¹H NMR (400 MHz, CDCl₃) δ=4.54-4.42 (m, 1H), 3.84-3.70 (m, 2H), 2.60-2.44 (m, 1H), 2.27-2.09 (m, 2H), 2.09-1.92 (m, 1H), 0.88 (d, J=4.5 Hz, 18H), 0.15-0.05 (m, 12H). ¹⁹F NMR (376 MHz, CDCl₃) δ=−182.78 (s, 1F).

Step O: tert-Butyl(((1S,2S)-2-((tert-butyldimethylsilyl)oxy)-1-fluoro-5-methylenecyclopentyl)methoxy)dimethylsilane. To a solution of methyl (triphenyl)phosphonium bromide (2.13 g, 5.97 mmol) in toluene (5 mL) was added potassium 2-methylbutan-2-olate (3.02 g, 5.97 mmol, 3.47 mL, 25% purity) and stirred at 25° C. for 1 h. Then (2S,3S)-3-((tert-butyldimethylsilyl)oxy)-2-(((tert-butyldimethylsilyl)oxy)methyl)-2-fluorocyclopentan-1-one (750 mg, 1.99 mmol) in toluene (4 mL) was added, and the mixture was stirred at 25° C. for 4 h. The reaction mixture was quenched with sat. aq. NH₄Cl solution (30 mL), and extracted by EA (50 mL*2). The resulting solution was dried over anhydrous Na₂SO₄, and concentrated at low pressure. Purification (FCC, SiO₂, PE) afforded tert-butyl(((1S,2S)-2-((tert-butyldimethylsilyl)oxy)-1-fluoro-5-methylenecyclopentyl)methoxy)dimethylsilane (686 mg, 91.95% yield) as a colorless oil. ¹H NMR (400 MHz, CDCl₃) δ 5.30-5.24 (m, 1H), 5.16 (td, J=2.1, 4.3 Hz, 1H), 4.22 (td, J=5.9, 11.1 Hz, 1H), 3.82-3.58 (m, 2H), 2.64-2.47 (m, 1H), 2.21-2.16 (m, 1H), 2.07-1.97 (m, 1H), 1.88-1.68 (m, 1H), 0.93-0.83 (m, 18H), 0.14-0.00 (m, 12H). ¹⁹F NMR (376 MHz, CDCl₃) δ−182.77 (s, 1F).

Step P: (1S,3S,4S)-4-((tert-Butyldimethylsilyl)oxy)-3-(((tert-butyldimethylsilyl)oxy)methyl)-3-fluoro-2-methylenecyclopentan-1-ol and (1R,3S,4S)-4-((tert-butyldimethylsilyl)oxy)-3-(((tert-butyldimethylsilyl)oxy)methyl)-3-fluoro-2-methylenecyclopentan-1-ol. To a solution of CH₃COOH (6.73 mg, 112.10 mol, 6.41 μL) in DCM (3 mL) was added selenium dioxide (12.44 mg, 112.10 mol) and 2-hydroperoxy-2-methylpropane (5.5 M, 407.62 μL) at 25° C., and stirred at 25° C. for 30 min. tert-butyl(((1S,2S)-2-((tert-butyldimethylsilyl)oxy)-1-fluoro-5-methylenecyclopentyl)methoxy)dimethylsilane (420 mg, 1.12 mmol) in DCM (2 mL) was added and stirred for 72 h. The reaction was diluted with DCM (5 mL), and 1.0 g of silica gel was added. The resulting mixture was concentrated at low pressure. The residue was purified by column chromatography (SiO₂, Petroleum ether/Ethyl acetate=200/1 to 20/1) to give (1S,3S,4S)-4-((tert-butyldimethylsilyl)oxy)-3-(((tert-butyldimethylsilyl)oxy)methyl)-3-fluoro-2-methylenecyclopentan-1-ol (98 mg, 22.38% yield) as colorless oil: ¹H NMR (400 MHz, CDCl₃) δ 5.54-5.45 (m, 2H), 4.74 (br, t, J=5.8 Hz, 1H), 4.43 (td, J=5.4, 7.7 Hz, 1H), 3.86-3.57 (m, 2H), 2.21-2.07 (m, 1H), 1.89-1.74 (m, 1H), 0.89 (d, J=3.0 Hz, 18H), 0.18-−0.05 (m, 12H); ¹⁹F NMR (376 MHz, CDCl₃) δ=−167.29 (s, 1F); and (1R,3S,4S)-4-((tert-butyldimethylsilyl)oxy)-3-(((tert-butyldimethylsilyl)oxy)methyl)-3-fluoro-2-methylenecyclopentan-1-ol (50 mg, 11.84% yield) as colorless oil: ¹H NMR (400 MHz, CDCl₃) δ=5.62 (dd, J=1.1, 3.3 Hz, 1H), 5.49 (dd, J=1.8, 2.6 Hz, 1H), 4.47-4.35 (m, 1H), 4.33 (br d, J=9.7 Hz, 1H), 3.67 (dd, J=11.7, 13.2 Hz, 1H), 3.54-3.39 (m, 1H), 2.76 (d, J=11.2 Hz, 1H), 2.13-2.00 (m, 1H), 1.97-1.82 (m, 1H), 0.89 (d, J=3.1 Hz, 18H), 0.15-−0.04 (m, 12H); ¹⁹F NMR (376 MHz, CDCl₃) δ=−167.63 (br, 1F).

### Method B:

### (1R,3S,4S)-4-((tert-Butyldimethylsilyl)oxy)-3-(((tert-butyldimethylsilyl)oxy)methyl)-3-fluoro-2-methylenecyclopentan-1-ol

Step A: (1R,3S,4S)-4-((tert-Butyldimethylsilyl)oxy)-3-(((tert-butyldimethylsilyl)oxy)methyl)-3-fluoro-2-methylenecyclopentyl 4-nitrobenzoate. To a solution of (1S,3S,4S)-4-((tert-butyldimethylsilyl)oxy)-3-(((tert-butyldimethylsilyl)oxy)methyl)-3-fluoro-2-methylenecyclopentan-1-ol (Method A, product from Step P, 185 mg, 473.53 mol) and para-nitrobenzoic acid (PNBA) (126.62 mg, 757.66 mol) in THF (2 mL) was added diisopropyl azodicarboxylate (DIAD) (287.26 mg, 1.42 mmol, 276.21 μL) and triphenylphosphine (PPh₃) (372.60 mg, 1.42 mmol) at 0° C. The mixture was stirred at 25° C. for 12 h. The reaction mixture was concentrated under reduced pressure. The residue was purified by column chromatography (SiO₂, PE/EA=100/1 to 70/1) to give (1R,3S,4S)-4-((tert-butyldimethylsilyl)oxy)-3-(((tert-butyldimethylsilyl)oxy)methyl)-3-fluoro-2-methylenecyclopentyl 4-nitrobenzoate (150 mg, 58.68% yield) as a colorless oil. LCMS: ESI-MS: m/z 562.2 [M+Na]⁺.

Step B: (1R,3S,4S)-4-((tert-Butyldimethylsilyl)oxy)-3-(((tert-butyldimethylsilyl)oxy)methyl)-3-fluoro-2-methylenecyclopentan-1-ol. (1R,3S,4S)-4-((tert-butyldimethylsilyl)oxy)-3-(((tert-butyldimethylsilyl)oxy)methyl)-3-fluoro-2-methylenecyclopentyl 4-nitrobenzoate (75 mg, 138.94 mol) was treated with NH₃ (7 M, 2 mL, in CH₃₀H). The mixture was stirred at 25° C. for 1 h. The reaction mixture was concentrated under reduced pressure. The residue was purified by column chromatography (SiO₂, PE/EA=100/1 to 70/1) to give (1R,3S,4S)-4-((tert-butyldimethylsilyl)oxy)-3-(((tert-butyldimethylsilyl)oxy)methyl)-3-fluoro-2-methylenecyclopentan-1-ol (51 mg, 46.98% yield) as colorless oil.

### Intermediate 3: (3R,4S)-4-(Benzyloxy)-3-((benzyloxy)methyl)-3-fluoro-2-methylenecyclopentan-1-ol

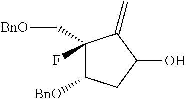 [SMILES: C=C1C(O)C[C@H](OCc2ccccc2)[C@]1(F)COCc1ccccc1]

Step A: (2R,3S)-3-(Benzyloxy)-2-((benzyloxy)methyl)-2-fluorocyclopentan-1-one. Dess-Martin Periodinane (3.72 g, 8.78 mmol) was added to a solution of (1S,2S,3S)-3-(benzyloxy)-2-((benzyloxy)methyl)-2-fluorocyclopentan-1-ol (Intermediate 2, product from Step J, 1.45 g, 4.39 mmol) in DCM (20 mL). The resulting mixture was stirred at 28° C. for 2 h. The reaction mixture was washed with water (2×100 mL). The resulting organic layer was separated, and washed with saturated sodium bicarbonate solution (150 mL) and brine (80 mL), and dried over anhydrous sodium sulfate. The resulting solution was concentrated at low pressure. The residue was purified by flash silica gel chromatography (ISCO®; 12 g SepaFlash® Silica Flash Column, Eluent of 0˜10% Ethyl acetate/Petroleum ether gradient @ 20 mL/min) to give (2R,3S)-3-(benzyloxy)-2-((benzyloxy)methyl)-2-fluorocyclopentan-1-one (1.29 g, 89.51% yield) as a colorless oil. ESI-MS: m/z 351.0 [M+Na]⁺.

Step B: ((((1R,2S)-2-(Benzyloxy)-1-fluoro-5-methylenecyclopentyl)methoxy)methyl)benzene. To a solution of methyl (triphenyl)phosphonium bromide (2.77 g, 7.77 mmol) in toluene (16 mL) was added potassium 2-methylbutan-2-olate (3.92 g, 7.77 mmol, 4.51 mL, 25% purity) at 25° C., and stirred at 25° C. for 1 h. (2R,3S)-3-(benzyloxy)-2-((benzyloxy)methyl)-2-fluorocyclopentan-1-one (850.00 mg, 2.59 mmol) in toluene (6 mL) was added at 0° C. and stirred at 25° C. for another 2 h. The reaction was quenched with saturated aq. NH₄Cl solution (50 mL), and extracted by EA (20 mL*2). The organic layer was washed with brine (50 mL). The organic layer was dried over anhydrous Na₂SO₄, concentrated at low pressure. The residue was purified by flash silica gel chromatography (ISCO®; 12 g SepaFlash® Silica Flash Column, Eluent of 0˜20% Ethylacetate/Petroleum ether gradient @ 20 mL/min) to give ((((1R,2S)-2-(benzyloxy)-1-fluoro-5-methylenecyclopentyl)methoxy)methyl)benzene (798 mg, 94.45% yield) as colorless oil. ¹H NMR (400 MHz, CDCl₃) δ=7.36-7.23 (m, 10H), 5.34-5.28 (m, 1H), 5.22-5.17 (m, 1H), 4.67-4.58 (m, 4H), 4.18-4.13 (m, 1H), 3.90-3.80 (m, 2H), 2.53-2.44 (m, 2H), 2.02-1.92 (m, 1H), 1.90-1.79 (m, 1H). LCMS: ESI-MS: m/z 349.1 [M+Na]⁺.

Step C: (3R,4S)-4-(Benzyloxy)-3-((benzyloxy)methyl)-3-fluoro-2-methylenecyclopentan-1-ol.

To a solution of SeO₂ (13.26 mg, 119.48 mol) in DCM (4 mL) was added tert-butyl hydroperoxide (TBHP) (5.5 M, 434.49 μL) and CH₃COOH (4.78 mg, 79.66 mol, 4.56 μL) at 25° C., and stirred at 25° C. for 30 min. ((((1R,2S)-2-(benzyloxy)-1-fluoro-5-methylenecyclopentyl)methoxy)methyl)benzene (260.00 mg, 796.56 mol) in DCM (2 mL) was added and stirred for 50 h. The reaction mixture was diluted with DCM (6 mL), and 2 g of silica gel was added. The mixture was concentrated at low pressure. The residue was purified by column chromatography (SiO₂, Petroleum ether/Ethyl acetate=30/1 to 8/1) to give (3R,4S)-4-(benzyloxy)-3-((benzyloxy)methyl)-3-fluoro-2-methylenecyclopentan-1-ol (0.115 g, 42.16% yield) as a colorless oil. ¹H NMR (400 MHz, CDCl₃) δ=7.41-7.18 (m, 10H), 5.74-5.47 (m, 2H), 4.84-4.55 (m, 5H), 4.41-4.23 (m, 1H), 3.93-3.43 (m, 3H), 2.38-2.17 (m, 1H), 1.98-1.86 (m, 1H). LCMS: ESI-MS: m/z 365.1 [M+Na]⁺.

### Intermediate 4. N,N-Di-BOC-7H-pyrrolo[2,3-d]pyrimidin-4-amine

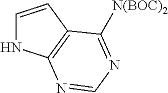

Step A: N,N,N-Tri-BOC-7H-pyrrolo[2,3-d]pyrimidin-4-amine. To a solution of 7H-pyrrolo[2,3-d]pyrimidin-4-amine (500 mg, 3.73 mmol) in THF (10 mL) was added 4-dimethylaminopyridine (DMAP) (45.54 mg, 372.75 mol) and di-tert-butyl dicarbonate (Boc₂O) (4.07 g, 18.64 mmol, 4.28 mL). The mixture was stirred at 25° C. for 12 h. The reaction mixture was concentrated at low pressure. The residue was dissolved in EA (10 mL). The resulting solution was washed with (0.5 N HCl, 10 mL) and brine (10 mL), and concentrated at low pressure. The residue was purified by flash silica gel chromatography (ISCO®; 12 g SepaFlash® Silica Flash Column, Eluent of 0˜30% Ethyl acetate/Petroleum ether gradient @ 25 mL/min) to give the title compound (0.68 g, 41.99% yield, 100% purity) as a white solid. ¹H NMR (400 MHz, CD₃OD) δ=8.85 (s, 1H), 7.90 (d, J=4.3 Hz, 1H), 6.63 (d, J=4.0 Hz, 1H), 1.70 (s, 9H), 1.42-1.31 (m, 18H). LCMS: ESI-MS: m/z 435.2, [M+H]⁺; 457.1 [M+Na]⁺.

Step B: N,N-Di-BOC-7H-pyrrolo[2,3-d]pyrimidin-4-amine. To a solution of N,N,N-Tri-BOC-7H-pyrrolo[2,3-d]pyrimidin-4-amine (0.650 g, 1.50 mmol) in MeOH (20 mL) was added saturated aq. NaHCO₃ solution (10 mL), and stirred at 50° C. for 1 h. The reaction mixture was diluted with water (10 mL), and extracted with EA (10 mL*3). The organic layer was washed with brine (10 mL), dried over anhydrous Na₂SO₄, filtered, and concentrated at low pressure. The residue was purified by flash silica gel chromatography (ISCO®; 4 g SepaFlash® Silica Flash Column, Eluent of 0˜30% Ethyl acetate/Petroleum ether gradient @ 20 mL/min) to give N,N-Di-BOC-7H-pyrrolo[2,3-d]pyrimidin-4-amine (366 mg, 73.17% yield) as a white solid. ¹H NMR (400 MHz, CD₃OD) 6=8.65 (s, 1H), 7.55 (d, J=3.5 Hz, 1H), 6.49 (d, J=3.5 Hz, 1H), 1.46-1.27 (m, 18H).

### Intermediate 5: 2-Isobutyramido-9H-purin-6-yl Diphenylcarbamate

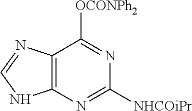

Step A: N-(6-Oxo-6,9-dihydro-1H-purin-2-yl)isobutyramide. To a solution of 2-amino-1,9-dihydro-6H-purin-6-one (5 g, 33.08 mmol) in DMF (50 mL) was added isobutyric anhydride (14.13 g, 89.33 mmol, 14.81 mL) in one portion at 30° C. under N₂. The reaction mixture was stirred at 155° C. for 4 h. The reaction mixture was cooled to r.t. and the precipitation was filtered to give a white solid. The filtered cake was washed with EtOH/H₂O (1:1, 50 mL*3) and dried under reduced pressure. N-(6-oxo-6,9-dihydro-1H-purin-2-yl)isobutyramide (13.7 g, 2 batches, 61.93 mmol, 93.59% yield) was obtained as a white solid. ¹H NMR (400 MHz, DMSO) δ=13.26 (br, 1H), 12.06 (br, 1H), 11.52 (br, 1H), 8.01 (s, 1H), 2.78-2.71 (m, 1H), 1.12-1.10 (m, 6H).

Step B: N-(9-Acetyl-6-oxo-6,9-dihydro-1H-purin-2-yl)isobutyramide. To a solution of N-(6-oxo-6,9-dihydro-1H-purin-2-yl)isobutyramide (3.6 g, 16.27 mmol) in DMF (20 mL) was added acetyl acetate (4.32 g, 42.31 mmol, 3.96 mL) in one portion at 30° C. under N₂. The reaction mixture was stirred at 100° C. for 2 h. The solvent was completely removed by evaporation under reduced pressure to give a white solid. The residue was suspended in EtOH (20 mL), filtered and the precipitate was washed with EtOH (5 mL*3) to afford a white solid. N-(9-acetyl-6-oxo-6,9-dihydro-1H-purin-2-yl)isobutyramide (2.9 g, 11.02 mmol, 67.69% yield) was obtained as a white solid. ¹H NMR (400 MHz, DMSO) δ=12.28 (br, 1H), 11.72 (br, 1H), 8.46 (s, 1H), 2.83-2.74 (m, 4H), 1.15 (s, 3H), 1.13 (s, 3H).

Step C: 9-Acetyl-2-isobutyramido-9H-purin-6-yl diphenylcarbamate. To a suspension of N-(9-acetyl-6-oxo-6,9-dihydro-1H-purin-2-yl)isobutyramide (2.9 g, 11.02 mmol) in pyridine (60 mL) was added N,N-diisopropylethylamine (Hunig's base or DIPEA or DIEA) (2.85 g, 22.03 mmol, 3.84 mL) in one portion at 25° C. under N₂. After the addition of diphenylcarbamic chloride (2.81 g, 12.12 mmol), the reaction mixture was stirred at 25° C. for 2 h. Water (4 mL) was added to the reaction mixture and the resulting mixture was stirred for 10 min. The solvent was completely removed by evaporation under reduced pressure to give a brown solid. The crude product was used into the next step directly without further purification. 9-acetyl-2-isobutyramido-9H-purin-6-yl diphenylcarbamate (6 g, crude) was obtained as a brown solid.

Step D: 2-Isobutyramido-9H-purin-6-yl diphenylcarbamate. A mixture of 9-acetyl-2-isobutyramido-9H-purin-6-yl diphenylcarbamate (5.05 g, 11.02 mmol) in EtOH (40 mL) and H₂O (40 mL) was stirred at 100° C. for 2 h. The reaction was cooled to r.t. and the precipitation was filtered to give a brown solid. The residue suspended in EtOH (30 mL), filtered and the filtered cake was washed with EtOH (10 mL*3) to afford a white solid. 2-isobutyramido-9H-purin-6-yl diphenylcarbamate (3.9 g, 9.37 mmol, 84.98% yield) was obtained as a white solid. ¹H NMR (400 MHz, DMSO) δ=10.59 (br, 1H), 8.43 (s, 1H), 7.48-7.30 (m, 11H), 2.80-7.77 (m, 1H), 1.09 (s, 3H), 1.07 (s, 3H).

### Intermediate 6: 3-Benzoyl-5-methylpyrimidine-2,4(1H,3H)-dione

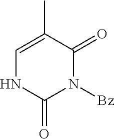

Step A: 1,3-Dibenzoyl-5-methylpyrimidine-2,4(1H,3H)-dione. To a solution of 5-methylpyrimidine-2,4(1H,3H)-dione (5 g, 39.65 mmol) in CH₃CN (100 mL) was added pyridine (14.70 g, 185.84 mmol, 15.00 mL) in one portion at 30° C. under N₂. After the addition of benzoyl chloride (BzCl) (19.51 g, 138.76 mmol, 16.12 mL), the reaction mixture was stirred at 30° C. for 12 h. The solvent was removed in vacuo to give a yellow oil. The residue was purified by silica gel column chromatography (Petroleum ether:Ethyl acetate=3:1 to 1:1) to give 1,3-dibenzoyl-5-methylpyrimidine-2,4(1H,3H)-dione (25 g, 2 batches, 74.78 mmol, 94.30% yield) as a pale yellow solid. LCMS: ESI-MS: m/z=357.0 [M+Na]⁺.

Step B: 3-Benzoyl-5-methylpyrimidine-2,4(1H,3H)-dione. To a solution of 1,3-dibenzoyl-5-methylpyrimidine-2,4(1H,3H)-dione (6 g, 17.95 mmol) in dioxane (40 mL) was added K₂CO₃ (0.5 M, 17.95 mL) in one portion at 30° C. under N₂. The reaction mixture was stirred at 30° C. for 1 h. Dioxane was removed under reduce pressure. The residue was purified by recrystallization from acetonitrile (5 mL) and water (50 mL) to give 3-benzoyl-5-methylpyrimidine-2,4(1H,3H)-dione (3.8 g, 16.51 mmol, 91.97% yield, 100% purity) as a white solid. ¹H NMR (400 MHz, CDCl₃) δ=11.40 (bs, 1H), 7.94 (br, d, J=7.6 Hz, 2H), 7.77-7.76 (m, 1H), 7.62-7.54 (m, 3H), 1.82 (s, 3H). LCMS: ESI-MS: m/z=252.9 [M+Na]⁺.

### Intermediate 7. ((1S,3R,5S)-1-((Bis(4-methoxyphenyl)(phenyl)methoxy)methyl)-3-hydroxy-2-methylene-5-(pivaloyloxy)cyclopentyl)methyl Pivalate

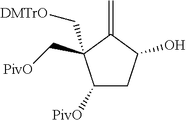

Step A: (4S,5R)-4-Hydroxy-5-(hydroxymethyl)dihydrofuran-2(3H)-one. To a solution of (4S,5R)-5-(hydroxymethyl)tetrahydrofuran-2,4-diol (35 g, 260.94 mmol) in H₂O (400 mL) was added Br₂ (125.10 g, 782.82 mmol, 40.36 mL) at 0° C., and stirred at 25° C. for 16 h. The reaction was quenched by addition of Na₂SO₃ (solid) at 0° C., and a clear yellow solution was obtained which was concentrated at reduced pressure at 35° C. to remove the solvent. The residue was dissolved in EtOH (500 mL) and adjusted to pH=7 using Na₂SO₃ (solid), and then filtered. The filtrate was concentrated in vacuum. The residue was dissolved in DCM/EtOH (900 mL/300 mL), and stirred at 25° C. for 30 min and filtered. The filtrate was concentrated in vacuum to give crude (4S,5R)-4-hydroxy-5-(hydroxymethyl)dihydrofuran-2(3H)-one (126 g, 953.72 mmol, 91.37% yield) as colorless oil. (4 Batches). ¹H NMR (400 MHz, D20) δ=4.53-4.45 (m, 2H), 3.85-3.76 (m, 1H), 3.75-3.65 (m, 1H), 2.98 (dd, J=7.0, 18.6 Hz, 1H), 2.58-2.45 (m, 1H).

Step B: (2R,3S)-5-Oxo-2-((pivaloyloxy)methyl)tetrahydrofuran-3-yl pivalate. To a solution of (4S,5R)-4-hydroxy-5-(hydroxymethyl)dihydrofuran-2(3H)-one (20 g, 151.38 mmol) in pyridine (60 mL) was added 2,2-dimethylpropanoyl chloride (41.98 g, 348.18 mmol, 42.84 mL) in drop wise at 0° C. The mixture was stirred at 45° C. for 12 h. The reaction mixture was quenched with MeOH (40 mL). The reaction mixture was concentrated under reduced pressure. The residue was dissolved in EA (100 mL) and the resulting solution was washed with H₂O (100 mL*2). The organic layer was dried over anhydrous Na₂SO₄, filtered and concentrated under reduced pressure. The residue was purified by column chromatograph (PE/EA from 35/1 to 5/1) to give (2R,3S)-5-oxo-2-((pivaloyloxy)methyl)tetrahydrofuran-3-yl pivalate (35 g, 116.53 mmol, 76.98% yield) as white solid. ¹H NMR (400 MHz, CDCl₃) δ=5.25 (td, J=1.7, 7.4 Hz, 1H), 4.63 (dt, J=1.4, 3.1 Hz, 1H), 4.41-4.34 (m, 1H), 4.31-4.24 (m, 1H), 3.02 (dd, J=7.6, 18.9 Hz, 1H), 2.61 (dd, J=1.8, 18.7 Hz, 1H), 1.22-1.18 (m, 18H).

Step C: (2R,3S)-5-Hydroxy-2-((pivaloyloxy)methyl)tetrahydrofuran-3-yl pivalate. To a solution of (2R,3S)-5-oxo-2-((pivaloyloxy)methyl)tetrahydrofuran-3-yl pivalate (10 g, 33.29 mmol) in THF (100 mL) was added diisobutylaluminium hydride (DIBAL-H, DIBAL) (1 M, 99.88 mL, 3 eq.) at −60° C., and stirred at −60° C. for 3 h. The reaction was quenched with MeOH (50 mL) at −30° C. and diluted with EA (100 mL). The reaction mixture was filtered and the filtrate was concentrated under reduced pressure. The residue was purified by column chromatograph (PE/EA=30/1 to 3/1) to give (2R,3S)-5-hydroxy-2-((pivaloyloxy)methyl)tetrahydrofuran-3-yl pivalate (4.2 g, 13.89 mmol, 41.72% yield) as colorless oil. ¹H NMR (400 MHz, CDCl₃) δ=5.59-5.65 (m, 1H), 5.12-5.23 (m, 1H), 4.38-4.39 (m, 1H), 4.13-4.24 (m, 2H), 2.33-2.39 (m, 1H), 2.04-2.07 (m, 1H), 1.19-1.26 (m, 18H). LCMS: ESI-MS: m/z=604.3 [2M+H]⁺.

Step D: (2R,3S)-2,5-Dihydroxyhept-6-yne-1,3-diyl bis(2,2-dimethylpropanoate). To a solution of (2R,3S)-5-hydroxy-2-((pivaloyloxy)methyl)tetrahydrofuran-3-yl pivalate (4.2 g, 13.89 mmol) in THF (40 mL) was added bromoethynyl magnesium (0.5 M, 83.34 mL) in dropwise at −78° C. The mixture was stirred at 25° C. for 2 h. The reaction mixture was quenched by addition of saturated NH₄Cl solution (50 mL) to achieve pH=7. The resulting mixture was extracted with EA (30 mL) and washed with brine (2*30 mL). The combined organic layers were dried over anhydrous Na₂SO₄, filtered and concentrated under reduced pressure. The residue was purified by column chromatography (PE/EA=15/1 to 3/1) to give (2R,3S)-2,5-dihydroxyhept-6-yne-1,3-diyl bis(2,2-dimethylpropanoate) (4 g, 12.18 mmol, 87.69% yield) as colorless oil. ¹H NMR (400 MHz, CDCl₃) δ=5.03-5.11 (m, 1H), 4.34-4.56 (m, 1H), 4.21-4.25 (m, 1H), 4.11-4.16 (m, 1H), 3.94-3.99 (m, 1H), 2.95-3.02 (br, s, 1H), 2.46-2.49 (dd, J=6.15, 2.13 Hz, 1H), 1.98-2.25 (m, 3H), 1.20-1.23 (m, 18H). LCMS: ESI-MS: m/z=351.1 [M+Na]⁺.

Step E: (2R,3S)-5-((tert-butyldimethylsilyl)oxy)-2-hydroxyhept-6-yne-1,3-diyl bis(2,2-dimethylpropanoate). To a solution of (2R,3S)-2,5-dihydroxyhept-6-yne-1,3-diyl bis(2,2-dimethylpropanoate) (4 g, 12.18 mmol) in DMF (6 mL) was added imidazole (2.49 g, 36.54 mmol) and tert-butyl-chloro-dimethyl-silane (2.75 g, 18.27 mmol, 2.24 mL), and stirred at 25° C. for 12 h. The reaction mixture was quenched by MeOH (10 mL), and extracted with EA (30 mL). The resulting solution was washed with brine (15 mL*2). The combined organic layers were dried over anhydrous Na₂SO₄, filtered and concentrated under reduced pressure. The residue was purified by column chromatography (PE/EA=35/1 to 3/1) to give (2R,3S)-5-((tert-butyldimethylsilyl)oxy)-2-hydroxyhept-6-yne-1,3-diyl bis(2,2-dimethylpropanoate) (2.5 g, 5.65 mmol, 46.37% yield) as yellow oil. ¹H NMR (400 MHz, CDCl₃) δ=5.03-5.15 (m, 1H), 4.40-4.53 (m, 1H), 4.06-4.17 (m, 2H), 3.96-4.01 (m, 1H), 2.73-2.91 (m, 1H), 2.42-2.45 (dd, J=11.04, 2.26 Hz, 1H), 2.02-2.20 (m, 2H), 1.19-1.27 (m, 18H), 0.89-0.91 (m, 9H), 0.09-0.19 (m, 6H). LCMS: ESI-MS: m/z=465.1 [M+Na]⁺.

Step F: (3S)-5-((tert-butyldimethylsilyl)oxy)-2-oxohept-6-yne-1,3-diyl bis(2,2-dimethylpropanoate). To a solution of (2R,3S)-5-((tert-butyldimethylsilyl)oxy)-2-hydroxyhept-6-yne-1,3-diyl bis(2,2-dimethylpropanoate) (16 g, 36.15 mmol) in DCM (100 mL) was added Dess-Martin periodinane (45.99 g, 108.44 mmol, 33.57 mL) at 0° C., and stirred at 25° C. for 2 h. The reaction mixture was quenched by addition of saturated NaHCO₃ and Na₂SO₃ solution (1:1, 100 mL). The resulting solution was washed with brine (100 mL*2). The combined organic layers were dried over anhydrous Na₂SO₄, filtered and concentrated under reduced pressure. The residue was purified by silica gel column (PE/EA=250/1 to 20/1) to give (3S)-5-((tert-butyldimethylsilyl)oxy)-2-oxohept-6-yne-1,3-diyl bis(2,2-dimethylpropanoate) (15 g, 34.04 mmol, 94.18% yield) as a colorless oil. ¹H NMR (400 MHz, CDCl₃) δ=5.24-5.10 (m, 1H), 4.53-4.40 (m, 1H), 4.23-3.98 (m, 1H), 3.00-2.86 (m, 1H), 2.79-2.64 (m, 1H), 2.41-2.27 (m, 1H), 2.24-2.00 (m, 2H), 1.24-1.02 (m, 18H), 0.88-0.70 (m, 9H), 0.15-0.07 (m, 6H). LCMS: ESI-MS: m/z=463.2 [M+Na]⁺.

Step G: (3S)-5-((tert-butyldimethylsilyl)oxy)-2-methylenehept-6-yne-1,3-diyl bis(2,2-dimethylpropanoate). To a solution of bromo methyl triphenylphosphorane (648.55 mg, 1.82 mmol) in THF (2.5 mL) was added n-BuLi (2.5 M, 680.82 μL) in dropwise at 0° C. under N₂. The mixture was stirred at 0° C. for 0.5 h. A solution of (3S)-5-((tert-butyldimethylsilyl)oxy)-2-oxohept-6-yne-1,3-diyl bis(2,2-dimethylpropanoate) (0.5 g, 1.13 mmol) in THF (2.5 mL) was added dropwise at 0° C. The reaction was quenched by saturated NH₄Cl solution (2 mL) slowly. The resulting mixture was extracted with EA (20 mL*2). The combined organic phase was washed with brine (20 mL), dried over anhydrous Na₂SO₄, filtered. The filtrate was concentrated in vacuum to give black oil. The residue was purified by column chromatography (SiO₂, Petroleum ether/Ethyl acetate=100/1 to 20/1) to give (3S)-5-((tert-butyldimethylsilyl)oxy)-2-methylenehept-6-yne-1,3-diyl bis(2,2-dimethylpropanoate) (0.3 g, 683.88 mol, 60.27% yield) as colorless oil. ¹H-NMR (400 MHz, CDCl₃) δ=5.33 (dd, J=4.2, 9.3 Hz, 1H), 5.17-5.01 (m, 2H), 4.54-4.43 (m, 2H), 4.33 (ddd, J=2.1, 6.0, 8.3 Hz, 1H), 2.34-2.27 (m, 1H), 2.08-1.89 (m, 2H), 1.19-0.99 (m, 18H), 0.84-0.70 (m, 9H), 0.02 (d, J=13.0 Hz, 6H). LCMS: ESI-MS: m/z=461.2 [M+Na]⁺.

Step H: (1S)-3-((tert-Butyldimethylsilyl)oxy)-1-((R)-2-((pivaloyloxy)methyl)oxiran-2-yl)pent-4-yn-1-yl pivalate. To a solution of (3S)-5-((tert-butyldimethylsilyl)oxy)-2-methylenehept-6-yne-1,3-diyl bis(2,2-dimethylpropanoate) (1.3 g, 2.96 mmol) in DCM (20 mL) was added meta-chloroperoxybenzoic acid (m-CPBA) (1.80 g, 8.89 mmol, 85% purity.) at 0° C. The mixture was stirred at 45° C. for 12 h. The reaction mixture was diluted with DCM (15 mL), and quenched with saturated NaHCO₃ solution (10 mL). The organic phase was washed with brine (20 mL), dried over anhydrous Na₂SO₄, filtered and concentrated under reduced pressure. The residue was purified by column chromatography (PE/EA=70/1 to 20/1) to give (1S)-3-((tert-butyldimethylsilyl)oxy)-1-((R)-2-((pivaloyloxy)methyl)oxiran-2-yl)pent-4-yn-1-yl pivalate (1 g, 2.20 mmol, 74.2% yield) as colorless oil. ¹H NMR (400 MHz, CDCl₃) δ=5.22-5.07 (m, 1H), 4.48-4.38 (m, 1H), 4.36-4.28 (m, 1H), 4.27-4.19 (m, 1H), 2.88-2.72 (m, 2H), 2.47-2.40 (m, 1H), 2.13-2.04 (m, 2H), 1.26-1.18 (m, 18H), 0.93-0.85 (m, 9H), 0.12 (d, J=10.0 Hz, 6H). LCMS: ESI-MS: m/z=455.2 [M+H]⁺.

Step I: ((1S,3R,5S)-3-((tert-Butyldimethylsilyl)oxy)-1-(hydroxymethyl)-2-methylene-5-(pivaloyloxy)cyclopentyl)methyl pivalate. A mixture of Zn (2.59 g, 39.59 mmol) and Cp₂TiCl₂ (3.28 g, 13.19 mmol, 3 eq.) in THF (70 mL) was stirred at 25° C. for 1 h under Ar. (1S)-3-((tert-Butyldimethylsilyl)oxy)-1-((R)-2-((pivaloyloxy)methyl)oxiran-2-yl)pent-4-yn-1-yl pivalate (2 g, 4.40 mmol) in THF (90 mL) was added under Ar and the mixture was stirred at 25° C. for 16 h. The mixture was quenched with saturated NH₄Cl solution (50 mL) and stirred for 2 h. The resulting solution was extracted with EA (60 mL*2). The combined organic layers were dried over anhydrous Na₂SO₄, filtered and concentrated under reduced pressure. The residue was purified by flash silica gel chromatography (ISCO®; 12 g SepaFlash® Silica Flash Column, Eluent of 0˜6.7% Ethyl acetate/Petroleum ether gradient @ 35 mL/min) to give ((1S,3R,5S)-3-((tert-butyldimethylsilyl)oxy)-1-(hydroxymethyl)-2-methylene-5-(pivaloyloxy)cyclopentyl)methyl pivalate (0.55 g, 1.20 mmol, 13.69% yield) as a colorless oil; ¹H NMR (400 MHz, CDCl₃) δ=5.33 (d, J=2.0 Hz, 1H), 5.15-5.13 (m, 1H), 5.12 (d, J=2.2 Hz, 1H), 4.51-4.41 (m, 1H), 4.14-4.01 (m, 2H), 3.76 (s, 2H), 2.54-2.44 (m, 1H), 1.76-1.66 (m, 1H), 1.20-1.16 (m, 12H), 0.93-0.87 (m, 12H), 0.12-0.06 (m, 9H); LCMS: ESI-MS: m/z=479.3 [M+H]⁺; and ((1R,3R,5S)-3-((tert-butyldimethylsilyl)oxy)-1-(hydroxymethyl)-2-methylene-5-(pivaloyloxy)cyclopentyl)methyl pivalate (1.8 g, 3.94 mmol, 44.80% yield) as a colorless oil.

Step J: ((1S,3R,5S)-1-((Bis(4-methoxyphenyl)(phenyl)methoxy)methyl)-3-((tert-butyldimethylsilyl)oxy)-2-methylene-5-(pivaloyloxy)cyclopentyl)methyl pivalate. To a solution of ((1S,3R,5S)-3-((tert-butyldimethylsilyl)oxy)-1-(hydroxymethyl)-2-methylene-5-(pivaloyloxy)cyclopentyl)methyl pivalate (1.1 g, 2.41 mmol) in DCM (12 mL) was added AgNO₃ (818.32 mg, 4.82 mmol, 810.21 μL), 2,4,6-trimethylpyridine (collidine) (583.76 mg, 4.82 mmol, 636.60 μL) and 4,4′-dimethoxytrityl chloride (DMTrCl) (1.22 g, 3.61 mmol). The mixture was stirred at 25° C. for 2 hr. The mixture was quenched with MeOH (1 mL) and the solvent was removed at low pressure. The residue was purified by flash silica gel chromatography (ISCO®; 4 g SepaFlash® Silica Flash Column, Eluent of 0˜5% Ethyl acetate/Petroleum ether gradient @ 22 mL/min) to give ((1S,3R,5S)-1-((bis(4-methoxyphenyl)(phenyl)methoxy)methyl)-3-((tert-butyldimethylsilyl)oxy)-2-methylene-5-(pivaloyloxy)cyclopentyl)methyl pivalate (1.38 g, 1.82 mmol, 75.48% yield, 100% purity) as a white foam. ¹H NMR (400 MHz, CDCl₃) δ=7.27-7.43 (m, 7H), 7.17-7.24 (m, 2H), 6.78-6.81 (m, 4H), 5.26-5.45 (m, 2H), 5.04-5.08 (dd, J=9.16, 6.40 Hz, 1H), 4.45-4.50 (m, 1H), 4.24-4.26 (d, J=11.04 Hz, 1H), 3.95-4.01 (m, 1H), 3.78 (s, 6H), 3.37-3.39 (d, J=9.03 Hz, 1H), 3.21-3.23 (d, J=9.29 Hz, 1H), 2.34-2.40 (dt, J=12.55, 6.53 Hz, 1H), 1.66-1.73 (dt, J=12.05, 8.91 Hz, 1H), 1.06-1.14 (m, 9H), 0.89-1.04 (m, 18H), 0.05-0.12 (m, 6H). LCMS: ESI-MS: m/z=781.4 [M+Na]⁺.

Step K: ((1S,3R,5S)-1-((bis(4-methoxyphenyl)(phenyl)methoxy)methyl)-3-hydroxy-2-methylene-5-(pivaloyloxy)cyclopentyl)methyl pivalate. ((1S,3R,5S)-1-((Bis(4-methoxyphenyl)(phenyl)methoxy)methyl)-3-((tert-butyldimethylsilyl)oxy)-2-methylene-5-(pivaloyloxy)cyclopentyl)methyl pivalate (1.38 g, 1.82 mmol) was treated with tetra-n-butylammonium fluoride (TBAF) (1 M, 5.45 mL) at 25° C. for 1.5 h. The mixture was extracted with EA (50 mL) and washed with brine (50 mL). The organic layer was dried over anhydrous Na₂SO₄, filtered and concentrated under reduced pressure. The residue was purified by flash silica gel chromatography (ISCO®; 12 g SepaFlash® Silica Flash Column, Eluent of 3˜17% Ethyl acetate/Petroleum ether gradient @35 mL/min) to give ((1S,3R,5S)-1-((bis(4-methoxyphenyl)(phenyl)methoxy)methyl)-3-hydroxy-2-methylene-5-(pivaloyloxy)cyclopentyl)methyl pivalate (0.989 g, 1.49 mmol, 81.84% yield, 97% purity) as colorless oil. ¹H-NMR (400 MHz, CDCl₃), δ=7.40-7.39 (m, 2H), 7.29-7.21 (m, 7H), 6.82-6.80 (m, 4H), 5.47-5.42 (m, 2H), 5.15-5.11 (m, 1H), 4.53-4.47 (m, 2H), 4.22-4.06 (m, 2H), 3.78 (s, 6H), 3.42-3.32 (m, 2H), 2.49-2.42 (m, 1H), 1.80-1.71 (m, 1H), 1.14 (s, 9H), 0.97 (s, 9H). LCMS: ESI-MS: m/z=667.2 [M+Na]⁺.

### Intermediate 8. 1-((4S,5R)-5-((S)-1-((tert-Butyldimethylsilyl)oxy)prop-2-yn-1-yl)-2,2-dimethyl-1,3-dioxolan-4-yl)-2-((4-methoxyphenyl)diphenylmethoxy)ethan-1-one

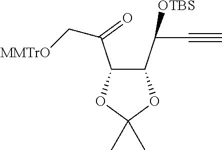

Step A. 1-((4S,5R)-5-((R)-1-Hydroxy-2-((4-methoxyphenyl)diphenylmethoxy)ethyl)-2,2-dimethyl-1,3-dioxolan-4-yl)prop-2-yn-1-ol. Dry 2,3-O-Isopropylidene-beta-D-ribofuranose (prepared according to Mandal, Sukhendu B. and Achari, Basude Synthetic Communications, 23(9), 1239-44; 1993) (7.6 g, 16 mmol) was dissolved in 160 mL of 0.5M ethynyl magnesium bromide in THF and left overnight at r.t. Reaction was quenched with solution NH₄Cl and product extracted with EtOAc. Organic layers were dried over NaSO₄, evaporated and purified by column chromatography using gradient of EtOAc in hexane from 10% to 40%. Obtained 7.47 g of the title compound (93.7%) as single isomer. ¹H-NMR (dmso-d6) δ: 7.44-7.40 (m, 4H), 7.38-7.28 (m, 8H), 6.84-6.82 (m, 2H), 5.87 (s, 1H), 4.42-4.40 (d, 1H), 4.18-3.90 (m, 3H), 3.65 (s, 3H), 3.21 (s, 1H), 3.08-2.98 (m, 2H), 1.22 (s, 3H), 1.18 (s, 3H).

Step B. (1R)-1-((4R,5R)-5-(1-((tert-Butyldimethylsilyl)oxy)prop-2-yn-1-yl)-2,2-dimethyl-1,3-dioxolan-4-yl)-2-((4-methoxyphenyl)diphenylmethoxy)ethan-1-ol. To the solution of 1-((4S,5R)-5-((R)-1-hydroxy-2-((4-methoxyphenyl)diphenylmethoxy)ethyl)-2,2-dimethyl-1,3-dioxolan-4-yl)prop-2-yn-1-ol (7.3 g, 15 mmol) in dry DMF (100 mL) was added imidazole (1.5 g, 22.5 mmol) and TBSCl (3.4 g 22 mmol). The reaction mixture was left overnight at r.t., quenched with water, and the product extracted with EtOAc. The organic layers were dried over NaSO₄, filtered, and concentrated under reduced pressure. Purification (FCC, SiO₂, using gradient of EtOAc in hexane from 0% to 25%) afforded the title compound (7.8 g, 86%). ¹H-NMR (DMSO-d6) δ: 7.45-7.41 (m, 4H), 7.36-7.24 (m, 8H), 6.88-6.82 (m, 2H), 5.30 (br.s, 1H), 4.80 (s, 1H), 4.20-3.95 (m, 3H), 3.70 (s, 3H), 3.36 (s, 1H), 3.05-2.95 (m, 2H), 1.38 (s, 3H), 1.18 (s, 3H), 0.90 (s, 9H), 0.10 (s, 6H).

Step C. 1-((4S,5R)-5-((S)-1-((tert-Butyldimethylsilyl)oxy)prop-2-yn-1-yl)-2,2-dimethyl-1,3-dioxolan-4-yl)-2-((4-methoxyphenyl)diphenylmethoxy)ethan-1-one. (1R)-1-((4R,5R)-5-(1-((tert-Butyldimethylsilyl)oxy)prop-2-yn-1-yl)-2,2-dimethyl-1,3-dioxolan-4-yl)-2-((4-methoxyphenyl)diphenylmethoxy)ethan-1-ol (7.8 g, 13 mmol) and Dess-Martin periodinate (8.3 g, 20 mmol) in DCM (70 mL) were stirred overnight at r.t. The reaction mixture was quenched with solution of Na₂S₂O₃ and NaHCO₃, and extracted with EtOAc. The organic phase was dried over NaSO₄, filtered, and concentrated under reduced pressure. Purification (FCC, SiO₂, gradient of EtOAc in hexane from 0% to 25%) afforded the title compound (6.6 g, 85%). ¹H-NMR (dmso-d6) δ: 7.40-7.20 (m, 12H), 6.88-6.81 (m, 2H), 4.60 (d, 1H), 4.47 (s, 1H), 4.36-4.31 (m, 1H), 3.68 (s, 3H), 3.19 (s, 1H), 1.98 (s, 1H), 1.25 (s, 3H), 1.10 (s, 3H), 0.87 (s, 9H), 0.00 (s, 6H).

### Intermediate 9. (6aS,8R,9aS)-8-Hydroxy-2,2,4,4-tetraisopropyl-7-methylenetetrahydrocyclopenta[f][1,3,5,2,4]trioxadisilocine-6a(6H)-carbonitrile

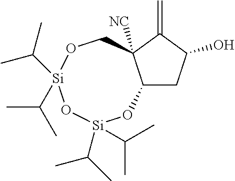 [SMILES: C=C1[C@H](O)C[C@@H]2O[Si](C(C)C)(C(C)C)O[Si](C(C)C)(C(C)C)OC[C@@]12C#N]

Step A. tert-Butyl(((S)-1-((4R,5R)-5-(3-((4-methoxyphenyl)diphenylmethoxy)prop-1-en-2-yl)-2,2-dimethyl-1,3-dioxolan-4-yl)prop-2-yn-1-yl)oxy)dimethylsilane. To a suspension of methyltriphenylphosphonium bromide (3.69 g, 10.3 mmol) in THF (12 mL, 0.87 M) cooled to 0° C., was added dropwise n-butyllithium (n-BuLi) (2.5 M hexanes, 3.8 mL, 9.49 mmol). After 30 min. at 0° C., a solution of 1-((4S,5R)-5-((S)-1-((tert-butyldimethylsilyl)oxy)prop-2-yn-1-yl)-2,2-dimethyl-1,3-dioxolan-4-yl)-2-((4-methoxyphenyl)diphenylmethoxy)ethan-1-one (Intermediate 8, 1.07 g, 1.88 mmol) in THF (18.8 mL, 0.100 AM) was added dropwise via cannula. The heterogeneous yellow solution was stirred at r.t. overnight. The now brown heterogeneous solution was cooled to 0° C. and quenched with MeOH and brine, and extracted with EtOAc. The organic layer was dried (Na₂SO₄), filtered and concentrated in vacuo to give a yellow oil. Purification (FCC, SiO₂, 0-15% EtOAc/hexanes) afforded tert-butyl(((S)-1-((4R,5R)-5-(3-((4-methoxyphenyl)diphenylmethoxy)prop-1-en-2-yl)-2,2-dimethyl-1,3-dioxolan-4-yl)prop-2-yn-1-yl)oxy)dimethylsilane as a colorless oil (0.97 g, 91%). ¹H NMR (400 MHz, CDCl₃): δ 7.46-7.20 (m, 12H), 6.83-6.81 (m, 2H), 5.42 (d, J=0.8, 1H), 4.76 (d, J=6.0, 1H), 4.22 (dd, J=7.2, 2.0, 1H), 3.92 (dd, J=7.2, 5.6, 1H), 3.79 (s, 3H), 3.67 (d, J=12, 1H), 3.59 (d, J=12, 1H), 2.36 (d, J=2.0, 1H), 1.43 (s, 3H), 1.37 (s, 3H), 0.76 (s, 9H), 0.06 (s, 3H), −0.05 (s, 3H).

Step B. tert-Butyl(((1S)-1-((4R,5S)-5-(2-(((4-methoxyphenyl)diphenylmethoxy)methyl)oxiran-2-yl)-2,2-dimethyl-1,3-dioxolan-4-yl)prop-2-yn-1-yl)oxy)dimethylsilane. To a solution of tert-butyl(((S)-1-((4R,5R)-5-(3-((4-methoxyphenyl)diphenylmethoxy)prop-1-en-2-yl)-2,2-dimethyl-1,3-dioxolan-4-yl)prop-2-yn-1-yl)oxy)dimethylsilane (4.74 g, 7.92 mmol) in CH₂Cl₂ (58 mL, 0.15 M) cooled to 0° C., was added 3-chloroperbenzoic acid (2.13 g, 9.50 mmol) in one portion. The colorless solution was stirred at r.t. overnight. The reaction mixture was quenched with NaHCO₃ (sat, aq), and the two layers were separated, and the aqueous layer was extracted with EtOAc. The combined organic extracts were dried (Na₂SO₄), filtered and concentrated in vacuo to give a crude yellow oil. Purification (FCC, SiO₂, 0-15% EtOAc/hexanes) afforded tert-butyl(((1S)-1-((4R,5S)-5-(2-(((4-methoxyphenyl)diphenylmethoxy)methyl)oxiran-2-yl)-2,2-dimethyl-1,3-dioxolan-4-yl)prop-2-yn-1-yl)oxy)dimethylsilane, the major epoxide (2.93 g, 60%) as a colorless oil and the minor epoxide (1.38 g, 28%) as a colorless oil. Major epoxide: ¹H NMR (400 MHz, CDCl₃): δ 7.47-7.44 (m, 4H), 7.35-7.21 (m, 8H), 6.84-6.82 (m, 2H), 4.83 (d, J=5.6, 1H), 4.38 (dd, J=6.4, 2.0, 1H), 3.96 (t, J=6.0, 1H), 3.79 (s, 3H), 3.61 (d, J=11, 1H), 3.18 (d, J=5.2, 1H), 3.12 (d, J=10, 1H), 2.78 (d, J=5.2, 1H), 2.32 (d, J=1.6, 1H), 1.44 (s, 3H), 1.40 (s, 3H), 0.78 (s, 9H), 0.00 (s, 6H).

Step C. ((3aR,4S,6S,6aR)-6-((tert-Butyldimethylsilyl)oxy)-4-(((4-methoxyphenyl)diphenylmethoxy)methyl)-2,2-dimethyl-5-methylenetetrahydro-4H-cyclopenta[d][1,3]dioxol-4-yl)methanol. A RBF (Note: all RBF used were dried under vacuum with a heat gun and cooled under a stream of Ar(g). THF was degassed) was charged with di(cyclopentadienyl)titanium(IV) dichloride (Cp₂TiCl₂) (2.41 g, 9.40 mmol) under argon and dry-stirred for 5-10 min, then evacuated, filled with Ar(g). This process was repeated 3×, taking care not to disturb the solid. Degassed THF (63 mL, 0.15 M) was added and the resultant THF solution of Cp₂TiCl₂ was evacuated, refilled with Ar and this process repeated 3×. Zinc (1.84 g, 28.2 mmol) was added next and once again, the resultant heterogeneous solution was degassed once or twice. The resultant dark green solution was stirred for 1 h. A solution of tert-butyl(((1S)-1-((4R,5S)-5-(2-(((4-methoxyphenyl)diphenylmethoxy)methyl)oxiran-2-yl)-2,2-dimethyl-1,3-dioxolan-4-yl)prop-2-yn-1-yl)oxy)dimethylsilane (1.93 g, 3.13 mmol) in degassed THF (31.3 mL, 0.10 M) was added in a slow stream via cannula, followed by 2 rinses. The resultant dark blue solution was degassed for the final time following the addition of the starting material, and stirred for 24 h, during which time it became a very dark blue solution. To this intense blue solution cooled to 0° C., was added 50 mL of sat. aq. NH₄Cl and stirred vigorously at r.t. overnight. The reaction mixture was filtered and concentrated in vacuo to remove the organic layer and extracted with EtOAc (2-3×), dried over (Na₂SO₄), filtered and concentrated in vacuo to give a yellow, foamy oil. This crude oil, ((3aR,4S,6S,6aR)-6-((tert-butyldimethylsilyl)oxy)-4-(((4-methoxyphenyl)diphenylmethoxy)methyl)-2,2-dimethyl-5-methylenetetrahydro-4H-cyclopenta[d][1,3]dioxol-4-yl)methanol was used in the next step without further purification. ¹H NMR (400 MHz, CDCl₃): δ 7.38-7.36 (m, 4H), 7.29-7.19 (m, 8H), 6.83-6.80 (m, 2H), 5.29 (d, J=2.0, 1H), 4.90 (d, J=2.8, 1H), 4.84 (m, 1H), 4.58 (t, J=5.6, 1H), 4.45 (d, J=5.6, 1H), 3.79 (s, 3H), 3.77 (dd, J=9.6, 4.8, 1H), 3.65 (dd, J=9.6, 9.6, 1H), 3.29 (d, J=8.4, 1H), 3.16 (d, J=8.4, 1H), 2.36 (dd, J=9.6, 4.8, 1H), 1.44 (s, 3H), 1.32 (s, 3H), 0.95 (s, 9H), 0.15 (s, 3H), 0.12 (s, 3H).

Step D. (3aR,4S,6S,6aR)-6-((tert-Butyldimethylsilyl)oxy)-4-(((4-methoxyphenyl)diphenylmethoxy)methyl)-2,2-dimethyl-5-methylenetetrahydro-4H-cyclopenta[d][1,3]dioxole-4-carbaldehyde. To a colorless solution of ((3aR,4S,6S,6aR)-6-((tert-butyldimethylsilyl)oxy)-4-(((4-methoxyphenyl)diphenylmethoxy)methyl)-2,2-dimethyl-5-methylenetetrahydro-4H-cyclopenta[d][1,3]dioxol-4-yl)methanol (1.93 g, 3.13 mmol) in CH₂Cl₂ (31.3 mL, 0.10 M) cooled to 0° C., was added Dess-Martin periodinane (1.68 g, 3.76 mmol) The reaction mixture was stirred at r.t. for 2 h, then filtered through Celite®, washed with 10 mL of 0.4 M NaHCO₃/0.4 M NaS₂O₃, back-extracted with CH₂Cl₂, dried (Na₂SO₄), concentrated in vacuo to give a crude oil. Purification (FCC, SiO₂, 10% EtOAc/hexanes) afforded (3aR,4S,6S,6aR)-6-((tert-butyldimethylsilyl)oxy)-4-(((4-methoxyphenyl)diphenylmethoxy)methyl)-2,2-dimethyl-5-methylenetetrahydro-4H-cyclopenta[d][1,3]dioxole-4-carbaldehyde as a colorless oil. This colorless oil was taken onto the next step (1.92 g, 3.13 mmol).

Step E. (E)-6-((tert-Butyldimethylsilyl)oxy)-4-(((4-methoxyphenyl)diphenylmethoxy)methyl)-2,2-dimethyl-5-methylenetetrahydro-4H-cyclopenta[d][1,3]dioxole-4-carbaldehyde oxime. To a solution of (3aR,4S,6S,6aR)-6-((tert-butyldimethylsilyl)oxy)-4-(((4-methoxyphenyl)diphenylmethoxy)methyl)-2,2-dimethyl-5-methylenetetrahydro-4H-cyclopenta[d][1,3]dioxole-4-carbaldehyde (1.92 g, 3.13 mmol) in pyridine (pyr) (31 mL, 0.10 M) was added hydroxylamine hydrochloride (0.87 g, 12.5 mmol) in one portion and stirred overnight. The reaction mixture was concentrated in vacuo and partitioned between EtOAc/H₂O. The aqueous layer was extracted with EtOAc (2×) and the combined organic extracts were dried (Na₂SO₄), filtered and concentrated in vacuo to give a light, yellow oil. Purification (FCC, SiO₂, 0-20% EtOAc/hexanes) afforded (E)-6-((tert-butyldimethylsilyl)oxy)-4-(((4-methoxyphenyl)diphenylmethoxy)methyl)-2,2-dimethyl-5-methylenetetrahydro-4H-cyclopenta[d][1,3]dioxole-4-carbaldehyde oxime as a colorless oil (1.97 g, 3.13 mmol).

Step F. (3aR,4S,6S,6aR)-6-((tert-Butyldimethylsilyl)oxy)-4-(((4-methoxyphenyl)diphenylmethoxy)methyl)-2,2-dimethyl-5-methylenetetrahydro-4H-cyclopenta[d][1,3]dioxole-4-carbonitrile. To a solution of (E)-6-((tert-butyldimethylsilyl)oxy)-4-(((4-methoxyphenyl)diphenylmethoxy)methyl)-2,2-dimethyl-5-methylenetetrahydro-4H-cyclopenta[d][1,3]dioxole-4-carbaldehyde oxime (1.97 g, 3.13 mmol) in acetonitrile (ACN) (31.3 mL, 0.10 M) cooled to 0° C., was added 1,1′-carbonyldiimidazole (CDI) (0.761 g, 4.70 mmol) in one portion. The reaction mixture was stirred at r.t. overnight. Then, the volume was reduced to ˜½ of the original volume, and the reaction mixture was heated to 35° C. to accelerate the reaction. After 2 h of heating, the reaction mixture was cooled and concentrated in vacuo and partitioned between EtOAc/3-4 mL of H₂O. The aqueous layer was extracted with EtOAc, dried (Na₂SO₄), filtered and concentrated in vacuo to give a crude oil. Purification (FCC, SiO₂, 0-10% EtOAc/hexanes) afforded (3aR,4S,6S,6aR)-6-((tert-butyldimethylsilyl)oxy)-4-(((4-methoxyphenyl)diphenylmethoxy)methyl)-2,2-dimethyl-5-methylenetetrahydro-4H-cyclopenta[d][1,3]dioxole-4-carbonitrile as a colorless oil (1.29 g, 67% over 4 steps).

Step G. (3aR,4S,6S,6aS)-6-Hydroxy-4-(((4-methoxyphenyl)diphenylmethoxy)methyl)-2,2-dimethyl-5-methylenetetrahydro-4H-cyclopenta[d][1,3]dioxole-4-carbonitrile. To a solution of (3aR,4S,6S,6aR)-6-((tert-butyldimethylsilyl)oxy)-4-(((4-methoxyphenyl)diphenylmethoxy)methyl)-2,2-dimethyl-5-methylenetetrahydro-4H-cyclopenta[d][1,3]dioxole-4-carbonitrile (1.29 g, 2.11 mmol) in THF (21 mL, 0.10 M) cooled to 0° C., was added TBAF (1.0 M THF, 3.16 mL, 3.16 mmol) dropwise. The reaction mixture was stirred at r.t. for 1.5 h. The reaction mixture was quenched with silica gel, filtered and concentrated in vacuo to give a crude oil. Purification (FCC, SiO₂, 0-50% EtOAc/hexanes) afforded (3aR,4S,6S,6aS)-6-hydroxy-4-(((4-methoxyphenyl)diphenylmethoxy)methyl)-2,2-dimethyl-5-methylenetetrahydro-4H-cyclopenta[d][1,3]dioxole-4-carbonitrile as a colorless oil (0.957 g, 91%). ¹H NMR (400 MHz, CDCl₃): δ 7.41-7.23 (m, 12H), 6.84 (m, 2H), 5.64 (s, 1H), 5.59 (s, 1H), 4.47 (t, J=5.2, 1H), 4.42 (d, J=5.2, 1H), 4.38 (m, 1H), 3.81 (s, 3H), 3.31 (d, J=9.6, 1H), 3.12 (d, J=9.6, 1H), 2.32 (d, J=12, 1H), 1.47 (s, 3H), 1.33 (s, 3H).

Step H. (3aR,4S,6S,6aR)-6-Cyano-6-(((4-methoxyphenyl)diphenylmethoxy)methyl)-2,2-dimethyl-5-methylenetetrahydro-4H-cyclopenta[d][1,3]dioxol-4-yl benzoate. To a colorless solution of (3aR,4S,6S,6aS)-6-hydroxy-4-(((4-methoxyphenyl)diphenylmethoxy)methyl)-2,2-dimethyl-5-methylenetetrahydro-4H-cyclopenta[d][1,3]dioxole-4-carbonitrile (0.957 g, 1.93 mmol) in THF (7.7 mL, 0.25 M) with DMAP (0.047 g, 0.385 mmol), and triethylamine (TEA) (2.69 mL, 19.3 mmol) cooled to 0° C., was added benzoyl chloride (0.268 mL, 2.31 mmol) dropwise. The heterogeneous solution was stirred vigorously at r.t. overnight. The reaction mixture was quenched with NaHCO₃ (sat, aq.) and the aqueous layer was extracted with EtOAc. The combined organic extracts were dried (NaSO₄), filtered and concentrated in vacuo to give a crude oil. Purification (FCC, SiO₂, 0-25% EtOAc/hexanes) afforded (3aR,4S,6S,6aR)-6-cyano-6-(((4-methoxyphenyl)diphenylmethoxy)methyl)-2,2-dimethyl-5-methylenetetrahydro-4H-cyclopenta[d][1,3]dioxol-4-yl benzoate as a white foamy solid (0.917 g, 79%).

Step I. (1S,3S,4R,5R)-3-Cyano-4.5-dihydroxy-3-(hydroxymethyl)-2-methylenecyclopentyl benzoate. At 0° C., to a round bottom flask (RBF) charged with (3aR,4S,6S,6aR)-6-cyano-6-(((4-methoxyphenyl)diphenylmethoxy)methyl)-2,2-dimethyl-5-methylenetetrahydro-4H-cyclopenta[d][1,3]dioxol-4-yl benzoate (0.100 g, 0.166 mmol) was added TFA:H₂O (2.66 mL:2.66 mL, 0.031 M) dropwise with good stirring. After 2.5 h at r.t., the reaction mixture was co-evaporated with 2 mL each of toluene and EtOH and this process was repeated. The crude oil thus obtained was taken up in EtOH and with stirring at 0° C., quenched with 0.50 g of resin bound amine base (˜10 equiv.). The reaction mixture was stirred for 5 min at r.t., filtered and rinsed with EtOH, concentrated in vacuo to give a yellow oil. Purification (FCC, SiO₂, 3-10% MeOH/DCM) afforded (1S,3S,4R,5R)-3-cyano-4,5-dihydroxy-3-(hydroxymethyl)-2-methylenecyclopentyl benzoate as a colorless oil (0.0391 g, 81%). ¹H NMR (400 MHz, CDCl₃): δ 8.02 (d, J=8.0, 1H), 7.49 (t, J=7.6, 1H), 7.36 (t, J=8.00, 2H), 5.60 (m, 1H), 5.55 (s, 1H), 5.48 (s, 1H), 4.82 (d, J=7.2, 1H), 4.47 (m, 1H), 4.40 (m, 1H), 4.31 (m, 1H), 4.01 (m, 1H), 3.91 (dd, J=11.6, 5.2, 1H), 3.77 (dd, J=11.6, 5.2, 1H).

Step J. (6aS,8S,9S,9aR)-6a-Cyano-9-hydroxy-2,2,4,4-tetraisopropyl-7-methylenehexahydrocyclopenta[f][1,3,5,2,4]trioxadisilocin-8-yl benzoate. To a colorless solution of (1S,3S,4R,5R)-3-cyano-4,5-dihydroxy-3-(hydroxymethyl)-2-methylenecyclopentyl benzoate (0.352 g, 1.22 mmol) in pyridine (4 mL, 0.3 M) cooled to 0° C., 1,1,3,3-tetraisopropyl-1,3-dichlorodisiloxane (0.438 mL, 1.33 mmol) was added dropwise. The reaction mixture was slowly warmed to r.t. in 20 min. The reaction mixture was stirred at r.t. overnight. Pyridine was removed under reduced pressure, partitioned between H₂O and EtOAc. The organic layer was washed with NaHCO₃ (sat, aq.), and dried (Na₂SO₄), filtered and concentrated in vacuo to give a colorless oil. Purification (FCC, SiO₂, 0-15% 5CV, 15% 2CV, 15-30% 5CV) afforded (6aS,8S,9S,9aR)-6a-cyano-9-hydroxy-2,2,4,4-tetraisopropyl-7-methylenehexahydrocyclopenta[f][1,3,5,2,4]trioxadisilocin-8-yl benzoate as a colorless oil (0.287 g, 44%). ¹H NMR (400 MHz, CDCl₃): δ 8.15 (d, J=6.8, 1H), 7.61 (t, J=6.8, 1H), 7.48 (t, J=7.2, 2H), 5.64-5.59 (m, 3H), 4.46 (m, 1H), 4.36 (m, 1H), 4.16 (d, J=11, 1H), 3.96 (d, J=11, 1H), 1.15-1.07 (m, 29H).

Step K. (6aS,8S,9S,9aR)-9-((1H-Imidazole-1-carbonothioyl)oxy)-6a-cyano-2,2,4,4-tetraisopropyl-7-methylenehexahydrocyclopenta[f][1,3,5,2,4]trioxadisilocin-8-yl benzoate. A solution of (6aS,8S,9S,9aR)-6a-cyano-9-hydroxy-2,2,4,4-tetraisopropyl-7-methylenehexahydrocyclopenta[f][1,3,5,2,4]trioxadisilocin-8-yl benzoate (0.0306 g, 0.0575 mmol) in dichloroethane (0.6 mL, 0.1 M) with 1,1′-thiocarbonyldiimidazole (0.051 g, 0.287 mmol) and 4-(dimethylamino)pyridine (0.015 g, 0.017 mmol) was heated at reflux for 30 min., cooled to 0° C. and, with stirring, 1-2 mL of MeOH was added. The reaction mixture was concentrated in vacuo to remove most of the solvent, diluted with EtOAc, and washed with H₂O. The aqueous layer was extracted with EtOAc, and the combined organic extracts were dried (Na₂SO₄), filtered and concentrated in vacuo to give a yellow oil. Purification (0-30% EtOAc/hexanes) afforded (6aS,8S,9S,9aR)-9-((1H-imidazole-1-carbonothioyl)oxy)-6a-cyano-2,2,4,4-tetraisopropyl-7-methylenehexahydrocyclopenta[f][1,3,5,2,4]trioxadisilocin-8-yl benzoate as a colorless oil (0.033 g, 89%). ¹H NMR (400 MHz, CDCl₃): δ 8.49 (s, 1H), 7.91 (m, 2H), 7.85 (bs, 1H), 7.57 (m, 1H), 7.40 (m, 1H), 7.06 (m, 1H), 6.49 (t, J=4.0, 1H), 6.18 (m, 1H), 5.71 (s, 1H), 5.63 (s, 1H), 4.63 (d, J=4.0, 1H), 4.25 (d, J=11, 1H), 4.10 (d, J=11, 1H), 1.11-1.03 (m, 28H).

Step L. (6aS,8R,9aS)-6a-Cyano-2,2,4,4-tetraisopropyl-7-methylenehexahydrocyclopenta[f][1,3,5,2,4]trioxadisilocin-8-yl benzoate. A solution of (6aS,8S,9S,9aR)-9-((1H-imidazole-1-carbonothioyl)oxy)-6a-cyano-2,2,4,4-tetraisopropyl-7-methylenehexahydrocyclopenta[f][1,3,5,2,4]trioxadisilocin-8-yl benzoate (0.259 g, 0.403 mmol) in toluene (tol) (13.5 mL, 0.03 M) with azobisisobutyronitrile (AIBN) (0.020 g, 0.121 mmol) and tributyltin hydride (0.324 mL, 1.21 mmol) was heated at reflux for 1.3 h and cooled to r.t. and concentrated in vacuo to give a crude oil. Purification (FCC, SiO₂, 0-10% EtOAc/hexanes) afforded (6aS,8R,9aS)-6a-cyano-2,2,4,4-tetraisopropyl-7-methylenehexahydrocyclopenta[f][1,3,5,2,4]trioxadisilocin-8-yl benzoate as a colorless oil (0.208 g, quantitative yield). ¹H NMR (400 MHz, CDCl₃): δ 8.09-8.07 (m, 2H), 7.60-7.56 (m, 1H), 7.47-7.44 (m, 2H), 5.70 (m, 1H), 5.65-5.64 (m, 1H), 5.55 (m, 1H), 4.25 (dd, J=12, 6.4, 1H), 4.17 (d, J=12, 1H), 4.10 (d, J=12, 1H), 2.77-2.71 (m, 1H), 2.31-2.23 (m, 1H), 1.14-1.04 (m, 28H).

Step M. (6aS,8R,9aS)-8-Hydroxy-2,2,4,4-tetraisopropyl-7-methylenetetrahydrocyclopenta[f][1,3,5,2,4]trioxadisilocine-6a(6H)-carbonitrile. To a solution of (6aS,8R,9aS)-6a-cyano-2,2,4,4-tetraisopropyl-7-methylenehexahydrocyclopenta[f][1,3,5,2,4]trioxadisilocin-8-yl benzoate (0.127 g, 0.245 mmol) in MeOH (6.5 mL, 0.037M) cooled to 0° C., was added NaOMe/MeOH (0.44M, 0.14 mL, 0.061 mmol) dropwise. The reaction mixture was stirred at r.t. for 6 h. The reaction mixture was quenched with NH₄Cl (sat, aq.), extracted with EtOAc, dried (Na₂SO₄), filtered and concentrated in vacuo to give the title compound as a white residue. This crude product was combined with a crude oil obtained from a previous batch (0.0132 g). Purification (FCC, SiO₂, 23% EtOAc/hexanes) afforded (6aS,8R,9aS)-8-hydroxy-2,2,4,4-tetraisopropyl-7-methylenetetrahydrocyclopenta[f][1,3,5,2,4]trioxadisilocine-6a(6H)-carbonitrile (0.069 g, 62%). ¹H NMR (400 MHz, CDCl₃): δ 5.61-5.60 (m, 1H), 5.48 (d, J=2.0, 1H), 4.45-4.37 (m, 1H), 4.15 (dd, J=11, 6.0, 1H), 4.12 (d, J=12, 1H), 3.98 (d, J=12, 1H), 2.53 (m, 1H), 2.01 (m, 1H), 1.70 (d, J=8, 1H), 1.13-1.03 (m, 28H).

### Intermediate 10. N,N-Di-BOC-6-chloro-9H-purin-2-amine

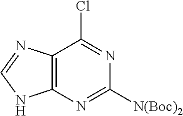

The title compound was prepared pared according to Porcheddu et al., European Journal of Organic Chemistry (2008) 34:5786-5797.

### Example 1: (1S,2S,4S)-4-(4-Amino-7H-pyrrolo[2,3-d]pyrimidin-7-yl)-2-fluoro-2-(hydroxymethyl)-3-methylenecyclopentan-1-ol

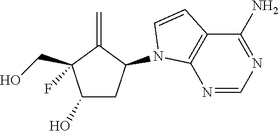 [SMILES: C=C1[C@@H](n2ccc3c(N)ncnc32)C[C@H](O)[C@@]1(F)CO]

Step A: N,N-Di-BOC-7-((1S,3S,4S)-4-((tert-butyldimethylsilyl)oxy)-3-(((tert-butyldimethylsilyl)oxy)methyl)-3-fluoro-2-methylenecyclopentyl)-7H-pyrrolo[2,3-d]pyrimidin-4-amine. To a solution of (1R,3S,4S)-4-((tert-butyldimethylsilyl)oxy)-3-(((tert-butyldimethylsilyl)oxy)methyl)-3-fluoro-2-methylenecyclopentan-1-ol (Intermediate 2, 250 mg, 639.91 mol) and N,N-Di-BOC-7H-pyrrolo[2,3-d]pyrimidin-4-amine (Intermediate 4, 288.86 mg, 863.88 mol) in THF (6 mL) was added PPh₃ (503.53 mg, 1.92 mmol, 3 eq.) and followed by DIAD (388.19 mg, 1.92 mmol, 373.26 μL, 3 eq.) in at 0° C. The mixture was stirred at 25° C. for 12 h. The reaction was diluted with EA (10 mL), and 1.5 g of silica gel was added. The resulting mixture was concentrated at low pressure. The residue was purified by flash silica gel chromatography (ISCO®; 4 g SepaFlash® Silica Flash Column, Eluent of 0˜30% Ethyl acetate/Petroleum ether gradient @ 18 mL/min) to give N,N-Di-BOC-7-((1S,3S,4S)-4-((tert-butyldimethylsilyl)oxy)-3-(((tert-butyldimethylsilyl)oxy)methyl)-3-fluoro-2-methylenecyclopentyl)-7H-pyrrolo[2,3-d]pyrimidin-4-amine (320 mg, 70.73% yield) as a colorless oil. ¹H NMR (400 MHz, CDCl₃) δ=8.74 (s, 1H), 7.22 (d, J=3.8 Hz, 1H), 6.41 (d, J=3.8 Hz, 1H), 5.94 (br, s, 1H), 5.52 (br, d, J=3.3 Hz, 1H), 5.05 (br, s, 1H), 4.61 (br, d, J=6.5 Hz, 1H), 4.11-3.83 (m, 2H), 2.48-2.27 (m, 2H), 1.44 (s, 18H), 0.98-0.89 (m, 18H), 0.24-0.04 (m, 12H). ¹⁹F NMR (376 MHz, CDCl₃) δ=−165.66 ppm LCMS: ESI-MS: m/z 707.20 [M+1]⁺, 729.20 [M+Na]⁺.

Step B: N,N-Di-BOC-(1S,2S,4S)-4-(4-Amino-7H-pyrrolo[2,3-d]pyrimidin-7-yl)-2-fluoro-2-(hydroxymethyl)-3-methylenecyclopentan-1-ol. To a solution of N,N-Di-BOC-7-((1S,3S,4S)-4-((tert-butyldimethylsilyl)oxy)-3-(((tert-butyldimethylsilyl)oxy)methyl)-3-fluoro-2-methylenecyclopentyl)-7H-pyrrolo[2,3-d]pyrimidin-4-amine (300 mg, 424.31 mol) in THF (1 mL) was treated with TBAF (1 M, 848.62 μL). The mixture was stirred at 25° C. for 2 h. The reaction mixture was diluted with EA (20 mL). The resulting solution was washed with water (20 mL*2), dried over anhydrous Na₂SO₄, and concentrated at low pressure. The residue was purified by column chromatography (SiO₂, Petroleum ether/Ethyl acetate=100/1 to 1/2) to give N,N-Di-BOC-(1S,2S,4S)-4-(4-amino-7H-pyrrolo[2,3-d]pyrimidin-7-yl)-2-fluoro-2-(hydroxymethyl)-3-methylenecyclopentan-1-ol (185 mg, 91.12% yield) as a colorless oil. LCMS: ESI-MS: m/z 479.2, [M+1]⁺, 501.2 [M+Na]⁺.

Step C: (1S,2S,4S)-4-(4-Amino-7H-pyrrolo[2,3-d]pyrimidin-7-yl)-2-fluoro-2-(hydroxymethyl)-3-methylenecyclopentan-1-ol. To a solution of N,N-Di-BOC-(1S,2S,4S)-4-(4-amino-7H-pyrrolo[2,3-d]pyrimidin-7-yl)-2-fluoro-2-(hydroxymethyl)-3-methylenecyclopentan-1-ol (180 mg, 376.17 mol) in DCM (2 mL) was added TFA (770.00 mg, 6.75 mmol, 0.5 mL). The mixture was stirred at 25° C. for 4 h. The reaction mixture was diluted with DCM (5 mL) and silica gel (500 mg) was added and concentrated at low pressure. The residue was purified by column chromatography (SiO₂, DCM/MeOH=100/1 to 10/1) to give the crude title compound; and further purified by Prep-HPLC (column: Xtimate C18 150*25 mm*5 um; mobile phase: [water (0.225% FA)-ACN]; B %: 1%-23%, 9 min) to give (1S,2S,4S)-4-(4-amino-7H-pyrrolo[2,3-d]pyrimidin-7-yl)-2-fluoro-2-(hydroxymethyl)-3-methylenecyclopentan-1-ol (37 mg, 34.88% yield, 98.69% purity) as a white solid and another crude desired product (9.0 mg) as a white solid. ¹H NMR (400 MHz, CD₃OD) δ=8.06 (s, 1H), 7.11 (d, J=3.5 Hz, 1H), 6.58 (d, J=3.8 Hz, 1H), 5.87 (br, d, J=2.8 Hz, 1H), 5.58 (t, J=3.3 Hz, 1H), 5.11-5.00 (m, 1H), 4.49 (q, J=4.9 Hz, 1H), 4.02-3.76 (m, 2H), 2.44-2.24 (m, 2H). ¹⁹F NMR (376 MHz, CD₃OD) S=−168.82 ppm (s, 1F). LCMS: ESI-MS: m/z 279.1 [M+1]*.

### Example 2: 2-Amino-7-((1S,3R,4S)-4-hydroxy-3-(hydroxymethyl)-2-methylenecyclopentyl)-3,7-dihydro-4H-pyrrolo[2,3-d]pyrimidin-4-one

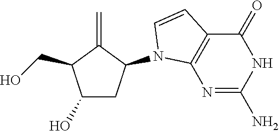 [SMILES: C=C1[C@H](CO)[C@@H](O)C[C@@H]1n1ccc2c(=O)[nH]c(N)nc21]

Step A: 4-Chloro-7-((1S,3R,4S)-2-methylene-4-((triisopropylsilyl)oxy)-3-(((triisopropylsilyl)oxy)methyl)cyclopentyl)-7H-pyrrolo[2,3-d]pyrimidin-2-amine. To a solution of (1R,3R,4S)-2-methylene-4-((triisopropylsilyl)oxy)-3-(((triisopropylsilyl)oxy)methyl)cyclopentan-1-ol (Intermediate 1, 450 mg, 985.01 mol) and 4-chloro-7H-pyrrolo[2,3-d]pyrimidin-2-amine (332.11 mg, 1.97 mmol) in THF (10 mL) was added PPh₃ (775.06 mg, 2.96 mmol) and DIAD (597.53 mg, 2.96 mmol, 574.55 μL) at 0° C. The mixture was stirred at 25° C. for 12 h. The reaction mixture was concentrated under reduced pressure. The residue was purified by flash silica gel chromatography (ISCO®; 4 g SepaFlash® Silica Flash Column, Eluent of 0˜5% Ethyl acetate/Petroleum ether gradient @20 mL/min) to give the title compound (350 mg, 2 bathes, 576.21 mol, 29.25% yield) as colorless oil. ¹H NMR (400 MHz, CDCl₃) δ=6.88-6.84 (m, 1H), 6.41-6.37 (m, 1H), 5.89-5.72 (m, 1H), 5.14 (s, 1H), 4.88 (s, 1H), 4.70 (s, 1H), 4.58 (s, 1H), 3.91-3.82 (m, 1H), 3.82-3.73 (m, 1H), 2.80 (s, 1H), 2.30-2.15 (m, 2H), 1.13-1.09 (m, 36H), 0.96-0.81 (m, 6H). LCMS: ESI-MS: m/z=607.10 [M+H]⁺.

Step B: 2-Amino-7-((1S,3R,4S)-4-hydroxy-3-(hydroxymethyl)-2-methylenecyclopentyl)-3,7-dihydro-4H-pyrrolo[2,3-d]pyrimidin-4-one. 4-Chloro-7-((1S,3R,4S)-2-methylene-4-((triisopropylsilyl)oxy)-3-(((triisopropylsilyl)oxy)methyl)cyclopentyl)-7H-pyrrolo[2,3-d]pyrimidin-2-amine (400 mg, 658.53 mol) was treated with HCl solution (10 mL, 6 M) and THF (10 mL) and stirred at 80° C. for 12 h. The mixture was diluted with MeOH and adjusted pH to 7 by treatment with saturated aq. NaHCO₃ solution. The solution was filtered and concentrated under reduced pressure. The residue was purified by column chromatography (SiO₂, DCM/MeOH=20/1 to 10/1) and further purified by Prep-HPLC (column: YMC-Actus Triart C18 100*30 mm*5 um; mobile phase: [water(0.05% HCl)-ACN]; B %: 0%-30%,10 min) to give the title compound (45.2 mg, 163.60 mol, 24.84% yield, 100% purity) as a white solid. ¹H NMR (400 MHz, CD₃OD) δ: 6.95 (d, J=3.5 Hz, 1H), 6.56 (d, J=3.5 Hz, 1H), 5.52 (t, J=9.20 Hz, 1H), 5.37 (s, 1H), 4.41-4.35 (m, 1H), 3.95 (dd, J=3.4, 10.7 Hz, 1H), 3.85 (dd, J=5.3, 10.5 Hz, 1H), 2.73 (s, 1H), 2.33-2.20 (m, 2H) LCMS: ESI-MS: m/z=276.90 [M+H]⁺.

### Example 3: 1-((1S,3S,4S)-3-Fluoro-4-hydroxy-3-(hydroxymethyl)-2-methylenecyclopentyl)-5-methylpyrimidine-2,4(1H,3H)-dione

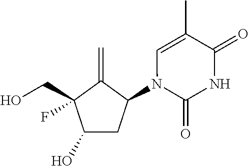 [SMILES: C=C1[C@@H](n2cc(C)c(=O)[nH]c2=O)C[C@H](O)[C@@]1(F)CO]

Step A: 3-Benzoyl-1-((1S,3S,4S)-4-((tert-butyldimethylsilyl)oxy)-3-(((tert-butyldimethylsilyl)oxy)methyl)-3-fluoro-2-methylenecyclopentyl)-5-methylpyrimidine-2,4(1H,3H)-dione and 3-Benzoyl-2-(((1S,3S,4S)-4-((tert-butyldimethylsilyl)oxy)-3-(((tert-butyldimethylsilyl)oxy)methyl)-3-fluoro-2-methylenecyclopentyl)oxy)-5-methylpyrimidin-4(3H)-one. To a solution of (1R,3S,4S)-4-((tert-butyldimethylsilyl)oxy)-3-(((tert-butyldimethylsilyl)oxy)methyl)-3-fluoro-2-methylenecyclopentan-1-ol (Intermediate 2, 200 mg, 511.93 mol) and 3-benzoyl-5-methyl-1H-pyrimidine-2,4-dione (185.32 mg, 804.97 mol) in THF (5 mL) was added PPh₃ (421.62 mg, 1.61 mmol) and bis(1,1-dimethylethyl)azodicarboxylate (DBAD) (370.70 mg, 1.61 mmol) at 0° C. The mixture was stirred at 25° C. for 12 h. The reaction mixture was diluted with EA (10 mL), and 1.0 g of silica gel was added. The mixture was concentrated at low pressure. The residue was purified by flash silica gel chromatography (ISCO®; 4 g*2 SepaFlash® Silica Flash Column, Eluent of 0-40% Ethyl acetate/Petroleum ether gradient 20 mL/min) to give 3-benzoyl-1-((1S,3S,4S)-4-((tert-butyldimethylsilyl)oxy)-3-(((tert-butyldimethylsilyl)oxy)methyl)-3-fluoro-2-methylenecyclopentyl)-5-methylpyrimidine-2,4(1H,3H)-dione (188 mg, 60.91% yield) as a white solid; ¹H NMR (400 MHz, CDCl₃) δ=7.92 (d, J=7.3 Hz, 2H), 7.75-7.58 (m, 1H), 7.58-7.45 (m, 2H), 7.05 (d, J=1.0 Hz, 1H), 5.67 (br, s, 1H), 5.64 (br, d, J=3.0 Hz, 1H), 5.34 (t, J=2.9 Hz, 1H), 4.39 (q, J=4.2 Hz, 1H), 3.82 (d, J=14.1 Hz, 2H), 2.39-2.15 (m, 1H), 2.00 (ddd, J=4.4, 9.2, 13.2 Hz, 1H), 1.93 (s, 3H), 0.90 (d, J=19.3 Hz, 18H), 0.17-0.05 (m, 12H). ¹⁹F NMR (376 MHz, CDCl₃) δ=−163.92 ppm; LCMS: ESI-MS: m/z 625.1 [M+Na]⁺; and 3-benzoyl-2-(((1S,3S,4S)-4-((tert-butyldimethylsilyl)oxy)-3-(((tert-butyldimethylsilyl)oxy)methyl)-3-fluoro-2-methylenecyclopentyl)oxy)-5-methylpyrimidin-4(3H)-one (155 mg, crude) as colorless oil: ¹H NMR (400 MHz, CDCl₃) δ=7.81 (d, J=7.3 Hz, 2H), 7.73-7.63 (m, 1H), 7.62 (d, J=0.8 Hz, 1H), 7.54-7.47 (m, 2H), 5.94 (br, t, J=5.9 Hz, 1H), 5.46 (br, s, 1H), 5.36 (br, s, 1H), 4.22-4.13 (m, 1H), 3.52-3.30 (m, 2H), 2.27 (br, s, 1H), 2.07-1.98 (m, 3H), 1.71 (br s, 1H), 0.83 (d, J=10.8 Hz, 18H), 0.04-−0.06 (m, 12H). ¹⁹F NMR (376 MHz, CDCl₃) δ=−168.52 ppm. LCMS: ESI-MS: m/z 625.1 [M+Na]⁺.

Step B: 1-((1S,3S,4S)-4-((tert-Butyldimethylsilyl)oxy)-3-(((tert-butyldimethylsilyl)oxy)methyl)-3-fluoro-2-methylenecyclopentyl)-5-methylpyrimidine-2,4(1H,3H,)-dione. To a solution of 3-benzoyl-1-((1S,3S,4S)-4-((tert-butyldimethylsilyl)oxy)-3-(((tert-butyldimethylsilyl)oxy)methyl)-3-fluoro-2-methylenecyclopentyl)-5-methylpyrimidine-2,4(1H,3H)-dione (70 mg, 116.11 mol) in MeOH (0.2 mL) was treated with NH₃ MeOH (7 M, 2 mL). The mixture was stirred at 25° C. for 12 h. The reaction mixture was concentrated at low pressure. The residue was purified by column chromatography (SiO₂, Petroleum ether/Ethyl acetate=10/1 to 112) to give 1-((1S,3S,4S)-4-((tert-butyldimethylsilyl)oxy)-3-(((tert-butyldimethylsilyl)oxy)methyl)-3-fluoro-2-methylenecyclopentyl)-5-methylpyrimidine-2,4(1H,3H)-dione (0.051 g, 88.06% yield, 100% purity) as a white solid. ¹H NMR (400 MHz, CDCl₃) δ=8.06 (s, 1H), 6.94 (s, 1H), 5.70-5.49 (m, 2H), 5.33-5.12 (m, 1H), 4.41 (q, J=4.4 Hz, 1H), 3.90-3.80 (m, 2H), 2.31-2.18 (m, 1H), 2.08-1.95 (m, 1H), 1.90 (s, 3H), 0.91 (d, J=8.8 Hz, 18H), 0.13-0.08 (m, 12H). ¹⁹F NMR (376 MHz, CDCl₃) δ=−164.18 ppm. LCMS: ESI-MS: m/z 499.3 [M+H]⁺.

Step C: 1-((1S,3S,4S′)-3-Fluoro-4-hydroxy-3-(hydroxymethyl)-2-methylenecyclopentyl)-5-methylpyrimidine-2,4(1H,3H)-dione. To a solution of 1-((1S,3S,4S)-4-((tert-butyldimethylsilyl)oxy)-3-(((tert-butyldimethylsilyl)oxy)methyl)-3-fluoro-2-methylenecyclopentyl)-5-methylpyrimidine-2,4(1H,3H)-dione (190 mg, 380.93 mol) in THF (0.2 mL) was treated with TBAF (1 M, 3.80 mL). The mixture was stirred at 25° C. for 1 h. The reaction mixture was concentrated at low pressure. The residue was purified by column chromatography (SiO₂, EA/acetone=20/1 to 2/1) twice to give 1-((1S,3S,4S)-3-fluoro-4-hydroxy-3-(hydroxymethyl)-2-methylenecyclopentyl)-5-methylpyrimidine-2,4(1H,3H)-dione (75 mg, 71.40% yield, 98.01% purity) as white solid. ¹H NMR (400 MHz, CD₃OD) δ=7.35 (s, 1H), 5.66-5.55 (m, 2H), 5.28 (t, J=3.0 Hz, 1H), 4.38 (q, J=5.0 Hz, 1H), 3.93-3.73 (m, 2H), 2.29-2.10 (m, 2H), 1.85 (s, 3H). ¹⁹F NMR (376 MHz, CD₃OD) δ=−168.28 ppm. LCMS: ESI-MS: m/z 271.1 [M+H]⁺.

### Example 4: 2-Amino-9-((1S,3S,4S)-3-fluoro-4-hydroxy-3-(hydroxymethyl)-2-methylenecyclopentyl)-1,9-dihydro-6H-purin-6-one

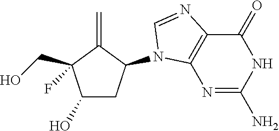 [SMILES: C=C1[C@@H](n2cnc3c(=O)[nH]c(N)nc32)C[C@H](O)[C@@]1(F)CO]

Step A: 9-((1S,3S,4S)-4-((tert-Butyldimethylsilyl)oxy)-3-(((tert-butyldimethylsilyl)oxy)methyl)-3-fluoro-2-methylenecyclopentyl)-2-isobutyramido-9H-purin-6-yl diphenylcarbamate. To a solution of (1R,3S,4S)-4-((tert-butyldimethylsilyl)oxy)-3-(((tert-butyldimethylsilyl)oxy)methyl)-3-fluoro-2-methylenecyclopentan-1-ol (Intermediate 2, 140 mg, 358.35 mol) and 2-isobutyramido-9H-purin-6-yl diphenylcarbamate (prepared according to Milecki et al., Journal of Labelled Compounds and Radiopharmaceuticals (2001) 44:763-783) (223.84 mg, 537.53 mol) in THF (5 mL) and dioxane (5 mL) was added PPh₃ (281.97 mg, 1.08 mmol) and followed by addition of DIAD (217.39 mg, 1.08 mmol, 209.03 μL) dropwise. The mixture was stirred at 25° C. for 12 h. The reaction mixture was concentrated at low pressure. The residue was purified by column chromatography (SiO₂, PE/EA=100/1 to 10/1) to give 9-((1S,3S,4S)-4-((tert-butyldimethylsilyl)oxy)-3-(((tert-butyldimethylsilyl)oxy)methyl)-3-fluoro-2-methylenecyclopentyl)-2-isobutyramido-9H-purin-6-yl diphenylcarbamate (272 mg, crude) as a white solid. ¹H NMR (400 MHz, CDCl₃) δ=7.98-7.89 (m, 2H), 7.51-7.31 (m, 8H), 7.26-7.20 (m, 2H), 5.67 (br d, J=2.0 Hz, 1H), 5.63-5.56 (m, 1H), 5.14 (br s, 1H), 4.63 (q, J=4.7 Hz, 1H), 4.15-4.01 (m, 1H), 3.87 (t, J=12.3 Hz, 1H), 2.99 (br s, 1H), 2.51 (br d, J=7.8 Hz, 1H), 2.40-2.28 (m, 1H), 1.29-1.28 (m, 6H), 0.92 (d, J=7.3 Hz, 18H), 0.33-−0.13 (m, 12H). ¹⁹F NMR (376 MHz, CDCl₃) δ=−165.80 (s, 1F). LCMS:ESI-MS: m/z 789.4 [M+H]⁺.

Step B: N-(9-((1S,3S,4S)-3-Fluoro-4-hydroxy-3-(hydroxymethyl)-2-methylenecyclopentyl)-6-oxo-6,9-dihydro-1H-purin-2-yl)isobutyramide. 9-((1S,3S,4S)-4-((tert-Butyldimethylsilyl)oxy)-3-(((tert-butyldimethylsilyl)oxy)methyl)-3-fluoro-2-methylenecyclopentyl)-2-isobutyramido-9H-purin-6-yl diphenylcarbamate (135 mg, 171.08 mol) was treated with TBAF (1 M, 1.71 mL). The mixture was stirred at 25° C. for 2 h under N₂ atmosphere. (The reaction was set up in two batches). The reaction mixture was concentrated under reduced pressure. The residue was purified by column chromatography (SiO₂, DCM/MeOH=40/1 to 10/1) to give N-(9-((1S,3S,4S)-3-fluoro-4-hydroxy-3-(hydroxymethyl)-2-methylenecyclopentyl)-6-oxo-6,9-dihydro-1H-purin-2-yl)isobutyramide (0.051 g, 40.39% yield, 99% purity) as a colorless oil. ¹H NMR (400 MHz, CD₃OD) δ=7.99 (s, 1H), 5.75-5.69 (m, 1H), 5.66 (br, d, J=3.5 Hz, 1H), 5.24 (d, J=2.6 Hz, 1H), 4.56-4.46 (m, 1H), 3.99-3.82 (m, 2H), 2.72 (td, J=6.9, 13.8 Hz, 1H), 2.46-2.36 (m, 2H), 1.22 (d, J=6.8 Hz, 6H). ¹⁹F NMR (376 MHz, CD₃OD) S=−168.90 (s, 1F). LCMS:ESI-MS: m/z 366.1 [M+H]⁺.

Step C: 2-Amino-9-((1S,3S,4S)-3-fluoro-4-hydroxy-3-(hydroxymethyl)-2-methylenecyclopentyl)-1,9-dihydro-6H-purin-6-one. To a solution of N-(9-((1S,3S,4S)-3-fluoro-4-hydroxy-3-(hydroxymethyl)-2-methylenecyclopentyl)-6-oxo-6,9-dihydro-1H-purin-2-yl)isobutyramide (51 mg, 139.59 mol) in MeOH (0.5 mL) was treated with NH₃ MeOH (7 M, 510.00 μL), and the mixture was stirred at 25° C. for 2 h. The reaction mixture was concentrated under reduced pressure. The residue was triturated with DCM (10 mL), and filtered off. The filter cake was dissolved in water (20 mL), and the aqueous phase was back-extracted with EA (10 mL). The aqueous phase is freeze-dried to give 2-amino-9-((1S,3S,4S)-3-fluoro-4-hydroxy-3-(hydroxymethyl)-2-methylenecyclopentyl)-1,9-dihydro-6H-purin-6-one (32.3 mg, 78.01% yield, 99.55% purity) as white solid. ¹H NMR (400 MHz, CD₃OD) δ=7.75 (s, 1H), 5.60 (d, J=3.3 Hz, 1H), 5.60-5.53 (m, 1H), 5.23-5.14 (m, 1H), 4.50 (q, J=5.0 Hz, 1H), 4.07-3.92 (m, 1H), 3.93-3.76 (m, 1H), 2.52-2.40 (m, 1H), 2.40-2.27 (m, 1H). ¹⁹F NMR (376 MHz, CD₃OD) δ=−169.02 (s, 1F). LCMS:ESI-MS: m/z 296.1 [M+H]⁺.

### Example 5: 2-Amino-9-((1R,3R,4S)-3-fluoro-4-hydroxy-3-(hydroxymethyl)-2-methylenecyclopentyl)-1,9-dihydro-6H-purin-6-one

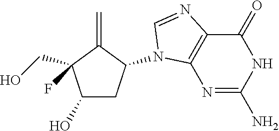 [SMILES: C=C1[C@H](n2cnc3c(=O)[nH]c(N)nc32)C[C@H](O)[C@]1(F)CO]

Step A: 6-(Benzyloxy)-9-((3R,4S)-4-(benzyloxy)-3-((benzyloxy)methyl)-3-fluoro-2-methylenecyclopentyl)-9H-purin-2-amine. To a solution of (3R,4S)-4-(benzyloxy)-3-((benzyloxy)methyl)-3-fluoro-2-methylenecyclopentan-1-ol (Intermediate 3, 160.00 mg, 467.29 mol) and PPh₃ (367.69 mg, 1.40 mmol, 3 eq.) and 6-benzyloxy-9H-purin-2-amine (169.10 mg, 700.94 mol) in anhydrous THF (5 mL) at 0° C. was added DIAD (283.47 mg, 1.40 mmol, 272.57 μL). After the addition, the reaction was stirred at 25° C. for 12 h. The reaction mixture was quenched with water (10 mL), and extracted with EA (10 mL*2). The organic layer was washed with brine (10 mL), dried over anhydrous Na₂SO₄, filtered, concentrated at low pressure. The residue was purified by column chromatography (SiO₂, Petroleum ether/Ethyl acetate=100/1 to 1/1) to give 6-(benzyloxy)-9-((3R,4S)-4-(benzyloxy)-3-((benzyloxy)methyl)-3-fluoro-2-methylenecyclopentyl)-9H-purin-2-amine (0.057 g, 21.17% yield, 98.15% purity) as a white solid. ¹H NMR (400 MHz, CDCl₃) δ=7.65 (d, J=16.6 Hz, 1H), 7.50 (d, J=7.0 Hz, 2H), 7.42-7.27 (m, 10H), 5.63-5.46 (m, 4H), 5.24-5.08 (m, 1H), 4.81 (s, 2H), 4.73-4.59 (m, 4H), 4.41 (br, dd, J=5.5, 9.5 Hz, 1H), 4.39-4.32 (m, 1H), 4.29-4.19 (m, 1H), 4.09-3.87 (m, 2H), 2.73-2.53 (m, 1H), 2.32-2.16 (m, 1H). ¹⁹F NMR (377 MHz, CDCl₃) δ=−147.42 (s, 1F), −149.82 (s, 1F). LCMS: ESI-MS: m/z 566.3 [M+H]⁺.

Step B: 2-Amino-9-((1R,3R,4S)-3-fluoro-4-hydroxy-3-(hydroxymethyl)-2-methylenecyclopentyl)-1,9-dihydro-6H-purin-6-one. To a solution of 6-(benzyloxy)-9-((3R,4S)-4-(benzyloxy)-3-((benzyloxy)methyl)-3-fluoro-2-methylenecyclopentyl)-9H-purin-2-amine (40.00 mg, 70.72 mol) in DCM (2 mL) was added BCl₃ (1 M, 636.45 μL) at −78° C. The mixture was stirred at −78° C. for 2 h. The reaction was quenched with (1 M NH₃/MeOH, 5 mL). The reaction mixture was concentrated at low pressure. The residue was purified by column chromatography (SiO₂, DCM/MeOH=100/1 to 5/1) and further purified by Prep-HPLC (column: Waters Xbridge 150*25 5 u; mobile phase: [water(10 mM NH₄HCO₃)-ACN]; B %: 0%-25%,6 min) to give 2-amino-9-((1R,3R,4S)-3-fluoro-4-hydroxy-3-(hydroxymethyl)-2-methylenecyclopentyl)-1,9-dihydro-6H-purin-6-one (0.005 g, 23.85% yield, 99.58% purity) as a white solid. ¹H NMR (400 MHz, CD₃OD) δ=7.82 (s, 1H), 5.67-5.58 (m, 1H), 5.49 (br, s, 1H), 5.12 (d, J=3.5 Hz, 1H), 4.42-4.34 (m, 1H), 4.12-4.02 (m, 2H), 2.74-2.66 (m, 1H), 2.23-2.14 (m, 1H). ¹⁹F NMR (377 MHz, CD₃OD) δ=−154.86 (s, 1F). LCMS: ESI-MS: m/z 296.1[M+H]⁺.

### Example 6: (1S,2S,4S)-4-(6-Amino-9H-purin-9-yl)-2-fluoro-2-(hydroxymethyl)-3-methylenecyclopentan-1-ol

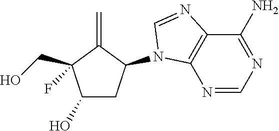 [SMILES: C=C1[C@@H](n2cnc3c(N)ncnc32)C[C@H](O)[C@@]1(F)CO]

Step A: N,N-Di-BOC-9-((1S,3S,4S)-4-((tert-butyldimethylsilyl)oxy)-3-(((tert-butyldimethylsilyl)oxy)methyl)-3-fluoro-2-methylenecyclopentyl)-9H-purin-6-amine. To a solution of (1R,3S,4S)-4-((tert-butyldimethylsilyl)oxy)-3-(((tert-butyldimethylsilyl)oxy)methyl)-3-fluoro-2-methylenecyclopentan-1-ol (Intermediate 2, Method A, product from Step P, 50.00 mg, 127.98 mol) and a (64.38 mg, 191.97 mol) in THF (1 mL) was added PPh₃ (100.71 mg, 383.95 mol) followed by DIAD (77.64 mg, 383.95 mol, 74.65 μL) in THF (0.3 mL) at 0° C. The mixture was stirred at 25° C. for 12 h. The reaction was quenched with water (10 mL), and extracted by EA (15 mL). The organic layer was dried over anhydrous Na₂SO₄, and concentrated at low pressure. The residue was purified by column chromatography (SiO₂, Petroleum ether/Ethyl acetate=100/1 to 3/1) to give N,N-Di-BOC-9-((1S,3S,4S)-4-((tert-butyldimethylsilyl)oxy)-3-(((tert-butyldimethylsilyl)oxy)methyl)-3-fluoro-2-methylenecyclopentyl)-9H-purin-6-amine (42 mg, 46.35% yield) as a colorless oil. ¹H NMR (400 MHz, CDCl₃) δ=8.81 (s, 1H), 8.11 (s, 1H), 5.81-5.71 (m, 1H), 5.61-5.56 (m, 1H), 5.12 (br s, 1H), 4.71-4.62 (m, 1H), 4.05-3.95 (m, 1H), 3.88 (t, J=12.0 Hz, 1H), 2.60-2.47 (m, 1H), 2.39 (dt, J=3.6, 8.7 Hz, 1H), 1.45 (s, 18H), 0.93 (d, J=7.5 Hz, 18H), 0.22-0.02 (m, 12H). ¹⁹F NMR (376 MHz, CDCl₃) δ=−165.83 (s, 1F).

Step B: N,N-Di-BOC-(1S,2S,4S)-4-(6-Amino-9H-purin-9-yl)-2-fluoro-2-(hydroxymethyl)-3-methylenecyclopentan-1-ol. N,N-Di-BOC-9-((1S,3S,4S)-4-((tert-Butyldimethylsilyl)oxy)-3-(((tert-butyldimethylsilyl)oxy)methyl)-3-fluoro-2-methylenecyclopentyl)-9H-purin-6-amine (42.00 mg, 59.32 mol) was dissolved in THF (0.2 mL) and followed by addition of TBAF (1 M, 118.64 μL). The mixture was stirred at 25° C. for 30 min. The reaction was diluted with EA (10 mL), and the reaction mixture was washed with brine (5 mL*2). The organic layer was dried over anhydrous Na₂SO₄, and concentrated at low pressure. The residue was purified by column chromatography (SiO₂, Petroleum ether/Ethyl acetate=100/1 to 0/1) to give N,N-Di-BOC-(1S,2S,4S)-4-(6-amino-9H-purin-9-yl)-2-fluoro-2-(hydroxymethyl)-3-methylenecyclopentan-1-ol (22 mg, 76.96% yield, 99.5% purity) as light yellow oil. ¹H NMR (400 MHz, CDCl₃) δ=8.76 (s, 1H), 8.14 (s, 1H), 5.82-5.64 (m, 2H), 5.17 (br, s, 1H), 4.73-4.53 (m, 1H), 4.30 (br, s, 1H), 4.23-4.14 (m, 1H), 4.08-3.97 (m, 1H), 2.86-2.63 (m, 1H), 2.86-2.63 (m, 1H), 2.57-2.46 (m, 1H), 1.50-1.46 (m, 18H). ¹⁹F NMR (376 MHz, CDCl₃) δ=−164.14 (s, 1F).

Step C: (1S,2S,4S)-4-(6-Amino-9H-purin-9-yl)-2-fluoro-2-(hydroxymethyl)-3-methylenecyclopentan-1-ol. To a solution of N,N-Di-BOC-(1S,2S,4S)-4-(6-amino-9H-purin-9-yl)-2-fluoro-2-(hydroxymethyl)-3-methylenecyclopentan-1-ol (220 mg, 458.81 mol) in DCM (3 mL) was added TFA (0.2 mL). The mixture was stirred at 25° C. for 1 h. The reaction mixture was concentrated at low pressure. The residue was dissolved in MeOH (10 mL). The resulting solution was adjusted to pH=7-8 by addition of 2 drops of NH₃ MeOH (7.0 M). 300 mg of silica gel was added, and the mixture was concentrated under reduced pressure. The residue was purified by column chromatography (SiO₂, DCM/MeOH=40/1 to 5/1) to give ˜130 mg of crude product as yellow oil which was further purified by Prep-HPLC (column: Waters Xbridge 150*255 u; mobile phase: [water (10 mM NH₄HCO₃)-ACN]; B %: 3%-23%, 6.2 min) to give (1S,2S,4S)-4-(6-amino-9H-purin-9-yl)-2-fluoro-2-(hydroxymethyl)-3-methylenecyclopentan-1-ol (90 mg, 70.24% yield, 100% purity) as white solid. ¹H NMR (400 MHz, CD₃OD) δ=8.18-8.15 (m, 2H), 5.80-5.75 (m, 1H), 5.67-5.66 (m, 1H), 5.22-5.21 (m, 1H), 4.56-4.52 (m, 1H), 4.13-4.05 (m, 1H), 3.94-3.87 (m, 1H), 2.57-2.46 (m, 1H), 2.45-2.42 (m, 1H). ¹⁹F NMR (376 MHz, CD₃OD) δ=−168.30 (s, 1F). LCMS: ESI-MS: m/z 279.8 [M+1]*.

### Example 7: (1S,2R,4R)-4-(6-Amino-9H-purin-9-yl)-2-fluoro-2-(hydroxymethyl)-3-methylenecyclopentan-1-ol

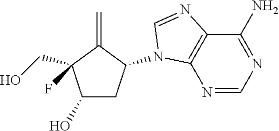 [SMILES: C=C1[C@H](n2cnc3c(N)ncnc32)C[C@H](O)[C@]1(F)CO]

Step A: N,N-Di-BOC-9-((1R,3R,4S)-4-(Benzyloxy)-3-((benzyloxy)methyl)-3-fluoro-2-methylenecyclopentyl)-9H-purin-6-amine and N,N-Di-BOC-9-((1S,3R,4S)-4-(Benzyloxy)-3-((benzyloxy)methyl)-3-fluoro-2-methylenecyclopentyl)-9H-purin-6-amine. To a solution of (3R,4S)-4-(benzyloxy)-3-((benzyloxy)methyl)-3-fluoro-2-methylenecyclopentan-1-ol (Intermediate 3, 470.00 mg, 1.37 mmol) and PPh₃ (1.08 g, 4.12 mmol) and tert-butyl N-tert-butoxycarbonyl-N-(9H-purin-6-yl)carbamate (690.49 mg, 2.06 mmol) in anhydrous THF (8 mL) at 0° C. was added a solution of DIAD (832.69 mg, 4.12 mmol, 800.66 μL) in THF (2 mL), and stirred at 25° C. for 12 h. The reaction mixture was quenched with water (2 mL), and extracted with EA (15 mL*2). The organic layer was washed with brine (10 mL). The organic layer was dried over anhydrous Na₂SO₄, and concentrated at low pressure. The residue was purified by column chromatography (SiO₂, Petroleum ether/Ethyl acetate=100/1 to 1/1) to give pure two isomers, which were purified by Prep-TLC (Toluene/EA=5/1) again to give N,N-Di-BOC-9-((1R,3R,4S)-4-(benzyloxy)-3-((benzyloxy)methyl)-3-fluoro-2-methylenecyclopentyl)-9H-purin-6-amine (210 mg, 22.26% yield, 96% purity) as a white solid: ¹H NMR (400 MHz, CDCl₃) δ=8.84 (s, 1H), 8.18 (s, 1H) 7.41-7.20 (m, 10H), 5.79 (br, s, 1H), 5.66 (br, s, 1H), 5.26-5.08 (m, 1H), 4.76-4.60 (m, 4H), 4.36-4.21 (m, 1H), 4.20-4.05 (m, 1H), 3.92 (br, dd, J=10.2, 18.2 Hz, 1H), 2.85-2.67 (m, 1H), 2.35-2.17 (m, 1H), 1.46 (s, 18H). ¹⁹F NMR (376 MHz, CDCl₃) δ=−149.82 (s, 1F); LCMS: ESI-MS: m/z 660.2[M+H]⁺; and N,N-Di-BOC-9-((1S,3R,4S)-4-(benzyloxy)-3-((benzyloxy)methyl)-3-fluoro-2-methylenecyclopentyl)-9H-purin-6-amine (255 mg, 23.85%) as white solid: ¹H NMR (400 MHz, CDCl₃) δ=8.75 (s, 1H), 7.97 (s, 1H), 7.39-7.28 (m, 10H), 5.79 (br, s, 1H), 5.66 (br s, 1H), 5.18 (br, s, 1H), 5.11-4.94 (m, 1H), 4.73-4.54 (m, 4H), 4.43 (br, d, J=9.8 Hz, 1H), 4.10-3.80 (m, 2H), 2.75-2.56 (m, 1H), 2.38-2.24 (m, 1H), 1.65-1.48 (m, 18H); ¹⁹F NMR (376 MHz, CDCl₃) δ=−147.36 (s, 1F); LCMS: ESI-MS: m/z 660.2 [M+H]⁺.

Step B: 9-((1R,3R,4S)-4-(Benzyloxy)-3-((benzyloxy)methyl)-3-fluoro-2-methylenecyclopentyl)-9H-purin-6-amine. The title compound was prepared in a manner analogous to Example 1, Step C using N,N-Di-BOC-9-((1R,3R,4S)-4-(benzyloxy)-3-((benzyloxy)methyl)-3-fluoro-2-methylenecyclopentyl)-9H-purin-6-amine instead of N,N-Di-BOC-(1S,2S,4S)-4-(4-amino-7H-pyrrolo[2,3-d]pyrimidin-7-yl)-2-fluoro-2-(hydroxymethyl)-3-methylenecyclopentan-1-ol.

Step C: (1S,2R,4R)-4-(6-Amino-9H-purin-9-yl)-2-fluoro-2-(hydroxymethyl)-3-methylenecyclopentan-1-ol. To a solution of 9-((1R,3R,4S)-4-(benzyloxy)-3-((benzyloxy)methyl)-3-fluoro-2-methylenecyclopentyl)-9H-purin-6-amine (29.00 mg, 63.11 mol) in DCM (1 mL) was added BCl₃ (1 M, 189.33 μL) at −78° C. The mixture was stirred at −78° C. for 1 h. The reaction was poured into diluted NH₃/MeOH (˜1 M, 5 mL), and concentrated at low pressure. The residue was purified by column chromatography (SiO₂, DCM/MeOH=100/1 to 20/1) to give (1S,2R,4R)-4-(6-amino-9H-purin-9-yl)-2-fluoro-2-(hydroxymethyl)-3-methylenecyclopentan-1-ol (5 mg, 29% yield) as a white solid. ¹H NMR (400 MHz, CD₃OD) δ=8.22 (s, 1H), 8.21-8.15 (m, 1H), 5.79-5.50 (m, 2H), 5.23-5.00 (m, 1H), 4.45-4.34 (m, 1H), 4.19-4.00 (m, 2H), 2.84-2.72 (m, 1H), 2.30-2.15 (m, 1H). ¹⁹F NMR (376 MHz, CD₃OD) δ=−154.89 (s, 1F). LCMS: ESI-MS: m/z 280.1[M+H]⁺.

### Example 8: (1S,2R,4S)-4-(6-Amino-9H-purin-9-yl)-2-fluoro-2-(hydroxymethyl)-3-methylenecyclopentan-1-ol

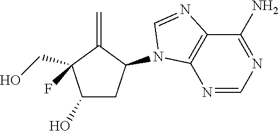 [SMILES: C=C1[C@@H](n2cnc3c(N)ncnc32)C[C@H](O)[C@]1(F)CO]

Step A: 9-((1S,3R,4S)-4-(Benzyloxy)-3-((benzyloxy)methyl)-3-fluoro-2-methylenecyclopentyl)-9H-purin-6-amine. To a solution of N,N-Di-BOC-9-((1S,3R,4S)-4-(benzyloxy)-3-((benzyloxy)methyl)-3-fluoro-2-methylenecyclopentyl)-9H-purin-6-amine (Example 7, product B from Step A, 330.00 mg, 500.19 mol) in DCM (4 mL) was added TFA (1 mL) at 0° C. The mixture was stirred at 25° C. for 2 h. The reaction mixture was diluted with DCM (2 mL), and 500 mg of silica gel was added, and concentrated at low pressure. The residue was purified by flash silica gel chromatography (ISCO®; 4 g SepaFlash® Silica Flash Column, Eluent of 0˜5% DCM/MeOH @ 20 mL/min) to give 9-((1S,3R,4S)-4-(benzyloxy)-3-((benzyloxy)methyl)-3-fluoro-2-methylenecyclopentyl)-9H-purin-6-amine (216 mg, 93.98% yield) as a white solid. ¹H NMR (400 MHz, CDCl₃) δ=8.83-8.15 (m, 1H), 8.07-7.75 (m, 1H), 7.62-7.14 (m, 10H), 6.03-5.50 (m, 4H), 5.34-5.08 (m, 1H), 4.84-4.60 (m, 4H), 4.52-4.34 (m, 1H), 4.06-3.76 (m, 2H), 2.66 (ddd, J=2.8, 8.5, 13.6 Hz, 1H), 2.41-2.21 (m, 1H). ¹⁹F NMR (377 MHz, CDCl₃) δ=−147.23 (s, 1F).

Step B: (1S,2R,4S)-4-(6-Amino-9H-purin-9-yl)-2-fluoro-2-(hydroxymethyl)-3-methylenecyclopentan-1-ol. To a solution of 9-((1S,3R,4S)-4-(benzyloxy)-3-((benzyloxy)methyl)-3-fluoro-2-methylenecyclopentyl)-9H-purin-6-amine (60.00 mg, 130.57 mol) in DCM (2 mL) was added BCl₃ (1 M, 783.44 μL) at −78° C., and stirred at −78° C. for 1 h. The reaction mixture was poured into NH₃/MeOH (˜1.0 M, 3 mL), and concentrated at low pressure. The residue was purified by column chromatography (SiO₂, DCM/MeOH=100/1 to 10/1) to give (1S,2R,4S)-4-(6-amino-9H-purin-9-yl)-2-fluoro-2-(hydroxymethyl)-3-methylenecyclopentan-1-ol (25 mg, 68.56% yield, 100% purity) as a white solid. ¹H NMR (400 MHz, CD₃OD) S=8.20 (s, 1H), 8.07 (s, 1H), 5.75 (br, s, 1H), 5.64 (dd, J=2.6, 5.1 Hz, 1H), 5.10 (dd, J=2.0, 4.5 Hz, 1H), 4.59-4.49 (m, 1H), 4.09-3.90 (m, 2H), 2.49-2.42 (m, 2H). ¹⁹F NMR (377 MHz, CDCl₃) δ=−147.23 (s, 1F). LCMS: ESI-MS: m/z 280.1 [M+H]⁺.

### Example 9: (1S,2R,4S)-4-(4-Amino-7H-pyrrolo[2,3-d]pyrimidin-7-yl)-2-(hydroxymethyl)-3-methylenecyclopentan-1-ol

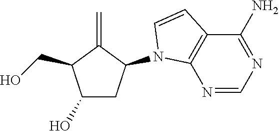 [SMILES: C=C1[C@H](CO)[C@@H](O)C[C@@H]1n1ccc2c(N)ncnc21]

Step A: 4-Chloro-7-((1S,3R,4S)-2-methylene-4-((triisopropylsilyl)oxy)-3-(((triisopropylsilyl)oxy)methyl)cyclopentyl)-7H-pyrrolo[2,3-d]pyrimidine. To a solution of (1R,3R,4S)-2-methylene-4-((triisopropylsilyl)oxy)-3-(((triisopropylsilyl)oxy)methyl)cyclopentan-1-ol (Intermediate 1, 470 mg, 1.03 mmol) and 4-chloro-7H-pyrrolo[2,3-d]pyrimidine (315.98 mg, 2.06 mmol) in THF (10 mL) was added PPh₃ (809.51 mg, 3.09 mmol) and DIAD (624.09 mg, 3.09 mmol, 600.09 μL) at 25° C. The reaction mixture was stirred at 25° C. for 12 h. The reaction mixture was concentrated under reduced pressure. Purification (FCC, SiO₂, Eluent of 0˜30% Ethylacetate/Petroleum ether gradient @ 18 mL/min) afforded 4-chloro-7-((1S,3R,4S)-2-methylene-4-((triisopropylsilyl)oxy)-3-(((triisopropylsilyl)oxy)methyl)cyclopentyl)-7H-pyrrolo[2,3-d]pyrimidine (412 mg, 67.60% yield) as a colorless oil. ¹H NMR (400 MHz, CDCl₃) δ=8.61 (s, 1H), 7.27-7.25 (m, 1H), 6.64-6.55 (m, 1H), 6.04-5.92 (m, 1H), 5.14 (t, J=2.2 Hz, 1H), 4.64 (t, J=2.2 Hz, 1H), 4.59 (br, s, 1H), 3.90-3.79 (m, 2H), 2.81 (br, s, 1H), 2.34-2.23 (m, 2H), 1.19-0.98 (m, 42H). LCMS: ESI-MS: m/z 592.1 [M+1]*.

Step B: 7-((1S,3R,4S)-2-Methylene-4-((triisopropylsilyl)oxy)-3-(((triisopropylsilyl)oxy)methyl)cyclopentyl)-7H-pyrrolo[2,3-d]pyrimidin-4-amine. To a solution of 4-chloro-7-((1S,3R,4S)-2-methylene-4-((triisopropylsilyl)oxy)-3-(((triisopropylsilyl)oxy)methyl)cyclopentyl)-7H-pyrrolo[2,3-d]pyrimidine (250 mg, 422.01 mol) in dioxane (1 mL) was added NH₃H₂O (16.25 g, 267.17 mmol, 17.86 mL, 28% purity). The mixture was stirred at 100° C. for 12 h. The reaction mixture was concentrated under reduced pressure. Purification (FCC, SiO₂, Petroleum ether/Ethyl acetate=1001 to 2/1) afforded 7-((1S,3R,4S)-2-methylene-4-((triisopropylsilyl)oxy)-3-(((triisopropylsilyl)oxy)methyl)cyclopentyl)-7H-pyrrolo[2,3-d]pyrimidin-4-amine (0.198 g, 40.94% yield) as a colorless oil. ¹H NMR (400 MHz, CD₃OD) δ=8.06 (s, 1H), 7.03 (d, J=3.4 Hz, 1H), 6.60-6.57 (m, 1H), 5.87 (br, dd, J=7.9, 10.4 Hz, 1H), 5.21-5.13 (m, 1H), 4.70-4.66 (m, 2H), 3.92-3.86 (m, 2H), 2.82 (br, s, 1H), 2.42-2.29 (m, 1H), 2.29-2.18 (m, 1H), 1.21-1.05 (m, 42H). LCMS: ESI-MS: m/z 573.2 [M+1]*.

Step C: (1S,2R,4S)-4-(4-amino-7H-pyrrolo[2,3-d]pyrimidin-7-yl)-2-(hydroxymethyl)-3-methylenecyclopentan-1-ol. To a solution of 7-((1S,3R,4S)-2-methylene-4-((triisopropylsilyl)oxy)-3-(((triisopropylsilyl)oxy)methyl)cyclopentyl)-7H-pyrrolo[2,3-d]pyrimidin-4-amine (190 mg, 331.61 mol) in MeOH (6 mL) was added NH₄F (245.63 mg, 6.63 mmol). The mixture was stirred at 80° C. for 12 h. The reaction mixture was cooled down, and the resulting solids were filtered off. The filtrate was concentrated under reduced pressure. Purification (FCC, SiO₂, DCM/MeOH from 100/1 to 10/1) and further purified by SFC separation (column: DAICEL CHIRALPAK AD-H(250 mm*30 mm, 5 um); mobile phase: [0.1% NH₃.H₂O, MeOH]; B %: 30%-30%, min, RT=4.488 min) afforded (1S,2R,4S)-4-(4-amino-7H-pyrrolo[2,3-d]pyrimidin-7-yl)-2-(hydroxymethyl)-3-methylenecyclopentan-1-ol (32 mg, 37.07% yield, 100% purity) as a white solid. ¹H NMR (400 MHz, CD₃OD) δ=8.04 (s, 1H), 7.12 (d, J=3.7 Hz, 1H), 6.58 (d, J=3.7 Hz, 1H), 5.90-5.71 (m, 1H), 5.19 (br, s, 1H), 4.65 (t, J=2.2 Hz, 1H), 4.46-4.30 (m, 1H), 3.89-3.66 (m, 2H), 2.70 (br s, 1H), 2.42-2.28 (m, 1H), 2.28-2.15 (m, 1H). LCMS: ESI-MS: m/z 261.1 [M+1]*.

### Example 10: 2-Amino-9-((1S,3S,4S)-3-(fluoromethyl)-4-hydroxy-3-(hydroxymethyl)-2-methylenecyclopentyl)-1,9-dihydro-6H-purin-6-one

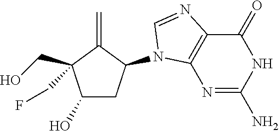 [SMILES: C=C1[C@@H](n2cnc3c(=O)[nH]c(N)nc32)C[C@H](O)[C@]1(CO)CF]

Step A: ((1S,3S,5S)-1-((bis(4-Methoxyphenyl)(phenyl)methoxy)methyl)-3-(6-((diphenylcarbamoyl)oxy)-2-isobutyramido-9H-purin-9-yl)-2-methylene-5-(pivaloyloxy)cyclopentyl)methyl pivalate. To a solution of ((1S,3R,5S)-1-((bis(4-methoxyphenyl)(phenyl)methoxy)methyl)-3-hydroxy-2-methylene-5-(pivaloyloxy)cyclopentyl)methyl pivalate (Intermediate 7, 900 mg, 1.40 mmol) and 2-isobutyramido-9H-purin-6-yl diphenylcarbamate (Intermediate 5, 871.88 mg, 2.09 mmol) in THF (20 mL) in dioxane (20 mL) was added PPh₃ (1.10 g, 4.19 mmol) in one portion at 25° C. under N₂. After the addition of DIAD (846.73 mg, 4.19 mmol, 814.17 μL) in dropwise at 25° C. The reaction mixture was stirred at 25° C. for 12 h. The solvent was removed in vacuum to give a yellow oil. The residue was purified by silica gel column chromatography (Petroleum ether:Ethyl acetate=5/1 to 3/1). ((1S,3S,5S)-1-((bis(4-methoxyphenyl)(phenyl)methoxy)methyl)-3-(6-((diphenylcarbamoyl)oxy)-2-isobutyramido-9H-purin-9-yl)-2-methylene-5-(pivaloyloxy)cyclopentyl)methyl pivalate (900 mg, 819.59 mol, 58.72% yield, 95% purity) was obtained as a pale yellow foam. ¹H NMR (400 MHz, CDCl₃) δ=8.63 (s, 1H), 7.95 (s, 1H), 7.41-7.35 (m, 12H), 7.29-7.23 (m, 7H), 6.83-6.81 (m, 4H), 5.61 (d, J=2.8 Hz, 1H), 5.43-5.36 (m, 2H), 5.24 (br d, J=11.6 Hz, 1H), 4.85 (d, J=2.4 Hz, 1H), 4.45 (d, J=11.8 Hz, 1H), 3.79 (m, 6H), 3.46 (d, J=8.8 Hz, 1H), 3.31 (d, J=9.0 Hz, 1H), 3.01-2.95 (m, 2H), 2.22-2.15 (m, 1H), 1.28-1.26 (m, 6H), 1.23 (s, 9H), 1.01 (s, 9H). LCMS: ESI-MS: m/z=1043.5 [M+H]⁺.

Step B: ((1S,3S,5S)-3-(6-((Diphenylcarbamoyl)oxy)-2-isobutyramido-9H-purin-9-yl)-1-(hydroxymethyl)-2-methylene-5-(pivaloyloxy)cyclopentyl)methyl pivalate. To a solution of ((1S,3S,5S)-1-((bis(4-methoxyphenyl)(phenyl)methoxy)methyl)-3-(6-((diphenylcarbamoyl)oxy)-2-isobutyramido-9H-purin-9-yl)-2-methylene-5-(pivaloyloxy)cyclopentyl)methyl pivalate (200.00 mg, 191.72 mol) in DCM (2 mL) was added TFA (154.00 mg, 1.35 mmol, 0.1 mL) in one portion at 0° C. under N₂. The reaction mixture was stirred at 0° C. for 20 min. The reaction mixture was poured into saturated aq. NaHCO₃ solution to be adjusted to pH>8. The mixture was extracted with EA (30 mL*3). The resulting solution was dried over anhydrous Na₂SO₄. The solvent was removed in vacuum to give a yellow oil. The residue was purified by silica gel column chromatography (Petroleum ether/Ethyl acetate=3/1 to 1/1). ((1S,3S,5S)-3-(6-((diphenylcarbamoyl)oxy)-2-isobutyramido-9H-purin-9-yl)-1-(hydroxymethyl)-2-methylene-5-(pivaloyloxy)cyclopentyl)methyl pivalate (320 mg, 3 batches, 414.66 mol, 72.10% yield, 96% purity) was obtained as a pale yellow foam. LCMS: ESI-MS: m/z=741.4 [M+H]⁺.

Step C: ((1R,3S,5S)-3-(6-((Diphenylcarbamoyl)oxy)-2-isobutyramido-9H-purin-9-yl)-2-methylene-5-(pivaloyloxy)-1-((((trifluoromethyl)sulfonyl)oxy)methyl)cyclopentyl)methyl pivalate. To a solution of ((1S,3S,5S)-3-(6-((diphenylcarbamoyl)oxy)-2-isobutyramido-9H-purin-9-yl)-1-(hydroxymethyl)-2-methylene-5-(pivaloyloxy)cyclopentyl)methyl pivalate (170 mg, 229.47 mol) and pyridine (181.51 mg, 2.29 mmol, 185.21 μL) in DCM (2 mL) was added trifluoromethanesulfonic anhydride (Tf₂O or triflic anhydride) (97.11 mg, 344.20 mol, 56.79 μL) in dropwise at 0° C. under N₂. The reaction mixture was stirred at 0° C. for 1 h. The solvent was removed in vacuo to give a yellow oil. The residue was purified by silica gel column chromatography (Petroleum ether/Ethyl acetate=1/2 to 0/1). ((1R,3S,5S)-3-(6-((diphenylcarbamoyl)oxy)-2-isobutyramido-9H-purin-9-yl)-2-methylene-5-(pivaloyloxy)-1-((((trifluoromethyl)sulfonyl)oxy)methyl)cyclopentyl)methyl pivalate (140 mg, 96.23 mol, 41.94% yield, 60% purity) was obtained as a pale yellow oil. LCMS: ESI-MS: m/z=873.3 [M+H]⁺.

Step D: ((1S,3S,5S)-1-(Fluoromethyl)-3-(2-isobutyramido-6-oxo-1,6-dihydro-9H-purin-9-yl)-2-methylene-5-(pivaloyloxy)cyclopentyl)methyl pivalate. To a solution of ((1R,3S,5S)-3-(6-((diphenylcarbamoyl)oxy)-2-isobutyramido-9H-purin-9-yl)-2-methylene-5-(pivaloyloxy)-1-((((trifluoromethyl)sulfonyl)oxy)methyl)cyclopentyl)methyl pivalate (320 mg, 366.59 mol) in THF (0.5 mL) was added TBAF (1 M, 3.2 mL) in one portion at 25° C. under N₂. The reaction mixture was stirred at 25° C. for 12 h. The solvent was removed in vacuo to give a yellow oil. The residue was purified by silica gel column chromatography (Petroleum ether/Ethyl acetate=1/2 to 0/1). ((1S,3S,5S)-1-(fluoromethyl)-3-(2-isobutyramido-6-oxo-1,6-dihydro-9H-purin-9-yl)-2-methylene-5-(pivaloyloxy)cyclopentyl)methyl pivalate (100 mg, 182.61 mol, 49.81% yield) was obtained as a pale yellow foam. LCMS: ESI-MS: m/z=548.2 [M+H]⁺.

Step E: 2-Amino-9-((1S,3S,4S)-3-(fluoromethyl)-4-hydroxy-3-(hydroxymethyl)-2-methylenecyclopentyl)-1,9-dihydro-6H-purin-6-one. To a solution of ((1S,3S,5S)-1-(fluoromethyl)-3-(2-isobutyramido-6-oxo-1,6-dihydro-9H-purin-9-yl)-2-methylene-5-(pivaloyloxy)cyclopentyl)methyl pivalate (100 mg, 182.61 mol) in MeOH (5 mL) was added NaOH (1 M, 1.00 mL) at 25° C. under N₂. The reaction mixture was stirred at 25° C. for 12 h. The reaction mixture was adjusted to pH=6-7 with 1.0 M HCl solution and the solvent was removed in vacuum to give 300 mg of crude product as a yellow oil. 470 mg of crude product was purified by prep-HPLC (column: Waters Xbridge 150*25 5 u; mobile phase: [water(10 mM NH₄HCO₃)-ACN]; B %: 0%-23%,6 min). 2-amino-9-((1S,3S,4S)-3-(fluoromethyl)-4-hydroxy-3-(hydroxymethyl)-2-methylenecyclopentyl)-1,9-dihydro-6H-purin-6-one (47.9 mg, 96% purity, 54.8% yield) was obtained as a white solid. ¹H NMR (400 MHz, CD₃OD) δ=7.79 (s, 1H), 5.59-5.54 (m, 1H), 5.23 (d, J=2.8 Hz, 1H), 4.82 (d, J=2.4 Hz, 1H), 4.77-4.47 (m, 2H), 4.40 (d, J=3.0 Hz, 1H), 3.79 (s, 2H), 2.54 (ddd, J=4.8, 11.0, 13.0 Hz, 1H), 2.24 (dd, J=8.2, 12.8 Hz, 1H). ¹⁹F NMR (376 MHz, CD₃OD) δ=−232.9. LCMS: ESI-MS: m/z=310.1 [M+H]⁺.

### Example 11: 4-Amino-1-((1S,3S,4S)-3-(fluoromethyl)-4-hydroxy-3-(hydroxymethyl)-2-methylenecyclopentyl)pyrimidin-2(1H)-one

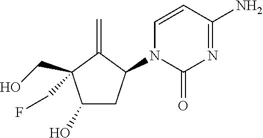 [SMILES: C=C1[C@@H](n2ccc(N)nc2=O)C[C@H](O)[C@]1(CO)CF]

Step A: ((1S,3S,5S)-3-(3-Benzoyl-2,4-dioxo-3,4-dihydropyrimidin-1(2H)-yl)-1-((bis(4-methoxyphenyl)(phenyl)methoxy)methyl)-2-methylene-5-(pivaloyloxy)cyclopentyl)methyl pivalate. To a solution of ((1S,3R,5S)-1-((bis(4-methoxyphenyl)(phenyl)methoxy)methyl)-3-hydroxy-2-methylene-5-(pivaloyloxy)cyclopentyl)methyl pivalate (Intermediate 7, 0.0516 g, 80.03 mol), PPh₃ (41.98 mg, 160.05 mol) and 3-benzoyl-1H-pyrimidine-2,4-dione (25.95 mg, 120.04 mol) in THF (1 mL) was added DIAD (32.36 mg, 160.05 mol, 31.12 μL) at 0° C. The mixture was stirred at 25° C. for 12 hr. The solvent was removed under reduced pressure to give a residue. The residue was purified by flash silica gel chromatography (ISCO®; 4 g SepaFlash® Silica Flash Column, Eluent of 4˜25% Ethyl acetate/Petroleum ethergradient @20 mL/min). ((1S,3S,5S)-3-(3-benzoyl-2,4-dioxo-3,4-dihydropyrimidin-1(2H)-yl)-1-((bis(4-methoxyphenyl)(phenyl)methoxy)methyl)-2-methylene-5-(pivaloyloxy)cyclopentyl)methyl pivalate (0.026 g, 30.84 mol, 38.54% yield) was obtained as a white foam. ¹H NMR (400 MHz, CDCl₃) δ=8.00-7.91 (m, 1H), 7.91-7.84 (m, 1H), 7.71-7.61 (m, 1H), 7.57-7.48 (m, 2H), 7.43-7.35 (m, 2H), 7.34-7.28 (m, 3H), 7.25-7.17 (m, 4H), 6.84-6.77 (m, 5H), 5.88 (d, J=8.2 Hz, 1H), 5.73-5.62 (m, 2H), 5.30-5.25 (m, 1H), 5.14 (s, 1H), 4.43-4.31 (m, 2H), 3.81 (d, J=2.8 Hz, 1H), 3.78 (d, J=0.8 Hz, 6H), 3.31 (s, 1H), 2.26-2.13 (m, 1H), 1.87-1.85 (m, 1H), 1.22-1.17 (m, 9H), 0.97-0.92 (m, 9H). LCMS: ESI-MS: m/z=865.5 [M+Na]⁺.

Step B: ((1S,3S,5S)-3-(3-Benzoyl-2,4-dioxo-3,4-dihydropyrimidin-1(2H)-yl)-1-(hydroxymethyl)-2-methylene-5-(pivaloyloxy)cyclopentyl)methyl pivalate. To a solution of ((1S,3S,5S)-3-(3-benzoyl-2,4-dioxo-3,4-dihydropyrimidin-1(2H)-yl)-1-((bis(4-methoxyphenyl)(phenyl)methoxy)methyl)-2-methylene-5-(pivaloyloxy)cyclopentyl)methyl pivalate (0.026 g, 30.84 mol) in DCM (0.5 mL) was added 2,2-dichloroacetic acid (39.77 mg, 308.43 mol, 25.33 μL). The mixture was stirred at 0° C. for 0.5 hr. The mixture was quenched with saturated aq. NaHCO₃ solution (1 mL) and extracted with DCM (5 mL*2). The combined organic layers were washed with brine (5 mL), dried over anhydrous Na₂SO₄, filtered and concentrated under reduced pressure. The residue was purified by flash silica gel chromatography (ISCO®; 4 g SepaFlash® Silica Flash Column, Eluent of 15-30% Ethyl acetate/Petroleum ethergradient @ 20 mL/min). ((1S,3S,5S)-3-(3-benzoyl-2,4-dioxo-3,4-dihydropyrimidin-1(2H)-yl)-1-(hydroxymethyl)-2-methylene-5-(pivaloyloxy)cyclopentyl)methyl pivalate (0.01 g, 18.50 mol, 59.97% yield) was obtained as a white foam. ¹H NMR (400 MHz, CDCl₃) δ=7.97-7.91 (m, 2H), 7.71-7.63 (m, 1H), 7.56-7.49 (m, 2H), 7.33 (d, J=8.2 Hz, 1H), 5.92-5.87 (m, 1H), 5.70 (s, 1H), 5.40 (d, J=2.6 Hz, 1H), 5.32-5.28 (m, 1H), 5.14 (d, J=2.2 Hz, 1H), 4.38 (d, J=11.6 Hz, 1H), 4.22 (d, J=11.6 Hz, 1H), 3.74-3.68 (m, 1H), 3.65-3.59 (m, 1H), 2.31 (dd, J=8.2, 12.6 Hz, 1H), 2.25-2.14 (m, 1H), 1.26-1.23 (m, 9H), 1.22-1.18 (m, 9H). LCMS: ESI-MS: m/z=563.2 [M+Na]⁺.

Step C: ((1R,3S,5S)-3-(3-Benzoyl-2,4-dioxo-3,4-dihydropyrimidin-1(2H)-yl)-2-methylene-5-(pivaloyloxy)-1-((((trifluoromethyl)sulfonyl)oxy)methyl)cyclopentyl)methyl pivalate. To a solution of ((1S,3S,5S)-3-(3-benzoyl-2,4-dioxo-3,4-dihydropyrimidin-1(2H)-yl)-1-(hydroxymethyl)-2-methylene-5-(pivaloyloxy)cyclopentyl)methyl pivalate (0.08 g, 147.98 mol) in DCM (0.5 mL) was added pyridine (117.05 mg, 1.48 mmol, 119.44 μL) and Tf₂O (83.50 mg, 295.97 mol, 48.83 μL, 2 eq.) at 0° C. The mixture was stirred at 0° C. for 1 hr. The mixture was quenched with saturated NaHCO₃ solution (5 mL) and extracted with DCM (5 mL*2). The combined organic layers were washed with brine (5 mL), dried over anhydrous Na₂SO₄, filtered and concentrated under reduced pressure. The crude was used for next step without purification. ((1R,3S,5S)-3-(3-benzoyl-2,4-dioxo-3,4-dihydropyrimidin-1(2H)-yl)-2-methylene-5-(pivaloyloxy)-1-((((trifluoromethyl)sulfonyl)oxy)methyl)cyclopentyl)methyl pivalate (0.09 g, crude) was obtained as brown oil. LCMS: ESI-MS: m/z=673.1 [M+H]f, 695.1 [M+Na]⁺.

Step D: ((1S,3S,5S)-3-(2,4-Dioxo-3,4-dihydropyrimidin-1(2H)-yl)-1-(fluoromethyl)-2-methylene-5-(pivaloyloxy)cyclopentyl)methyl pivalate. To a solution of ((1R,3S,5S)-3-(3-benzoyl-2,4-dioxo-3,4-dihydropyrimidin-1(2H)-yl)-2-methylene-5-(pivaloyloxy)-1-((((trifluoromethyl)sulfonyl)oxy)methyl)cyclopentyl)methyl pivalate (0.09 g, 133.80 mol) in THF (1 mL) was added TBAF (1 M, 401.39 μL). The mixture was stirred at 60° C. for 3 h. The mixture was diluted with EA (5 mL) and washed with H₂O (5 mL*2). The organic layer was dried over anhydrous Na₂SO₄, filtered and concentrated under reduced pressure. The residue was purified by flash silica gel chromatography (ISCO®; 4 g SepaFlash® Silica Flash Column, Eluent of 0˜1% MeOH/DCMgradient @ 20 mL/min). ((1S,3S,5S)-3-(2,4-dioxo-3,4-dihydropyrimidin-1(2H)-yl)-1-(fluoromethyl)-2-methylene-5-(pivaloyloxy)cyclopentyl)methyl pivalate (0.035 g, 79.82 mol, 59.66% yield) was obtained as a white foam. LCMS: ESI-MS: m/z=461.1 [M+Na]⁺.

Step E: ((1S,3S,5S)-3-(4-Amino-2-oxo-3,4-dihydropyrimidin-1(2H)-yl)-1-(fluoromethyl)-2-methylene-5-(pivaloyloxy)cyclopentyl)methyl pivalate. To a solution of ((1S,3S,5S)-3-(2,4-dioxo-3,4-dihydropyrimidin-1(2H)-yl)-1-(fluoromethyl)-2-methylene-5-(pivaloyloxy)cyclopentyl)methyl pivalate (0.035 g, 79.82 mol) in CH₃CN (0.4 mL) was added DMAP (19.50 mg, 159.64 mol), Et₃N (16.15 mg, 159.64 mol, 22.22 μL) and 2,4,6-triisopropylbenzenesulfonyl chloride (48.35 mg, 159.64 mol). The mixture was stirred at 25° C. for 0.5 h. NH₃—H₂O (413.64 mg, 10.39 mmol, 454.55 μL, 28% purity) was added to the mixture, which was stirred at 25° C. for 1.5 h. The mixture was diluted with EA (5 mL) and washed with NH₄Cl (3 mL*3). The organic layer was dried over anhydrous Na₂SO₄, filtered and concentrated under reduced pressure. The residue was purified by flash silica gel chromatography (ISCO®; 4 g SepaFlash® Silica Flash Column, Eluent of 0˜3.2% MeOH/DCMgradient @ 20 mL/min). ((1S,3S,5S)-3-(4-amino-2-oxo-3,4-dihydropyrimidin-1(2H)-yl)-1-(fluoromethyl)-2-methylene-5-(pivaloyloxy)cyclopentyl)methyl pivalate (0.025 g, 57.14 mol, 71.59% yield) was obtained as a white foam. ¹H NMR (400 MHz, CDCl₃) δ=7.13 (d, J=7.4 Hz, 1H), 6.04 (d, J=6.6 Hz, 1H), 5.69 (s, 1H), 5.30-5.27 (m, 1H), 5.17 (s, 1H), 4.89 (s, 1H), 4.61-4.42 (m, 2H), 4.28-4.20 (m, 1H), 2.83 (td, J=6.9, 13.8 Hz, 1H), 2.15 (d, J=7.4 Hz, 1H), 1.21 (s, 9H), 1.18 (s, 9H). LCMS: ESI-MS: m/z=438.1 [M+H]⁺.

Step F: 4-Amino-1-((1S,3S,4S)-3-(fluoromethyl)-4-hydroxy-3-(hydroxymethyl)-2-methylenecyclopentyl)pyrimidin-2(1H)-one. To a solution of ((1S,3S,5S)-3-(4-amino-2-oxo-3,4-dihydropyrimidin-1(2H)-yl)-1-(fluoromethyl)-2-methylene-5-(pivaloyloxy)cyclopentyl)methyl pivalate (0.078 g, 178.28 mol) in MeOH (1 mL) was added NaOH (1 M, 1.56 mL). The mixture was stirred at 25° C. for 12 hr. The mixture was neutralized with HCl solution (1 mL, 1 M) and the solvent was removed under reduced pressure. The residue was purified by flash silica gel chromatography (ISCO®; 4 g SepaFlash® Silica Flash Column, Eluent of 3-18% MeOH/DCMgradient @ 20 mL/min). The product was further purification by Prep-HPLC (column: Waters Xbridge 150*25 5 u; mobile phase: [water (10 mM NH₄HCO₃)-ACN]; B %: 0%-25%, 6 min). 4-amino-1-((1S,3S,4S)-3-(fluoromethyl)-4-hydroxy-3-(hydroxymethyl)-2-methylenecyclopentyl)pyrimidin-2(1H)-one (0.018 g, 66.85 mol, 37.49% yield, 100% purity) was obtained as a white solid. ¹H NMR (400 MHz, CD₃OD) δ=7.58 (d, J=7.3 Hz, 1H), 5.88 (d, J=7.3 Hz, 1H), 5.74 (s, 1H), 5.23 (d, J=3.0 Hz, 1H), 4.75-4.58 (m, 1H), 4.57-4.40 (m, 1H), 4.31 (d, J=2.8 Hz, 1H), 3.78-3.64 (m, 2H), 2.24-2.07 (m, 2H). LCMS: ESI-MS: m/z=270.2 [M+H]⁺, 539.3[2M+H]⁺.

### Example 12: 1-((1S,3S,4S)-3-(fluoromethyl)-4-hydroxy-3-(hydroxymethyl)-2-methylenecyclopentyl)-5-methylpyrimidine-2,4(1H,3H)-dione

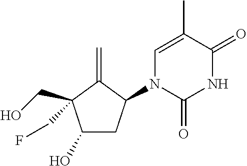 [SMILES: C=C1[C@@H](n2cc(C)c(=O)[nH]c2=O)C[C@H](O)[C@]1(CO)CF]

Step A: ((1S,3S,5S)-3-(3-Benzoyl-5-methyl-2,4-dioxo-3,4-dihydropyrimidin-1(2H)-yl)-1-((bis(4-methoxyphenyl)(phenyl)methoxy)methyl)-2-methylene-5-(pivaloyloxy)cyclopentyl)methyl pivalate. To a solution of ((1S,3R,5S)-1-((bis(4-methoxyphenyl)(phenyl)methoxy)methyl)-3-hydroxy-2-methylene-5-(pivaloyloxy)cyclopentyl)methyl pivalate (Intermediate 7, 500 mg, 775.44 mol) and 3-benzoyl-5-methylpyrimidine-2,4(1H,3H)-dione (Intermediate 6, 267.78 mg, 1.16 mmol) in THF (10 mL) was added PPh₃ (508.47 mg, 1.94 mmol) in one portion at 25° C. under N₂. After addition of DIAD (392.00 mg, 1.94 mmol, 376.93 μL) in dropwise at 0° C., the reaction mixture was stirred at 25° C. for 12 h. The reaction mixture was concentrated at low pressure. The residue was purified by a silica gel column chromatography (Petroleum ether/Ethyl acetate=10/1 to 3/1). ((1S,3S,5S)-3-(3-benzoyl-5-methyl-2,4-dioxo-3,4-dihydropyrimidin-1(2H)-yl)-1-((bis(4-methoxyphenyl)(phenyl)methoxy)methyl)-2-methylene-5-(pivaloyloxy)cyclopentyl)methyl pivalate (200 mg, 233.37 mol, 30.10% yield) was obtained as a pale yellow foam. ¹H NMR (400 MHz, CDCl₃) δ=7.94 (d, J=8 Hz, 2H), 7.70 (m, 1H), 7.53-7.51 (m, 2H), 7.38-7.36 (m, 2H), 7.30-7.25 (m, 7H), 7.24 (s, 1H), 6.83-6.81 (m, 4H), 5.71 (s, 1H), 5.57 (s, 1H), 5.28 (s, 1H), 5.10 (s, 1H), 4.41 (s, 2H), 3.78 (s, 6H), 3.58-3.29 (m, 2H), 2.19-2.16 (d, J=10 Hz, 2H), 1.98 (s, 3H), 1.20 (s, 9H), 0.96 (s, 9H). LCMS: ESI-MS: m/z=879.5 [M+Na]⁺.

Step B: ((1S,3S,5S)-3-(3-Benzoyl-5-methyl-2,4-dioxo-3,4-dihydropyrimidin-1(2H)-yl)-1-(hydroxymethyl)-2-methylene-5-(pivaloyloxy)cyclopentyl)methyl pivalate. To a solution of ((1S,3S,5S)-3-(3-benzoyl-5-methyl-2,4-dioxo-3,4-dihydropyrimidin-1(2H)-yl)-1-((bis(4-methoxyphenyl)(phenyl)methoxy)methyl)-2-methylene-5-(pivaloyloxy)cyclopentyl)methyl pivalate (90.00 mg, 105.02 mol) in DCM (1 mL) was added TFA (77.00 mg, 675.30 mol, 0.05 mL) in one portion at 0° C. under N₂. The reaction mixture was stirred at 0° C. for 1 h. The reaction mixture was poured into saturated aq. NaHCO₃ solution (10 mL), and extracted with EA (20 mL*3). The combined organic phase was washed with brine (10 mL), dried over Na₂SO₄ and concentrated in vacuum to give a yellow oil. The residue was purified by silica gel column chromatography (Petroleum ether/Ethyl acetate=3/1 to 2/1). ((1S,3S,5S)-3-(3-benzoyl-5-methyl-2,4-dioxo-3,4-dihydropyrimidin-1(2H)-yl)-1-(hydroxymethyl)-2-methylene-5-(pivaloyloxy)cyclopentyl)methyl pivalate (33 mg, 59.50 mol, 56.66% yield, 100% purity) was obtained as a pale yellow oil. LCMS: ESI-MS: m/z=577.2 [M+Na]⁺.

Step C: ((1R,3S,5S)-3-(3-benzoyl-5-methyl-2,4-dioxo-3,4-dihydropyrimidin-1(2H)-yl)-2-methylene-5-(pivaloyloxy)-1-((((trifluoromethyl)sulfonyl)oxy)methyl)cyclopentyl)methyl pivalate. To a solution of ((1S,3S,5S)-3-(3-benzoyl-5-methyl-2,4-dioxo-3,4-dihydropyrimidin-1(2H)-yl)-1-(hydroxymethyl)-2-methylene-5-(pivaloyloxy)cyclopentyl)methyl pivalate (200 mg, 360.60 mol) and pyridine (285.23 mg, 3.61 mmol, 291.06 μL) in DCM (5 mL) was added Tf₂O (203.48 mg, 721.20 mol, 118.99 μL) in dropwise at 0° C. under N₂. The reaction mixture was stirred at 0° C. for 1 h. The reaction mixture was concentrated at low pressure. The residue was purified by a silica gel column chromatography (Petroleum ether:Ethyl acetate=3:1 to 2:1). ((1R,3S,5S)-3-(3-benzoyl-5-methyl-2,4-dioxo-3,4-dihydropyrimidin-1(2H)-yl)-2-methylene-5-(pivaloyloxy)-1-((((trifluoromethyl)sulfonyl)oxy)methyl)cyclopentyl)methyl pivalate (220 mg, 320.38 mol, 88.85% yield) was obtained as a pale yellow foam. LCMS: ESI-MS: m/z=709.2 [M+Na]⁺.

Step D: ((1S,3S,5S)-1-(fluoromethyl)-3-(5-methyl-2,4-dioxo-3,4-dihydropyrimidin-1(2H)-yl)-2-methylene-5-(pivaloyloxy)cyclopentyl)methyl pivalate. To a solution of ((1R,3S,5S)-3-(3-benzoyl-5-methyl-2,4-dioxo-3,4-dihydropyrimidin-1(2H)-yl)-2-methylene-5-(pivaloyloxy)-1-((((trifluoromethyl)sulfonyl)oxy)methyl)cyclopentyl)methyl pivalate (210 mg, 305.81 mol) in THF (1 mL) was treated with TBAF (1 M, 4 mL) at 25° C. under N₂. The reaction mixture was stirred at 25° C. for 12 h. The reaction mixture was concentrated at low pressure. The residue was purified by silica gel column chromatography (Petroleum ether/Ethyl acetate=2/1 to 1/1). ((1S,3S,5S)-1-(fluoromethyl)-3-(5-methyl-2,4-dioxo-3,4-dihydropyrimidin-1(2H)-yl)-2-methylene-5-(pivaloyloxy)cyclopentyl)methyl pivalate (95 mg, 178.45 mol, 58.35% yield, 85% purity) was obtained as a pale yellow oil. LCMS: ESI-MS: m/z=453.2 [M+H]⁺.

Step E: 1-((1S,3S,4S)-3-(Fluoromethyl)-4-hydroxy-3-(hydroxymethyl)-2-methylenecyclopentyl)-5-methylpyrimidine-2,4(1H,3H)-dione. To a solution of ((1S,3S,5S)-1-(fluoromethyl)-3-(5-methyl-2,4-dioxo-3,4-dihydropyrimidin-1(2H)-yl)-2-methylene-5-(pivaloyloxy)cyclopentyl)methyl pivalate (95 mg, 209.94 mol.) in MeOH (5 mL) was added NaOH (1 M, 945.82 μL) at 25° C. under N₂. The reaction mixture was stirred at 25° C. for 12 h. The reaction mixture was adjusted to pH=6-7 by adding 1.0 M HCl solution. The resulting mixture was concentrated in vacuum to give 200 mg of crude product as a pale yellow solid. 500 mg of crude product was purified by Prep-HPLC (column: Waters Xbridge 150*25 5 u; mobile phase: [water (10 mM NH₄HCO₃)-ACN]; B %: 0%-27%,6 min) to give a white solid. The solid was re-purified by SFC (column: DAICEL CHIRALPAK IC (250 mm*30 mm, 5 um); mobile phase: [0.1% NH₃H₂O IPA]; B %: 45%-45%, min). 1-((1S,3S,4S)-3-(fluoromethyl)-4-hydroxy-3-(hydroxymethyl)-2-methylenecyclopentyl)-5-methylpyrimidine-2,4(1H,3H)-dione (52.7 mg, 98% purity) was obtained as a white solid. ¹H NMR (400 MHz, CD₃OD) δ=7.40 (d, J=1.2 Hz, 1H), 5.68 (br, dd, J=8.4, 11.0 Hz, 1H), 5.26 (d, J=3.0 Hz, 1H), 4.96 (d, J=2.6 Hz, 1H), 4.72-4.41 (m, 2H), 4.31 (d, J=3.4 Hz, 1H), 3.79-3.67 (m, 2H), 2.25-2.21 (m, 1H), 2.12-2.09 (m, 1H), 1.86 (d, J=1.0 Hz, 3H). ¹⁹F NMR (376 MHz, CD₃OD) δ=−232.41. LCMS: ESI-MS: m/z=285.1 [M+H]⁺.

### Example 13: (1S,2S,4S)-4-(6-Amino-9H-purin-9-yl)-2-(fluoromethyl)-2-(hydroxymethyl)-3-methylenecyclopentan-1-ol

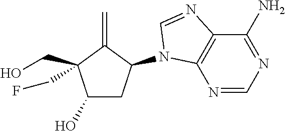 [SMILES: C=C1[C@@H](n2cnc3c(N)ncnc32)C[C@H](O)[C@]1(CO)CF]

Step A: ((1S,3S,5S)-1-((bis(4-Methoxyphenyl)(phenyl)methoxy)methyl)-3-(6-chloro-9H-purin-9-yl)-2-methylene-5-(pivaloyloxy)cyclopentyl)methyl pivalate. To a solution of ((1S,3R,5S)-1-((bis(4-methoxyphenyl)(phenyl)methoxy)methyl)-3-hydroxy-2-methylene-5-(pivaloyloxy)cyclopentyl)methyl pivalate (Intermediate 7, 0.989 g, 1.53 mmol), 6-chloro-9H-purine (711.19 mg, 4.60 mmol) and PPh₃ (61.02 mg, 232.63 mol) in THF (15 mL) was added DIAD (930.47 mg, 4.60 mmol, 894.68 μL) in THF (15 mL) dropwise slowly at 0° C. The mixture was stirred at 0° C. for 3 h., and then stirred at 25° C. for 12 h. The reaction mixture was concentrated at low pressure. The residue was purified by flash silica gel chromatography (ISCO®; 4 g SepaFlash® Silica Flash Column, Eluent of 5˜26% Ethyl acetate/Petroleum ether gradient @22 mL/min) to give ((1S,3S,5S)-1-((bis(4-methoxyphenyl)(phenyl)methoxy)methyl)-3-(6-chloro-9H-purin-9-yl)-2-methylene-5-(pivaloyloxy)cyclopentyl)methyl pivalate (0.760 g, 972.70 mol, 63.42% yield) as colorless oil. ¹H NMR (400 MHz, CDCl₃) δ=8.78-8.75 (m, 1H), 8.20 (s, 1H), 7.42-7.37 (m, 2H), 7.33-7.27 (m, 6H), 7.26-7.20 (m, 1H), 6.83 (d, J=8.4 Hz, 4H), 6.38-6.27 (m, 1H), 5.64 (t, J=9.2 Hz, 1H), 5.55 (d, J=2.6 Hz, 1H), 5.46 (dd, J=3.6, 5.4 Hz, 1H), 4.83 (d, J=2.2 Hz, 1H), 4.64 (d, J=11.5 Hz, 1H), 4.42 (d, J=11.7 Hz, 1H), 3.80 (s, 6H), 3.39 (s, 2H), 2.81-2.69 (m, 1H), 2.34 (ddd, J=3.6, 8.4, 13.7 Hz, 1H), 1.28 (d, J=6.4 Hz, 9H), 1.19 (s, 9H). LCMS: ESI-MS: m/z=781.3 [M+H]⁺.

Step B: ((1S,3S,5S)-3-(6-Chloro-9H-purin-9-yl)-1-(hydroxymethyl)-2-methylene-5-(pivaloyloxy)cyclopentyl)methyl pivalate. To a solution of ((1S,3S,5S)-1-((bis(4-methoxyphenyl)(phenyl)methoxy)methyl)-3-(6-chloro-9H-purin-9-yl)-2-methylene-5-(pivaloyloxy)cyclopentyl)methyl pivalate (0.736 g, 941.98 mol) in DCM (20 mL) was added TFA (770.00 mg, 6.75 mmol, 0.5 mL) and Et₃SiH (1.46 g, 12.52 mmol, 2 mL) at 0° C. The mixture was stirred at 0° C. for 0.5 hr. The mixture was quenched with pyridine (1 mL) and the reaction mixture was concentrated at low pressure. The residue was purified by flash silica gel chromatography (ISCO®; 4 g SepaFlash® Silica Flash Column, Eluent of 10˜45% Ethyl acetate/Petroleum ether gradient @ 22 mL/min) to give ((1S,3S,5S)-3-(6-chloro-9H-purin-9-yl)-1-(hydroxymethyl)-2-methylene-5-(pivaloyloxy)cyclopentyl)methyl pivalate (0.3 g, 626.35 mol, 66.49% yield, 100% purity) as white foam. ¹H NMR (400 MHz, CDCl₃) δ=8.74 (s, 1H), 8.23 (s, 1H), 5.75-5.65 (m, 1H), 5.49 (d, J=3.8 Hz, 1H), 5.38 (d, J=2.5 Hz, 1H), 4.84 (d, J=2.0 Hz, 1H), 4.56-4.47 (m, 1H), 4.46-4.38 (m, 1H), 3.82-3.60 (m, 2H), 2.88 (ddd, J=5.0, 11.0, 13.9 Hz, 1H), 2.41 (ddd, J=1.5, 8.0, 13.9 Hz, 1H), 1.27 (s, 9H), 1.24 (s, 9H). LCMS: ESI-MS: m/z=479.1 [M+H]⁺.

Step C: ((1R,3S,5S)-3-(6-Chloro-9H-purin-9-yl)-2-methylene-5-(pivaloyloxy)-1-((((trifluoromethyl)sulfonyl)oxy)methyl)cyclopentyl)methyl pivalate. To a solution of ((1S,3S,5S)-3-(6-chloro-9H-purin-9-yl)-1-(hydroxymethyl)-2-methylene-5-(pivaloyloxy)cyclopentyl)methyl pivalate (0.272 g, 567.89 mol) in DCM (3 mL) was added Tf₂O (240.33 mg, 851.83 mol, 140.55 μL) and pyridine (224.60 mg, 2.84 mmol, 229.18 μL) at 0° C. The mixture was stirred at 0° C. for 2 hr. The mixture was diluted with DCM (5 mL) and washed with NaHCO₃(5 mL*2). The organic layer was dried over anhydrous Na₂SO₄, filtered and concentrated under reduced pressure. The residue was purified by flash silica gel chromatography (ISCO®; 4 g SepaFlash® Silica Flash Column, Eluent of 3˜19% Ethyl acetate/Petroleum ether gradient @ 22 mL/min) to give ((1R,3S,5S)-3-(6-chloro-9H-purin-9-yl)-2-methylene-5-(pivaloyloxy)-1-((((trifluoromethyl)sulfonyl)oxy)methyl)cyclopentyl)methyl pivalate (0.235 g, 384.60 umol, 67.72% yield) as a colorless oil. LCMS: ESI-MS: m/z=611.1 [M+H]⁺.

Step D: ((1S,3S,5S′)-3-(6-Chloro-9H-purin-9-yl)-1-(fluoromethyl)-2-methylene-5-(pivaloyloxy)cyclopentyl)methyl pivalate. To a solution of ((1R,3S,5S)-3-(6-chloro-9H-purin-9-yl)-2-methylene-5-(pivaloyloxy)-1-((((trifluoromethyl)sulfonyl)oxy)methyl)cyclopentyl)methyl pivalate (0.235 g, 384.60 mol) in THF (2 mL) was added TBAF (1 M, 1.15 mL). The mixture was stirred at 25° C. for 12 hr. The mixture was diluted with EA (5 mL) and washed with saturated NH₄Cl solution (3 mL). The combined organic layer was dried over anhydrous Na₂SO₄, filtered and concentrated under reduced pressure. The residue was purified by flash silica gel chromatography (ISCO®; 4 g SepaFlash® Silica Flash Column, Eluent of 5˜20% Ethyl acetate/Petroleum ether gradient @ 22 mL/min). The product was analyzed with SFC (ES5716-281-P1_G2) and further separated by SFC (column: OJ (250 mm*30 mm, 5 um); mobile phase: [0.1% NH₃H₂O ETOH]; B %: 20%-20%, min) to give (RT=1.492 min) to give ((1S,3S,5S)-3-(6-chloro-9H-purin-9-yl)-1-(fluoromethyl)-2-methylene-5-(pivaloyloxy)cyclopentyl)methyl pivalate (0.083 g, 150.14 mol, 39.04% yield, 87% purity) as a white foam. ¹H NMR (400 MHz, CDCl₃) δ=8.77-8.72 (m, 1H), 8.22 (s, 1H), 5.75-5.62 (m, 1H), 5.51 (d, J=3.1 Hz, 1H), 5.39-5.22 (m, 1H), 4.88 (s, 1H), 4.72-4.64 (m, 1H), 4.61-4.51 (m, 1H), 4.42 (s, 2H), 2.92-2.81 (m, 1H), 2.42 (dd, J=7.8, 13.8 Hz, 1H), 1.26 (d, J=10.8 Hz, 18H). LCMS: ESI-MS: m/z=481.1 [M+H]⁺.

Step F: ((1S,3S,5S)-3-(6-Amino-9H-purin-9-yl)-1-(fluoromethyl)-2-methylene-5-(pivaloyloxy)cyclopentyl)methyl pivalate. To a solution of ((1S,3S,5S)-3-(6-chloro-9H-purin-9-yl)-1-(fluoromethyl)-2-methylene-5-(pivaloyloxy)cyclopentyl)methyl pivalate (0.083 g, 172.57 mol) in THF (1 mL) was added NH₃ in THF (88.17 mg, 5.18 mmol, 7 M, 15 mL). The mixture was stirred at 25° C. for 12 hr. The reaction mixture was concentrated at low pressure to give ((1S,3S,5S)-3-(6-amino-9H-purin-9-yl)-1-(fluoromethyl)-2-methylene-5-(pivaloyloxy)cyclopentyl)methyl pivalate (0.053 g, 114.84 mol, 66.54% yield) as a white solid. ¹H NMR (400 MHz, CDCl₃) δ=8.32 (s, 1H), 7.84 (s, 1H), 5.67-5.56 (m, 1H), 5.69-5.55 (m, 2H), 5.47 (d, J=3.5 Hz, 1H), 5.32-5.22 (m, 2H), 4.88 (s, 1H), 4.68-4.57 (m, 1H), 4.44-4.29 (m, 2H), 2.78 (ddd, J=5.2, 11.1, 14.1 Hz, 1H), 2.36 (dd, J=8.2, 13.9 Hz, 1H), 1.23 (d, J=6.6 Hz, 18H). LCMS: ESI-MS: m/z=462.3 [M+H]⁺.

Step F: (1S,2S,4S)-4-(6-Amino-9H-purin-9-yl)-2-(fluoromethyl)-2-(hydroxymethyl)-3-methylenecyclopentan-1-ol. To a solution of ((1S,3S,5S)-3-(6-amino-9H-purin-9-yl)-1-(fluoromethyl)-2-methylene-5-(pivaloyloxy)cyclopentyl)methyl pivalate (0.053 g, 114.84 mol) in MeOH (0.5 mL) was added CH₃₀Na (18.61 mg, 344.51 mol). The mixture was stirred at 85° C. for 12 h. The solvent was removed at low pressure. The residue was purified by column chromatography (SiO₂, DCM/MeOH=30/1 to 15/1) and further purified by Prep-HPLC (column: Phenomenex Kinetex XB-C18 150 mm*30 mm, 5 μm; mobile phase: [water (10 mM NH₄HCO₃)-ACN]; B %: 1%-28%, 8 min) to give (1S,2S,4S)-4-(6-amino-9H-purin-9-yl)-2-(fluoromethyl)-2-(hydroxymethyl)-3-methylenecyclopentan-1-ol (0.023 g, 78.42 mol, 68.29% yield, 100% purity) as a white solid. ¹H NMR (400 MHz, CD₃OD) δ=8.22 (s, 1H), 8.16 (s, 1H), 5.73 (t, J=9.0 Hz, 1H), 5.25 (s, 1H), 4.72-4.55 (m, 1H), 4.71-4.55 (m, 1H), 4.53-4.37 (m, 2H), 3.85 (s, 2H), 2.73-2.55 (m, 1H), 2.40-2.21 (m, 1H). LCMS: ESI-MS: m/z=293.9 [M+H]⁺.

### Example 14: (1S,2S,4S)-4-(6-Amino-9H-purin-9-yl)-2-(chloromethyl)-2-(hydroxymethyl)-3-methylenecyclopentan-1-ol

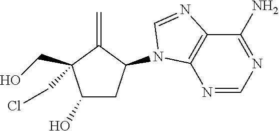 [SMILES: C=C1[C@@H](n2cnc3c(N)ncnc32)C[C@H](O)[C@]1(CO)CCl]

Step A: N,N-Di-BOC-((1S,3S,5S)-3-(6-amino-9H-purin-9-yl)-1-((bis(4-methoxyphenyl)(phenyl)methoxy)methyl)-2-methylene-5-(pivaloyloxy)cyclopentyl)methyl pivalate. To a solution of ((1S,3R,5S)-1-((bis(4-methoxyphenyl)(phenyl)methoxy)methyl)-3-hydroxy-2-methylene-5-(pivaloyloxy)cyclopentyl)methyl pivalate (Intermediate 7, 1.1 g, 1.71 mmol), tert-butyl N-tert-butoxycarbonyl-N-(9H-purin-6-yl)carbamate (1.72 g, 5.12 mmol) and PPh₃ (61.02 mg, 232.63 mol) in THF (15 mL) was added DIAD (1.03 g, 5.12 mmol, 995.08 μL) in THF (15 mL) dropwise slowly at 0° C. The mixture was stirred at 0° C. for 3 h and then at 25° C. for 12 h. The solvent was removed at low pressure. The residue was purified by flash silica gel chromatography (ISCO®; 40 g SepaFlash® Silica Flash Column, Eluent of 5˜26% Ethyl acetate/Petroleum ethergradient @ 35 mL/min). N,N-Di-BOC-((1S,3S,5S)-3-(6-amino-9H-purin-9-yl)-1-((bis(4-methoxyphenyl)(phenyl)methoxy)methyl)-2-methylene-5-(pivaloyloxy)cyclopentyl)methyl pivalate (0.895 g, 902.32 mol, 52.89% yield, 97% purity) was obtained as a colorless oil. ¹H NMR (400 MHz, CDCl₃) δ=8.87 (s, 1H), 8.14 (s, 1H), 7.43-7.37 (m, 2H), 7.33-7.29 (m, 2H), 7.33-7.29 (m, 1H), 7.28 (d, J=1.8 Hz, 3H), 7.24 (d, J=7.1 Hz, 1H), 6.83 (dd, J=1.4, 8.9 Hz, 4H), 5.67 (t, J=9.0 Hz, 1H), 5.53 (d, J=2.6 Hz, 1H), 5.46 (dd, J=3.4, 5.4 Hz, 1H), 4.81 (d, J=2.4 Hz, 1H), 4.63 (d, J=11.5 Hz, 1H), 4.41 (d, J=11.7 Hz, 1H), 3.80 (s, 6H), 3.42-3.32 (m, 2H), 2.80-2.67 (m, 1H), 2.39-2.27 (m, 1H), 1.45 (s, 18H), 1.19 (s, 9H), 1.06-1.00 (m, 9H). LCMS: ESI-MS: m/z=962.5 [M+H]⁺.

Step B: N,N-Di-BOC((1S,3S,5S)-3-(6-amino-9H-purin-9-yl)-1-(hydroxymethyl)-2-methylene-5-(pivaloyloxy)cyclopentyl)methyl pivalate. To a solution of N,N-Di-BOC-((1S,3S,5S)-3-(6-amino-9H-purin-9-yl)-1-((bis(4-methoxyphenyl)(phenyl)methoxy)methyl)-2-methylene-5-(pivaloyloxy)cyclopentyl)methyl pivalate (0.775 g, 805.50 mol) in DCM (5 mL) was added TFA (765.06 mg, 6.71 mmol, 496.79 μL) at 0° C. The mixture was stirred at 0° C. for 5 h. The mixture was quenched with saturated aq. NaHCO₃ solution (5 mL) and extracted with DCM (5 mL*2). The combined organic layers were dried over anhydrous Na₂SO₄, filtered and concentrated under reduced pressure. The residue was purified by flash silica gel chromatography (ISCO®; 12 g SepaFlash® Silica Flash Column, Eluent of 10˜45% Ethyl acetate/Petroleum ethergradient @ 35 mL/min). N,N-Di-BOC((1S,3S,5S)-3-(6-amino-9H-purin-9-yl)-1-(hydroxymethyl)-2-methylene-5-(pivaloyloxy)cyclopentyl)methyl pivalate (0.19 g, 287.98 mol, 35.75% yield) was obtained as a white foam. ¹H NMR (400 MHz, CDCl₃) δ=8.84 (s, 1H), 8.16 (s, 1H), 5.71 (dd, J=8.2, 10.9 Hz, 1H), 5.50 (d, J=3.8 Hz, 1H), 5.35 (d, J=2.8 Hz, 1H), 4.81 (d, J=2.3 Hz, 1H), 4.57-4.48 (m, 1H), 4.46-4.39 (m, 1H), 3.82-3.74 (m, 1H), 3.70-3.63 (m, 1H), 2.88 (ddd, J=5.1, 11.0, 13.9 Hz, 1H), 2.41 (dd, J=8.5, 13.3 Hz, 1H), 1.47 (s, 18H), 1.28 (s, 9H), 1.25 (s, 9H). LCMS: ESI-MS: m/z=660.3 [M+H]⁺.

Step C: N,N-Di-BOC-((1R,3S,5S)-3-(6-amino-9H-purin-9-yl)-2-methylene-5-(pivaloyloxy)-1-((((trifluoromethyl)sulfonyl)oxy)methyl)cyclopentyl)methyl pivalate. To a solution of N,N-Di-BOC((1S,3S,5S)-3-(6-amino-9H-purin-9-yl)-1-(hydroxymethyl)-2-methylene-5-(pivaloyloxy)cyclopentyl)methyl pivalate (0.220 g, 333.45 mol) in DCM (1.1 mL) was added pyridine (263.76 mg, 3.33 mmol, 269.14 μL) and Tf₂O (188.16 mg, 666.90 mol, 110.03 μL). The mixture was stirred at 0° C. for 2 hr. The mixture was diluted with DCM (2 mL) and washed with NaHCO₃(3 mL*2). The organic layer was dried over anhydrous Na₂SO₄, filtered and concentrated under reduced pressure. The residue was purified by flash silica gel chromatography (ISCO®; 4 g SepaFlash® Silica Flash Column, Eluent of 5˜19% Ethyl acetate/Petroleum ethergradient @ 20 mL/min). N,N-Di-BOC-((1R,3S,5S)-3-(6-amino-9H-purin-9-yl)-2-methylene-5-(pivaloyloxy)-1-((((trifluoromethyl)sulfonyl)oxy)methyl)cyclopentyl)methyl pivalate (0.175 g, 221.01 □mol, 66.28% yield) was obtained as light oil. LCMS: ESI-MS: m/z=792.5 [M+H]⁺.

Step D: N,N-Di-BOC-((1S,3S,5S)-3-(6-amino-9H-purin-9-yl)-1-(chloromethyl)-2-methylene-5-(pivaloyloxy)cyclopentyl)methyl pivalate. To a solution of N,N-Di-BOC-((1R,3S,5S)-3-(6-amino-9H-purin-9-yl)-2-methylene-5-(pivaloyloxy)-1-((((trifluoromethyl)sulfonyl)oxy)methyl)cyclopentyl)methyl pivalate (0.175 g, 221.01 mol) in DMF (1.5 mL) was added LiCl (37.47 mg, 884.03 mol, 18.10 μL). The mixture was stirred at 40° C. for 2 h. The mixture was diluted with EA (5 mL) and washed with H₂O (5 mL*2). The organic layer was dried over Na₂SO₄, filtered and concentrated under reduced pressure. The residue was purified by flash silica gel chromatography (ISCO®; 4 g SepaFlash® Silica Flash Column, Eluent of 3˜30% Ethyl acetate/Petroleum ethergradient @mL/min). N,N-Di-BOC-((1S,3S,5S)-3-(6-amino-9H-purin-9-yl)-1-(chloromethyl)-2-methylene-5-(pivaloyloxy)cyclopentyl)methyl pivalate (0.06 g, 88.47 umol, 40.03% yield) was obtained as light oil. ¹H NMR (400 MHz, CDCl₃) δ=8.82 (s, 1H), 8.16 (s, 1H), 5.76-5.64 (m, 1H), 5.39 (d, J=4.2 Hz, 1H), 5.31 (s, 1H), 4.88 (s, 1H), 4.60 (d, J=11.2 Hz, 1H), 4.42 (d, J=11.2 Hz, 1H), 3.90-3.71 (m, 2H), 2.87-2.72 (m, 1H), 2.52-2.33 (m, 1H), 1.53-1.44 (m, 18H), 1.33-1.28 (m, 9H), 1.22 (s, 9H). LCMS: ESI-MS: m/z=700.5 [M+Na]⁺.

Step F: ((1S,3S,5S)-3-(6-Amino-9H-purin-9-yl)-1-(chloromethyl)-2-methylene-5-(pivaloyloxy)cyclopentyl)methyl pivalate. N,N-Di-BOC-((1S,3S,5S)-3-(6-amino-9H-purin-9-yl)-1-(chloromethyl)-2-methylene-5-(pivaloyloxy)cyclopentyl)methyl pivalate (0.045 g, 66.35 mol) was dissolved in a solution of TFA (154.00 mg, 1.35 mmol, 0.1 mL) in DCM (0.5 mL), and the mixture was stirred at 25° C. for 3 h. The mixture was quenched with saturated NaHCO₃ solution (3 mL) and extracted with DCM (5 mL*2). The combined organic layers were dried over anhydrous Na₂SO₄, filtered and concentrated under reduced pressure. The residue was purified by flash silica gel chromatography (ISCO®; 4.0 g SepaFlash® Silica Flash Column, Eluent of 0˜6% MeOH/DCMgradient @ 20 mL/min). ((1S,3S,5S)-3-(6-amino-9H-purin-9-yl)-1-(chloromethyl)-2-methylene-5-(pivaloyloxy)cyclopentyl)methyl pivalate (0.025 g, 52.30 mol, 78.83% yield) was obtained as a white solid. ¹H NMR (400 MHz, CDCl₃) δ=8.30 (s, 1H), 7.87 (s, 1H), 5.70 (s, 2H), 5.62 (dd, J=8.3, 11.1 Hz, 1H), 5.36 (d, J=3.7 Hz, 1H), 5.31-5.27 (m, 1H), 4.91 (d, J=2.2 Hz, 1H), 4.57 (d, J=11.5 Hz, 1H), 4.40 (d, J=11.2 Hz, 1H), 3.91-3.68 (m, 2H), 2.72 (ddd, J=4.9, 11.5, 14.1 Hz, 1H), 2.39 (dd, J=7.7, 14.6 Hz, 1H), 1.28 (s, 9H), 1.21 (s, 9H). LCMS: ESI-MS: m/z=478.4 [M+H]⁺.

Step F: (1S,2S,4S)-4-(6-Amino-9H-purin-9-yl)-2-(chloromethyl)-2-(hydroxymethyl)-3-methylenecyclopentan-1-ol. To a solution of ((1S,3S,5S)-3-(6-amino-9H-purin-9-yl)-1-(chloromethyl)-2-methylene-5-(pivaloyloxy)cyclopentyl)methyl pivalate (0.045 g, 94.15 mol) in MeOH (0.5 mL) was added CH₃₀Na (10.17 mg, 188.29 mol). The mixture was stirred at 25° C. for 12 hr. The solvent was removed at low pressure. The residue was purified by column chromatography (SiO₂, DCM/MeOH=30/1 to 20/1). The crude was further purified by Prep-HPLC (column: Waters Xbridge 150*25 5; mobile phase: [water (10 mM NH₄HCO₃)-ACN]; B %: 0%-30%, 6 min). (1S,2S,4S)-4-(6-amino-9H-purin-9-yl)-2-(chloromethyl)-2-(hydroxymethyl)-3-methylenecyclopentan-1-ol (0.0124 g, 39.63 mol, 42.10% yield, 99% purity) was obtained as a white solid. ¹H NMR (400 MHz, CD₃OD) δ=8.22 (s, 1H), 8.17 (s, 1H), 5.80-5.68 (m, 1H), 5.28 (d, J=2.8 Hz, 1H), 4.83 (d, J=2.3 Hz, 1H), 4.34 (d, J=3.3 Hz, 1H), 3.94-3.84 (m, 3H), 3.84-3.76 (m, 1H), 2.62 (ddd, J=4.5, 10.9, 13.2 Hz, 1H), 2.33 (dd, J=8.9, 12.2 Hz, 1H). LCMS: ESI-MS: m/z=309.9 [M+H]⁺.

### Example 15: (1S,3S,5S)-5-Hydroxy-1-(hydroxymethyl)-3-(5-methyl-2,4-dioxo-3,4-dihydropyrimidin-1(2H)-yl)-2-methylenecyclopentane-1-carbonitrile

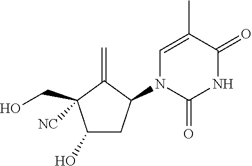 [SMILES: C=C1[C@@H](n2cc(C)c(=O)[nH]c2=O)C[C@H](O)[C@@]1(C#N)CO]

Step A: ((1R,3S,5S)-3-(3-Benzoyl-5-methyl-2,4-dioxo-3,4-dihydropyrimidin-1(2H)-yl)-1-((bis(4-methoxyphenyl)(phenyl)methoxy)methyl)-2-methylene-5-(pivaloyloxy)cyclopentyl)methyl pivalate. To a solution of ((1S,3R,5S)-1-((bis(4-methoxyphenyl)(phenyl)methoxy)methyl)-3-hydroxy-2-methylene-5-(pivaloyloxy)cyclopentyl)methyl pivalate (Intermediate 7, 120 mg, 186.11 mol) and 3-benzoyl-5-methylpyrimidine-2,4(1H,3H)-dione (Intermediate 6, 64.27 mg, 279.16 mol) in THF (3 mL) was added PPh₃ (122.03 mg, 465.27 mol) at 25° C. under N₂. After addition of DIAD (94.08 mg, 465.27 mol, 90.46 μL) in dropwise at 0° C., the reaction mixture was stirred at 25° C. for another 12 h. The solvent was removed in vacuum to give a yellow oil. The residue was purified by silica gel column chromatography (Petroleum ether/Ethyl acetate=10/1 to 3/1). ((1R,3S,5S)-3-(3-Benzoyl-5-methyl-2,4-dioxo-3,4-dihydropyrimidin-1(2H)-yl)-1-((bis(4-methoxyphenyl)(phenyl)methoxy)methyl)-2-methylene-5-(pivaloyloxy)cyclopentyl)methyl pivalate (100 mg, 61.44% yield, 98% purity) was obtained as a white foam. ¹H NMR (400 MHz, CDCl₃) δ=7.93-7.91 (m, 2H), 7.68-7.60 (m, 1H), 7.55-7.44 (m, 2H), 7.49-7.37 (m, 2H), 7.31-7.26 (m, 7H), 7.15 (d, J=1.0 Hz, 1H), 6.84 (d, J=8.8 Hz, 4H), 5.78-5.75 (m, 1H), 5.56 (br, d, J=4.0 Hz, 1H), 5.12 (dd, J=2.3, 19.6 Hz, 2H), 4.11-4.02 (m, 2H), 3.80-3.77 (m, 6H), 3.60-3.58 (m, 1H), 3.29 (d, J=9.0 Hz, 1H), 2.63 (s, 3H), 2.45-2.43 (m, 1H), 2.24 (br, dd, J=8.8, 13.1 Hz, 1H), 1.13 (s, 9H), 1.03 (s, 9H). LCMS: ESI-MS: m/z=879.3 [M+Na]⁺.

Step B: 1-((1S,3S,4S)-3-((Bis(4-methoxyphenyl)(phenyl)methoxy)methyl)-4-hydroxy-3-(hydroxymethyl)-2-methylenecyclopentyl)-5-methylpyrimidine-2,4(1H,3H)-dione. To a solution of ((1R,3S,5S)-3-(3-benzoyl-5-methyl-2,4-dioxo-3,4-dihydropyrimidin-1(2H)-yl)-1-((bis(4-methoxyphenyl)(phenyl)methoxy)methyl)-2-methylene-5-(pivaloyloxy)cyclopentyl)methyl pivalate (4.4 g, 5.13 mmol) in MeOH (90 mL) was added NaOH solution (4 M, 44.00 mL) at 25° C. under N₂. The reaction mixture was stirred at 60° C. for 4 h. The reaction mixture was adjusted to pH ˜8 with 4 M HCl solution. The resulting mixture was extracted with EA (70 mL*3). The combined organic phase was washed with brine (50 mL), dried over anhydrous Na₂SO₄ and concentrated in vacuum to give a yellow oil. The residue was purified by silica gel column chromatography (DCM/MeOH=50/1 to 20/1). 1-((1S,3S,4S)-3-((bis(4-methoxyphenyl)(phenyl)methoxy)methyl)-4-hydroxy-3-(hydroxymethyl)-2-methylenecyclopentyl)-5-methylpyrimidine-2,4(1H,3H)-dione (3 g, 5.13 mmol, 99.94% yield) was obtained as a white foam. LCMS: ESI-MS: m/z=607.1 [M+Na]⁺.

Step C: 1-((1S,3R,4S)-3-((Bis(4-methoxyphenyl)(phenyl)methoxy)methyl)-3-(((tert-butyldimethylsilyl)oxy)methyl)-4-hydroxy-2-methylenecyclopentyl)-5-methylpyrimidine-2,4(1H,3H)-dione. To a solution of 1-((1S,3S,4S)-3-((bis(4-methoxyphenyl)(phenyl)methoxy)methyl)-4-hydroxy-3-(hydroxymethyl)-2-methylenecyclopentyl)-5-methylpyrimidine-2,4(1H,3H)-dione (3 g, 5.13 mmol) and 1H-imidazole (1.05 g, 15.39 mmol) in DMF (9 mL) was added TBSCl (928.06 mg, 6.16 mmol, 754.52 μL) at 20° C. under N₂. The reaction mixture was stirred at 20° C. for 12 h. The reaction was poured into water (50 mL), and extracted with EA (70 mL*3). The combined organic phase was washed with brine (50 mL), dried over anhydrous Na₂SO₄, and concentrated in vacuum to give a yellow oil. The residue was purified by silica gel column chromatography (Petroleum ether/Ethyl acetate=3/1 to 1/1). 1-((1S,3R,4S)-3-((bis(4-methoxyphenyl)(phenyl)methoxy)methyl)-3-(((tert-butyldimethylsilyl)oxy)methyl)-4-hydroxy-2-methylenecyclopentyl)-5-methylpyrimidine-2,4(1H,3H)-dione (3.3 g, 4.58 mmol, 89.26% yield, 97% purity) was obtained as white foam. ¹H NMR (400 MHz, CDCl₃) δ=8.37 (br, s, 1H), 7.44-7.43 (m, 2H), 7.36-7.32 (m, 7H), 7.12 (d, J=1.2 Hz, 1H), 6.91-6.88 (m, 4H), 5.93-5.89 (m, 1H), 4.93 (m, 2H), 4.58 (br d, J=2.5 Hz, 1H), 3.86 (s, 6H), 3.82 (d, J=10.2 Hz, 1H), 3.68 (d, J=8.8 Hz, 1H), 3.54 (d, J=10.2 Hz, 1H), 3.34 (s, 1H), 3.18 (d, J=8.6 Hz, 1H), 2.38-2.31 (m, 2H), 1.50 (s, 3H), 0.87 (s, 9H), 0.06 (s, 3H), 0.00 (s, 3H). LCMS: ESI-MS: m/z=721.3 [M+Na]⁺.

Step D: 1-((1S,3R,4S)-4-(bis(4-methoxyphenyl)(phenyl)methoxy)-3-((bis(4-methoxyphenyl)(phenyl)methoxy)methyl)-3-(((tert-butyldimethylsilyl)oxy)methyl)-2-methylenecyclopentyl)-5-methylpyrimidine-2,4(1H,3H)-dione. To a solution of 1-((1S,3R,4S)-3-((bis(4-methoxyphenyl)(phenyl)methoxy)methyl)-3-(((tert-butyldimethylsilyl)oxy)methyl)-4-hydroxy-2-methylenecyclopentyl)-5-methylpyrimidine-2,4(1H,3H)-dione (3.3 g, 4.72 mmol) and collidine (1.43 g, 11.80 mmol, 1.56 mL) in DCE (60 mL) was added AgNO₃ (2.01 g, 11.80 mmol, 1.99 mL) at 20° C. under N₂. After the addition of 4,4′-dimethoxytrityl chloride (DMTrCl) (3.20 g, 9.44 mmol), the reaction mixture was stirred at 60° C. for 4 h. The reaction mixture was poured into water (50 mL) and the inorganic material was filtered off. The filtrate was separated and the water phase was extracted with DCM (70 mL*2). The combined organic phase was washed with brine (50 mL) dried over anhydrous Na₂SO₄ and concentrated in vacuum to give a yellow oil. The residue was purified by silica gel column chromatography (Petroleum ether/Ethyl acetate=5/1 to 2/1). 1-((1S,3R,4S)-4-(bis(4-methoxyphenyl)(phenyl)methoxy)-3-((bis(4-methoxyphenyl)(phenyl)methoxy)methyl)-3-(((tert-butyldimethylsilyl)oxy)methyl)-2-methylenecyclopentyl)-5-methylpyrimidine-2,4(1H,3H)-dione (3.3 g, 3.26 mmol, 69.10% yield, 99% purity) was obtained as a pale yellow foam. ¹H NMR (400 MHz, CDCl₃) δ=8.12 (s, 1H), 7.50-7.03 (m, 18H), 6.74-6.60 (m, 8H), 6.42 (s, 1H), 5.68 (br s, 1H), 4.93 (br s, 1H), 4.66 (m, 1H), 4.09-4.02 (m, 2H), 3.99 (br d, J=9.9 Hz, 1H), 3.86-3.83 (m, 1H), 3.76-3.69 (m, 12H), 3.43-3.41 (m, 1H), 2.72 (br, d, J=8.8 Hz, 1H), 1.90-1.84 (m, 1H), 1.45 (s, 3H), 0.83 (s, 9H), 0.01 (m, 6H). LCMS: ESI-MS: m/z=1024.8 [M+Na]⁺.

Step E: 1-((1S,3S,4S)-4-(Bis(4-methoxyphenyl)(phenyl)methoxy)-3-((bis(4-methoxyphenyl)(phenyl)methoxy)methyl)-3-(hydroxymethyl)-2-methylenecyclopentyl)-5-methylpyrimidine-2,4(1H,3H)-dione. A solution of 1-((1S,3R,4S)-4-(bis(4-methoxyphenyl)(phenyl)methoxy)-3-((bis(4-methoxyphenyl)(phenyl)methoxy)methyl)-3-(((tert-butyldimethylsilyl)oxy)methyl)-2-methylenecyclopentyl)-5-methylpyrimidine-2,4(1H,3H)-dione (700 mg, 699.11 mol) in TBAF (1 M, 20 mL) and AcOH (882.00 mg, 14.69 mmol, 840 μL) was stirred at 50° C. for 12 h. TEA (5 mL) was added to the reaction mixture. The resulting mixture was concentrated in vacuum to give a yellow oil. The residue was purified by silica gel column chromatography (Petroleum ether:Ethyl acetate=1:1 to 0:1). 1-((1S,3S,4S)-4-(bis(4-methoxyphenyl)(phenyl)methoxy)-3-((bis(4-methoxyphenyl)(phenyl)methoxy)methyl)-3-(hydroxymethyl)-2-methylenecyclopentyl)-5-methylpyrimidine-2,4(1H,3H)-dione (1 g, 2 batches, 1.08 mmol, 77.40% yield, 96% purity) was obtained as pale yellow foam. ¹H NMR (400 MHz, CDCl₃) δ=11.21 (br, s, 1H), 7.41 (m, 2H), 7.29-7.19 (m, 12H), 7.07 (m, 4H), 6.88-6.78 (m, 8H), 6.44 (s, 1H), 5.40 (m, 1H), 4.83 (d, J=2.3 Hz, 1H), 4.68 (m, 2H), 4.00-3.96 (m, 2H), 3.92-3.84 (m, 2H), 3.71 (s, 12H), 2.86 (br d, J=9.0 Hz, 1H), 1.80-1.75 (m, 1H), 1.43 (s, 3H).

LCMS: ESI-MS: m/z=909.8 [M+Na]⁺.

Step F: (1S,3S,5S)-5-(Bis(4-methoxyphenyl)(phenyl)methoxy)-1-((bis(4-methoxyphenyl)(phenyl)methoxy)methyl)-3-(5-methyl-2,4-dioxo-3,4-dihydropyrimidin-1(2H)-yl)-2-methylenecyclopentane-1-carbaldehyde. To a solution of 1-((1S,3S,4S)-4-(bis(4-methoxyphenyl)(phenyl)methoxy)-3-((bis(4-methoxyphenyl)(phenyl)methoxy)methyl)-3-(hydroxymethyl)-2-methylenecyclopentyl)-5-methylpyrimidine-2,4(1H,3H)-dione (100 mg, 112.74 mol) in DCM (2 mL) and TEA (34.22 mg, 338.21 mol, 47.07 μL) was added DMP (143.45 mg, 338.21 mol, 104.71 μL) in one portion at 20° C. The reaction mixture was stirred at 20° C. for 4 h. The reaction mixture was diluted with DCM (20 mL), then poured into saturated Na₂S₂O₃ solution (5 mL) and saturated NaHCO₃ solution (5 mL) and stirred for 5 min. The organic phase was separated and washed with brine (5 mL), and dried over anhydrous Na₂SO₄. The solvent was removed in vacuum to give a pale yellow foam. (1S,3S,5S)-5-(Bis(4-methoxyphenyl)(phenyl)methoxy)-1-((bis(4-methoxyphenyl)(phenyl)methoxy)methyl)-3-(5-methyl-2,4-dioxo-3,4-dihydropyrimidin-1(2H)-yl)-2-methylenecyclopentane-1-carbaldehyde (120 mg, crude) was obtained as a pale yellow foam used into the next step directly without further purification. LCMS: ESI-MS: m/z=907.4 [M+Na]⁺.

Step G: (E)-5-(Bis(4-methoxyphenyl)(phenyl)methoxy)-1-((bis(4-methoxyphenyl)(phenyl)methoxy)methyl)-3-(5-methyl-2,4-dioxo-3,4-dihydropyrimidin-1(2H)-yl)-2-methylenecyclopentane-1-carbaldehyde oxime. To a solution of (1S,3S,5S)-5-(bis(4-methoxyphenyl)(phenyl)methoxy)-1-((bis(4-methoxyphenyl)(phenyl)methoxy)methyl)-3-(5-methyl-2,4-dioxo-3,4-dihydropyrimidin-1(2H)-yl)-2-methylenecyclopentane-1-carbaldehyde (110 mg, 124.29 mol) in pyridine (1 mL) was added NH₂OH HCl (17.27 mg, 248.59 mol) in one portion at 20° C. under N₂. The reaction mixture was stirred at 20° C. for 12 h. The solvent was removed at low pressure and the residue was diluted with EA (20 mL), washed with water (5 mL), and brine (5 mL). The organic phase was dried over anhydrous Na₂SO₄, and concentrated in vacuum to give a yellow foam. (E)-5-(Bis(4-methoxyphenyl)(phenyl)methoxy)-1-((bis(4-methoxyphenyl)(phenyl)methoxy)methyl)-3-(5-methyl-2,4-dioxo-3,4-dihydropyrimidin-1(2H)-yl)-2-methylenecyclopentane-1-carbaldehyde oxime (85 mg, 94.44 mol, 75.98% yield) was obtained as a pale yellow foam which was used into the next step directly without further purification. LCMS: ESI-MS: m/z=922.8 [M+Na]⁺.

Step H: (1S,3S,5S)-5-(Bis(4-methoxyphenyl)(phenyl)methoxy)-1-((bis(4-methoxyphenyl)(phenyl)methoxy)methyl)-3-(5-methyl-2,4-dioxo-3,4-dihydropyrimidin-1(2H)-yl)-2-methylenecyclopentane-1-carbonitrile. To a solution of (E)-5-(bis(4-methoxyphenyl)(phenyl)methoxy)-1-((bis(4-methoxyphenyl)(phenyl)methoxy)methyl)-3-(5-methyl-2,4-dioxo-3,4-dihydropyrimidin-1(2H)-yl)-2-methylenecyclopentane-1-carbaldehyde oxime (800 mg, 888.87 mol) in ACN (10 mL) was added CDI (288.26 mg, 1.78 mmol) in one portion at 20° C. under N₂. The reaction mixture was stirred at 30° C. for 36 h. The reaction mixture was diluted with EA (100 mL), and the resulting solution was washed with water (30 mL), brine (30 mL), and dried over anhydrous Na₂SO₄. The resulting solution was concentrated in vacuum to give a yellow oil. The residue was purified by silica gel column chromatography (Petroleum ether/Ethyl acetate=2/1 to 1/1). (1S,3S,5S)-5-(bis(4-methoxyphenyl)(phenyl)methoxy)-1-((bis(4-methoxyphenyl)(phenyl)methoxy)methyl)-3-(5-methyl-2,4-dioxo-3,4-dihydropyrimidin-1(2H)-yl)-2-methylenecyclopentane-1-carbonitrile (500 mg, 63.14% yield, 99% purity) was obtained as a pale yellow foam. LCMS: ESI-MS: m/z=904.5 [M+Na]⁺.

Step I: (1S,3S,5S)-5-Hydroxy-1-(hydroxymethyl)-3-(5-methyl-2.4-dioxo-3,4-dihydropyrimidin-1(2H)-yl)-2-methylenecyclopentane-1-carbonitrile. To a solution of (1S,3S,5S)-5-(bis(4-methoxyphenyl)(phenyl)methoxy)-1-((bis(4-methoxyphenyl)(phenyl)methoxy)methyl)-3-(5-methyl-2,4-dioxo-3,4-dihydropyrimidin-1(2H)-yl)-2-methylenecyclopentane-1-carbonitrile (400 mg, 453.51 mol) in DCM (4 mL) was added TFA (616.00 mg, 5.40 mmol, 0.4 mL) at 20° C., and stirred at 20° C. for 2 h. The solvent was removed in vacuum. The residue was dissolved in acetonitrile (20 mL), and the solution was adjusted pH to 8 with saturated NaHCO₃ solution. The resulting mixture was concentrated in vacuum to give 600 mg of crude product as a yellow oil. 900 mg of crude product was purified by silica gel column chromatography (DCM/MeOH=15/1 to 10/1). (1S,3S,5S)-5-Hydroxy-1-(hydroxymethyl)-3-(5-methyl-2,4-dioxo-3,4-dihydropyrimidin-1(2H)-yl)-2-methylenecyclopentane-1-carbonitrile (152.2 mg, 97.75% purity, 80.7% yield) was obtained as white solid. ¹H NMR (400 MHz, CD₃OD) δ=7.34 (s, 1H), 5.64-5.58 (m, 2H), 5.23 (s, 1H), 4.43 (d, J=3.8 Hz, 1H), 3.87-3.84 (m, 1H), 3.79-3.86 (m, 1H), 2.34-2.29 (m, 1H), 2.28-2.18 (m, 1H), 1.86 (s, 3H). LCMS: ESI-MS: m/z=278.1 [M+H]⁺.

### Example 16: (1S,3S,5S)-3-(4-Amino-2-oxopyrimidin-1(2H)-yl)-5-hydroxy-1-(hydroxymethyl)-2-methylenecyclopentane-1-carbonitrile

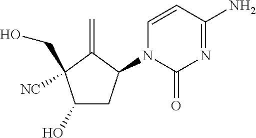 [SMILES: C=C1[C@@H](n2ccc(N)nc2=O)C[C@H](O)[C@@]1(C#N)CO]

Step A: ((1R,3S,5S)-3-(3-Benzoyl-2,4-dioxo-3,4-dihydropyrimidin-1(2H)-yl)-1-((bis(4-methoxyphenyl)(phenyl)methoxy)methyl)-2-methylene-5-(pivaloyloxy)cyclopentyl)methyl pivalate. To a solution of ((1S,3R,5S)-1-((bis(4-methoxyphenyl)(phenyl)methoxy)methyl)-3-hydroxy-2-methylene-5-(pivaloyloxy)cyclopentyl)methyl pivalate (Intermediate 7, 3 g, 4.65 mmol), PPh₃ (2.44 g, 9.31 mmol) and 3-benzoyl-1H-pyrimidine-2,4-dione (1.51 g, 6.98 mmol) in THF (60 mL) was added DIAD (1.88 g, 9.31 mmol, 1.81 mL) at 0° C. The mixture was stirred at 25° C. for 12 hr. The solvent was removed under reduced pressure. The residue was purified by flash silica gel chromatography (ISCO®; 4 g SepaFlash® Silica Flash Column, Eluent of 6˜23% Ethyl acetate/Petroleum ethergradient @ 20 mL/min). ((1R,3S,5S)-3-(3-Benzoyl-2,4-dioxo-3,4-dihydropyrimidin-1(2H)-yl)-1-((bis(4-methoxyphenyl)(phenyl)methoxy)methyl)-2-methylene-5-(pivaloyloxy)cyclopentyl)methyl pivalate (3.3 g, 3.91 mmol, 84.14% yield) was obtained as a white foam. ¹H NMR (400 MHz, CDCl₃) δ=7.99-7.90 (m, 2H), 7.69-7.63 (m, 1H), 7.57-7.49 (m, 2H), 7.43-7.36 (m, 2H), 7.27 (s, 7H), 7.06 (d, J=8.2 Hz, 1H), 6.86 (d, J=8.8 Hz, 4H), 5.70 (t, J=9.0 Hz, 1H), 5.48 (d, J=8.0 Hz, 2H), 5.23 (d, J=2.2 Hz, 1H), 5.11 (d, J=2.0 Hz, 1H), 4.27 (d, J=11.0 Hz, 1H), 4.11-4.05 (m, 1H), 3.82 (s, 6H), 3.47-3.37 (m, 2H), 2.25-2.18 (m, 1H), 2.13 (dd, J=4.8, 10.4 Hz, 1H), 1.16 (s, 9H), 1.09 (s, 9H). LCMS: ESI-MS: m/z=865.5 [M+Na]⁺.

Step B: 1-((1S,3S,4S)-3-((bis(4-methoxyphenyl)(phenyl)methoxy)methyl)-4-hydroxy-3-(hydroxymethyl)-2-methylenecyclopentyl)pyrimidine-2,4(1H,3H)-dione. To a solution of ((1R,3S,5S)-3-(3-benzoyl-2,4-dioxo-3,4-dihydropyrimidin-1(2H)-yl)-1-((bis(4-methoxyphenyl)(phenyl)methoxy)methyl)-2-methylene-5-(pivaloyloxy)cyclopentyl)methyl pivalate (1.73 g, 2.05 mmol) in MeOH (40 mL) was added NaOH (4 M, 7.70 mL). The mixture was stirred at 60° C. for 12 hr. The reaction mixture was neutralized with HCl solution (1 M) and extracted with EA (20 mL*3). The combined organic layers were dried over anhydrous Na₂SO₄, filtered and concentrated under reduced pressure. The residue was purified by flash silica gel chromatography (ISCO®; 4 g SepaFlash® Silica Flash Column, Eluent of 0˜2% MeOH/DCM gradient @ 20 mL/min). 1-((1S,3S,4S)-3-((Bis(4-methoxyphenyl)(phenyl)methoxy)methyl)-4-hydroxy-3-(hydroxymethyl)-2-methylenecyclopentyl)pyrimidine-2,4(1H,3H)-dione (0.793 g, 1.39 mmol, 67.71% yield) was obtained as a white foam. ¹H NMR (400 MHz, CDCl₃) δ=8.95 (s, 1H), 7.40 (d, J=7.6 Hz, 2H), 7.35-7.28 (m, 6H), 7.27-7.21 (m, 1H), 6.89 (d, J=8.0 Hz, 1H), 6.86 (d, J=8.6 Hz, 4H), 5.78-5.62 (m, 1H), 5.48 (d, J=8.0 Hz, 1H), 4.93 (d, J=2.6 Hz, 1H), 4.86 (d, J=2.0 Hz, 1H), 4.56 (d, J=3.6 Hz, 1H), 3.83-3.76 (m, 8H), 3.41 (d, J=9.2 Hz, 1H), 3.24 (d, J=9.2 Hz, 1H), 2.25 (dd, J=8.8, 12.2 Hz, 1H), 1.96-1.80 (m, 1H). LCMS: ESI-MS: m/z=593.1 [M+Na]⁺.

Step C: 1-((1S,3R,4S)-3-((Bis(4-methoxyphenyl)(phenyl)methoxy)methyl)-3-(((tert-butyldimethylsilyl)oxy)methyl)-4-hydroxy-2-methylenecyclopentyl)pyrimidine-2,4(1H,3H)-dione. To a solution of 1-((1S,3S,4S)-3-((bis(4-methoxyphenyl)(phenyl)methoxy)methyl)-4-hydroxy-3-(hydroxymethyl)-2-methylenecyclopentyl)pyrimidine-2,4(1H,3H)-dione (0.793 g, 1.39 mmol) in DMF (1.8 mL) was added imidazole (283.82 mg, 4.17 mmol) and TBSCl (314.18 mg, 2.08 mmol, 255.43 μL) at 0° C. The mixture was stirred at 25° C. for 2 h. The mixture was diluted with EA (15 mL) and washed with H₂O (10 mL*2). The organic layer was dried over anhydrous Na₂SO₄, filtered and concentrated under reduced pressure. The residue was purified by flash silica gel chromatography (ISCO®; 4 g SepaFlash® Silica Flash Column, Eluent of 0˜1% MeOH/DCMgradient @ 20 mL/min). 1-((1S,3R,4S)-3-((Bis(4-methoxyphenyl)(phenyl)methoxy)methyl)-3-(((tert-butyldimethylsilyl)oxy)methyl)-4-hydroxy-2-methylenecyclopentyl)pyrimidine-2,4(1H,3H)-dione (0.825 g, 1.20 mmol, 86.68% yield) was obtained as a white foam. ¹H NMR (400 MHz, CDCl₃) δ=8.40 (s, 1H), 7.42-7.35 (m, 2H), 7.33-7.27 (m, 4H), 7.26-7.19 (m, 3H), 7.04 (d, J=8.0 Hz, 1H), 6.84 (d, J=8.8 Hz, 4H), 5.86-5.72 (m, 1H), 5.42 (dd, J=2.3, 8.0 Hz, 1H), 4.97 (d, J=2.8 Hz, 1H), 4.88 (d, J=2.2 Hz, 1H), 4.45-4.39 (m, 1H), 3.83-3.80 (m, 6H), 3.76 (d, J=10.0 Hz, 1H), 3.70-3.64 (m, 1H), 3.46 (d, J=8.8 Hz, 1H), 3.21 (dd, J=3.1, 5.4 Hz, 2H), 2.33-2.23 (m, 1H), 2.04-1.98 (m, 1H), 0.84 (s, 9H), 0.03 (s, 3H), −0.01 (s, 3H). LCMS: ESI-MS: m/z=707.2 [M+Na]⁺.

Step D: 1-((1S,3R,4S)-4-(Bis(4-methoxyphenyl)(phenyl)methoxy)-3-((bis(4-methoxyphenyl)(phenyl)methoxy)methyl)-3-(((tert-butyldimethylsilyl)oxy)methyl)-2-methylenecyclopentyl)pyrimidine-2,4(1H,3H)-dione. To a solution of 1-((1S,3R,4S)-3-((bis(4-methoxyphenyl)(phenyl)methoxy)methyl)-3-(((tert-butyldimethylsilyl)oxy)methyl)-4-hydroxy-2-methylenecyclopentyl)pyrimidine-2,4(1H,3H)-dione (0.1 g, 146.01 mol) in dichloroethane (DCE) (0.3 mL) was added AgNO₃ (49.61 mg, 292.02 mol, 49.11 L), collidine (35.39 mg, 292.02 mol, 38.59 μL), and 1-[chloro-(4-methoxyphenyl)-phenyl-methyl]-4-methoxy-benzene (74.21 mg, 219.01 mol). The mixture was stirred at 50° C. for 1 hr. The mixture was quenched with MeOH (2 mL) and the solvent was removed at low pressure. The residue was purified by flash silica gel chromatography (ISCO®; 4 g SepaFlash® Silica Flash Column, Eluent of 15˜45% Ethyl acetate/Petroleum ether gradient @ 20 mL/min). 1-((1S,3R,4S)-4-(Bis(4-methoxyphenyl)(phenyl)methoxy)-3-((bis(4-methoxyphenyl)(phenyl)methoxy)methyl)-3-(((tert-butyldimethylsilyl)oxy)methyl)-2-methylenecyclopentyl)pyrimidine-2,4(1H,3H)-dione (0.108 g, 107.21 mol, 73.42% yield, 98% purity) was obtained as a white foam. ¹H NMR (400 MHz, CD₃OD) δ=7.48 (d, J=7.0 Hz, 2H), 7.35 (dd, J=9.0, 13.8 Hz, 4H), 7.30-7.21 (m, 6H), 7.19-7.18 (m, 1H), 7.15 (d, J=8.0 Hz, 2H), 7.09 (dd, J=6.0, 8.8 Hz, 4H), 6.85-6.70 (m, 8H), 5.38 (s, 1H), 5.11 (d, J=8.0 Hz, 1H), 4.94 (d, J=2.2 Hz, 1H), 4.92-4.90 (m, 1H), 4.20 (t, J=6.8 Hz, 1H), 4.07 (s, 1H), 3.96 (d, J=9.8 Hz, 1H), 3.76 (dd, J=2.4, 6.8 Hz, 12H), 3.54 (d, J=9.6 Hz, 1H), 3.06 (d, J=9.6 Hz, 1H), 2.09-2.03 (m, 1H), 1.28 (d, J=5.8 Hz, 1H), 0.89 (s, 9H), 0.07 (d, J=4.2 Hz, 6H).

Step E: 1-((1S,3S,4S)-4-(Bis(4-methoxyphenyl)(phenyl)methoxy)-3-((bis(4-methoxyphenyl)(phenyl)methoxy)methyl)-3-(hydroxymethyl)-2-methylenecyclopentyl)pyrimidine-2,4(1H,3H)-dione. 1-((1S,3R,4S)-4-(Bis(4-methoxyphenyl)(phenyl)methoxy)-3-((bis(4-methoxyphenyl)(phenyl)methoxy)methyl)-3-(((tert-butyldimethylsilyl)oxy)methyl)-2-methylenecyclopentyl)pyrimidine-2,4(1H,3H)-dione (0.1 g, 101.29 mol) was treated with TBAF (2 M, 3 mL). [TBAF was neutralized with CH₃COOH (176.40 mg, 2.94 mmol, 168.00 μL)]. The reaction mixture was stirred at 50° C. for 12 h. The mixture was diluted with EA (5 mL) and washed with H₂O (5 mL*2). The organic layer was dried over anhydrous Na₂SO₄, filtered and concentrated under reduced pressure. The residue was purified by flash silica gel chromatography (ISCO®; 4 g SepaFlash® Silica Flash Column, Eluent of 0˜2% MeOH/DCMgradient @ 20 mL/min). 1-((1S,3S,4S)-4-(Bis(4-methoxyphenyl)(phenyl)methoxy)-3-((bis(4-methoxyphenyl)(phenyl)methoxy)methyl)-3-(hydroxymethyl)-2-methylenecyclopentyl)pyrimidine-2,4(1H,3H)-dione (0.06 g, 68.73 mol, 67.85% yield) was obtained as a white foam. ¹H NMR (400 MHz, CD₃OD) δ=7.53 (d, J=7.0 Hz, 2H), 7.40 (dd, J=8.8, 13.8 Hz, 4H), 7.35-7.27 (m, 5H), 7.20-7.09 (m, 8H), 6.93-6.77 (m, 8H), 5.59 (s, 1H), 5.32 (s, 1H), 5.08 (d, J=8.0 Hz, 1H), 4.96 (d, J=1.8 Hz, 1H), 4.83 (d, J=2.0 Hz, 1H), 4.23 (dd, J=6.0, 8.5 Hz, 1H), 4.03-3.90 (m, 2H), 3.86-3.74 (m, 12H), 3.61 (d, J=9.6 Hz, 1H), 3.19 (d, J=9.6 Hz, 1H), 2.13-2.06 (m, 1H), 1.37-1.30 (m, 1H). LCMS: ESI-MS: m/z=895.3 [M+Na]⁺.

Step F: (1S,3S,5S)-5-(Bis(4-methoxyphenyl)(phenyl)methoxy)-1-((bis(4-methoxyphenyl)(phenyl)methoxy)methyl)-3-(2,4-dioxo-3,4-dihydropyrimidin-1(2H)-yl)-2-methylenecyclopentane-1-carbaldehyde. To a solution of 1-((1S,3S,4S)-4-(bis(4-methoxyphenyl)(phenyl)methoxy)-3-((bis(4-methoxyphenyl)(phenyl)methoxy)methyl)-3-(hydroxymethyl)-2-methylenecyclopentyl)pyrimidine-2,4(1H,3H)-dione (0.06 g, 68.73 mol) in DCM (0.7 mL) was added DMP (58.30 mg, 137.46 mol, 42.56 μL). The mixture was stirred at 25° C. for 1 hr. The mixture was quenched with saturated NaHCO₃ solution (1 mL) and saturated Na₂S₂O₃ solution (1 mL). The mixture was extracted with DCM (10 mL*2), and the combined organic layers were washed with brine (10 mL). The resulting solution was dried over anhydrous Na₂SO₄, filtered and concentrated under reduced pressure to give a residue (1S,3S,5S)-5-(bis(4-methoxyphenyl)(phenyl)methoxy)-1-((bis(4-methoxyphenyl)(phenyl)methoxy)methyl)-3-(2,4-dioxo-3,4-dihydropyrimidin-1(2H)-yl)-2-methylenecyclopentane-1-carbaldehyde (0.06 g, crude) as white foam. LCMS: ESI-MS: m/z=893.8 [M+Na]⁺.

Step G: (E)-5-(Bis(4-methoxyphenyl)(phenyl)methoxy)-1-((bis(4-methoxyphenyl)(phenyl)methoxy)methyl)-3-(2,4-dioxo-3,4-dihydropyrimidin-1(2H)-yl)-2-methylenecyclopentane-1-carbaldehyde oxime. To a solution of (1S,3S,5S)-5-(bis(4-methoxyphenyl)(phenyl)methoxy)-1-((bis(4-methoxyphenyl)(phenyl)methoxy)methyl)-3-(2,4-dioxo-3,4-dihydropyrimidin-1(2H)-yl)-2-methylenecyclopentane-1-carbaldehyde (60 mg, 68.89 mol) in pyridine (0.7 mL) was added hydroxylamine; hydrochloride (9.57 mg, 137.78 mol). The mixture was stirred at 25° C. for 0.5 h. The reaction mixture was concentrated under reduced pressure. The residue was diluted with H₂O (5 mL) and extracted with EA (5 mL*2). The combined organic layers were washed with brine (15 mL), dried over Na₂SO₄, filtered and concentrated under reduced pressure to give the crude (E)-5-(bis(4-methoxyphenyl)(phenyl)methoxy)-1-((bis(4-methoxyphenyl)(phenyl)methoxy)methyl)-3-(2,4-dioxo-3,4-dihydropyrimidin-1(2H)-yl)-2-methylenecyclopentane-1-carbaldehyde oxime (60 mg, crude) as brown foam. LCMS: ESI-MS: m/z=908.3 [M+Na]⁺.

Step H: (1S,3S,5S)-5-(Bis(4-methoxyphenyl)(phenyl)methoxy)-1-((bis(4-methoxyphenyl)(phenyl)methoxy)methyl)-3-(2,4-dioxo-3,4-dihydropyrimidin-1(2H)-yl)-2-methylenecyclopentane-1-carbonitrile. To a solution of (E)-5-(bis(4-methoxyphenyl)(phenyl)methoxy)-1-((bis(4-methoxyphenyl)(phenyl)methoxy)methyl)-3-(2,4-dioxo-3,4-dihydropyrimidin-1(2H)-yl)-2-methylenecyclopentane-1-carbaldehyde oxime (0.71 g, 801.36 mol) in CH₃CN (8 mL) was added CDI (259.88 mg, 1.60 mmol). The mixture was stirred at 45° C. for 16 h. The mixture was diluted with EA (20 mL) and washed with H₂O (15 mL*2). The combined organic layer was dried over anhydrous Na₂SO₄, filtered and concentrated under reduced pressure. The residue was purified by flash silica gel chromatography (ISCO®; 12 g SepaFlash® Silica Flash Column, Eluent of 17˜62% Ethyl acetate/Petroleum ether gradient @ 30 mL/min). (1S,3S,5S)-5-(Bis(4-methoxyphenyl)(phenyl)methoxy)-1-((bis(4-methoxyphenyl)(phenyl)methoxy)methyl)-3-(2,4-dioxo-3,4-dihydropyrimidin-1(2H)-yl)-2-methylenecyclopentane-1-carbonitrile (0.57 g, 656.70 mol, 81.95% yield) was obtained as a white solid. ¹H NMR (400 MHz, CD₃OD) δ=7.51 (d, J=7.5 Hz, 2H), 7.44-7.37 (m, 4H), 7.34 (d, J=8.8 Hz, 2H), 7.30-7.18 (m, 10H), 7.00 (d, J=8.0 Hz, 1H), 6.80 (ddd, J=2.8, 4.1, 9.0 Hz, 8H), 5.55 (s, 1H), 5.44 (d, J=8.0 Hz, 1H), 5.21-5.11 (m, 2H), 4.05 (t, J=3.5 Hz, 1H), 3.77 (d, J=1.0 Hz, 6H), 3.72 (d, J=9.3 Hz, 6H), 3.41-3.35 (m, 1H), 3.29 (s, 1H), 1.06-0.97 (m, 1H), 0.95-0.84 (m, 1H). LCMS: ESI-MS: m/z=890.3 [M+Na]⁺.

Step I: (1S,3S,5S)-3-(4-Amino-2-oxopyrimidin-1(2H)-yl)-5-(bis(4-methoxyphenyl)(phenyl)methoxy)-1-((bis(4-methoxyphenyl)(phenyl)methoxy)methyl)-2-methylenecyclopentane-1-carbonitrile. To a solution of (1S,3S,5S)-5-(bis(4-methoxyphenyl)(phenyl)methoxy)-1-((bis(4-methoxyphenyl)(phenyl)methoxy)methyl)-3-(2,4-dioxo-3,4-dihydropyrimidin-1(2H)-yl)-2-methylenecyclopentane-1-carbonitrile (0.57 g, 656.70 mol) in CH₃CN (3 mL) was added Et₃N (132.90 mg, 1.31 mmol, 182.81 μL), DMAP (160.46 mg, 1.31 mmol) and 2,4,6-triisopropylbenzenesulfonyl chloride (397.77 mg, 1.31 mmol). The mixture was stirred at 25° C. for 0.5 h. NH₃—H₂O (1.82 g, 14.54 mmol, 2 mL, 28% purity, 22.14 eq.) was added to the mixture, and the reaction mixture was stirred at 25° C. for 1.5 h. The mixture was diluted with EA (30 mL) and washed with saturated NH₄Cl solution (15 mL*4). The organic layer was over anhydrous Na₂SO₄, filtered and concentrated under reduced pressure. The residue was purified by flash silica gel chromatography (ISCO®; 20 g SepaFlash® Silica Flash Column, Eluent of 0˜3.2% MeOH/DCMgradient @mL/min). (1S,3S,5S)-3-(4-Amino-2-oxopyrimidin-1(2H)-yl)-5-(bis(4-methoxyphenyl)(phenyl)methoxy)-1-((bis(4-methoxyphenyl)(phenyl)methoxy)methyl)-2-methylenecyclopentane-1-carbonitrile (0.439 g, 496.22 mol, 75.56% yield, 98% purity) was obtained as a white foam. ¹H NMR (400 MHz, METHANOL-d4) δ=7.51 (d, J=7.3 Hz, 2H), 7.43-7.37 (m, 4H), 7.34 (d, J=8.8 Hz, 2H), 7.30-7.16 (m, 10H), 7.01 (d, J=7.5 Hz, 1H), 6.84-6.75 (m, 8H), 5.64 (d, J=7.3 Hz, 1H), 5.48 (d, J=1.5 Hz, 1H), 5.18 (s, 1H), 5.02 (s, 1H), 4.07-4.03 (m, 1H), 3.77 (d, J=1.8 Hz, 6H), 3.72 (d, J=8.5 Hz, 6H), 3.41-3.33 (m, 2H), 1.16-1.09 (m, 1H), 1.01-0.90 (m, 1H). LCMS: ESI-MS: m/z=867.2 [M+H]⁺.

Step J: (1S,3S,5S)-3-(4-Amino-2-oxopyrimidin-1(2H)-yl)-5-hydroxy-1-(hydroxymethyl)-2-methylenecyclopentane-1-carbonitrile. To a solution of (1S,3S,5S)-3-(4-amino-2-oxopyrimidin-1(2H)-yl)-5-(bis(4-methoxyphenyl)(phenyl)methoxy)-1-((bis(4-methoxyphenyl)(phenyl)methoxy)methyl)-2-methylenecyclopentane-1-carbonitrile (389.00 mg, 448.68 mol) in DCM (2 mL) was added TFA (7.67 g, 13.46 mmol, 4.98 mL, 20% purity). The mixture was stirred at 25° C. for 0.5 hr. The mixture was quenched with NH₃ (7 M, in MeOH, 1 mL) and the solvent was removed under reduced pressure. The residue was purified by flash silica gel chromatography (ISCO®; 4 g SepaFlash® Silica Flash Column, Eluent of 5˜17% MeOH/DCMgradient @ 20 mL/min). The crude was further purified by Pre-HPLC (column: Waters Xbridge 150*25 5 u; mobile phase: [water (10 mM NH₄HCO₃)-ACN]; B %: 0%-25%, 6 min). (1S,3S,5S)-3-(4-amino-2-oxopyrimidin-1(2H)-yl)-5-hydroxy-1-(hydroxymethyl)-2-methylenecyclopentane-1-carbonitrile (0.0998 g, 376.73 mol, 83.96% yield, 99% purity) was obtained as a white solid. ¹H NMR (400 MHz, CD₃OD) δ=7.54 (d, J=7.3 Hz, 1H), 5.88 (d, J=7.3 Hz, 1H), 5.63 (t, J=9.2 Hz, 1H), 5.55 (d, J=2.3 Hz, 1H), 5.15 (s, 1H), 4.43 (d, J=3.5 Hz, 1H), 3.90-3.83 (m, 1H), 3.80-3.73 (m, 1H), 2.40-2.28 (m, 1H), 2.25-2.16 (m, 1H). LCMS: ESI-MS: m/z=263.1154 [M+H]⁺.

### Example 17: (1S,3S,5S)-3-(6-Amino-9H-purin-9-yl)-5-hydroxy-1-(hydroxymethyl)-2-methylenecyclopentane-1-carbonitrile

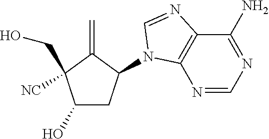 [SMILES: C=C1[C@@H](n2cnc3c(N)ncnc32)C[C@H](O)[C@@]1(C#N)CO]

Step A: ((1R,3R,5S)-1-((Bis(4-methoxyphenyl)(phenyl)methoxy)methyl)-3-((tert-butyldimethylsilyl)oxy)-2-methylene-5-(pivaloyloxy)cyclopentyl)methyl pivalate. To a solution of ((1R,3R,5S)-3-((tert-butyldimethylsilyl)oxy)-1-(hydroxymethyl)-2-methylene-5-(pivaloyloxy)cyclopentyl)methyl pivalate (Intermediate 7, product from step I, 8.8 g, 19.27 mmol) in DCM (90 mL) was added AgNO₃ (6.55 g, 38.54 mmol, 6.48 mL) and collidine (4.67 g, 38.54 mmol, 5.09 mL) in one portion at 30° C. under N₂. 1-[chloro-(4-methoxyphenyl)-phenyl-methyl]-4-methoxy-benzene (9.79 g, 28.90 mmol) was added to the mixture at 30° C. The reaction mixture was stirred at 30° C. for 1 h. The inorganic material was removed by filtration and the filtered cake was washed with DCM (100 mL*2). The filtrate was washed with water (50 mL*2), and brine (50 mL). The organic phase was dried over anhydrous Na₂SO₄, filtered and concentrated in vacuum. The residue was purified by flash silica gel chromatography (ISCO®; 80 g SepaFlash® Silica Flash Column, Eluent of 0˜7% Ethyl acetate/Petroleum ether gradient) to give ((1R,3R,5S)-1-((bis(4-methoxyphenyl)(phenyl)methoxy)methyl)-3-((tert-butyldimethylsilyl)oxy)-2-methylene-5-(pivaloyloxy)cyclopentyl)methyl pivalate (10.2 g, 12.63 mmol, 65.55% yield, 94% purity) as yellow oil. ¹H-NMR (400 MHz, CDCl₃) δ=7.40-7.15 (m, 9H), 6.85-6.75 (d, J=8.8 Hz, 4H), 5.30-5.22 (d, J=2 Hz, 1H), 5.15-5.05 (dd, J=6.4, 8 Hz, 1H), 5.00-4.95 (d, J=2.8 Hz, 1H), 4.50-4.40 (m, 1H), 4.30-4.20 (q, J=10.8 Hz, 2H), 3.80 (s, 6H), 3.04 (s, 2H), 2.50-2.40 (m, 1H), 1.75-1.62 (m, 1H), 1.20-1.00 (m, 18H), 0.95-0.85 (m, 9H), 0.10-0.04 (d, J=10 Hz, 6H). LCMS: ESI-MS: m/z=781.40 [M+Na]⁺.

Step B: ((1R,3R,5S)-1-((Bis(4-methoxyphenyl)(phenyl)methoxy)methyl)-3-hydroxy-2-methylene-5-(pivaloyloxy)cyclopentyl)methyl pivalate. To a mixture of ((1R,3R,5S)-1-((bis(4-methoxyphenyl)(phenyl)methoxy)methyl)-3-((tert-butyldimethylsilyl)oxy)-2-methylene-5-(pivaloyloxy)cyclopentyl)methyl pivalate (17.9 g, 23.58 mmol) in THF (230 mL) was added TBAF (1 M, 35.37 mL) at 30° C. The reaction mixture was stirred at 30° C. for 1 h. The solvent was removed under reduced pressure. The residue was purified by silica gel column chromatography (Petroleum ether/Ethyl acetate=5/1 to 4/1) to give ((1R,3R,5S)-1-((bis(4-methoxyphenyl)(phenyl)methoxy)methyl)-3-hydroxy-2-methylene-5-(pivaloyloxy)cyclopentyl)methyl pivalate (14 g, 21.28 mmol, 90.23% yield, 98% purity) as yellow oil. ¹H NMR (400 MHz, CDCl₃) δ=7.40-7.15 (m, 9H), 6.85-6.75 (d, J=8.8 Hz, 4H), 5.50-5.45 (d, J=1.6 Hz, 1H), 5.20-5.08 (m, 2H), 4.55-4.40 (m, 2H), 4.20-4.13 (d, J=11.2 Hz, 1H), 3.80 (s, 6H), 3.10-3.03 (dd, J=8.8.14 Hz, 2H), 2.46-2.41 (m, 1H), 1.78-1.68 (m, 1H), 1.20-1.00 (m, 18H). LCMS: ESI-MS: m/z=667.20 [M+Na]⁺.

Step C: N,N-Di-BOC-((1R,3S,5S)-3-(6-amino-9H-purin-9-yl)-1-((bis(4-methoxyphenyl)(phenyl)methoxy)methyl)-2-methylene-5-(pivaloyloxy)cyclopentyl)methyl pivalate. To a solution of ((1R,3R,5S)-1-((bis(4-methoxyphenyl)(phenyl)methoxy)methyl)-3-hydroxy-2-methylene-5-(pivaloyloxy)cyclopentyl)methyl pivalate (9 g, 13.96 mmol) in THF (180 mL) was added PPh₃ (9.52 g, 36.29 mmol) and N,N-Di-BOC-9H-purin-6-amine (14.00 g, 41.75 mmol) at 30° C., and then DIAD (7.34 g, 36.29 mmol, 7.06 mL) was added in dropwise at 0° C. The mixture was stirred at 30° C. for 12 h. The solvent was removed under reduced pressure. The residue was purified by silica gel column chromatography (Petroleum ether/Ethyl acetate=4/1) to give N,N-Di-BOC-((1R,3S,5S)-3-(6-amino-9H-purin-9-yl)-1-((bis(4-methoxyphenyl)(phenyl)methoxy)methyl)-2-methylene-5-(pivaloyloxy)cyclopentyl)methyl pivalate (15 g, 10.76 mmol, 77.07% yield, 69% purity) as yellow oil. ¹H NMR (400 MHz, CDCl₃) δ=8.77 (s, 1H), 7.79 (s, 1H), 7.45-7.20 (m, 16H), 6.85-6.78 (d, J=8.8 Hz, 6H), 6.376 (s, 4H), 5.80-5.70 (m, 1H), 5.55-5.45 (m, 1H), 5.22 (s, 1H), 4.40-4.33 (m, 2H), 4.28-4.21 (d, J=10.8 Hz, 1H), 3.79 (s, 9H), 3.45 (s, 2H), 2.54-2.36 (m, 2H), 2.32-2.23 (m, 2H), 1.50-1.40 (m, 27H), 1.30-1.17 (m, 57H), 1.10 (s, 11H). LCMS: ESI-MS: m/z=962.50 [M+H]⁺.

Step D: tert-Butyl (9-((1S,3 S,4S)-3-((bis(4-methoxyphenyl)(phenyl)methoxy)methyl)-4-hydroxy-3-(hydroxymethyl)-2-methylenecyclopentyl)-9H-purin-6-yl)carbamate. To a solution of N,N-Di-BOC-((1R,3S,5S)-3-(6-amino-9H-purin-9-yl)-1-((bis(4-methoxyphenyl)(phenyl)methoxy)methyl)-2-methylene-5-(pivaloyloxy)cyclopentyl)methyl pivalate (10 g, 10.39 mmol) in MeOH (150 mL) was added NaOH solution (4 M, 33.33 mL), and stirred at 25° C. for 12 hr. H₂O (100 mL) was added and the mixture was extracted with EA (200 mL*4). The combined organic layers were dried over anhydrous Na₂SO₄, filtered and concentrated under reduced pressure. The residue was purified by flash silica gel chromatography (ISCO®; 80 g SepaFlash® Silica Flash Column, Eluent of 0˜3% MeOH/DCM gradient @ 60 mL/min) to give tert-butyl (9-((1S,3S,4S)-3-((bis(4-methoxyphenyl)(phenyl)methoxy)methyl)-4-hydroxy-3-(hydroxymethyl)-2-methylenecyclopentyl)-9H-purin-6-yl)carbamate (3.7 g, 5.33 mmol, 51.28% yield) as a white foam. LCMS: ESI-MS: m/z=694.3 [M+H]⁺.

Step E: tert-Butyl (9-((1S,3R,4S)-3-((bis(4-methoxyphenyl)(phenyl)methoxy)methyl)-3-(((tert-butyldimethylsilyl)oxy)methyl)-4-hydroxy-2-methylenecyclopentyl)-9H-purin-6-yl)carbamate. To a solution of tert-butyl (9-((1S,3S,4S)-3-((bis(4-methoxyphenyl)(phenyl)methoxy)methyl)-4-hydroxy-3-(hydroxymethyl)-2-methylenecyclopentyl)-9H-purin-6-yl)carbamate (3.5 g, 5.04 mmol) in DMF (7 mL) was added imidazole (1.03 g, 15.13 mmol) and tert-butyl-chloro-dimethyl-silane (1.14 g, 7.57 mmol, 927.26 μL) at 0° C. The mixture was stirred at 25° C. for 2 h. The mixture was diluted with EA (50 mL) and washed with H₂O (50 mL*2). The organic layer was dried over anhydrous Na₂SO₄, filtered and concentrated under reduced pressure. The residue was purified by flash silica gel chromatography (ISCO®; 20 g SepaFlash® Silica Flash Column, Eluent of 0˜2% MeOH/DCM gradient @ 35 mL/min) to give tert-butyl (9-((1S,3R,4S)-3-((bis(4-methoxyphenyl)(phenyl)methoxy)methyl)-3-(((tert-butyldimethylsilyl)oxy)methyl)-4-hydroxy-2-methylenecyclopentyl)-9H-purin-6-yl)carbamate (3.7 g, 4.21 mmol, 83.50% yield, 92% purity) as a white foam. LCMS: ESI-MS: m/z=808.5 [M+H]⁺.

Step F: tert-Butyl (9-((1S,3R,4S)-4-(bis(4-methoxyphenyl)(phenyl)methoxy)-3-((bis(4-methoxyphenyl)(phenyl)methoxy)methyl)-3-(((tert-butyldimethylsilyl)oxy)methyl)-2-methylenecyclopentyl)-9H-purin-6-yl)carbamate. To a solution of tert-butyl (9-((1S,3R,4S)-3-((bis(4-methoxyphenyl)(phenyl)methoxy)methyl)-3-(((tert-butyldimethylsilyl)oxy)methyl)-4-hydroxy-2-methylenecyclopentyl)-9H-purin-6-yl)carbamate (4.7 g, 5.82 mmol) in DCE (13 mL) was added AgNO₃ (1.98 g, 11.63 mmol, 1.96 mL), collidine (1.41 g, 11.63 mmol, 1.54 mL), and 1-[chloro-(4-methoxyphenyl)-phenyl-methyl]-4-methoxy-benzene (2.96 g, 8.72 mmol). The mixture was stirred at 50° C. for 2 hr. The mixture was quenched with MeOH (10 mL) and the solvent was removed at low pressure. The residue was purified by flash silica gel chromatography (ISCO®; 40 g SepaFlash® Silica Flash Column, Eluent of 8-30% Ethyl acetate/Petroleum ether gradient @ 40 mL/min) to give tert-butyl (9-((1S,3R,4S)-4-(bis(4-methoxyphenyl)(phenyl)methoxy)-3-((bis(4-methoxyphenyl)(phenyl)methoxy)methyl)-3-(((tert-butyldimethylsilyl)oxy)methyl)-2-methylenecyclopentyl)-9H-purin-6-yl)carbamate (5.8 g, 5.12 mmol, 88.01% yield, 98% purity) as a white foam. ¹H NMR (400 MHz, CDCl₃) δ=8.67 (s, 1H), 8.08 (s, 1H), 7.64 (s, 1H), 7.48-7.42 (m, 2H), 7.32 (dd, J=8.8, 13.2 Hz, 4H), 7.25-7.17 (m, 7H), 7.17-7.07 (m, 5H), 6.79-6.65 (m, 8H), 5.57 (t, J=6.9 Hz, 1H), 5.04 (s, 1H), 4.76 (d, J=1.8 Hz, 1H), 4.27-4.17 (m, 2H), 4.01 (d, J=9.5 Hz, 1H), 3.77 (d, J=2.0 Hz, 6H), 3.74-3.69 (m, 6H), 3.54 (d, J=9.5 Hz, 1H), 3.05-2.99 (m, 1H), 2.12-2.06 (m, 1H), 1.56 (s, 9H), 1.50-1.40 (m, 1H), 0.99-0.86 (m, 9H), 0.10 (d, J=5.3 Hz, 6H). LCMS: ESI-MS: m/z=1110.6 [M+H]⁺.

Step G: tert-Butyl (9-((1S,3S,4S)-4-(bis(4-methoxyphenyl)(phenyl)methoxy)-3-((bis(4-methoxyphenyl)(phenyl)methoxy)methyl)-3-(hydroxymethyl)-2-methylenecyclopentyl)-9H-purin-6-yl)carbamate. tert-Butyl (9-((1S,3R,4S)-4-(bis(4-methoxyphenyl)(phenyl)methoxy)-3-((bis(4-methoxyphenyl)(phenyl)methoxy)methyl)-3-(((tert-butyldimethylsilyl)oxy)methyl)-2-methylenecyclopentyl)-9H-purin-6-yl)carbamate (4.4 g, 3.96 mmol, 1 eq.) was treated with TBAF (1.5 M, 40 mL, 15.14 eq.) and stirred at 50° C. for 3 h. The mixture was diluted with EA (40 mL) and washed with H₂O (30 mL*2). The organic layer was dried over anhydrous Na₂SO₄, filtered and concentrated under reduced pressure. The residue was purified by flash silica gel chromatography (ISCO®; 40 g SepaFlash® Silica Flash Column, Eluent of 20˜55% Ethyl acetate/Petroleum ethergradient @ 40 mL/min) to give tert-butyl (9-((1S,3S,4S)-4-(bis(4-methoxyphenyl)(phenyl)methoxy)-3-((bis(4-methoxyphenyl)(phenyl)methoxy)methyl)-3-(hydroxymethyl)-2-methylenecyclopentyl)-9H-purin-6-yl)carbamate (2.8 g, 2.70 mmol, 68.10% yield, 96% purity) as white foam. ¹H NMR (400 MHz, CD₃OD) δ=8.45 (s, 1H), 7.86 (s, 1H), 7.43 (dd, J=1.4, 8.0 Hz, 2H), 7.32-7.17 (m, 12H), 7.15-7.08 (m, 4H), 6.79-6.66 (m, 8H), 5.41 (d, J=4.4 Hz, 1H), 5.06 (d, J=1.5 Hz, 1H), 4.97 (d, J=1.5 Hz, 1H), 4.28 (dd, J=6.0, 9.0 Hz, 1H), 4.15-4.12 (m, 1H), 4.08-4.05 (m, 1H), 3.76 (d, J=2.4 Hz, 6H), 3.72 (d, J=3.3 Hz, 6H), 3.48 (d, J=9.5 Hz, 1H), 3.35-3.32 (m, 1H), 2.21 (td, J=8.7, 13.9 Hz, 1H), 1.76-1.64 (m, 1H), 1.59 (s, 9H). LCMS: ESI-MS: m/z=996.4 [M+H]⁺.

Step H: tert-Butyl (9-((1S,3S,4S)-4-(bis(4-methoxyphenyl)(phenyl)methoxy)-3-((bis(4-methoxyphenyl)(phenyl)methoxy)methyl)-3-formyl-2-methylenecyclopentyl)-9H-purin-6-yl)carbamate. To a solution of tert-butyl (9-((1S,3S,4S)-4-(bis(4-methoxyphenyl)(phenyl)methoxy)-3-((bis(4-methoxyphenyl)(phenyl)methoxy)methyl)-3-(hydroxymethyl)-2-methylenecyclopentyl)-9H-purin-6-yl)carbamate (0.4 g, 401.55 mol) in DCM (4 mL) was added DMP (340.62 mg, 803.09 mol, 248.63 μL). The mixture was stirred at 25° C. for 1 hr. The mixture was quenched with saturated NaHCO₃ solution (5 mL) and saturated Na₂S₂O₃ solution (5 mL). The reaction mixture was extracted with DCM (20 mL*2). The combined organic layers were washed with brine (10 mL), dried over anhydrous Na₂SO₄, filtered and concentrated under reduced pressure to give crude tert-butyl (9-((1S,3S,4S)-4-(bis(4-methoxyphenyl)(phenyl)methoxy)-3-((bis(4-methoxyphenyl)(phenyl)methoxy)methyl)-3-formyl-2-methylenecyclopentyl)-9H-purin-6-yl)carbamate (0.39 g, crude) as yellow foam. LCMS: ESI-MS: m/z=994.5 [M+H]⁺.

Step I: tert-Butyl (9-((1S,3S,4S)-4-(bis(4-methoxyphenyl)(phenyl)methoxy)-3-((bis(4-methoxyphenyl)(phenyl)methoxy)methyl)-3-((E)-(hydroxyimino)methyl)-2-methylenecyclopentyl)-9H-purin-6-yl)carbamate. To a solution of tert-butyl (9-((1S,3S,4S)-4-(bis(4-methoxyphenyl)(phenyl)methoxy)-3-((bis(4-methoxyphenyl)(phenyl)methoxy)methyl)-3-formyl-2-methylenecyclopentyl)-9H-purin-6-yl)carbamate (0.39 g, 392.30 mol) in pyridine (4 mL) was added NH₂OH—HCl (54.52 mg, 784.60 mol). The mixture was stirred at 25° C. for 0.5 h. The reaction mixture was concentrated under reduced pressure. The residue was diluted with H₂O (5 mL) and extracted with EA (5 mL*2). The combined organic layers were washed with brine (10 mL), dried over anhydrous Na₂SO₄, filtered and concentrated under reduced pressure to give tert-butyl (9-((1S,3S,4S)-4-(bis(4-methoxyphenyl)(phenyl)methoxy)-3-((bis(4-methoxyphenyl)(phenyl)methoxy)methyl)-3-((E)-(hydroxyimino)methyl)-2-methylenecyclopentyl)-9H-purin-6-yl)carbamate (0.39 g, crude) as yellow foam. LCMS: ESI-MS: m/z=1009.4 [M+H]⁺.

Step J: tert-Butyl (9-((1S,3S,4S)-4-(bis(4-methoxyphenyl)(phenyl)methoxy)-3-((bis(4-methoxyphenyl)(phenyl)methoxy)methyl)-3-cyano-2-methylenecyclopentyl)-9H-purin-6-yl)carbamate. To a stirred solution of tert-butyl (9-((1S,3S,4S)-4-(bis(4-methoxyphenyl)(phenyl)methoxy)-3-((bis(4-methoxyphenyl)(phenyl)methoxy)methyl)-3-((E)-(hydroxyimino)methyl)-2-methylenecyclopentyl)-9H-purin-6-yl)carbamate (0.45 g, 445.92 mol) in CH₃CN (4 mL) was added CDI (144.61 mg, 891.84 mol). The mixture was stirred at 25° C. for 12 hr. The mixture was diluted with EA (5 mL) and washed with H₂O (5 mL*2). The combined organic layer was dried over anhydrous Na₂SO₄, filtered and concentrated under reduced pressure. The residue was purified by column chromatography (SiO₂, Petroleum ether/Ethyl acetate=5/1 to 2/1) and further purified by Prep-HPLC (column: Waters Xbridge 150*25 5 u; mobile phase: [water (10 mM NH₄HCO₃)-ACN]; B %: 70%-100%, 6 min) to give tert-butyl (9-((1S,3S,4S)-4-(bis(4-methoxyphenyl)(phenyl)methoxy)-3-((bis(4-methoxyphenyl)(phenyl)methoxy)methyl)-3-cyano-2-methylenecyclopentyl)-9H-purin-6-yl)carbamate (0.34 g, 336.18 mol, 75.39% yield, 98% purity) as a white solid. ¹H NMR (400 MHz, CD₃OD) δ=8.14 (s, 1H), 8.06 (s, 1H), 7.55 (d, J=7.3 Hz, 2H), 7.46-7.40 (m, 4H), 7.37 (d, J=9.0 Hz, 2H), 7.31-7.15 (m, 10H), 6.84-6.75 (m, 8H), 5.62 (s, 1H), 5.49 (br t, J=8.4 Hz, 1H), 4.97 (s, 1H), 4.59 (br, s, 1H), 4.41 (t, J=4.0 Hz, 1H), 3.76 (s, 6H), 3.72 (d, J=2.0 Hz, 6H), 3.67 (d, J=9.9 Hz, 1H), 3.39 (d, J=9.9 Hz, 1H), 1.70 (ddd, J=4.7, 8.7, 13.9 Hz, 1H), 1.56 (s, 9H), 1.23-1.15 (m, 1H). LCMS: ESI-MS: m/z=991.5 [M+H]⁺.

Step K: (1S,3S,5S)-3-(6-Amino-9H-purin-9-yl)-5-hydroxy-1-(hydroxymethyl)-2-methylenecyclopentane-1-carbonitrile. To a solution of tert-butyl (9-((1S,3S,4S)-4-(bis(4-methoxyphenyl)(phenyl)methoxy)-3-((bis(4-methoxyphenyl)(phenyl)methoxy)methyl)-3-cyano-2-methylenecyclopentyl)-9H-purin-6-yl)carbamate (0.34 g, 343.04 mol) in DCM (3 mL) was added TFA (4.62 g, 8.10 mmol, 3 mL, 20% purity). The mixture was stirred at 25° C. for 2 hr. The mixture was quenched with NH₃ (7 M, in MeOH, 2 mL) and the solvent was removed under reduced pressure. The residue was purified by column chromatography (SiO₂, DCM/MeOH=30/1 to 15/1) and further purified by Prep-HPLC (column: Waters Xbridge 150*25 5 u; mobile phase: [water (10 mM NH₄HCO₃)-ACN]; B %: 0%-30%, 6 min) to give (1S,3S,5S)-3-(6-amino-9H-purin-9-yl)-5-hydroxy-1-(hydroxymethyl)-2-methylenecyclopentane-1-carbonitrile (0.062 g, 216.56 mol, 63.13% yield, 100% purity) as a white solid. ¹H NMR (400 MHz, CD₃OD) δ=8.20 (s, 1H), 8.16 (s, 1H), 5.85-5.72 (m, 1H), 5.58 (d, J=1.8 Hz, 1H), 5.06 (s, 1H), 4.57 (br, J=3.1 Hz, 1H), 4.11 (d, J=11.5 Hz, 1H), 3.87 (d, J=11.5 Hz, 1H), 2.80 (ddd, J=4.5, 10.8, 13.3 Hz, 1H), 2.39 (ddd, J=1.7, 8.2, 13.5 Hz, 1H). LCMS: ESI-MS: m/z=287.1294 [M+H]⁺.

### Example 18: (1S,3S,5S)-3-(2-Amino-6-oxo-1,6-dihydro-9H-purin-9-yl)-5-hydroxy-1-(hydroxymethyl)-2-methylenecyclopentane-1-carbonitrile

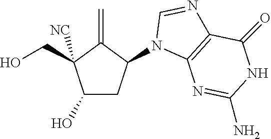 [SMILES: C=C1[C@@H](n2cnc3c(=O)[nH]c(N)nc32)C[C@H](O)[C@@]1(C#N)CO]

Step A. N,N-Di-BOC-(6aS,8S,9aS)-8-(2-amino-6-chloro-9H-purin-9-yl)-2.2.4.4-tetraisopropyl-7-methylenetetrahydrocyclopenta[f][1.3.5.2.4]trioxadisilocine-6a(6H)-carbonitrile. To a solution of (6aS,8R,9aS)-8-hydroxy-2,2,4,4-tetraisopropyl-7-methylenetetrahydrocyclopenta[f][1,3,5,2,4]trioxadisilocine-6a(6H)-carbonitrile (Intermediate 9, 0.054 g, 0.131 mmol), N,N-Di-BOC-6-chloro-9H-purin-2-amine (Intermediate 10, 0.145 g, 0.393 mmol), and triphenylphosphine (0.103 g, 0.393 mmol) in THF (1.3 mL, 0.1 M) was added diisopropyl azodicarboxylate (0.080 mL, 0.393 mmol). The reaction mixture was heated at reflux for 1 h and 10 min, then cooled and concentrated in vacuo to give an orange oil. Two batches were combined and purified (FCC, SiO₂, 0-30% EtOAc/hexanes) to provide N,N-Di-BOC-(6aS,8S,9aS)-8-(2-amino-6-chloro-9H-purin-9-yl)-2,2,4,4-tetraisopropyl-7-methylenetetrahydrocyclopenta[f][1,3,5,2,4]trioxadisilocine-6a(6H)-carbonitrile as a foamy, white solid (0.108 g).

Step B. (1S,3S,5S)-3-(2-Amino-6-oxo-1,6-dihydro-9H-purin-9-yl)-5-hydroxy-1-(hydroxymethyl)-2-methylenecyclopentane-1-carbonitrile. To a round bottom flask charged with N,N-Di-BOC-(6aS,8S,9aS)-8-(2-amino-6-chloro-9H-purin-9-yl)-2,2,4,4-tetraisopropyl-7-methylenetetrahydrocyclopenta[f][1,3,5,2,4]trioxadisilocine-6a(6H)-carbonitrile (0.108 g, 0.141 mmol) cooled to 0° C. was added TFA/H₂O (1.25 mL:1.25 mL: 0.0566 M) dropwise. The colorless solution was stirred at r.t. for 48 h, then coevaporated with EtOH (3×), concentrated in vacuo and taken up in THF. To this solution as added TBAF (1 equiv.) at 0° C., then stirred at rt. Another equivalent of TBAF was added after 2 h, then after 1 h, concentrated in vacuo to give a thick yellow oil. The crude oil was purified on the reverse phase HPLC Phenomenex Synergyi 4 micron Hydro-RP 80 A 250×21.2 mm (0-50% acetonitrile with triethylammonium acetate (TEAA) buffer in H₂O with TEAA buffer) to give (1S,3S,5S)-3-(2-amino-6-oxo-1,6-dihydro-9H-purin-9-yl)-5-hydroxy-1-(hydroxymethyl)-2-methylenecyclopentane-1-carbonitrile as a white solid (0.0277 g, 59% over 2 steps). ¹H NMR (400 MHz, CDCl₃): δ 7.77 (s, 1H), 5.64 (m, 1H), 5.56 (d, J=2.8, 1H), 5.53 (m, 1H), 5.08 (d, J=2.8, 1H), 4.01 (d, J=12, 1H), 3.82 (d, J=12, 1H), 2.66 (m, 1H), 2.34 (m, 1H). MS, m/Z 302.95 [M+1]*.

### Example 19: (1S,2R,4S)-4-(6-Amino-9H-purin-9-yl)-2-ethynyl-2-(hydroxymethyl)-3-methylenecyclopentan-1-ol

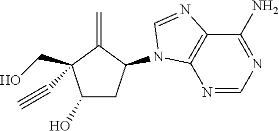 [SMILES: C#C[C@]1(CO)C(=C)[C@@H](n2cnc3c(N)ncnc32)C[C@@H]1O]

Step A: tert-Butyl (9-((1S,3S,4S)-4-(bis(4-methoxyphenyl)(phenyl)methoxy)-3-((bis(4-methoxyphenyl)(phenyl)methoxy)methyl)-3-formyl-2-methylenecyclopentyl)-9H-purin-6-yl)carbamate. To a solution of tert-butyl (9-((1S,3S,4S)-4-(bis(4-methoxyphenyl)(phenyl)methoxy)-3-((bis(4-methoxyphenyl)(phenyl)methoxy)methyl)-3-(hydroxymethyl)-2-methylenecyclopentyl)-9H-purin-6-yl)carbamate (Example 17, Product from Step G, 0.4 g, 401.55 mol) in DCM (4 mL) was added DMP (340.62 mg, 803.09 mol, 248.63 μL). The mixture was stirred at 25° C. for 1 h. The reaction mixture was filtered and quenched by addition of saturated NaHCO₃ solution and saturated Na₂SO₃ solution (1:1, 5 mL). The resulting solution was washed with brine (2 mL*5). The combined organic layers were dried over anhydrous Na₂SO₄, filtered and concentrated under reduced pressure. Purification (FCC, SiO₂, Petroleum ether/Ethyl acetate=10/1 to 1/1) afforded tert-butyl (9-((1S,3S,4S)-4-(bis(4-methoxyphenyl)(phenyl)methoxy)-3-((bis(4-methoxyphenyl)(phenyl)methoxy)methyl)-3-formyl-2-methylenecyclopentyl)-9H-purin-6-yl)carbamate (388 mg, 390.29 mol, 97.20% yield) as white foam. LCMS: ESI-MS: m/z=994.5 [M+H]⁺.

Step B: tert-Butyl (9-((1S,3R,4S)-4-(bis(4-methoxyphenyl)(phenyl)methoxy)-3-((bis(4-methoxyphenyl)(phenyl)methoxy)methyl)-3-YI ynyl-2-methylenecyclopentyl)-9H-purin-6-yl)carbamate. To a solution of K₂CO₃ (187.68 mg, 1.36 mmol) in MeCN was added 1-dimethoxyphosphorylpropan-2-one (75.19 mg, 452.65 mol, 62.14 μL) and TosN₃ (89.27 mg, 452.65 mol) at 25° C. under N₂. The mixture was stirred at 25° C. for 2 h. tert-Butyl (9-((1S,3S,4S)-4-(bis(4-methoxyphenyl)(phenyl)methoxy)-3-((bis(4-methoxyphenyl)(phenyl)methoxy)methyl)-3-formyl-2-methylenecyclopentyl)-9H-purin-6-yl)carbamate (0.225 g, 226.33 mol) in MeOH (0.3 mL) and MeCN (0.3 mL) was added. The mixture was stirring at 25° C. for 48 h. The mixture was quenched with H₂O (3 mL) and extracted with EA (5 mL). The organic layer was dried over anhydrous Na₂SO₄, filtered and concentrated under reduced pressure. Purification (FCC, SiO₂, Petroleum ether/Ethyl acetate=20/1 to 2/1) afforded tert-butyl (9-((1S,3R,4S)-4-(bis(4-methoxyphenyl)(phenyl)methoxy)-3-((bis(4-methoxyphenyl)(phenyl)methoxy)methyl)-3-ethynyl-2-methylenecyclopentyl)-9H-purin-6-yl)carbamate (100 mg, 96.96 mol, 42.84% yield, 96% purity) as white foam. LCMS: ESI-MS: m/z=990.5 [M+H]⁺.

Step C: (1S,2R,4S)-4-(6-Amino-9H-purin-9-yl)-2-ethynyl-2-(hydroxymethyl)-3-methylenecyclopentan-1-ol. To a solution of tert-butyl (9-((1S,3R,4S)-4-(bis(4-methoxyphenyl)(phenyl)methoxy)-3-((bis(4-methoxyphenyl)(phenyl)methoxy)methyl)-3-ethynyl-2-methylenecyclopentyl)-9H-purin-6-yl)carbamate (100 mg, 101.00 mol) in DCM (0.6 mL) was added TFA (770.00 mg, 6.75 mmol, 0.5 mL) and DCM (0.1 mL) (V_{TFA}: V_{DCM}=5:1) at 25° C. The mixture was stirring at 25° C. for 2 h. The mixture was neutralized by NH₃ in MeOH (7 M) to pH=7. The resulting solution was concentrated under reduced pressure. The residue was purified by flash silica gel chromatography (SiO₂, DCM/MeOH=30/1 to 10/1) and further purified by Prep-HPLC (column: Xbridge BEH C18, 250*50 mm, 10 m; mobile phase: [water (10 mM NH₄HCO₃)-ACN]; B %: 0%-30%, 9 min) to give (1S,2R,4S)-4-(6-amino-9H-purin-9-yl)-2-ethynyl-2-(hydroxymethyl)-3-methylenecyclopentan-1-ol (33.1 mg, 100% purity) as a white solid. ¹H NMR (400 MHz, CD₃OD) δ=8.24 (s, 1H), 8.18 (s, 1H), 5.80-5.73 (m, 1H), 5.51 (d, J=2.5 Hz, 1H), 4.99 (d, J=2.5 Hz, 1H), 4.45-4.42 (m, 1H), 3.98 (d, J=11.3 Hz, 1H), 3.82 (d, J=11.0 Hz, 1H), 2.72-2.62 (m, 2H), 2.41 (ddd, J=3.0, 8.4, 13.2 Hz, 1H). LCMS: ESI-MS: m/z=285.9 [M+H]⁺.

### Example 20: (1S,2R,4S)-4-(6-Amino-9H-purin-9-yl)-2-(hydroxymethyl)-3-methylene-2-vinylcyclopentan-1-ol

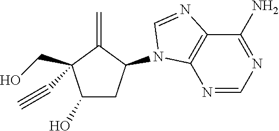 [SMILES: C#C[C@]1(CO)C(=C)[C@@H](n2cnc3c(N)ncnc32)C[C@@H]1O]

Step A: tert-Butyl (9-((1S,3R,4S)-4-(bis(4-methoxyphenyl)(phenyl)methoxy)-3-((bis(4-methoxyphenyl)(phenyl)methoxy)methyl)-2-methylene-3-vinylcyclopentyl)-9H-purin-6-yl)carbamate. To a solution of methyl (triphenyl)phosphonium; bromide (280.28 mg, 784.60 mol) in toluene (0.5 mL) was added potassium 2-methyl-2-butoxide (396.19 mg, 784.60 mol, 455.39 μL, 25% purity) at 25° C., and stirred at 25° C. for 1 h. Then, a solution of tert-butyl (9-((1S,3S,4S)-4-(bis(4-methoxyphenyl)(phenyl)methoxy)-3-((bis(4-methoxyphenyl)(phenyl)methoxy)methyl)-3-formyl-2-methylenecyclopentyl)-9H-purin-6-yl)carbamate (0.26 g, 261.53 mol) in toluene (0.5 mL) was added to the mixture, and the mixture was stirred at 25° C. for 4 h. The mixture was quenched with saturated NH₄Cl solution (5 mL) and extracted with EA (5 mL*2). The combined organic layers were dried over anhydrous Na₂SO₄, filtered and concentrated under reduced pressure. The residue was purified by flash silica gel chromatography (ISCO®; 4 g SepaFlash® Silica Flash Column, Eluent of 20-40% Ethyl acetate/Petroleum ether gradient @ 20 mL/min) to give tert-butyl (9-((1S,3R,4S)-4-(bis(4-methoxyphenyl)(phenyl)methoxy)-3-((bis(4-methoxyphenyl)(phenyl)methoxy)methyl)-2-methylene-3-vinylcyclopentyl)-9H-purin-6-yl)carbamate (0.18 g, 177.79 mol, 67.98% yield, 98% purity) as white foam. ¹H NMR (400 MHz, CD₃OD) δ=8.44 (s, 1H), 7.86 (s, 1H), 7.45 (d, J=7.3 Hz, 2H), 7.31-7.08 (m, 16H), 6.79-6.64 (m, 8H), 6.58 (dd, J=10.6, 17.6 Hz, 1H), 5.42 (d, J=10.8 Hz, 1H), 5.31 (br d, J=6.0 Hz, 1H), 5.23 (d, J=17.2 Hz, 1H), 5.07 (s, 1H), 4.81 (s, 1H), 4.28 (dd, J=5.2, 9.6 Hz, 1H), 3.75 (dd, J=6.0, 7.9 Hz, 12H), 3.60 (d, J=9.7 Hz, 1H), 3.22 (d, J=9.7 Hz, 1H), 1.96-1.83 (m, 1H), 1.69 (br, d, J=13.2 Hz, 1H), 1.59 (s, 9H). LCMS: ESI-MS: m/z=992.4 [M+H]⁺.

Step B: (1S,2R,4S)-4-(6-Amino-9H-purin-9-yl)-2-(hydroxymethyl)-3-methylene-2-vinylcyclopentan-1-ol. To a solution of tert-butyl (9-((1S,3R,4S)-4-(bis(4-methoxyphenyl)(phenyl)methoxy)-3-((bis(4-methoxyphenyl)(phenyl)methoxy)methyl)-2-methylene-3-vinylcyclopentyl)-9H-purin-6-yl)carbamate (0.4 g, 403.16 mol) in DCM (2 mL) was added TFA (7.00 g, 12.28 mmol, 4.55 mL, 20% purity). The mixture was stirred at 25° C. for 8 hr. The mixture was quenched with NH₃ (7 M, in MeOH, 4 mL) and the solvent was removed under reduced pressure. The residue was purified by column chromatography (SiO₂, DCM/MeOH=30/1 to 15/1) to give (1S,2R,4S)-4-(6-amino-9H-purin-9-yl)-2-(hydroxymethyl)-3-methylene-2-vinylcyclopentan-1-ol (0.09 g, 310.11 mol, 76.92% yield, 99% purity) as a white solid. ¹H NMR (400 MHz, CD₃OD) δ=8.33 (s, 1H), 8.26 (s, 1H), 6.06 (dd, J=10.9, 17.5 Hz, 1H), 5.72-5.60 (m, 1H), 5.35-5.22 (m, 3H), 5.06 (d, J=2.2 Hz, 1H), 4.57 (t, J=6.2 Hz, 1H), 3.80 (d, J=2.0 Hz, 2H), 2.49-2.39 (m, 1H), 2.27 (ddd, J=6.5, 8.4, 13.3 Hz, 1H). LCMS: ESI-MS: m/z=287.9 [M+H]⁺.

### Example 21: Synthesis of Nucleoside 5′-triphosphates

Dry nucleoside (0.05 mmol) was dissolved in dry PO(OMe)₃ (1 mL). N-Methylimidazole (0.009 mL, 0.11 mmol) was added followed by POCl₃ (0.009 mL, 0.11 mmol). The reaction mixture was stirred at rt for 20-40 min. The reaction was monitored by LCMS (by the appearance of corresponding nucleoside 5′-monophosphate). Upon completion of the reaction tetrabutylammonium salt of pyrophosphate (150 mg) was added, followed by DMF (0.5 mL) to get a homogeneous solution. The reaction mixture was stirred at rt for 1.5 h, then diluted with water (10 mL). Purification (column HiLoad 16/10 with Q Sepharose High Performance: Separation was done in a linear gradient of NaCl from 0 (buffer A) to 1N in 50 mM TRIS-buffer (pH7.5) (buffer B). Triphosphate was eluted at 75-80% of buffer B. Corresponding fractions were concentrated. Desalting was achieved by RP HPLC on Synergy 4 micron Hydro-RP column (Phenominex). A linear gradient of acetonitrile from 0 to 30% in 10 mM triethylammonium acetate buffer (pH 7.5) was used for elution. The corresponding fractions were combined, concentrated and lyophilized 3 times to remove excess of buffer to afford the desired nucleoside 5′-triphosphate.

### Example 22: ((1R,3S,5S)-3-(4-Amino-7H-pyrrolo[2,3-d]pyrimidin-7-yl)-5-hydroxy-2-methylenecyclopentyl)methyl Tetrahydrogen Triphosphate

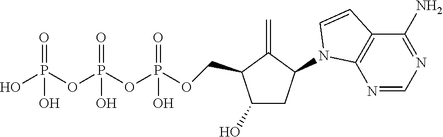 [SMILES: C=C1[C@H](COP(=O)(O)OP(=O)(O)OP(=O)(O)O)[C@@H](O)C[C@@H]1n1ccc2c(N)ncnc21]

The title compound was prepared in a manner analogous to Example 21 using (1S,2R,4S)-4-(4-amino-7H-pyrrolo[2,3-d]pyrimidin-7-yl)-2-(hydroxymethyl)-3-methylenecyclopentan-1-ol as the nucleoside starting material.

### Example 23. ((1R,3S,5S)-3-(2-Amino-4-oxo-3,4-dihydro-7H-pyrrolo[2,3-d]pyrimidin-7-yl)-5-hydroxy-2-methylenecyclopentyl)methyl Tetrahydrogen Triphosphate

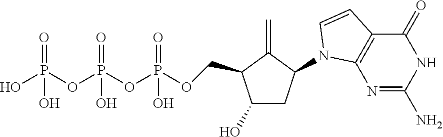 [SMILES: C=C1[C@H](COP(=O)(O)OP(=O)(O)OP(=O)(O)O)[C@@H](O)C[C@@H]1n1ccc2c(=O)[nH]c(N)nc21]

The title compound was prepared in a manner analogous to Example 12 using 2-amino-7-((1S,3R,4S)-4-hydroxy-3-(hydroxymethyl)-2-methylenecyclopentyl)-3,7-dihydro-4H-pyrrolo[2,3-d]pyrimidin-4-one (Example 2) as the nucleoside starting material.

### Example 24. ((1S,3S,5S)-1-Fluoro-5-hydroxy-3-(5-methyl-2,4-dioxo-3,4-dihydropyrimidin-1(2H)-yl)-2-methylenecyclopentyl)methyl Tetrahydrogen Triphosphate

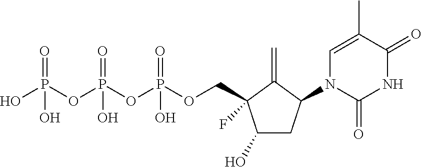 [SMILES: C=C1[C@@H](n2cc(C)c(=O)[nH]c2=O)C[C@H](O)[C@@]1(F)COP(=O)(O)OP(=O)(O)OP(=O)(O)O]

The title compound was prepared in a manner analogous to Example 21 using 1-((1S,3S,4S)-3-Fluoro-4-hydroxy-3-(hydroxymethyl)-2-methylenecyclopentyl)-5-methylpyrimidine-2,4(1H,3H)-dione (Example 3) as the nucleoside starting material.

### Example 25. ((1S,3S,5S)-3-(2-Amino-6-oxo-1,6-dihydro-9H-purin-9-yl)-1-fluoro-5-hydroxy-2-methylenecyclopentyl)methyl Tetrahydrogen Triphosphate

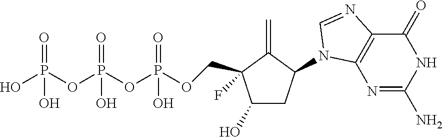 [SMILES: C=C1[C@@H](n2cnc3c(=O)[nH]c(N)nc32)C[C@H](O)[C@@]1(F)COP(=O)(O)OP(=O)(O)OP(=O)(O)O]

The title compound was prepared in a manner analogous to Example 21 using 2-Amino-9-((1S,3S,4S)-3-fluoro-4-hydroxy-3-(hydroxymethyl)-2-methylenecyclopentyl)-1,9-dihydro-6H-purin-6-one (Example 4) as the nucleoside starting material.

### Example 26. ((1S,3S,5S)-3-(6-Amino-9H-purin-9-yl)-1-fluoro-5-hydroxy-2-methylenecyclopentyl)methyl Tetrahydrogen Triphosphate

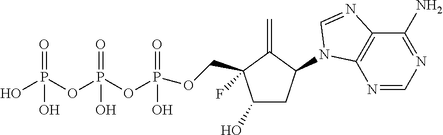 [SMILES: C=C1[C@@H](n2cnc3c(N)ncnc32)C[C@H](O)[C@@]1(F)COP(=O)(O)OP(=O)(O)OP(=O)(O)O]

The title compound was prepared in a manner analogous to Example 21 using (1S,2S,4S)-4-(6-Amino-9H-purin-9-yl)-2-fluoro-2-(hydroxymethyl)-3-methylenecyclopentan-1-ol (Example 6) as the nucleoside starting material.

### Example 27: ((1S,3S,5S)-3-(4-Amino-7H-pyrrolo[2,3-d]pyrimidin-7-yl)-1-fluoro-5-hydroxy-2-methylenecyclopentyl)methyl Tetrahydrogen Triphosphate

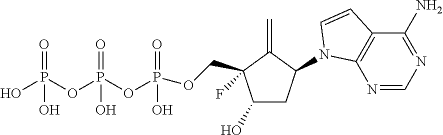 [SMILES: C=C1[C@@H](n2ccc3c(N)ncnc32)C[C@H](O)[C@@]1(F)COP(=O)(O)OP(=O)(O)OP(=O)(O)O]

The title compound was prepared in a manner analogous to Example 21 using (1S,2S,4S)-4-(4-Amino-7H-pyrrolo[2,3-d]pyrimidin-7-yl)-2-fluoro-2-(hydroxymethyl)-3-methylenecyclopentan-1-ol (Example 1) as the nucleoside starting material.

### Example 28. ((1S,3S,5S)-3-(2-Amino-6-oxo-1,6-dihydro-9H-purin-9-yl)-1-(fluoromethyl)-5-hydroxy-2-methylenecyclopentyl)methyl Tetrahydrogen Triphosphate

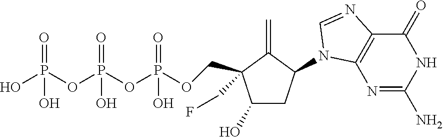 [SMILES: C=C1[C@@H](n2cnc3c(=O)[nH]c(N)nc32)C[C@H](O)[C@@]1(CF)COP(=O)(O)OP(=O)(O)OP(=O)(O)O]

The title compound was prepared in a manner analogous to Example 21 using 2-Amino-9-((1S,3S,4S)-3-(fluoromethyl)-4-hydroxy-3-(hydroxymethyl)-2-methylenecyclopentyl)-1,9-dihydro-6H-purin-6-one (Example 10) as the nucleoside starting material.

### Example 29. ((1S,3S,5S)-3-(4-Amino-2-oxopyrimidin-1(2H)-yl)-1-(fluoromethyl)-5-hydroxy-2-methylenecyclopentyl)methyl Tetrahydrogen Triphosphate

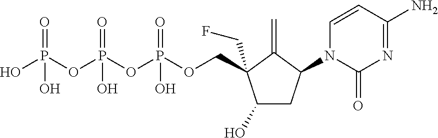 [SMILES: C=C1[C@@H](n2ccc(N)nc2=O)C[C@H](O)[C@@]1(CF)COP(=O)(O)OP(=O)(O)OP(=O)(O)O]

The title compound was prepared in a manner analogous to Example 21 using 4-Amino-1-((1S,3S,4S)-3-(fluoromethyl)-4-hydroxy-3-(hydroxymethyl)-2-methylenecyclopentyl)pyrimidin-2(1H)-one (Example 11) as the nucleoside starting material.

### Example 30. ((1S,3S,5S)-1-(Fluoromethyl)-5-hydroxy-3-(5-methyl-2,4-dioxo-3,4-dihydropyrimidin-1(2H)-yl)-2-methylenecyclopentyl)methyl Tetrahydrogen Triphosphate

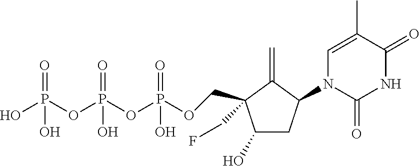 [SMILES: C=C1[C@@H](n2cc(C)c(=O)[nH]c2=O)C[C@H](O)[C@@]1(CF)COP(=O)(O)OP(=O)(O)OP(=O)(O)O]

The title compound was prepared in a manner analogous to Example 21 using 1-((1S,3S,4S)-3-(fluoromethyl)-4-hydroxy-3-(hydroxymethyl)-2-methylenecyclopentyl)-5-methylpyrimidine-2,4(1H,3H)-dione (Example 12) as the nucleoside starting material.

### Example 31. ((1S,3S,5S)-3-(6-Amino-9H-purin-9-yl)-1-(chloromethyl)-5-hydroxy-2-methylenecyclopentyl)methyl Tetrahydrogen Triphosphate

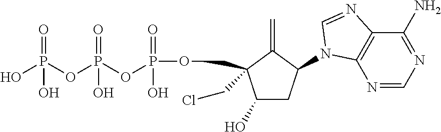 [SMILES: C=C1[C@@H](n2cnc3c(N)ncnc32)C[C@H](O)[C@@]1(CCl)COP(=O)(O)OP(=O)(O)OP(=O)(O)O]

The title compound was prepared in a manner analogous to Example 21 using (1S,2S,4S)-4-(6-Amino-9H-purin-9-yl)-2-(chloromethyl)-2-(hydroxymethyl)-3-methylenecyclopentan-1-ol (Example 14) as the nucleoside starting material.

### Example 32. ((1S,3S,5S)-1-Cyano-5-hydroxy-3-(5-methyl-2,4-dioxo-3,4-dihydropyrimidin-1(2H)-yl)-2-methylenecyclopentyl)methyl Tetrahydrogen Triphosphate

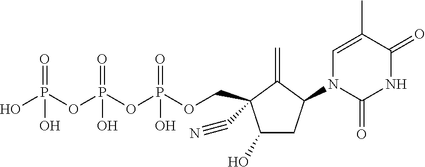 [SMILES: C=C1[C@@H](n2cc(C)c(=O)[nH]c2=O)C[C@H](O)[C@@]1(C#N)COP(=O)(O)OP(=O)(O)OP(=O)(O)O]

The title compound was prepared in a manner analogous to Example 21 using (1S,3S,5S)-5-Hydroxy-1-(hydroxymethyl)-3-(5-methyl-2,4-dioxo-3,4-dihydropyrimidin-1(2H)-yl)-2-methylenecyclopentane-1-carbonitrile (Example 15) as the nucleoside starting material.

### Example 33. ((1S,3S,5S)-3-(4-Amino-2-oxopyrimidin-1(2H)-yl)-1-cyano-5-hydroxy-2-methylenecyclopentyl)methyl Tetrahydrogen Triphosphate

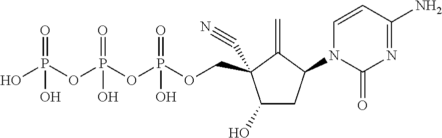 [SMILES: C=C1[C@@H](n2ccc(N)nc2=O)C[C@H](O)[C@@]1(C#N)COP(=O)(O)OP(=O)(O)OP(=O)(O)O]

The title compound was prepared in a manner analogous to Example 21 using (1S,3S,5S)-3-(4-Amino-2-oxopyrimidin-1(2H)-yl)-5-hydroxy-1-(hydroxymethyl)-2-methylenecyclopentane-1-carbonitrile (Example 16) as the nucleoside starting material.

### Example 34. ((1S,3S,5S)-3-(6-Amino-9H-purin-9-yl)-1-cyano-5-hydroxy-2-methylenecyclopentyl)methyl Tetrahydrogen Triphosphate

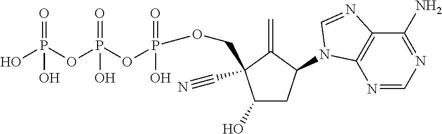 [SMILES: C=C1[C@@H](n2cnc3c(N)ncnc32)C[C@H](O)[C@@]1(C#N)COP(=O)(O)OP(=O)(O)OP(=O)(O)O]

The title compound was prepared in a manner analogous to Example 21 using (1S,3S,5S)-3-(6-Amino-9H-purin-9-yl)-5-hydroxy-1-(hydroxymethyl)-2-methylenecyclopentane-1-carbonitrile (Example 17) as the nucleoside starting material.

### Example 35. ((1S,3S,5S)-3-(2-Amino-6-oxo-1,6-dihydro-9H-purin-9-yl)-1-cyano-5-hydroxy-2-methylenecyclopentyl)methyl Tetrahydrogen Triphosphate

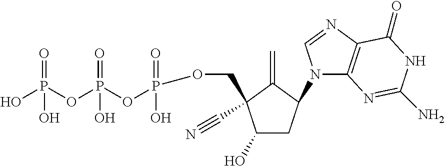 [SMILES: C=C1[C@@H](n2cnc3c(=O)[nH]c(N)nc32)C[C@H](O)[C@@]1(C#N)COP(=O)(O)OP(=O)(O)OP(=O)(O)O]

The title compound was prepared in a manner analogous to Example 21 using (1S,3S,5S)-3-(2-Amino-6-oxo-1,6-dihydro-9H-purin-9-yl)-5-hydroxy-1-(hydroxymethyl)-2-methylenecyclopentane-1-carbonitrile (Example 18) as the nucleoside starting material.

### Example 36. ((1R,3S,5S)-3-(6-Amino-9H-purin-9-yl)-1-ethynyl-5-hydroxy-2-methylcyclopentyl)methyl Tetrahydrogen Triphosphate

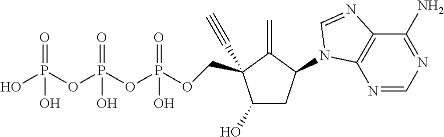 [SMILES: C#C[C@]1(COP(=O)(O)OP(=O)(O)OP(=O)(O)O)C(=C)[C@@H](n2cnc3c(N)ncnc32)C[C@@H]1O]

The title compound was prepared in a manner analogous to Example 21 using (1S,2R,4S)-4-(6-Amino-9H-purin-9-yl)-2-ethynyl-2-(hydroxymethyl)-3-methylenecyclopentan-1-ol (Example 19) as the nucleoside starting material.

### Example 37. ((1R,3S,5S)-3-(6-Amino-9H-purin-9-yl)-5-hydroxy-2-methylene-1-vinylcyclopentyl)methyl Tetrahydrogen Triphosphate

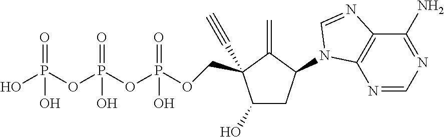 [SMILES: C#C[C@]1(COP(=O)(O)OP(=O)(O)OP(=O)(O)O)C(=C)[C@@H](n2cnc3c(N)ncnc32)C[C@@H]1O]

The title compound was prepared in a manner analogous to Example 21 using (1S,2R,4S)-4-(6-Amino-9H-purin-9-yl)-2-(hydroxymethyl)-3-methylene-2-vinylcyclopentan-1-ol (Example 20) as the nucleoside starting material.

TABLE 1

| # | MS [M-1] | P(α) and P(γ) | P(β) |
|---|---|---|---|
| 22 | 499.3 | −10.28 (d); −11.6 (t) | −23.06 (d) |
| 23 | 515.2 | −6.38 (d); −11.07 (d) | −22.64 (t) |
| 24 | 509.7 | −8.62 (br.s); −11.72 (d) | −22.98 (t) |
| 25 | 534.6 | −11.03 (d); −11.52 (d) | −23.38 (t) |
| 26 | 518.6 | −11.01 (d); −11.79 (d) | −23.34 (t) |
| 27 | 517.4 | −6.39 (d); −11.64 (d) | −22.58 (t) |
| 28 | 548.2 | −6.32 (d); −12.04 (d) | −22.56 (t) |
| 29 | 508.8 | −9.09 (br.s); −11.48 (d) | −23.11 (t) |
| 30 | 523.5 | −11.05 (d); −11.59 (d) | −23.46 (t) |
| 31 | 547.8 | −10.93 (d); −11.46 (d) | −23.36 (t) |
| 32 | 516.5 | −10.66 (d); −12.52 (d) | −23.35 (t) |
| 33 | 501.6 | −6.36 (d); −12.34 (d) | −22.59 (t) |
| 34 | 525.7 | −6.31 (d); −12.26 (d) | −22.52 (t) |
| 35 | 541.6 | −10.14 (d); −12.15 (d) | −23.19 (t) |
| 36 | 524.3 | −6.45 (br.s); −11.79 (d) | −22.42 (t) |
| 37 | 526.7 | −10.54 (d); −11.99 (d) | −23.31 (t) |

### Example 38. Isopropyl ((((1R,3S,5S)-3-(6-amino-9H-purin-9-yl)-1-ethynyl-5-hydroxy-2-methylenecyclopentyl)methoxy)(phenoxy)phosphoryl)-L-alaninate

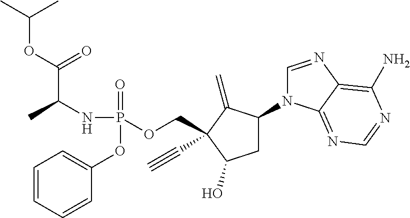 [SMILES: C#C[C@]1(COP(=O)(N[C@@H](C)C(=O)OC(C)C)Oc2ccccc2)C(=C)[C@@H](n2cnc3c(N)ncnc32)C[C@@H]1O]

Step A. (E)-N′-(9-((1S,3R,4S)-3-Ethynyl-4-hydroxy-3-(hydroxymethyl)-2-methylenecyclopentyl)-9H-purin-6-yl)-N,N-dimethylformimidamide. To a r.t. solution of (1S,2R,4S)-4-(6-amino-9H-purin-9-yl)-2-ethynyl-2-(hydroxymethyl)-3-methylenecyclopentan-1-ol (Example 19, 11.6 mg, 0.0419 mmol) in MeOH (1.0 mL) was added N,N-dimethylformamide dimethyl acetal (0.1 mL, 89.4 mg, 0.75 mmol). The reaction mixture was purged with Ar, and stirred at r.t. for 16 h. The solvent was evaporated to give the title compound, which was further dried under high vacuum overnight. Crude (E)-N′-(9-((1S,3R,4S)-3-Ethynyl-4-hydroxy-3-(hydroxymethyl)-2-methylenecyclopentyl)-9H-purin-6-yl)-N,N-dimethylformimidamide was used in the next step without further purification.

Step B. Isopropyl ((((1R,3S,5S)-3-(6-(((E)-(dimethylamino)methylene)amino)-9H-purin-9-yl)-1-ethynyl-5-hydroxy-2-methylenecyclopentyl)methoxy)(phenoxy)phosphoryl)-L-alaninate. (E)-N′-(9-((1S,3R,4S)-3-ethynyl-4-hydroxy-3-(hydroxymethyl)-2-methylenecyclopentyl)-9H-purin-6-yl)-N,N-dimethylformimidamide was dissolved in anhydrous THF (1.0 mL), and N-methylimidazole (NMI) (30 mg, 29 μL, 0.36 mmol) was added at rt. (2S)-Isopropyl 2-((chloro(phenoxy)phosphoryl)amino)propanoate (45 mg, 0.22 mmol) was then added to the reaction mixture and the reaction mixture was stirred at r.t. for 16 h. The reaction mixture was concentrated at 35° C. under reduced pressure, and then dried under high vacuum. The title compound was isolated as a mixture of isomers at the phosphorous center (Rₚ and Sₚ): Isopropyl ((((1R,3S,5S)-3-(6-(((E)-(dimethylamino)methylene)amino)-9H-purin-9-yl)-1-ethynyl-5-hydroxy-2-methylenecyclopentyl)methoxy)(phenoxy)phosphoryl)-L-alaninate was used crude in the next step without further purification.

Step C. Isopropyl ((((1R,3S,5S)-3-(6-amino-9H-purin-9-yl)-1-ethynyl-5-hydroxy-2-methylenecyclopentyl)methoxy)(phenoxy)phosphoryl)-L-alaninate. A solution of Isopropyl ((((1R,3S,5S)-3-(6-(((E)-(dimethylamino)methylene)amino)-9H-purin-9-yl)-1-ethynyl-5-hydroxy-2-methylenecyclopentyl)methoxy)(phenoxy)phosphoryl)-L-alaninate and 0.377 M TFA solution in MeOH—H₂O (2.0 mL) was stirred at rt for 16 h. The reaction mixture was concentrated under reduced pressure. Purification (FCC, SiO₂, MeOH/DCM, 0 to 20%) and preparative HPLC (CH₃CN—H₂O, 5 to 95%, including 0.1% formic acid) afforded the title compound as a mixture of isomers at the phosphorous center (Rₚ and Sₚ) as a white fluffy solid (4.4 mg). ³¹P NMR (400 MHz, CDCl₃): δ 3.36, 3.13. MS, m/Z 555.6 (M+1)*.

### Example 39. Neopentyl ((((1S,3S,5S)-3-(6-amino-9H-purin-9-yl)-1-fluoro-5-hydroxy-2-methylenecyclopentyl)methoxy)(phenoxy)phosphoryl)-L-alaninate

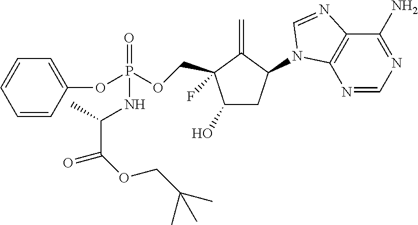 [SMILES: C=C1[C@@H](n2cnc3c(N)ncnc32)C[C@H](O)[C@@]1(F)COP(=O)(N[C@@H](C)C(=O)OCC(C)(C)C)Oc1ccccc1]

Step A. N,N-Di-BOC-(1S,2S,4S)-4-(6-amino-9H-purin-9-yl)-2-fluoro-2-(hydroxymethyl)-3-methylenecyclopentan-1-ol. The title compound was prepared in a manner similar to (1S,2S,4S)-4-(6-Amino-9H-purin-9-yl)-2-fluoro-2-(hydroxymethyl)-3-methylenecyclopentan-1-ol (Example 6).

Step B. N,N-Di-BOC-((((1S,3S,5S)-3-(6-amino-9H-purin-9-yl)-1-fluoro-5-hydroxy-2-methylenecyclopentyl)methoxy)(phenoxy)phosphoryl)-L-alaninate. To solution of the N,N-Di-BOC-(1S,2S,4S)-4-(6-amino-9H-purin-9-yl)-2-fluoro-2-(hydroxymethyl)-3-methylenecyclopentan-1-ol (1.0 equiv.) in dry THF was added isoPrMgCl (Isopropylmagnesium chloride) (1.0 M solution in THF, 1.5 eq.) followed by addition of the appropriate phosphorochloridate (2.0 equiv.) dissolved in anhydrous THF (3 mL). The reaction mixture was stirred for 5 h, quenched with water, and extracted with ethyl acetate. The organic layer was dried (Na₂SO₄), filtered, and concentrated under reduced pressure. Purification (FCC, SiO₂, MeOH/DCM from 2% to 15%) afforded the title compound as a mixture of isomers at the phosphorous center (Rₚ and Sₚ): N,N-Di-BOC-((((1S,3S,5S)-3-(6-amino-9H-purin-9-yl)-1-fluoro-5-hydroxy-2-methylenecyclopentyl)methoxy)(phenoxy)phosphoryl)-L-alaninate (13%). MS (M−1) δ51.3.

Step C. Neopentyl ((((1S,3S,5S)-3-(6-amino-9H-purin-9-yl)-1-fluoro-5-hydroxy-2-methylenecyclopentyl)methoxy)(phenoxy)phosphoryl)-L-alaninate. N,N-Di-BOC-((((1S,3S,5S)-3-(6-amino-9H-purin-9-yl)-1-fluoro-5-hydroxy-2-methylenecyclopentyl)methoxy)(phenoxy)phosphoryl)-L-alaninate was treated with the mixture of acetonitrile and HCl/dioxane 9:1 (v/v) for 9 h at rt. The reaction mixture was concentrated under reduced pressure. Purification (Reverse Phase HPLC, Phenomenex Synergi 4 micron Hydro-RP 80A 250×21.2 cm in gradient from 25% to 95% acetonitrile in water, 10 mM TEAA) afforded the title compound as a mixture of isomers at the phosphorous center (Rₚ and Sₚ). P³¹—NMR (CD₃OD) 6 ppm: 3.44, 3.77. MS [M−1] 578.2.

### Example 40. Isopropyl ((((1S,3S,5S)-3-(6-amino-9H-purin-9-yl)-1-(fluoromethyl)-5-hydroxy-2-methylenecyclopentyl)methoxy)(phenoxy)phosphoryl)-L-alaninate

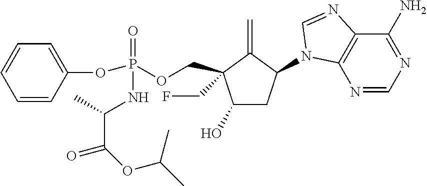 [SMILES: C=C1[C@@H](n2cnc3c(N)ncnc32)C[C@H](O)[C@@]1(CF)COP(=O)(N[C@@H](C)C(=O)OC(C)C)Oc1ccccc1]

(1S,2S,4S)-4-(6-Amino-9H-purin-9-yl)-2-(fluoromethyl)-2-(hydroxymethyl)-3-methylenecyclopentan-1-ol (Example 13, 11 mg, 0.037 mmol) was dissolved in the mixture of dry acetonitrile (0.9 mL) and N-methylimidazole (0.1 mL. (2S)-Isopropyl 2-((chloro(phenoxy)phosphoryl)amino)propanoate (prepared according to J. Med. Chem. 2014, 57, 1531-1542) was added (30 mg, 0.1 mmol). The reaction mixture was heated to 60° C. for 4 h, then continued to heat at 40° C. for an additional 48 h. The reaction mixture was diluted with ethyl acetate (30 mL). The organic phase was washed with 10% citric acid, brine, dried (Na₂SO₄), filtered, and concentrated under reduced pressure. Purification (FCC, SiO₂, MeOH in DCM from 2% to 10%) followed by re-purification (Reverse phase HPLC on Synergy 4 micron Hydro-RP column (Phenominex)/a linear gradient of acetonitrile from 30 to 100% in 10 mM triethylammonium acetate buffer (pH 7.5) was used for elution) afforded the title compound as a mixture of isomers at the phosphorous center (Rₚ and Sₚ) with total yield 10%. P³¹—NMR (CD₃OD) 6 ppm: 3.06, 3.31. MS [M−1] 564.6.

### Example 41. Isopropyl ((((1S,3S,5S)-1-cyano-5-hydroxy-3-(5-methyl-2.4-dioxo-3.4-dihydropyrimidin-1(2H)-yl)-2-methylenecyclopentyl)methoxy)(phenoxy)phosphoryl)-L-alaninate

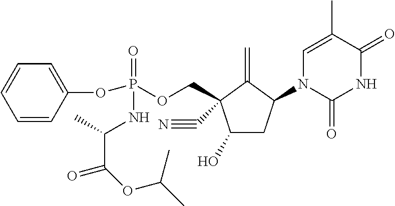 [SMILES: C=C1[C@@H](n2cc(C)c(=O)[nH]c2=O)C[C@H](O)[C@@]1(C#N)COP(=O)(N[C@@H](C)C(=O)OC(C)C)Oc1ccccc1]

The title compound as a mixture of isomers at the phosphorous center (Rₚ and Sₚ) was prepared in a manner analogous to Example 38, Step B, using (1S,3S,5S)-5-Hydroxy-1-(hydroxymethyl)-3-(5-methyl-2,4-dioxo-3,4-dihydropyrimidin-1(2H)-yl)-2-methylenecyclopentane-1-carbonitrile (Example 15) in Step B. ³¹P NMR (400 MHz, CDCl₃): δ 3.20, 3.01. MS, m/Z 547.1 [M+1]⁺.

### Example 42. Isopropyl ((((1S,3S,5S)-3-(4-amino-2-oxopyrimidin-1(2H)-yl)-1-cyano-5-hydroxy-2-methylenecyclopentyl)methoxy)(phenoxy)phosphoryl)-L-alaninate

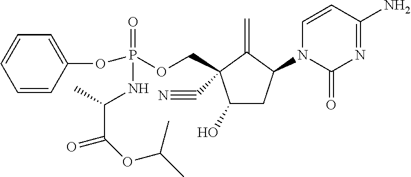 [SMILES: C=C1[C@@H](n2ccc(N)nc2=O)C[C@H](O)[C@@]1(C#N)COP(=O)(N[C@@H](C)C(=O)OC(C)C)Oc1ccccc1]

The title compound as a mixture of isomers at the phosphorous center (Rₚ and Sₚ) was prepared in a manner analogous to Example 38, using (1S,3S,5S)-3-(4-Amino-2-oxopyrimidin-1(2H)-yl)-5-hydroxy-1-(hydroxymethyl)-2-methylenecyclopentane-1-carbonitrile (Example 16) instead of (1S,2R,4S)-4-(6-amino-9H-purin-9-yl)-2-ethynyl-2-(hydroxymethyl)-3-methylenecyclopentan-1-ol (Example 19) in Step A. ³¹P NMR (400 MHz, CDCl₃): δ 3.12, 2.95. MS, m/Z 532.0 (M+1)*.

### Example 43. Isopropyl ((R*)-(((1S,3S,5S)-3-(2-amino-6-oxo-1,6-dihydro-9H-purin-9-yl)-1-cyano-5-hydroxy-2-methylenecyclopentyl)methoxy)(phenoxy)phosphoryl)-L-alaninate

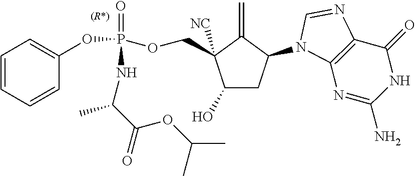 [SMILES: C=C1[C@@H](n2cnc3c(=O)[nH]c(N)nc32)C[C@H](O)[C@@]1(C#N)CO[P@](=O)(N[C@@H](C)C(=O)OC(C)C)Oc1ccccc1]

To a cooled, 0° C., suspension of (1S,3S,5S)-3-(2-amino-6-oxo-1,6-dihydro-9H-purin-9-yl)-5-hydroxy-1-(hydroxymethyl)-2-methylenecyclopentane-1-carbonitrile (Example 18, 0.016 g, 0.053 mmol) in THF (1.0 mL, 0.05 M) was added tert-butylmagnesium chloride (1.0 M THF, 0.12 mL, 0.12 mmol) dropwise. The reaction mixture was stirred at r.t. for 30 min. The reaction mixture was then cooled to 0° C., and a solution of isopropyl (chloro(phenoxy)phosphoryl)-L-alaninate (30 mg, 0.1 mmol) in THF (0.13 mL) was added dropwise. The reaction mixture was stirred at r.t. for 2 h, then concentrated in vacuo to give a white solid. Purification (reverse phase HPLC, Phenomenex Synergyi 4 micron Hydro-RP 80 A 250×21.2 mm, (0-99% over 25 min, acetonitrile with TEAA buffer in H₂O with TEAA buffer) afforded two isomers (at the phosphorous center): Isopropyl ((R*)-(((1S,3S,5S)-3-(2-amino-6-oxo-1,6-dihydro-9H-purin-9-yl)-1-cyano-5-hydroxy-2-methylenecyclopentyl)methoxy)(phenoxy)phosphoryl)-L-alaninate ¹H NMR (400 MHz, CDCl₃): δ 7.80 (s, 1H), 7.30-7.10 (m, 5H), 5.60 (m, 1H), 5.53 (m, 1H), 5.08 (m, 1H), 4.98 (sept, J=6.0, 1H), 4.89 (dd, J=11, 4.8, 1H), 4.59 (d, J=3.6, 1H), 4.42 (dd, J=10, 3.6, 1H), 3.99 (dq, J=8.8, 7.6, 1H), 2.84 (dd, J=13, 3.6, 1H), 2.31 (dd, J=13, 8.0, 1H), 1.39 (m, 3H), 1.23 (m, 6H). ³¹P NMR (400 MHz, CDCl₃): δ 4.05. MS, m/Z 572.10 [M+1]⁺; and Isopropyl ((S*)-(((1S,3S,5S)-3-(2-amino-6-oxo-1,6-dihydro-9H-purin-9-yl)-1-cyano-5-hydroxy-2-methylenecyclopentyl)methoxy)(phenoxy)phosphoryl)-L-alaninate (Example 44).

### Example 44. Isopropyl ((S*)-(((1S,3S,5S)-3-(2-amino-6-oxo-1,6-dihydro-9H-purin-9-yl)-1-cyano-5-hydroxy-2-methylenecyclopentyl)methoxy)(phenoxy)phosphoryl)-L-alaninate

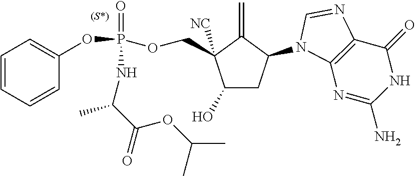 [SMILES: C=C1[C@@H](n2cnc3c(=O)[nH]c(N)nc32)C[C@H](O)[C@@]1(C#N)CO[P@@](=O)(N[C@@H](C)C(=O)OC(C)C)Oc1ccccc1]

The title compound was isolated as the other isomer from Example 43, pure but unknown stereochemistry (at the phosphorous center). ¹H NMR (400 MHz, CDCl₃): δ 7.80 (s, 1H), 7.42-7.22 (m, 5H), 5.61-5.54 (m, 2H), 5.15-5.11 (m, 2H), 5.02 (sept, J=6.0, 1H), 4.39 (d, J=4.0, 1H), 4.27 (dd, J=11, 4, 1H), 3.97 (dq, J=10, 7.2, 1H), 2.90 (dd, J=14, 4.0, 1H), 2.22 (dd, J=14, 8.4, 1H), 1.36 (m, 3H), 1.23 (m, 6H). ³¹P NMR (400 MHz, CDCl₃): δ 3.72. MS, m/Z 572.10 [M+1]*.

### Biology Assays

### Example A. HIV Single-Cycle Assay

24 h prior to infection, CEM human T lymphoblast cells (ATCC, Manassas, Va.) were plated in assay media (MEM supplemented with 10% FBS, 1% penicillin/streptomycin (all Mediatech, Manassas, Va.) and 1% DMSO (Sigma-Aldrich, St Louis, Mo.)) were plated at a density of 5×10⁵ cells/mL (5×10⁴ cells/well) in white 96-well plates. Serially diluted compounds were added to cells and incubated overnight at 37° C., 5% CO₂. The following day, cells were infected with VSV-G pseudotyped HIV NL4-3, in which parts of the env and nef were genes replaced with Renilla-luciferase, and infected cells were incubated for 72 h at 37° C., 5% CO₂. Viral inoculum was titrated to achieve a Renilla-luciferase signal of approximately 100× fold over background. Antiviral activity was measured by addition of 100 ul of Renilla-Glo® reagent (Promega, Madison, Wis.) to infected cells. After a 10-min incubation at RT, luminescence was measured on a Victor X₃ multi-label plate reader (Perkin Elmer, Waltham, Mass.). Cytotoxicity of uninfected parallel cultures was determined by addition of 100 μL CellTiter-Glo®reagent (Promega, Madison, Wis.), and incubation for 10 mins at RT. Luminescence was measured on a Victor X₃ multi-label plate reader.

### Example B. Inhibition of HIV Reverse Transcriptase

Recombinant full-length HIV-1 Reverse Transcriptase (HIVrt) was purchased from Abcam, catalog #ab63979. The last 385 nucleotide region of the HCV anti-genome complementary to the 5′ untranslated region (c5′UTR) was synthesized using T7 RNA polymerase Megascript kit from Ambion (Cat #AM1333). A DNA oligo served as an internal initiation primer and was purchased from IDT. Unless otherwise specified, reaction samples consisted of 20 nM c5′UTR RNA, 100 nM DNA primer, and 1 nM HIVrt, mixed together in a buffer containing 50 mM Tris pH 7.5, 100 mM KCl, 4 mM dithiothreitol (DTT), and 12.5 mM MgCl₂. Reactions were initiated at 30° C. by adding 0.1 μM adenosine triphosphate (dATP), 0.1 μM cytosine triphosphate (dCTP), 1 μM guanosine triphosphate (dGTP), and 0.32 μM ³H-thymidine triphosphate (³H-TTP), in a final volume of 50 μL. After 40-mins incubation, the reaction was terminated by adding 60 μL of chilled 20% (w/v) trichloroacetic acid with 500 μM ATP to precipitate nucleic acids. After incubation at 4° C. for 1 h, the sample underwent filtration on a multiscreen BV 1.2-m 96-well plate (Millipore). 40 μL Microscint-20 (Perkin Elmer) was added to the well and the counts in the sample were determined by a Trilux Microbeta microplate scintillation reader (Wallac).

All data were analyzed with GraphPad Prism. The compound concentration at which the enzyme-catalyzed rate was reduced by 50% (IC₅₀) was calculated by fitting the data to the equation Y=% Min+(% Max−% Min)/(1+X/IC₅₀), where Y corresponds to the percent relative enzyme activity, % Min is the residual relative activity at saturating compound concentration, % Max is the relative maximum enzyme activity, and X corresponds to the compound concentration. The Kᵢ was calculated using the Cheng-Prusoff equation assuming competitive inhibition relative to natural dNTP incorporation: K=IC₅₀/(1+[dNTP]/Kₘ), where [dNTP] is the concentration of natural dNTP and Kₘ is the apparent Kₘ for dNTP. The standard HIVrt RNA-dependent DNA polymerization (RdDp) assay was used to determine the IC₅₀ values.

### Example C. Inhibition of HBV Polymerase

Recombinant full-length HBV polymerase(HBVpol) was expressed in SF9 cells and purified according to Lanford et al (Nucleotide priming and reverse transcriptase activity of hepatitis B virus polymerase expressed in insect cells. (Lanford et al., J Virol. 1995; 69(7):4431-4439). The last 385 nucleotide region of the HCV anti-genome complementary to the 5′ untranslated region (c5′UTR) was synthesized using T7 RNA polymerase Megascript kit from Ambion (Cat #AM1333). A DNA oligo served as an internal initiation primer and was purchased from IDT. Unless otherwise specified, reaction samples consisted of 50 nM c5′UTR RNA, 500 nM DNA primer, and 1 uL HIVrt, mixed together in a buffer containing 50 mM Tris pH 7.5, 100 mM KCl, 4 mM dithiothreitol (DTT), 10% DMSO and 12.5 mM MgCl₂. Reactions were initiated at 30° C. by adding 46 nM adenosine triphosphate (dATP), 17 nM cytosine triphosphate (dCTP), 57 nM guanosine triphosphate (dGTP), and 0.32 μM ³H-thymidine triphosphate (³H-TTP), in a final volume of 50 μL. After 120-mins incubation, the reaction was terminated by adding 60 μL of chilled 20% (w/v) trichloroacetic acid with 500 μM ATP to precipitate nucleic acids. After incubation at 4° C. for 1 h, the sample underwent filtration on a multiscreen BV 1.2-m 96-well plate (Millipore). 40 μL Microscint-20 (Perkin Elmer) was added to the well and the counts in the sample were determined by a Trilux Microbeta microplate scintillation reader (Wallac).

All data were analyzed with GraphPad Prism. The compound concentration at which the enzyme-catalyzed rate was reduced by 50% (IC₅₀) was calculated by fitting the data to the equation Y=% Min+(% Max−% Min)/(1+X/IC₅₀), where Y corresponds to the percent relative enzyme activity, % Min is the residual relative activity at saturating compound concentration, % Max is the relative maximum enzyme activity, and X corresponds to the compound concentration. The Kᵢ was calculated using the Cheng-Prusoff equation assuming competitive inhibition relative to natural dNTP incorporation: K=IC₅₀/(1+[dNTP]/Kₘ), where [dNTP] is the concentration of natural dNTP and Kₘ is the apparent Kₘ for dNTP. The standard HBVpol RNA-dependent DNA polymerization (RdDp) assay was used to determine the IC₅₀ values.

### Example D. Inhibition of HBV in Hepg2.117 Cells

HepG2.117 cells (use passage less than 25 passages) were cultured in DMEM/F12 50/50 medium (Corning, REF 10-092-CM) with 10% FBS (Corning REF 35-011-CV), 250 ug/mL G418 Sulfate (Corning, REF 30-234-CI), 2 ug/mL Tetracycline (TEKNOVA, cat #T3325) and 1× Penicillin/Streptomycin (Corning, 30-002-CI), (Corning, 30-002-CI). For each assay, cells were plated in assay medium: DMEM/F12 50/50 (Corning, REF 10-092-CM), 2% Tet-system approved FBS (Clontech, Cat #631106) and 1× Penicillin/Streptomycin (Corning, 30-002-CI).

### Determination of Anti-HBV Activity

Determination of 50% inhibitory concentration (EC₅₀) of compounds in HepG2.117 cells were performed by the following procedure. On the first day, cells were washed with PBS two times after trypsinizing the cells. Then cells were washed once with the assay medium. Cells were seeded at 30,000-35,000 cells per 100 μL per well in Biocoat collage coated flat bottom 96 well plates. Test compounds were solubilized in 100% DMSO to 100× the desired final testing concentration. Each compound was then serially diluted (1:3) up to 9 different concentrations. Compounds in 100% DMSO are reduced to 10% DMSO by diluting 1:10 in assay media. After incubation of the cells in a 37 C, 5% CO₂ incubator for 4 h, 10 uL test compounds diluted in assay media were added into cell plate. The final DMSO concentration was 1%. The cells were incubated at 37° C. for 96 h.

The antiviral activity was measured using a Real Time quantitative polymerase chain reaction (RT qPCR) assay directly measuring the HBV viral DNA copy numbers from the supernatant of HepG2.117 cells. The HBV Core primers and probes used in qPCR: core forward primer was 5′-CTGTGCCTTGGGTGGCTTT-3′ (SEQ. ID. NO. 1); the core reverse primer was 5′-AAGGAAAGAAGTCAGAAGGCAAAA-3′ (SEQ. ID. NO. 2); the core probe was 5′/FAM/AGCTCCAAA/ZEN/TCCTTTATAAGGGTCGATGTCCATG/31ABKFQ/−3′ (SEQ. ID. NO. 3). The core forward and core reverse probes were used at a final concentration of 1 μM and the core probe was used at a final concentration of 0.5 μM. The RT qPCR assay was set up with 10 μL 2× Quanta Perfecta qPCR ToughMix ROX, 0.1 μL 200× primer/probe mix, 4.0 μL HepG2.117 cell supernatant (or standard for control wells) and 5.9 μL dH₂O, for a total reagent volume of 20 μL per well. The standard was prepared by diluting HBV DNA Plasmid, Psi Check, in 10 mM TE buffer in a 1:5 ratio in 6 concentrations: 1E6, 0.2E6, 0.04E6, 0.008E6, 0.0016E6, 0.00032E6 of viral DNA copy numbers. The RT qPCR (Applied Biosystems and “Quant Studio 6 Flex” from Life Technology) was run for 5 mins at 95° C., then 15 mins at 95° C. and 20 mins at 60° C. for each cycle, 40 cycles in total.

HBV viral DNA copy numbers are normalized to the level observed in the absence of inhibitor, which was defined as 100%. EC₅₀ was defined as the concentration of compound at which the HBV viral DNA copy numbers from the HepG2.117 cells was reduced 50% relative to its level in the absence of compound.

### Determination of Cytotoxicity in HepG2 Cells

Cell cytotoxicity (CC₅₀) against HepG2 cells was measured using a luminescent cell viability assay to determine the number of viable cells in the culture based on quantitation of the adenosine triphosphate (ATP) present after a 4-day incubation period. On the first day, HepG2 cells were seeded at 15,000/100 uL/well with assay media containing DEME (Corning, REF 10-013-CV), 3% FBS (Coning REF 35-011-CV), 1× Penicillin/Streptomycin (Corning, 30-002-CI), and 1× Non-Essential Amino Acid in Biocoat college 96-well flat bottom plates. Cells were incubated in a 37° C., 5% CO₂ incubator for 4 h before compound dosing. The compound dilution and dosing procedure were identical to that outlined with respect to determine anti-HBV activity. After 96 h incubation, cell viability is normalized to the level observed in the absence of inhibitor, which was defined as 100%. No cytotoxic effect on the HepG2 cells was defined as a 50% cytotoxic concentration (CC₅₀)>100 μM.

TABLE 2

| Example | HIV | HBV |
|---|---|---|
| # | EC₅₀ (uM) | EC₅₀ (uM) |
| 1 | NT | 0.0109 |
| 2 | >33.3333 | >10 |
| 3 | >33.3333 | >10 |
| 4 | 0.0356 | <0.0009 |
| 5 | >33.3333 | 2.4722 |
| 6 | 2.6836 | 0.0617 |
| 7 | >33.3333 | >10 |
| 8 | NT | NT |
| 9 | >33.33 | 7.4104 |
| 10 | 0.6621 | 0.166 |
| 11 | 0.9106 | 9.1782 |
| 12 | >33.33 | >10 |
| 13 | 5.0227 | >5.5298 |
| 14 | 1.0784 | 3.4464 |
| 15 | >33.3333 | >10 |
| 16 | 5.8324 | >10 |
| 17 | 2.2094 | >10 |
| 18 | 0.1203 | 0.0466 |
| 19 | 0.0665 | >10 |
| 20 | 0.5475 | >10 |

TABLE 3

| | HIVrt | HBVrt |
|---|---|---|
| Ex. # | IC₅₀ (uM) | IC₅₀ (uM) |
| 22 | 0.1577 | NT |
| 23 | 0.3829 | 0.0088 |
| 24 | 0.2666 | 0.0029 |
| 25 | 0.2506 | 0.0099 |
| 26 | 0.081 | 0.0082 |
| 27 | 0.0659 | 0.0072 |
| 28 | 0.749 | NT |
| 29 | 0.3255 | 0.38 |
| 30 | 1.3343 | NT |
| 31 | 0.3633 | NT |
| 32 | 0.4763 | >1 |
| 33 | 0.0779 | 0.22 |
| 34 | >10 | NT |
| 35 | 0.2601 | NT |
| 36 | 0.0331 | NT |
| 37 | >10 | NT |

TABLE 4

| Ex # | HIV EC₅₀ (uM) | HBV EC₅₀ (uM) |
|---|---|---|
| 38 | 0.0506 | 2.3743 |
| 39 | 0.0139 | 0.0003 |
| 40 | 0.144 | 0.0137 |
| 41 | 10.2491 | >10 |
| 42 | 10.4341 | >10 |
| 43 | 4.248 | 0.591 |
| 44 | 2.292 | 0.459 |

Furthermore, although the foregoing has been described in some detail by way of illustrations and examples for purposes of clarity and understanding, it will be understood by those of skill in the art that numerous and various modifications can be made without departing from the spirit of the present disclosure. Therefore, it should be clearly understood that the forms disclosed herein are illustrative only and are not intended to limit the scope of the present disclosure, but rather to also cover all modification and alternatives coming with the true scope and spirit of the invention.

What is claimed is:

1. A compound of Formula (I), or a pharmaceutically acceptable salt thereof, having the structure: 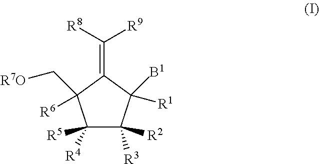
wherein:
B¹ is an optionally substituted C-linked heterocyclic base or an optionally substituted N-linked heterocyclic base;
R¹ is selected from the group consisting of hydrogen, halogen, cyano, an optionally substituted C₁₋₆ alkyl, an unsubstituted C₂₋₆ alkenyl and an unsubstituted C₂₋₆ alkynyl, wherein when the C₁₋₆ alkyl is substituted, the C₁₋₆ alkyl is substituted with at least one halogen;
R² is hydrogen or fluoro;
R³ is hydrogen or fluoro;
R⁴ is selected from the group consisting of hydrogen, halogen, hydroxy, cyano and an optionally substituted C₁₋₄ alkyl, wherein when the C₁₋₄ alkyl is substituted, the C₁₋₄ alkyl is substituted with a hydroxy or at least one halogen;
R⁵ is hydrogen or hydroxy;
R⁶ is selected from the group consisting of hydrogen, halogen, cyano, an optionally substituted C₁₋₄ alkyl, an optionally substituted C₂₋₄ alkenyl and an unsubstituted C₂₋₄ alkynyl, wherein when the C₁₋₄ alkyl or the C₂₋₄ alkenyl are substituted, the C₁₋₄ alkyl and C₂₋₄ alkenyl are independently substituted with at least one halogen;
R⁷ is selected from the group consisting of hydrogen, an optionally substituted acyl, an optionally substituted O-linked α-amino acid, 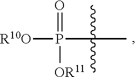 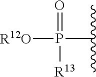
and 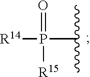
R¹⁰ and R¹¹ are independently selected from the group consisting of absent, hydrogen, 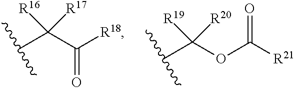
and 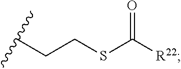
or
R¹⁰ is 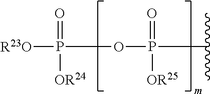
and R¹¹ is absent or hydrogen;
R¹² is absent, hydrogen, an optionally substituted aryl or an optionally substituted heteroaryl;
R¹³ is an optionally substituted N-linked α-amino acid or an optionally substituted N-linked α-amino acid ester derivative;
R¹⁴ and R¹⁵ are independently an optionally substituted N-linked α-amino acid or an optionally substituted N-linked α-amino acid ester derivative;
R¹⁶, R¹⁷, R¹⁹ and R²⁰ are independently selected from the group consisting of hydrogen, an optionally substituted C₁₋₂₄ alkyl and an optionally substituted aryl;
R¹⁸ and R²¹ are independently selected from the group consisting of hydrogen, an optionally substituted C₁₋₂₄ alkyl, an optionally substituted aryl, an optionally substituted —O—C₁₋₂₄ alkyl and an optionally substituted —O-aryl;
R²² is selected from the group consisting of hydrogen, an optionally substituted C₁₋₂₄ alkyl and an optionally substituted aryl;
R²³, R²⁴ and R²⁵ are independently absent or hydrogen;
R⁸ and R⁹ are independently hydrogen or halogen; and
m is 0 or 1; and
provided that a compound of Formula (I), or a pharmaceutically acceptable salt thereof, is not a compound of (i), (ii) or (iii), or a pharmaceutically acceptable salt thereof:
(i) when R¹ is hydrogen; R² is hydrogen or fluoro; R³ is hydrogen or fluoro; R⁴ is hydroxy; R⁵ is hydrogen; R⁶ is hydrogen or fluoro; R⁸ and R⁹ are each hydrogen and B¹ is selected from the group consisting of 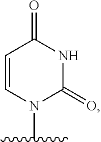 [SMILES: *n1ccc(=O)[nH]c1=O] 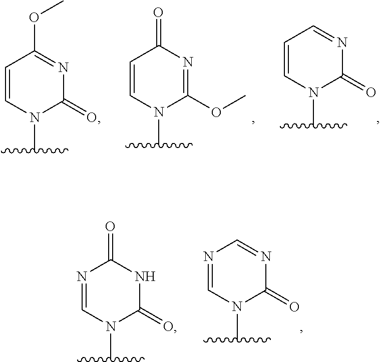 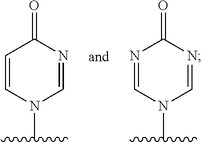 [SMILES: *n1ccc(=O)nc1 ; *n1cnc(=O)nc1]
then R⁷ is not selected from the group consisting of: (a) hydrogen; (b) 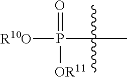
wherein R¹⁰ and R¹¹ are each hydrogen or each absent; (c) 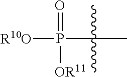
wherein R¹⁰ is 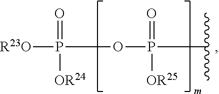
R¹¹, R²³, R²⁴ or R²⁵ are independently absent or hydrogen, and m is 0 or 1; and (d) 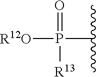
wherein R¹² is an unsubstituted phenyl or an unsubstituted naphthyl, and R¹³ is alanine isopropyl ester, alanine isobutyl ester or alanine neopentyl ester;
(ii) when R¹ is hydrogen; R⁴ is hydroxy; R⁵ is hydrogen; R⁶ is hydrogen; R⁸ and R⁹ are each hydrogen; B¹ is selected from the group consisting of 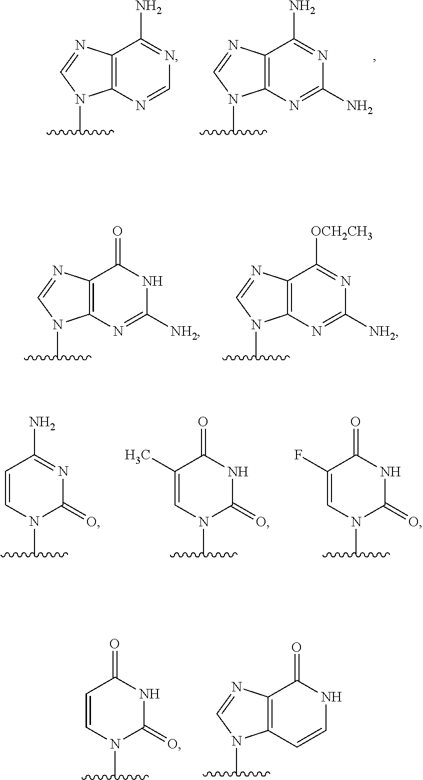 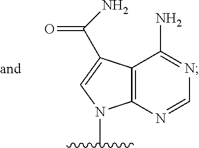 [SMILES: *n1cc(C(N)=O)c2c(N)ncnc21]
R⁷ is selected from the group consisting of: (a) 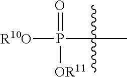
wherein R¹⁰ is 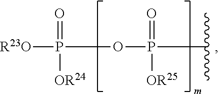
R¹¹, R²³, R²⁴ or R²⁵ are independently absent or hydrogen, and m is 1; and (b) 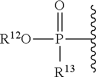
wherein R¹² is an unsubstituted phenyl, and R¹³ is alanine isopropyl ester, then (a) R² is not hydrogen when R³ are fluoro; and (b) R² is not fluoro when R³ are hydrogen; and
(iii) when R¹ is hydrogen; R⁴ is hydroxy; R⁵ is hydrogen; R⁶ is hydrogen; R⁷ is hydrogen and R⁸ and R⁹ are each hydrogen; then B¹ is not selected from the group consisting of

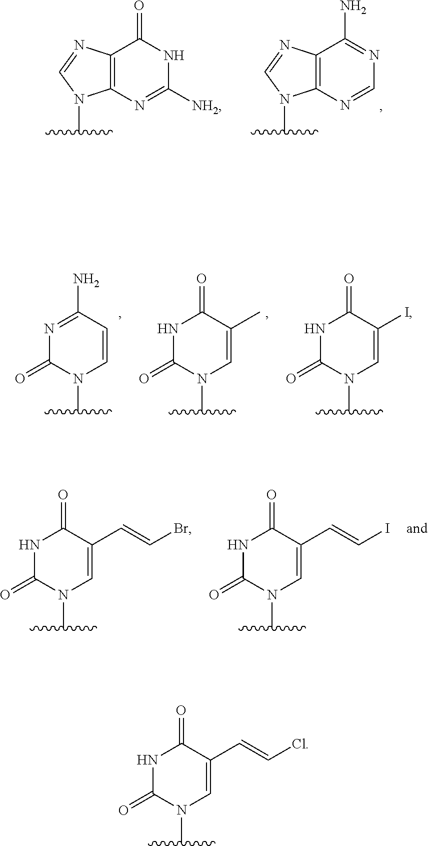

2. The compound of claim 1, wherein the compound of Formula (I) is selected from the group consisting of: 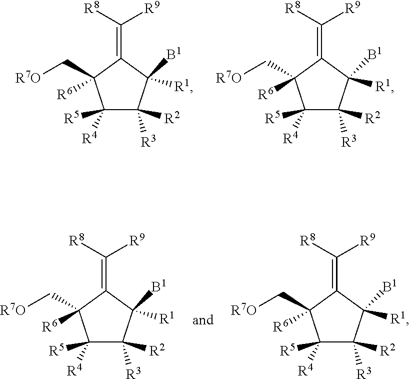
or a pharmaceutically acceptable salt of any of the foregoing.

3. The compound of claim 1 or 2, wherein B¹ is selected from the group consisting of: 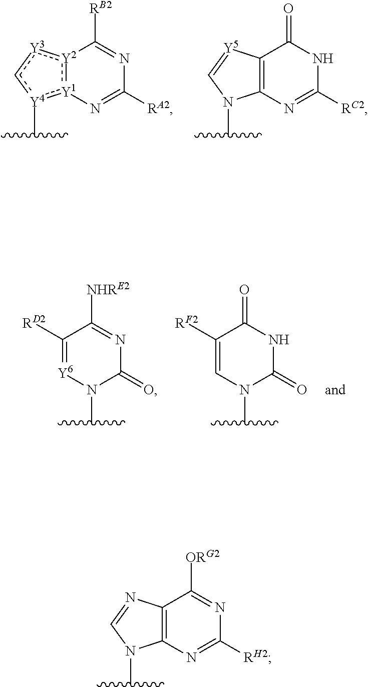
wherein:
R^{A2} is selected from the group consisting of hydrogen, halogen and NHR^{J2}, wherein R^{J2} is selected from the group consisting of hydrogen, —C(═O)R^{K2} and —C(═O)OR^{L2};
R^{B2} is halogen or NHR^{M2}, wherein R^{M2} is selected from the group consisting of hydrogen, an unsubstituted C₁₋₆ alkyl, an unsubstituted C₂₋₆ alkenyl, an unsubstituted C₃₋₈ cycloalkyl, —C(═O)R^{N2} and —C(═O)OR^{O2};
R^{C2} is hydrogen or NHR^{P2}, wherein R^{P2} is selected from the group consisting of hydrogen, —C(═O)R^{Q2} and —C(═O)OR^{R2};
R^{D2} is selected from the group consisting of hydrogen, halogen, an unsubstituted C₁₋₆ alkyl, an unsubstituted C₂₋₆ alkenyl and an unsubstituted C₂₋₆ alkynyl;
R^{E2} is selected from the group consisting of hydrogen, hydroxy, an unsubstituted C₁₋₆ alkyl, an unsubstituted C₃₋₈ cycloalkyl, —C(═O)R^{S2} and —C(═O)OR^{T2};
R^{F2} is selected from the group consisting of hydrogen, halogen, an unsubstituted C₁₋₆alkyl, an unsubstituted C₂₋₆ alkenyl and an unsubstituted C₂₋₆ alkynyl;
Y¹, Y² and Y⁴ are independently N or C, provided that at least one of Y¹, Y² and Y⁴ is N;
Y³ is N or CR^{U2}, wherein R^{U2} is selected from the group consisting of hydrogen, halogen, an unsubstituted C₁₋₆-alkyl, an unsubstituted C₂₋₆-alkenyl and an unsubstituted C₂₋₆-alkynyl;
Y⁵ and Y⁶ are independently N or CH;
each [symbol] is independently a single or double bond, provided that the single bonds and the double bonds are situated in the ring so that each ring is aromatic;
R^{G2} is an unsubstituted C₁₋₆ alkyl;
R^{H2} is hydrogen or NR^{V2}, wherein R^{V2} is independently selected from the group consisting of hydrogen, —C(═O)R^{W2} and —C(═O)OR^{X2}; and
R^{K2}, R^{L2}, R^{N2}, R^{O2}, R^{Q2}, R^{R2} R^{S2}, R^{T2}, R^{W2} and R^{X2} are independently selected from the group consisting of an unsubstituted C₁₋₆ alkyl, an unsubstituted C₂₋₆ alkenyl, an unsubstituted C₂₋₆ alkynyl, an optionally substituted C₃₋₆ cycloalkyl, an optionally substituted C₃₋₆ cycloalkenyl, an optionally substituted C₆₋₁₀ aryl, an optionally substituted heteroaryl, an optionally substituted heterocyclyl, an optionally substituted aryl(C₁₋₆ alkyl), an optionally substituted heteroaryl(C₁₋₆ alkyl) and an optionally substituted heterocyclyl(C₁₋₆ alkyl);

4. The compound of claim 1 or 2, wherein B¹ is an optionally substituted C-linked heterocyclic base.

5. The compound of claim 3, wherein B¹ is

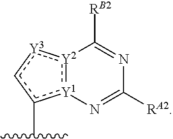

6. The compound of claim 3, wherein B¹ is selected from the group consisting of:

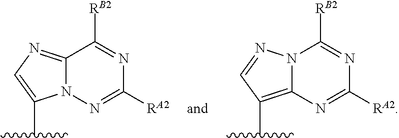

7. The compound of claim 4, wherein B¹ is selected from the group consisting of:

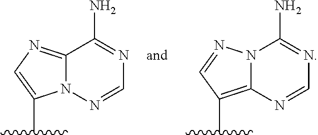 [SMILES: *c1cnc2c(N)ncnn12 ; *c1cnn2c(N)ncnc12]

8. The compound of claim 1 or 2, wherein B¹ is an optionally substituted N-linked heterocyclic base.

9. The compound of claim 8, wherein B¹ is an optionally substituted purine.

10. The compound of claim 8, wherein B¹ is an optionally substituted pyrimidine.

11. The compound of claim 3, wherein B¹ is selected from the group consisting of:

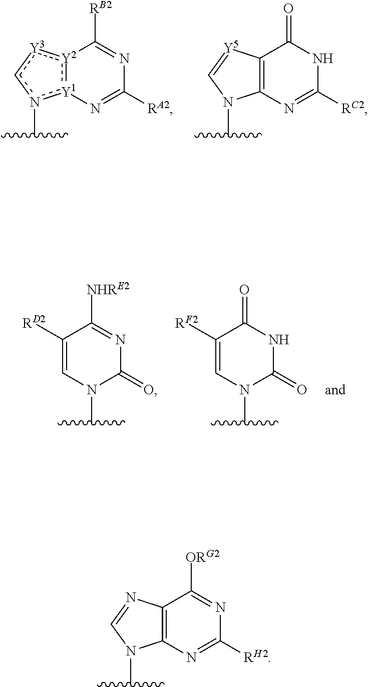

12. The compound of claim 8, wherein B¹ is selected from the group consisting of:

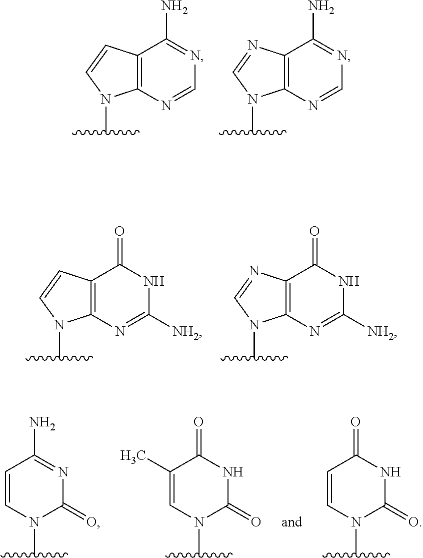

13. The compound of any one of claims 1-12, wherein R⁶ is halogen.

14. The compound of claim 13, wherein the halogen is fluoro.

15. The compound of any one of claims 1-12, wherein R⁶ is cyano.

16. The compound of any one of claims 1-12, wherein R⁶ is an optionally substituted C₁₋₄ alkyl, wherein when the C₁₋₄ alkyl is substituted, the C₁₋₄ alkyl is substituted with a halogen.

17. The compound of any one of claims 1-12, wherein R⁶ is an unsubstituted C₁₋₄ alkyl.

18. The compound of any one of claims 1-12, wherein R⁶ is a fluoro-substituted C₁₋₄ alkyl.

19. The compound of any one of claims 1-12, wherein R⁶ is a chloro-substituted C₁₋₄ alkyl.

20. The compound of any one of claims 1-12, wherein R⁶ is an optionally substituted C₂₋₄ alkenyl, wherein when the C₂₋₄ alkenyl is substituted, C₂₋₄ alkenyl is substituted with a halogen.

21. The compound of any one of claims 1-12, wherein R⁶ is an unsubstituted C₂₋₄ alkenyl.

22. The compound of any one of claims 1-12, wherein R⁶ is a fluoro-substituted C₂₋₄ alkenyl.

23. The compound of any one of claims 1-12, wherein R⁶ is a chloro-substituted C₂₋₄ alkenyl.

24. The compound of any one of claims 1-12, wherein R⁶ is hydrogen.

25. The compound of any one of claims 1-24, wherein R⁵ is hydrogen.

26. The compound of any one of claims 1-24, wherein R⁵ is hydroxy.

27. The compound of any one of claims 1-26, wherein R⁴ is halogen.

28. The compound of any one of claims 1-26, wherein R⁴ is hydroxy.

29. The compound of any one of claims 1-26, wherein R⁴ is cyano.

30. The compound of any one of claims 1-26, wherein R⁴ is an optionally substituted C₁₋₄ alkyl, wherein when the C₁₋₄ alkyl is substituted, the C₁₋₄ alkyl is substituted with a hydroxy or a halogen.

31. The compound of claim 30, wherein R⁴ is an unsubstituted C₁₋₄ alkyl.

32. The compound of claim 30, wherein R⁴ is a fluoro-substituted C₁₋₄ alkyl.

33. The compound of claim 30, wherein R⁴ is a chloro-substituted C₁₋₄ alkyl.

34. The compound of claim 30, wherein R⁴ is a hydroxy-substituted C₁₋₄ alkyl.

35. The compound of any one of claims 1-26, wherein R⁴ is hydrogen.

36. The compound of any one of claims 1-35, wherein R¹ is hydrogen.

37. The compound of any one of claims 1-35, wherein R¹ is halogen.

38. The compound of any one of claims 1-35, wherein R¹ is cyano.

39. The compound of any one of claims 1-35, wherein R¹ is an optionally substituted C₁₋₆ alkyl, wherein when the C₁₋₆ alkyl is substituted, the C₁₋₆ alkyl is substituted with a halogen.

40. The compound of any one of claims 1-35, wherein R¹ is an unsubstituted C₂₋₆ alkenyl.

41. The compound of any one of claims 1-35, wherein R¹ is an unsubstituted C₂₋₆ alkynyl.

42. The compound of any one of claims 1-41, wherein R² is hydrogen.

43. The compound of any one of claims 1-41, wherein R² is fluoro.

44. The compound of any one of claims 1-43, wherein R³ is hydrogen.

45. The compound of any one of claims 1-43, wherein R³ is fluoro.

46. The compound of any one of claims 1-45, wherein R⁸ and R⁹ are each hydrogen.

47. The compound of any one of claims 1-45, wherein R⁸ and R⁹ are each halogen.

48. The compound of any one of claims 1-45, wherein R⁸ and R⁹ is hydrogen, and the other of R⁸ and R⁹ is halogen.

49. The compound of any one of claims 1-48, wherein R⁷ is hydrogen.

50. The compound of any one of claims 1-48, wherein R⁷ is an optionally substituted acyl.

51. The compound of claim 50, wherein R⁷ is an unsubstituted acyl.

52. The compound of any one of claims 1-48, wherein R⁷ is an optionally substituted O-linked α-amino acid.

53. The compound of any one of claims 1-48, wherein R⁷ is an unsubstituted O-linked α-amino acid.

54. The compound of claim 53, wherein R⁷ is selected from unsubstituted O-linked alanine, unsubstituted O-linked valine, unsubstituted O-linked leucine and unsubstituted O-linked glycine.

55. The compound of any one of claims 1-48, wherein R⁷ is

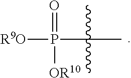

56. The compound of claim 55, wherein R¹⁰ and R¹¹ are each absent or hydrogen.

57. The compound of claim 53, wherein R¹⁰ is 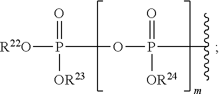
and R¹¹ is absent or hydrogen.

58. The compound of claim 55, wherein m is 0; R¹⁰, R²² and R²³ are independently absent or hydrogen.

59. The compound of claim 55, wherein m is 1; R¹⁰, R²², R²³ and R²⁴ are independently absent or hydrogen.

60. The compound of claim 53, wherein one of R⁹ and R¹⁰ is absent, hydrogen or 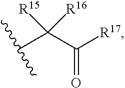
and the other R⁹ and R¹⁰ is

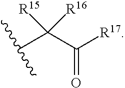

61. The compound of claim 53, wherein R⁹ and R¹⁰ are each

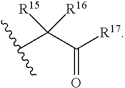

62. The compound of claim 53, wherein one of R⁹ and R¹⁰ is absent, hydrogen or 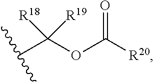
and the other of R⁹ and R¹⁰ is

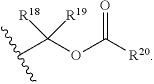

63. The compound of claim 53, wherein R⁹ and R¹⁰ are each

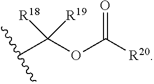

64. The compound of claim 53, wherein one of R⁹ and R¹⁰ is absent, hydrogen or 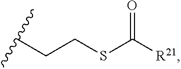
and the other of R⁹ and R¹⁰ is

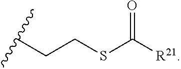

65. The compound of claim 53, wherein R⁹ and R¹⁰ are each

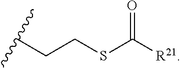

66. The compound of any one of claims 1-48, wherein R⁷ is

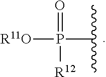

67. The compound of claim 64, wherein R¹¹ is an optionally substituted aryl.

68. The compound of claim 65, wherein the optionally substituted aryl is an optionally substituted phenyl or an optionally substituted naphthyl.

69. The compound of claim 66, wherein the optionally substituted phenyl is an unsubstituted phenyl.

70. The compound of claim 64, wherein R¹¹ is an optionally substituted heteroaryl.

71. The compound of claim 68, wherein R¹¹ is an optionally substituted monocyclic heteroaryl.

72. The compound of any one of claims 64-69, wherein R¹² is an optionally substituted N-linked α-amino acid.

73. The compound of any one of claims 64-69, wherein R¹² is an optionally substituted N-linked α-amino acid ester derivative.

74. The compound of claim 70 or 71, wherein R¹² is N-linked alanine, N-linked alanine isopropyl ester, N-linked alanine cyclohexyl ester and N-linked alanine neopentyl ester.

75. The compound of any one of claims 1-48, wherein R⁷ is

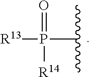

76. The compound of claim 74, wherein R¹³ and R¹⁴ are independently an optionally substituted N-linked α-amino acid ester derivative.

77. The compound of claim 74, wherein R¹³ and R¹⁴ are independently an optionally substituted N-linked α-amino acid ester derivative.

78. The compound of claim 74 or 75, wherein R¹³ and R¹⁴ are independently selected from the group consisting of N-linked alanine, N-linked alanine isopropyl ester, N-linked alanine cyclohexyl ester and N-linked alanine neopentyl ester.

79. The compound of claim 1 or 2, selected from the group consisting of: 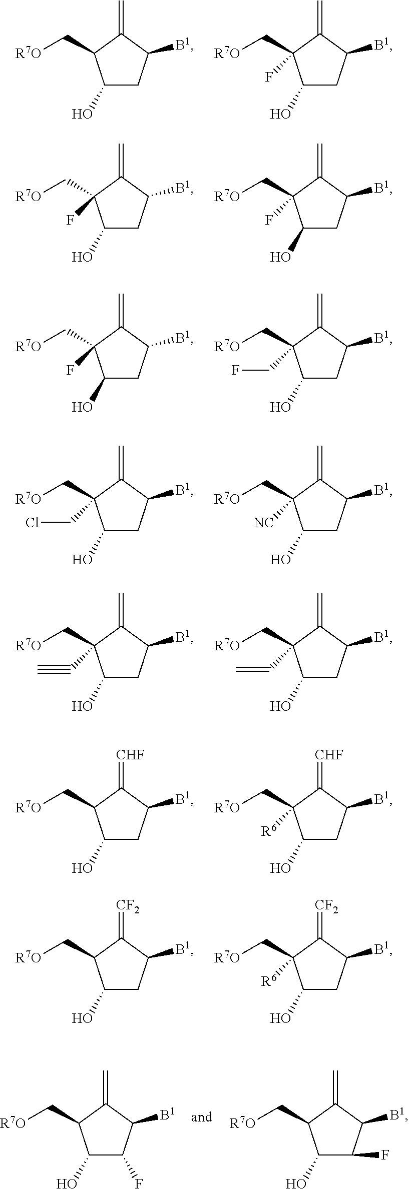
and
or a pharmaceutically acceptable salt of any of the foregoing.

80. The compound of claim 1 or 2, selected from the group consisting of: 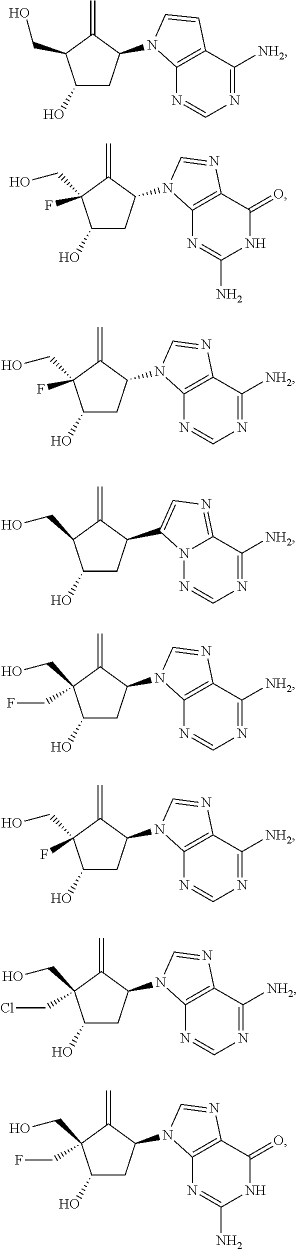 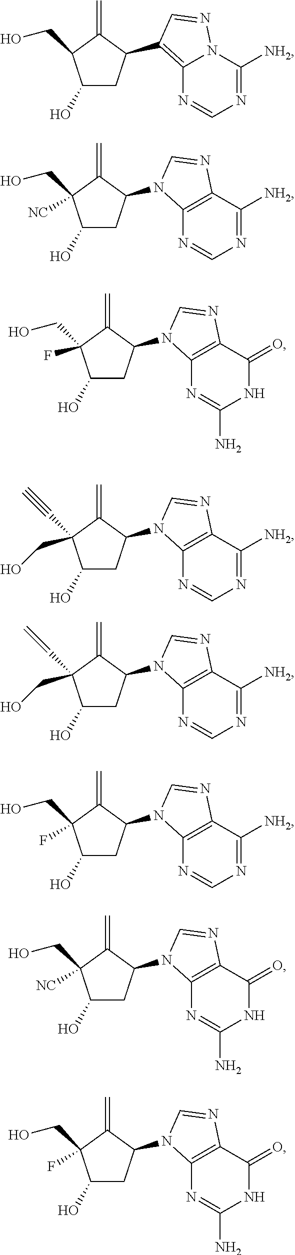 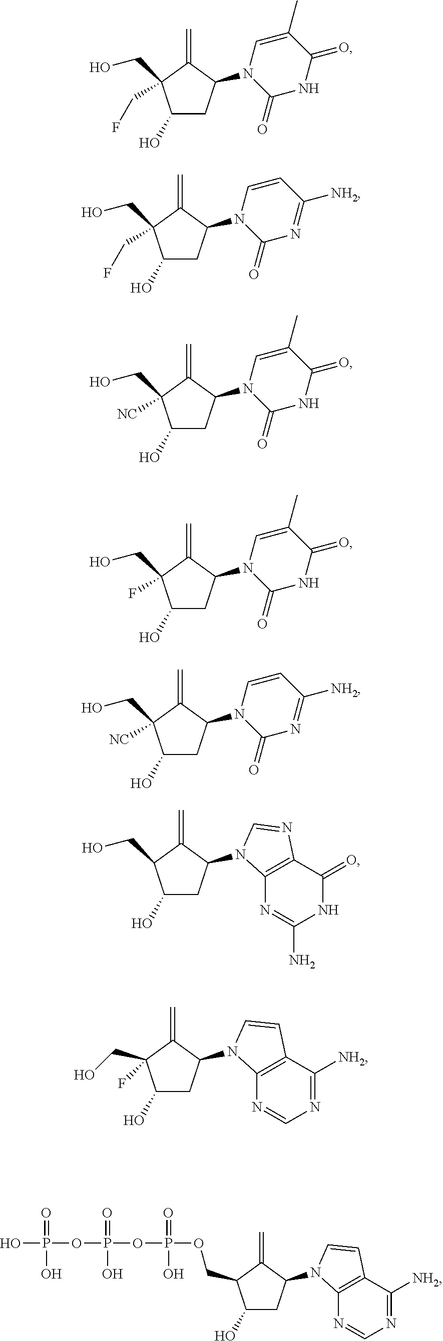 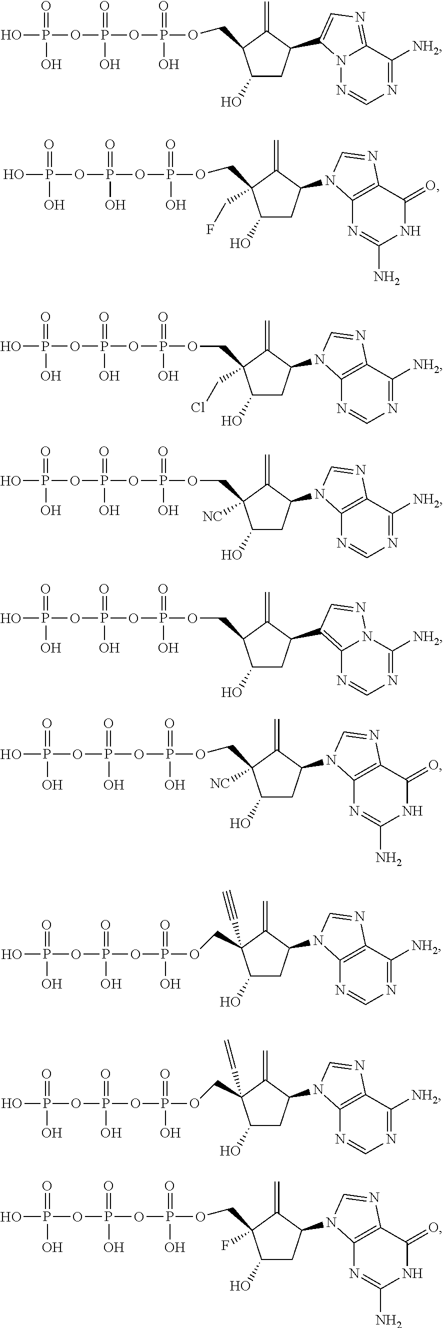 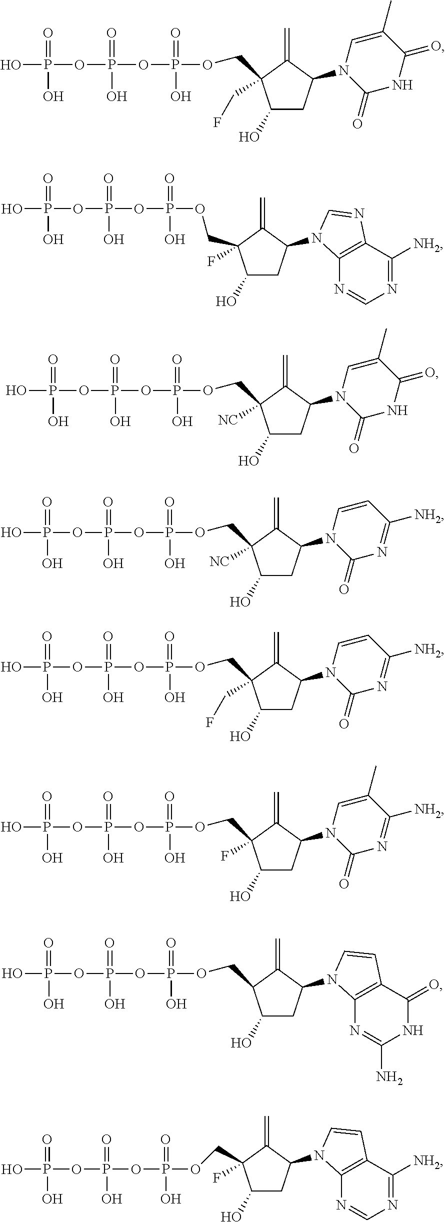 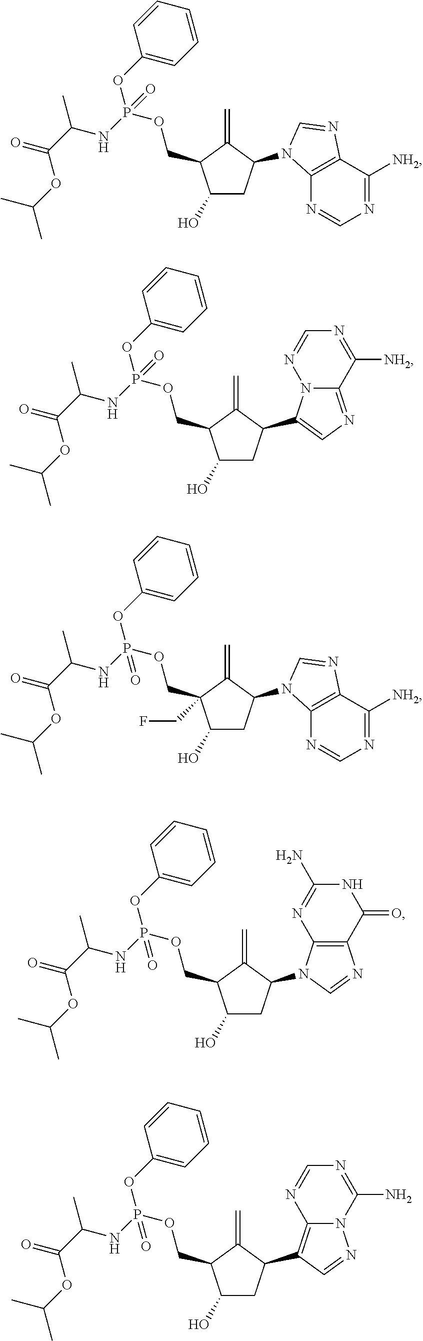 [SMILES: C=C1[C@H](COP(=O)(NC(C)C(=O)OC(C)C)Oc2ccccc2)[C@@H](O)C[C@@H]1n1cnc2c(N)ncnc21 ; C=C1[C@@H](c2cnc3c(N)ncnn23)C[C@H](O)[C@H]1COP(=O)(NC(C)C(=O)OC(C)C)Oc1ccccc1 ; C=C1[C@@H](n2cnc3c(N)ncnc32)C[C@H](O)[C@@]1(CF)COP(=O)(NC(C)C(=O)OC(C)C)Oc1ccccc1 ; C=C1[C@H](COP(=O)(NC(C)C(=O)OC(C)C)Oc2ccccc2)[C@@H](O)C[C@@H]1n1cnc2c(=O)[nH]c(N)nc21 ; C=C1[C@@H](c2cnn3c(N)ncnc23)C[C@H](O)[C@H]1COP(=O)(NC(C)C(=O)OC(C)C)Oc1ccccc1] 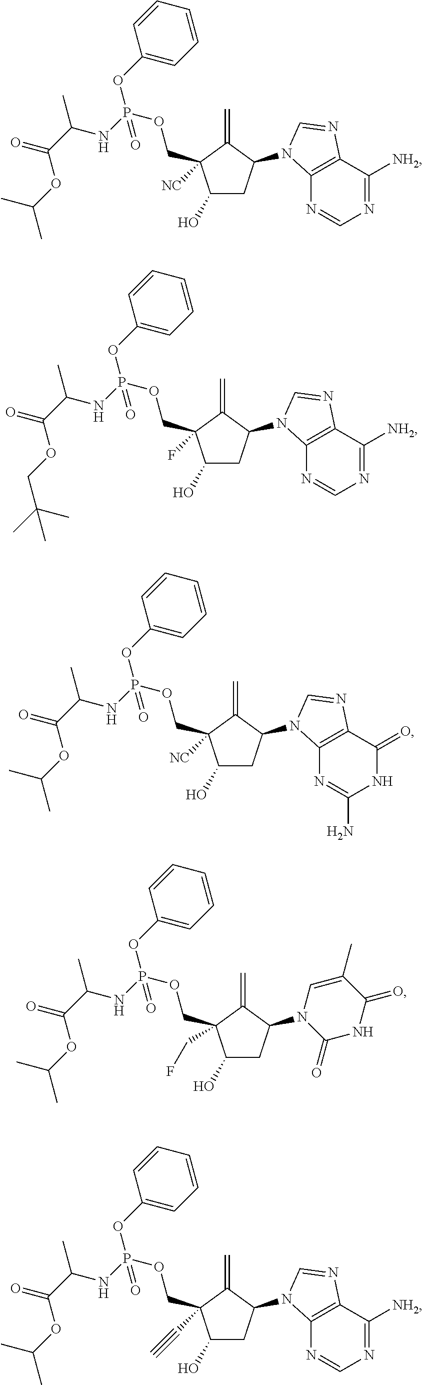 [SMILES: C=C1[C@@H](n2cnc3c(N)ncnc32)C[C@H](O)[C@@]1(C#N)COP(=O)(NC(C)C(=O)OC(C)C)Oc1ccccc1 ; C=C1[C@@H](n2cnc3c(N)ncnc32)C[C@H](O)[C@@]1(F)COP(=O)(NC(C)C(=O)OCC(C)(C)C)Oc1ccccc1 ; C=C1[C@@H](n2cnc3c(=O)[nH]c(N)nc32)C[C@H](O)[C@@]1(C#N)COP(=O)(NC(C)C(=O)OC(C)C)Oc1ccccc1 ; C=C1[C@@H](n2cc(C)c(=O)[nH]c2=O)C[C@H](O)[C@@]1(CF)COP(=O)(NC(C)C(=O)OC(C)C)Oc1ccccc1 ; C#C[C@]1(COP(=O)(NC(C)C(=O)OC(C)C)Oc2ccccc2)C(=C)[C@@H](n2cnc3c(N)ncnc32)C[C@@H]1O] 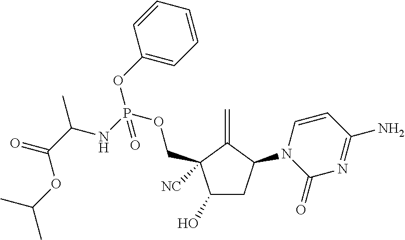 [SMILES: C=C1[C@@H](n2ccc(N)nc2=O)C[C@H](O)[C@@]1(C#N)COP(=O)(NC(C)C(=O)OC(C)C)Oc1ccccc1]
and 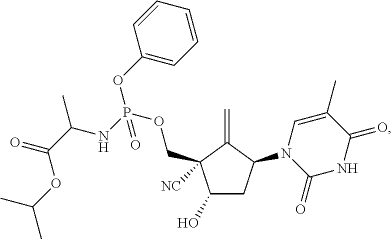 [SMILES: C=C1[C@@H](n2cc(C)c(=O)[nH]c2=O)C[C@H](O)[C@@]1(C#N)COP(=O)(NC(C)C(=O)OC(C)C)Oc1ccccc1]
or a pharmaceutically acceptable salt of any of the foregoing.

81. A pharmaceutical composition comprising an effective amount of a compound of any one of claims 1-80, or a pharmaceutically acceptable salt thereof, and a pharmaceutically acceptable carrier, diluent, excipient or combination thereof.

82. Use of a compound of any one of claims 1-80, or a pharmaceutically acceptable salt thereof, or the pharmaceutical composition of claim 81, for preparing a medicament for treating a HBV and/or HDV infection.

83. Use of a compound of any one of claims 1-80, or a pharmaceutically acceptable salt thereof, or the pharmaceutical composition of claim 81, for preparing a medicament for reducing the reoccurrence of a HBV and/or HDV infection.

84. Use of a compound of any one of claims 1-80, or a pharmaceutically acceptable salt thereof, or the pharmaceutical composition of claim 81, for preparing a medicament for inhibiting replication of a HBV and/or HDV virus.

85. The use of any one of claims 82-84, further comprising the use of one or more agents selected from the group consisting of a HBV and/or HDV polymerase inhibitor, an immunomodulatory agent, an interferon, a pegylated interferon, a viral fusion/entry inhibitor, a viral maturation inhibitor, a capsid assembly modulator, a reverse transcriptase inhibitor, a cyclophilin/TNF inhibitor, a FXR agonist, a TLR-agonist, an siRNA or ASO cccDNA inhibitor, a gene silencing agent, an HBx inhibitor, an sAg secretion inhibitor, and an HBV vaccine, or a pharmaceutically acceptable salt of any of the aforementioned.

86. A method of ameliorating or treating a HBV and/or HDV infection, comprising administering to a subject suffering from the HBV and/or HDV infection an effective amount of a compound of any one of claims 1-80, or a pharmaceutically acceptable salt thereof, or the pharmaceutical composition of claim 81.

87. A method of ameliorating or treating a HBV and/or HDV infection, comprising contacting a cell infected with HBV and/or HDV with a compound of any one of claims 1-80, or a pharmaceutically acceptable salt thereof, or the pharmaceutical composition of claim 81.

88. A method of reducing the reoccurrence of a HBV and/or HDV infection, comprising contacting a cell infected with HBV and/or HDV with a compound of any one of claims 1-80, or a pharmaceutically acceptable salt thereof, or the pharmaceutical composition of claim 81.

89. A method of inhibiting replication of a HBV and/or HDV virus, comprising contacting a cell infected with HBV and/or HDV with a compound of any one of claims 1-80, or a pharmaceutically acceptable salt thereof, or the pharmaceutical composition of claim 81.

90. The method of any one of claims 86-89, further comprising the use of one or more agents selected from the group consisting of a HBV and/or HDV polymerase inhibitor, an immunomodulatory agent, an interferon, a pegylated interferon, a viral fusion/entry inhibitor, a viral maturation inhibitor, a capsid assembly modulator, a reverse transcriptase inhibitor, a cyclophilin/TNF inhibitor, a FXR agonist, a TLR-agonist, an siRNA or ASO cccDNA inhibitor, a gene silencing agent, an HBx inhibitor, an sAg secretion inhibitor, and an HBV vaccine, or a pharmaceutically acceptable salt of any of the aforementioned.

91. Use of a compound of any one of claims 1-80, or a pharmaceutically acceptable salt thereof, or the pharmaceutical composition of claim 81, for preparing a medicament for ameliorating or treating a HIV infection.

92. Use of a compound of any one of claims 1-80, or a pharmaceutically acceptable salt thereof, or the pharmaceutical composition of claim 81, for preparing a medicament for inhibiting replication of a HIV virus.

93. The use of any one of claims 91-92, further comprising the use of one or more antiretroviral therapy (ART) agents selected from the group consisting of a non-nucleoside reverse transcriptase inhibitor (NNRTI), a nucleoside reverse transcriptase inhibitor (NRTI), a protease inhibitor (PI), a fusion/entry inhibitor (also called a CCR5 antagonist), an integrase strand transfer inhibitor (INSTI), and an HIV other antiretroviral therapy, or a pharmaceutically acceptable salt of any of the aforementioned.

94. A method of ameliorating or treating a HIV infection comprising administering to a subject suffering from the HIV infection an effective amount of a compound of any one of claims 1-80, or a pharmaceutically acceptable salt thereof, or the pharmaceutical composition of claim 81.

95. A method for inhibiting replication of a HIV virus comprising contacting a cell infected with the HIV virus with a compound of any one of claims 1-80, or a pharmaceutically acceptable salt thereof, or the pharmaceutical composition of claim 81.

96. A method for ameliorating or treating a HIV infection comprising contacting a cell infected with the HIV with a compound of any one of claims 1-80, or a pharmaceutically acceptable salt thereof, or the pharmaceutical composition of claim 81.

97. The method of any one of claims 94-96, further comprising one or more antiretroviral therapy (ART) agents selected from the group consisting of a non-nucleoside reverse transcriptase inhibitor (NNRTI), a nucleoside reverse transcriptase inhibitor (NRTI), a protease inhibitor (PI), a fusion/entry inhibitor (also called a CCR5 antagonist), an integrase strand transfer inhibitor (INSTI), and an HIV other antiretroviral therapy, or a pharmaceutically acceptable salt of any of the aforementioned.
